(19) United States
(12) Patent Application Publication
(10) Pub. No.: US 2023/0055961 A1
(43) Pub. Date: Feb. 23, 2023

(54) PENTAFLUOROBENZENESULFONAMIDE DERIVATIVES AND USES THEREOF

(71) Applicants: Dunad Therapeutics Ltd., Great Cambourne (GB); 2692372 ONTARIO INC., Mississauga (CA)

(72) Inventors: Dziyana KRASKOUSKAYA, Mississauga (CA); Siawash AHMAR, Great Wymondley (GB); Patrick T. GUNNING, Toronto (CA); Jeff OMEARA, Etobicoke (CA); David Alexander ROSA, Halton Hills (CA); Ji Sung PARK, North York (CA); Graham L. SIMPSON, Great Cambourne (GB); Ayah ABDELDAYEM, Ontario (CA)

(21) Appl. No.: 17/748,431

(22) Filed: May 19, 2022

Related U.S. Application Data
(63) Continuation of application No. PCT/IB2020/000981, filed on Nov. 20, 2020.
(60) Provisional application No. 62/939,543, filed on Nov. 22, 2019.
(60) Provisional application No. 63/053,527, filed on Jul. 17, 2020.

(51) Int. Cl. C07D 487/04

(52) U.S. Cl. CPC C07D 487/04

(57) ABSTRACT

Provided herein are pentafluorobenzenesulfonamide compounds, pharmaceutical compositions comprising said compounds, and methods for using said compounds for the treatment of diseases.

### CROSS-REFERENCE

This application is a continuation of International Application No. PCT/IB2020/000981, filed Nov. 20, 2020, which claims benefit of U.S. Provisional Patent Application No. 62/939,543 filed on Nov. 22, 2019 and U.S. Provisional Patent Application No. 63/053,527 filed on Jul. 17, 2020, each incorporated herein by reference in its entirety.

### BACKGROUND

Chemical modification is an important tool to alter structure and function of proteins. One way to achieve chemical modification of proteins is to use covalent small molecule inhibitors. As a result, covalent small molecule inhibitors of proteins are considered to be useful in multiple applications, including therapeutics.

### BRIEF SUMMARY OF THE INVENTION

Provided herein are covalent small molecule inhibitors. Also provided herein are covalent small molecule inhibitors of tubulin polymerization. Also provided herein are pharmaceutical compositions comprising said compounds, and methods for using said compounds for the treatment of diseases.

One embodiment provides a compound, or a salt or solvate thereof, having the structure of Formula (I):

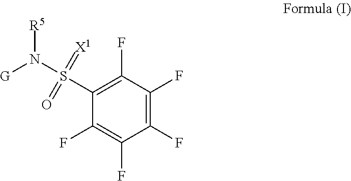

wherein,
G is an organic residue (e.g., a natural ligand, such as comprising one or more substituted and/or unsubstituted (e.g., unsaturated) carbocycle and/or substituted and/or or unsubstituted (e.g., unsaturated) heterocycle), or -L²-G¹, wherein L² is a >C═X, substituted or unsubstituted unsaturated alkylene (e.g., alkenylene or alkynylene, such as with an unsaturated carbon alpha to the N-atom of Formula (I)), substituted or unsubstituted unsaturated carbocycle, or substituted or unsubstituted unsaturated heterocycle, wherein X is O, S, or NR³, and G¹ is hydrogen or an organic residue (e.g., a natural ligand), provided that when X¹ is O, then G is not a substituted or unsubstituted phenyl;

### X¹ is O or NR;

R is hydrogen, substituted or unsubstituted alkyl, substituted or unsubstituted heteroalkyl, substituted or unsubstituted aryl, or substituted or unsubstituted heteroaryl;
R³ is hydrogen, -L¹R⁴, —C(═O)L¹R⁴, —C(═O)OL¹R⁴, or —C(═O)NR⁴L¹R⁴, wherein each L¹ is independently substituted or unsubstituted alkyl, substituted or unsubstituted heteroalkyl, substituted or unsubstituted aryl, or substituted or unsubstituted heteroaryl; and each R⁴ is independently hydrogen, substituted or unsubstituted C₃-C₈ cycloalkyl, substituted or unsubstituted C₂-C₇ heterocycloalkyl, substituted or unsubstituted aryl, or substituted or unsubstituted heteroaryl; R⁵ is hydrogen, —CN, —C(═O)R⁶, —C(═O)OR⁶, —C(═O)NR³R⁶, substituted or unsubstituted alkyl, substituted or unsubstituted heteroalkyl, substituted or unsubstituted aryl, or substituted or unsubstituted heteroaryl, provided that when X¹ is O, then R⁵ is not substituted or unsubstituted phenyl; and each R⁶ is independently hydrogen, substituted or unsubstituted alkyl, or substituted or unsubstituted heteroalkyl.

In some embodiments, G and R5 on a single N are taken together with the single N to form G′. In certain embodiments, G′ is as described for any G¹ group herein (with the exception that G′ is not H). In certain embodiments, G′ is a substituted or unsubstituted heterocycle (e.g., heterocycloalkyl).

In certain embodiments, G¹ or G′ comprises one or more cyclic ring systems selected from substituted or unsubstituted carbocycles and substituted or unsubstituted heterocycles (e.g., provided that G′ comprises at least one substituted or unsubstituted heterocycle). In some embodiments, G¹ or G′ comprises two or more cyclic ring systems selected from substituted or unsubstituted carbocycles and substituted or unsubstituted heterocycles (e.g., provided that G′ comprises at least one substituted or unsubstituted heterocycle). In some embodiments, the two or more cyclic ring systems are connected via a bond. In some embodiments, the two or more cyclic ring systems are connected via one or more linker and/or bond (e.g., wherein there are three cyclic ring systems, two of the ring systems are connected via bond, while the other two ring systems are connected by linker).

In some embodiments, G comprises two or more cyclic ring systems, such as wherein the ring systems are connected via a bond. In some embodiments, the two or more cyclic ring systems are connected via one or more linker and/or bond (e.g., wherein there are three cyclic ring systems, two of the ring systems are connected via bond, while the other two ring systems are connected by linker).

In some embodiments, provided herein is a compound of Formula (I), such as described herein, with the exception that the G of Formula (I) is R⁵ and the X¹ of Formula (I) is ═N-G, wherein G and R⁵ are as described in Formula (I). In other words, in certain embodiments, —S(═O)(═X¹)NR⁵G is substituted with —S(═O)(═NG)NR⁵₂.

One embodiment provides a pharmaceutical composition comprising a pentafluorobenzenesulfonamide derivative compound as described herein, or a salt or solvate thereof, and one or more of pharmaceutically acceptable excipients.

One embodiment provides a protein modified with a pentafluorobenzenesulfonamide derivative compound as described herein, wherein the compound forms a covalent bond with a sulfur atom of a cysteine residue of the protein.

One embodiment provides a method of modifying (e.g., attaching to and/or degrading) a polypeptide with a pentafluorobenzenesulfonamide derivative compound as described herein, comprising contacting the polypeptide with the compound to form a covalent bond with a sulfur atom of a cysteine residue of the polypeptide.

One embodiment provides a method of binding a compound to a polypeptide, comprising contacting the polypeptide with a pentafluorobenzenesulfonamide derivative compound as described herein.

### INCORPORATION BY REFERENCE

All publications, patents, and patent applications mentioned in this specification are herein incorporated by reference for the specific purposes identified herein.

### BRIEF DESCRIPTION OF THE DRAWINGS

The accompanying figures, which are incorporated in and constitute a part of this specification, illustrate several aspects and together with the description serve to explain and not to limit the scope of current disclosure.

FIG. 1 illustrates representative covalent modification of the BTK enzyme with compound 5-1 demonstrated by mass spectrometry.

FIG. 2 illustrates representative covalent modification of the BTK enzyme with Compound 5-1 demonstrated by enzyme kinetic analysis (time-dependent inhibition).

FIG. 3 illustrate residual activity of the BTK enzyme in the presence of compound 5-1.

FIG. 4 illustrates residual activity of the BTK enzyme in the presence of ARQ-531.

FIG. 5 illustrates modification of 467QRPIFIITEYMANGCLLNYLR487 enzyme with Compound 5-15.

FIG. 6A illustrates modification of 467QRPIFIITEYMANGCLLNYLR487 enzyme with Compound 5-3; FIG. 6B illustrates modification of 526NCLVNDQGVVK536 enzyme with Compound 5-3.

FIG. 7 illustrates modification of 467QRPIFIITEYMANGCLLNYLR487 enzyme with Compound 5-13.

FIG. 8 illustrates modification of 467QRPIFIITEYMANGCLLNYLR487 enzyme with Compound 5-14.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH2 moiety.

“Hydroxy” or “hydroxyl” refers to the —OH moiety.

“Alkyl” refers to a non-aromatic straight or branched hydrocarbon chain radical consisting solely of carbon and hydrogen atoms, partially or fully saturated, cyclic or acyclic, having from one to fifteen carbon atoms (e.g., C₁-C₁₄ alkyl). In certain embodiments, an alkyl comprises one to thirteen carbon atoms (e.g., C₁-C₁₂ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an alkyl includes alkenyl, alkynyl, cycloalkyl, carbocycloalkyl, cycloalkylalkyl, haloalkyl, and fluoroalkyl, as defined herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene).

Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene).

Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is as defined above. Unless stated otherwise specifically in the specification, an alkoxy group is optionally substituted, as defined above for an alkyl group.

“Alkoxyalkyl” refers to an alkyl moiety comprising at least one alkoxy substituent, where alkyl is as defined above. Unless stated otherwise specifically in the specification, an alkoxyalkyl group is optionally substituted, as defined above for an alkyl group.

“Alkylamino” refers to a moiety of the formula —NHRₐ or —NRₐR_{b} where Rₐ and R_{b} are each independently an alkyl group as defined above. Unless stated otherwise specifically in the specification, an alkylamino group is optionally substituted, as defined above for an alkyl group.

“Alkylaminoalkyl” refers to an alkyl moiety comprising at least one alkylamino substituent. The alkylamino substituent can be on a tertiary, secondary or primary carbon. Unless stated otherwise specifically in the specification, an alkylaminoalkyl group is optionally substituted, as defined above for an alkyl group.

“Aminoalkyl” refers to an alkyl moiety comprising at least one amino substituent. The amino substituent can be on a tertiary, secondary or primary carbon. Unless stated otherwise specifically in the specification, an aminoalkyl group is optionally substituted, as defined above for an alkyl group.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Arylene” refers to a divalent aryl group which links one part of the molecule to another part of the molecule. Unless stated specifically otherwise, an arylene is optionally substituted, as defined above for an aryl group.

“Aralkyl” refers to a radical of the formula —R^{c}-aryl where R^{C} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

The term “carbocycle” or “carbocyclic” refers to a ring or ring system where the atoms forming the backbone of the ring are all carbon atoms. The term thus distinguishes carbocyclic group from a “heterocycle” or “heterocyclic” in which the ring backbone contains at least one atom which is different from carbon. In some embodiments, carbocycles are monocyclic, bicyclic, polycyclic, spirocyclic or bridged compounds. Carbocycle includes aromatic and partially or fully saturated ring systems. In some embodiments, carbocycle comprises cycloalkyl and aryl.

“Cyclic ring” refers to a carbocycle or heterocycle, including aromatic, non-saturated, and saturated carbocycle and heterocycle. A “cyclic ring” is optionally monocyclic or polycyclic (e.g., bicyclic).

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyl is saturated (i.e., containing single C—C bonds only) or unsaturated (i.e., containing one or more double bonds or triple bonds). Examples of monocyclic cycloalkyls include, e.g., cyclopropyl, cyclobutyl, cyclopentyl, cyclohexyl, cycloheptyl, and cyclooctyl. An unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Cycloalkylalkyl” refers to a radical of the formula —R^{c}-cycloalkyl where R^{c} is an alkylene chain as defined above. The alkylene chain and the cycloalkyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

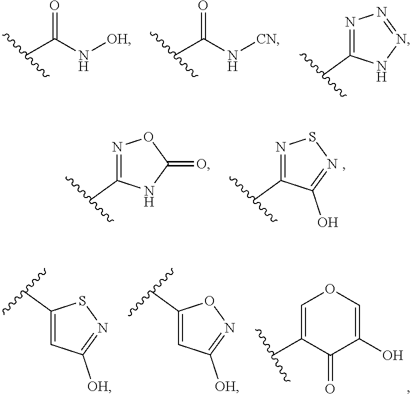

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), phosphorous (e.g. >P—, >P(═O)—, or —P(═O)₂), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. Representative heteroalkyl groups include, but are not limited to —OCH₂OMe, —OCH₂CH₂OH, —CH₂CH₂OMe, or —OCH₂CH₂OCH₂CH₂NH2.

In some embodiments, heteroalkyl includes alkoxy, alkoxyalkyl, alkylamino, alkylaminoalkyl, aminoalkyl, heterocycloalkyl, heterocycloalkyl, and heterocycloalkylalkyl, as defined herein.

Unless stated otherwise specifically in the specification, a heteroalkyl group is optionally substituted, as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic or tetracyclic ring system, which optionally includes fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the ring(s). In some embodiments, heterocycloalkyl comprises 2-12 C atoms, 0-6 N atoms, 0-4 O atoms, and 0-4 S atoms. In some embodiments, heterocycloalkyl comprises 2-10 C atoms, 0-4 N atoms, 0-2 O atoms, and 0-2 S atoms. In some embodiments, heterocycloalkyl comprises 2-8 C atoms, 0-3 N atoms, 0-1 O atoms, and 0-1 S atoms. In some embodiments, heterocycloalkyl is a saturated or partially unsaturated 3-7 membered monocyclic, 6-10 membered bicyclic, or 13-16 membered polycyclic (e.g., tricyclic or tetracyclic) ring system having 1, 2, 3, or 4 heteroatom ring members each independently selected from N, O, and S. In some embodiments, heterocycloalkyl comprises 1 or 2 heteroatom ring members each independently selected from N, O, and S. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocycloalkyl” or “N-attached heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals. Examples of such N-heterocycloalkyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocycloalkyl” or “C-attached heterocycloalkyl” refers to a heterocycloalkyl radical as defined above containing at least one heteroatom and where the point of attachment of the heterocycloalkyl radical to the rest of the molecule is through a carbon atom in the heterocycloalkyl radical. A C-heterocycloalkyl radical is optionally substituted as described above for heterocycloalkyl radicals. Examples of such C-heterocycloalkyl radicals include, but are not limited to, 2-morpholinyl, 2- or 3- or 4-piperidinyl, 2-piperazinyl, 2- or 3-pyrrolidinyl, and the like.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroarylene” refers to a divalent heteroaryl group which links one part of the molecule to another part of the molecule. Unless stated specifically otherwise, a heteroarylene is optionally substituted, as defined above for a heteroaryl group.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

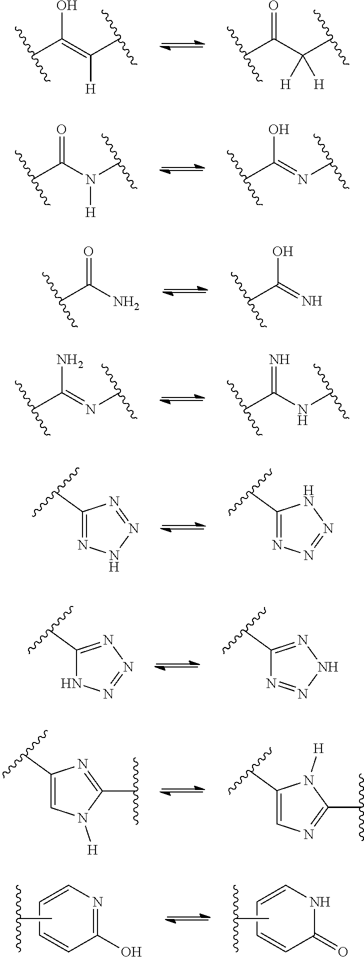

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d3 (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

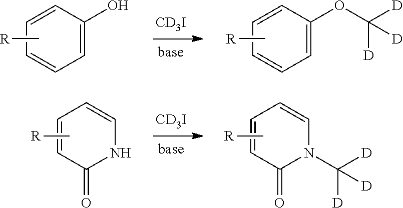

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

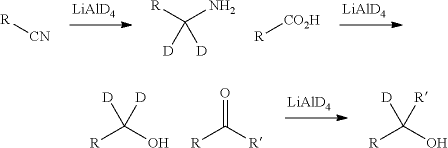

Deuterium gas and palladium catalyst are employed to reduce unsaturated carbon-carbon linkages and to perform a reductive substitution of aryl carbon-halogen bonds as illustrated, by way of example only, in the reaction schemes below.

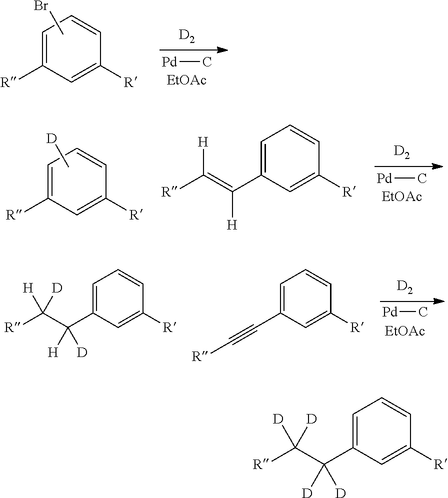

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable tH hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of cyclin-dependent kinases (CDKs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal ofPharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Covalent Small Molecule Inhibitors

Chemical modification is an important tool to alter structure and function of proteins.

One way to achieve chemical modification of proteins is to use covalent small molecule inhibitors. As a result, covalent small molecule inhibitors of proteins are considered to be useful in multiple applications, including therapeutics. Covalent inhibition of a target protein minimizes the required systemic drug exposure. In some embodiments, protein activity can only be restored by de novo protein synthesis, resulting in a prolonged therapeutic effect long after the compound is cleared from the blood. Strategically placing an electrophilic moiety on the inhibitor will allow it to undergo attack by a nucleophilic amino acid residue upon binding to the target protein, forming a reversible or irreversible bond that is much stronger than typical noncovalent interactions. However, the ability to form a covalent bond with the target enzyme has raised concerns about indiscriminate reactivity with off-target proteins, even though some of the most prescribed drugs are covalent irreversible binders. This led to the disfavor of covalent modifiers as drug candidates until the recent successful development of irreversible covalent kinase inhibitors ibrutinib and afatinib, which form an irreversible covalent bond between an acrylamide warhead and a nonconserved cysteine residue on the ATP-binding site but also with nontargeted cellular thiols. The ability to form covalent adducts with off-target proteins has been linked to an increased risk of unpredictable idiosyncratic toxicity along with the daily drug dose administered to patients. Accordingly, there is a need to reduce the risk of non-target covalent interactions by incorporating less reactive electrophilic moieties into covalent small molecule inhibitors. In some embodiments, described herein is a covalent small molecule inhibitor. In some embodiments, described herein is a pharmaceutical composition comprising a covalent small molecule inhibitor and one or more of pharmaceutically acceptable excipients. In other embodiments, a covalent small molecule inhibitor is used to treat or prevent a disease or condition in a subject in need thereof.

In some embodiments, a covalent small molecule inhibitor is a pentafluorobenzenesulfonamide derivative compound. In some embodiments, a pentafluorobenzenesulfonamide derivative compound as described herein is used to treat or prevent a disease or condition in a subject in need thereof.

Tubulin

Microtubules are composed of alpha/beta-tubulin heterodimers and constitute a crucial component of the cell cytoskeleton. In addition, microtubules play a pivotal role during cell division, in particular when the replicated chromosomes are separated during mitosis.

Interference with the ability to form microtubules from alpha/beta-tubulin heterodimeric subunits generally leads to cell cycle arrest. This event can, in certain cases, induce programmed cell death.

Microtubules are subcellular organelles located in most eukaryotic cells and are involved in a variety of cell functions including mitosis, intracellular movement, cell movement and maintenance of cell shape. Microtubule assembly involves polymerization of tubulin and additional construction with other components of the microtubule (referred to as “microtubule-associated proteins” or MAPs).

Tubulin itself consists of two 50 kDa subunits (alpha- and beta-tubulin) which combine in a heterodimer. The heterodimer binds two molecules of guanosine triphosphate (GTP). One of the GTP molecules is tightly bound and cannot be removed without denaturing the heterodimer, while the other GTP molecule is freely exchangeable with other GTPs. This exchangeable GTP is believed to be involved in tubulin function. In particular, the tubulin heterodimer can combine in a head-to-tail arrangement in the presence of GTP to form a long protein fiber, known as a protofilament. These protofilaments can then group together to form a protein sheet which then curls into a tube-like structure known as a microtubule. Interference with this process of microtubule construction affects the downstream processes of mitosis and maintenance of cell shape. Most of the naturally-occurring antimitotic agents have been shown to exert their effect by binding to tubulin, rather than MAPs or other proteins involved in mitosis. For example, tubulin is the biochemical target for several clinically useful anticancer drugs, including vincristine, vinblastine and paclitaxel. Another natural product, colchicine, was instrumental in the purification of tubulin as a result of its potent binding, with beta-tubulin being the target for colchicine. Colchicine and other colchicine site agents bind at a site on beta-tubulin that results in inhibition of a cross-link between cys-239 and cys-354 (wherein the numbering refers to the (2 isotype) by such non-specific divalent sulfhydryl reactive agents as N,N′-ethylenebis-iodoacetamide. However, simple alkylation of cys-239 does not appear to inhibit colchicine binding to tubulin. In addition to colchicine, other natural products are known that bind at the colchicine site and inhibit microtubule assembly, for example, podophyllotoxin, steganacin and combretastatin. Still other agents bind to sites on tubulin referred to as the Vinca alkaloid site and the Rhizoxin/Maytansine site. However, none of the noted natural products are thought to operate by covalent modification of tubulin.

Based on the essential role of tubulin in the processes of cell transport and cell division, compounds which alter the tubulin activity are considered to be useful in treating or preventing various disorders. Accordingly, in some embodiments, also described herein is a covalent small molecule inhibitor of tubulin. In some embodiments also described herein is a pharmaceutical composition comprising a covalent small molecule inhibitor of tubulin and one or more of pharmaceutically acceptable excipients. In other embodiments, a covalent small molecule inhibitor of tubulin is used to treat or prevent a disease or condition in a subject in need thereof.

In some embodiments, a covalent small molecule inhibitor of tubulin is a pentafluorobenzenesulfonamide derivative compound. In some embodiments, a pentafluorobenzenesulfonamide derivative compound as described herein is used to treat or prevent a disease or condition in a subject in need thereof.

In other embodiments, a pharmaceutical composition comprising a pentafluorobenzenesulfonamide derivative compound as described herein and one or more of pharmaceutically acceptable excipients is used to treat or prevent a disease or condition in a subject in need thereof.

In some embodiments, disclosed herein is a method of treating a disease comprising administering to a subject in need thereof a therapeutically effective amount of a pentafluorobenzenesulfonamide derivative compound as described herein.

In other embodiments, disclosed herein is a method of treating a disease comprising administering to a subject in need thereof a therapeutically effective amount of a pharmaceutical composition comprising a pentafluorobenzenesulfonamide derivative compound as described herein and one or more of pharmaceutically acceptable excipients.

In some embodiments, disclosed herein is a protein modified with a pentafluorobenzenesulfonamide derivative compound as described herein, wherein the compound forms a covalent bond with a sulfur atom of a cysteine residue of the protein. In some embodiments, disclosed herein is a method of modifying (e.g., attaching to and/or degrading) a polypeptide with a pentafluorobenzenesulfonamide derivative compound as described herein, comprising contacting the polypeptide with the compound to form a covalent bond with a sulfur atom of a cysteine residue of the polypeptide. In some embodiments, disclosed herein is a method of binding a compound to a polypeptide, comprising contacting the polypeptide with a pentafluorobenzenesulfonamide derivative compound as described herein. In some embodiments, the protein or polypeptide described herein is tubulin.

Pentafluorobenzenesulfonamide Derivative Compounds

In one aspect, provided herein is a pentafluorobenzenesulfonamide derivative compound.

In some embodiments, a pentafluorobenzenesulfonamide derivative compound is a tubulin inhibitory compound.

One embodiment provides a compound, or pharmaceutically acceptable salt or solvate thereof, having the structure of Formula (I):

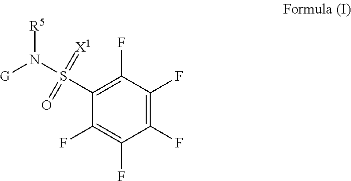

wherein,
G is an organic residue (e.g., a natural ligand, such as a radical comprising one or more substituted or unsubstituted unsaturated carbocycle, and/or substituted or unsubstituted unsaturated heterocycle), or -L²-G¹, wherein L² is a >C═X, substituted or unsubstituted unsaturated alkylene (e.g., alkenylene or alkynylene, such as with an unsaturated carbon alpha to the N-atom of Formula (I)), substituted or unsubstituted unsaturated carbocycle, or substituted or unsubstituted unsaturated heterocycle, wherein X is O, S, or NR³, and G¹ is hydrogen or an organic residue (e.g., a natural ligand), provided that when X¹ is O, then G is not a substituted or unsubstituted phenyl;

### X¹ is O or NR;

R is hydrogen, substituted or unsubstituted alkyl, substituted or unsubstituted heteroalkyl, substituted or unsubstituted aryl, or substituted or unsubstituted heteroaryl;
R³ is hydrogen, -L¹R⁴, —C(═O)L¹R⁴, —C(═O)OL¹R⁴, or —C(═O)NR⁴L¹R⁴, wherein each L¹ is independently substituted or unsubstituted alkyl, substituted or unsubstituted heteroalkyl, substituted or unsubstituted aryl, or substituted or unsubstituted heteroaryl; and each R⁴ is independently hydrogen, substituted or unsubstituted C₃-C₈ cycloalkyl, substituted or unsubstituted C₂-C₇ heterocycloalkyl, substituted or unsubstituted aryl, or substituted or unsubstituted heteroaryl;
R⁵ is hydrogen, —CN, —C(═O)R⁶, —C(═O)OR⁶, —C(═O)NR³R⁶, substituted or unsubstituted alkyl, substituted or unsubstituted heteroalkyl, substituted or unsubstituted aryl, or substituted or unsubstituted heteroaryl, provided that when X¹ is O, then R⁵ is not substituted or unsubstituted phenyl; and
each R⁶ is independently hydrogen, substituted or unsubstituted alkyl, or substituted or unsubstituted heteroalkyl.

In some embodiments, G in Formula (I) is -L²-G¹, wherein L² is a >C═X, substituted or unsubstituted unsaturated alkylene (e.g., alkenylene or alkynylene, such as with an unsaturated carbon alpha to the N-atom of Formula (I)), substituted or unsubstituted arylene, or substituted or unsubstituted heteroarylene, wherein X is O, S, or NR³, and G¹ is an organic residue (e.g., a natural ligand). In some embodiments, L² is a substituted or unsubstituted unsaturated alkylene (e.g., alkenylene or alkynylene, such as with an unsaturated carbon alpha to the N-atom of Formula (I)), substituted or unsubstituted arylene, or substituted or unsubstituted heteroarylene, and G¹ is an organic residue (e.g., a natural ligand).

In some embodiments, G in Formula (I) is substituted or unsubstituted unsaturated carbocycle or substituted or unsubstituted unsaturated heterocycle, wherein G and R⁵ on a single N are optionally taken together to form a substituted or unsubstituted heterocycloalkyl. In some embodiments, G and R⁵ are optionally taken together to form a substituted or unsubstituted heterocycloalkyl (or substituted or unsubstituted heteroaryl), such as wherein such substituted or unsubstituted heterocycloalkyl (or substituted or unsubstituted heteroaryl) is substituted or unsubstituted heterocycloalkyl-G¹ (or substituted or unsubstituted heteroaryl-G¹).

In some embodiments, G in Formula (I) comprises one or more cyclic ring systems selected from substituted or unsubstituted unsaturated carbocycles and substituted or unsubstituted unsaturated heterocycles. In some embodiments, G in Formula (I) comprises two or more cyclic ring systems selected from substituted or unsubstituted unsaturated carbocycles and substituted or unsubstituted unsaturated heterocycles.

In some embodiments, G comprises two or more cyclic ring systems, such as wherein the ring systems are connected via a bond. In some embodiments, the two or more cyclic ring systems are connected via one or more linker and/or bond (e.g., wherein there are three cyclic ring systems, two of the ring systems are connected via bond, while the other two ring systems are connected by linker).

In some embodiments, G¹ comprises one or more cyclic ring systems selected from substituted or unsubstituted carbocycles and substituted or unsubstituted heterocycles. In some embodiments, G¹ comprises two or more cyclic ring systems selected from substituted or unsubstituted carbocycles and substituted or unsubstituted heterocycles.

In some embodiments, the two or more cyclic ring systems are connected via a bond. In some embodiments, the two or more cyclic ring systems are connected via one or more linker and/or bond.

In some embodiments, the linker is —O—, —NR⁷—, —N(R⁷)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁷—, —NR⁷C(═O)—, —OC(═O)NR⁷—, —NR⁷C(═O)O—, —NR⁷C(═O)NR⁷—, —NR⁷S(═O)₂—, —S(═O)₂NR⁷—, —C(═O)NR⁷S(═O)₂—, —S(═O)₂NR⁷C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁷—, —NR⁷—(C₁-C₄ alkylene)-, —(C₁-C₄ alkylene)-N(R⁷)₂⁺—, or —N(R⁷)₂⁺—(C₁-C₄ alkylene)-; and

each R⁷ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

In some embodiments, the cyclic ring system comprises substituted or unsubstituted monocyclic aryl or substituted or unsubstituted monocyclic heteroaryl. In some embodiments, the cyclic ring system comprises substituted or unsubstituted bicyclic aryl or substituted or unsubstituted bicyclic heteroaryl.

In some embodiments, a pentafluorobenzenesulfonamide derivative compound described herein comprises a protein binder or ligand. In some embodiments, G (or G¹) in Formula (I) is or comprises a protein binder or ligand. In some embodiments, G (or G¹) in Formula (I) is selected from:

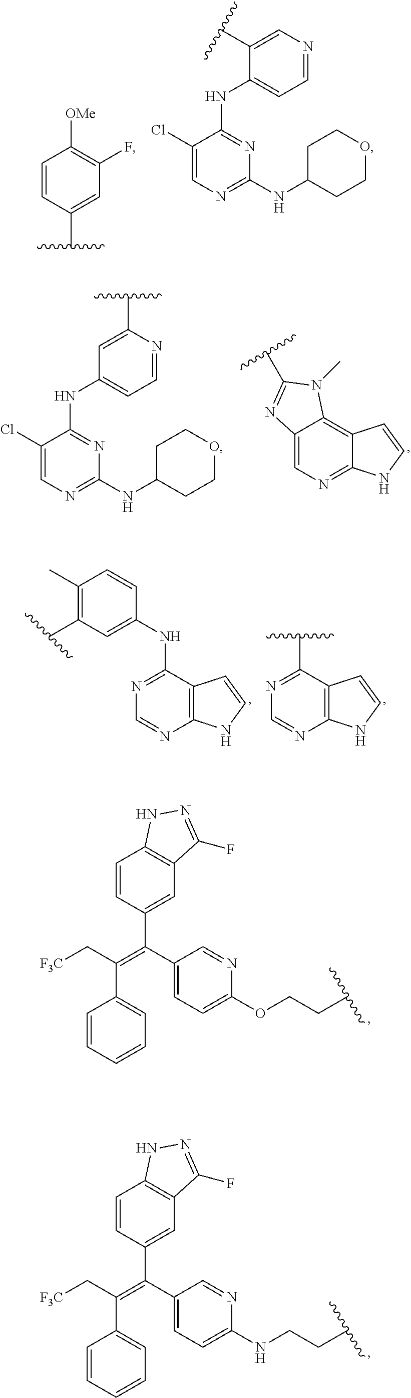

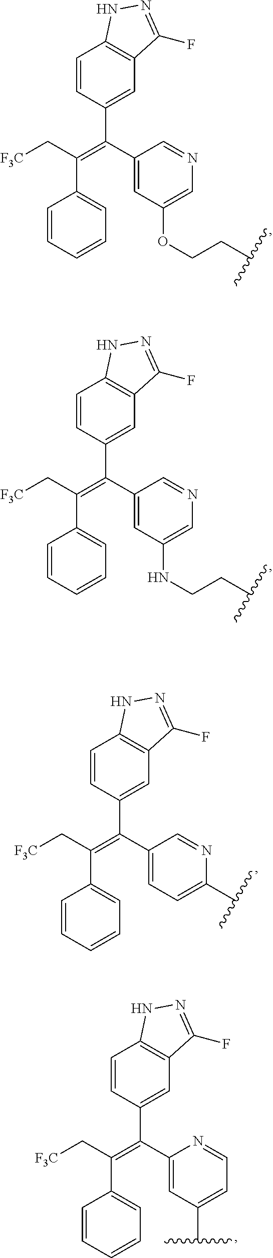 [SMILES: *CCOc1cncc(/C(=C(/CC(F)(F)F)c2ccccc2)c2ccc3[nH]nc(F)c3c2)c1 ; *CCNc1cncc(/C(=C(/CC(F)(F)F)c2ccccc2)c2ccc3[nH]nc(F)c3c2)c1 ; *c1ccc(/C(=C(/CC(F)(F)F)c2ccccc2)c2ccc3[nH]nc(F)c3c2)cn1 ; *c1ccnc(/C(=C(/CC(F)(F)F)c2ccccc2)c2ccc3[nH]nc(F)c3c2)c1]

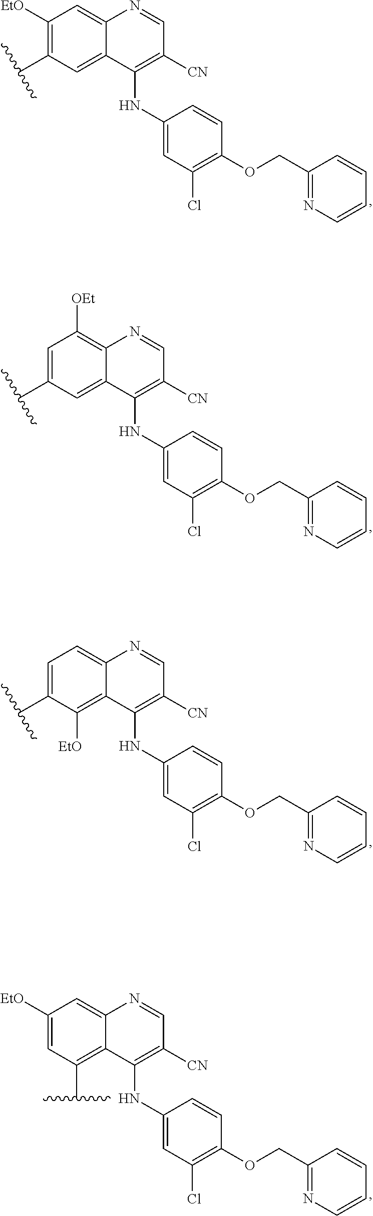 [SMILES: *c1cc2c(Nc3ccc(OCc4ccccn4)c(Cl)c3)c(C#N)cnc2cc1OCC ; *c1cc(OCC)c2ncc(C#N)c(Nc3ccc(OCc4ccccn4)c(Cl)c3)c2c1 ; *c1ccc2ncc(C#N)c(Nc3ccc(OCc4ccccn4)c(Cl)c3)c2c1OCC ; *c1cc(OCC)cc2ncc(C#N)c(Nc3ccc(OCc4ccccn4)c(Cl)c3)c12]

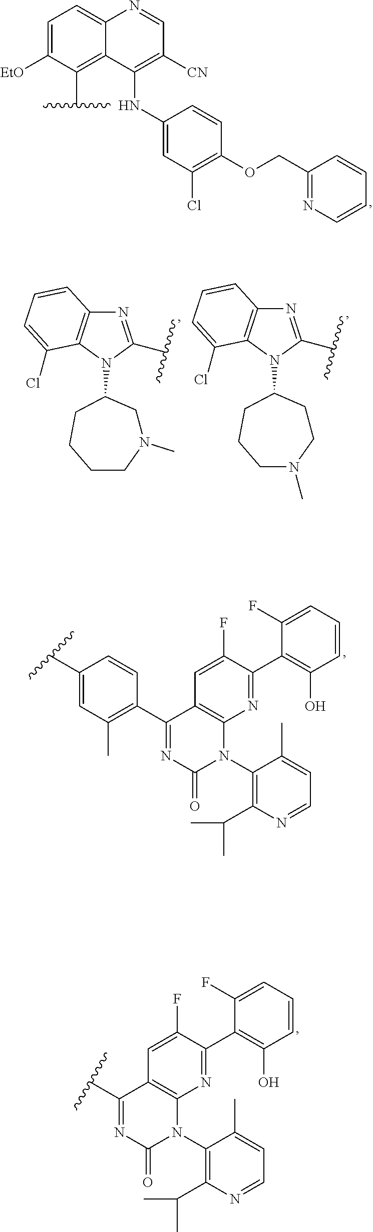

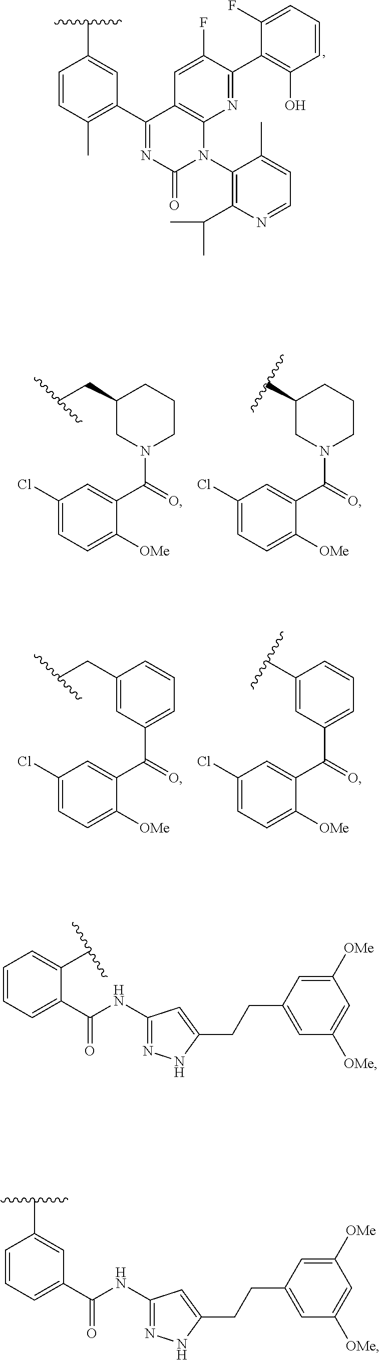

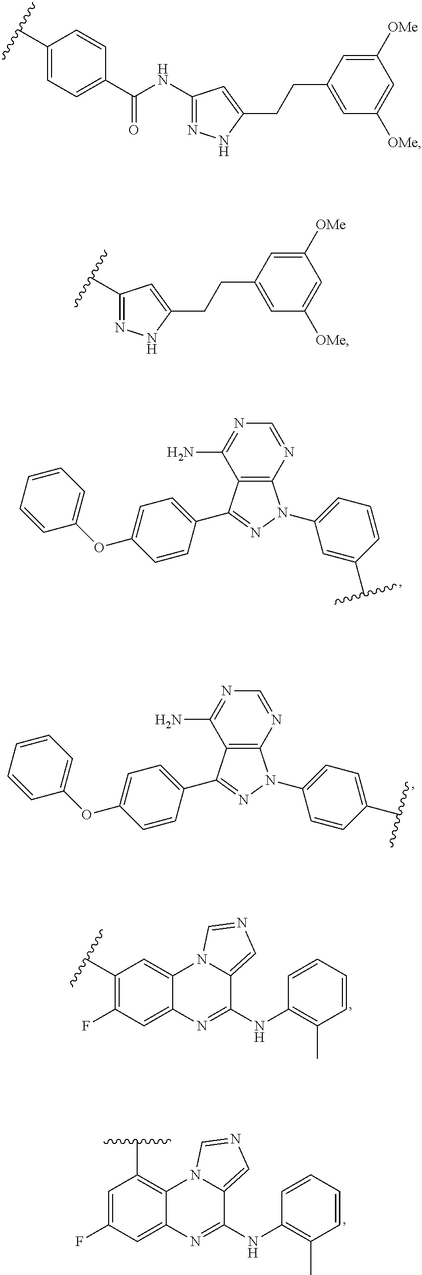 [SMILES: *c1ccc(C(=O)Nc2cc(CCc3cc(OC)cc(OC)c3)[nH]n2)cc1 ; *c1cc(CCc2cc(OC)cc(OC)c2)[nH]n1 ; *c1cccc(-n2nc(-c3ccc(Oc4ccccc4)cc3)c3c(N)ncnc32)c1 ; *c1ccc(-n2nc(-c3ccc(Oc4ccccc4)cc3)c3c(N)ncnc32)cc1 ; *c1cc2c(cc1F)nc(Nc1ccccc1C)c1cncn12 ; *c1cc(F)cc2nc(Nc3ccccc3C)c3cncn3c12]

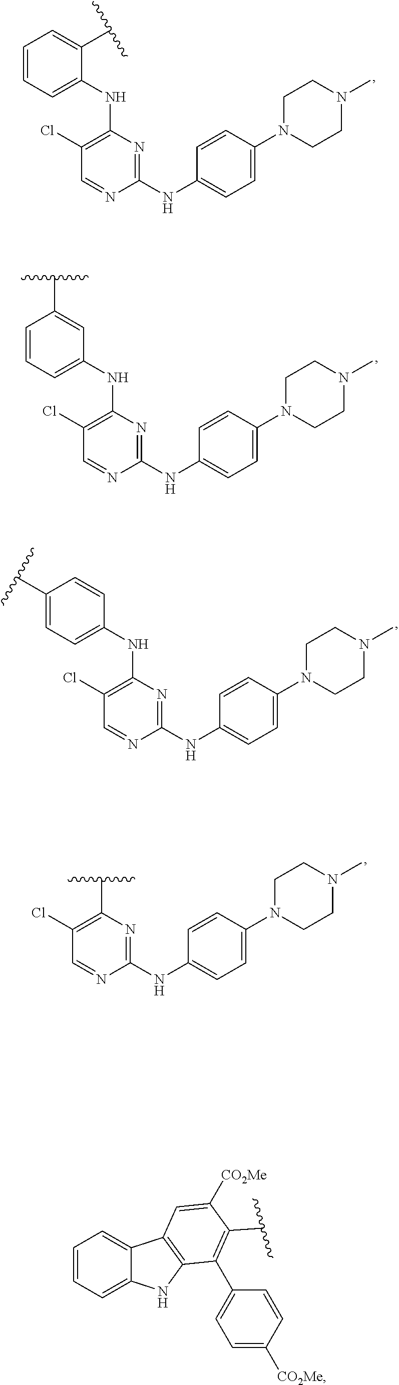 [SMILES: *c1ccccc1Nc1nc(Nc2ccc(N3CCN(C)CC3)cc2)ncc1Cl ; *c1cccc(Nc2nc(Nc3ccc(N4CCN(C)CC4)cc3)ncc2Cl)c1 ; *c1ccc(Nc2nc(Nc3ccc(N4CCN(C)CC4)cc3)ncc2Cl)cc1 ; *c1nc(Nc2ccc(N3CCN(C)CC3)cc2)ncc1Cl ; *c1c(C(=O)OC)cc2c([nH]c3ccccc32)c1-c1ccc(C(=O)OC)cc1]

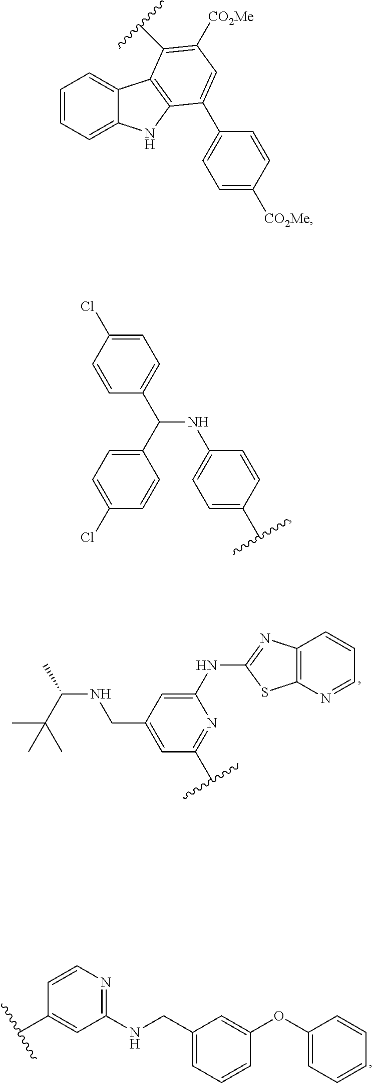 [SMILES: *c1c(C(=O)OC)cc(-c2ccc(C(=O)OC)cc2)c2[nH]c3ccccc3c12 ; *c1ccc(NC(c2ccc(Cl)cc2)c2ccc(Cl)cc2)cc1 ; *c1cc(CN[C@@H](C)C(C)(C)C)cc(Nc2nc3cccnc3s2)n1 ; *c1ccnc(NCc2cccc(Oc3ccccc3)c2)c1]

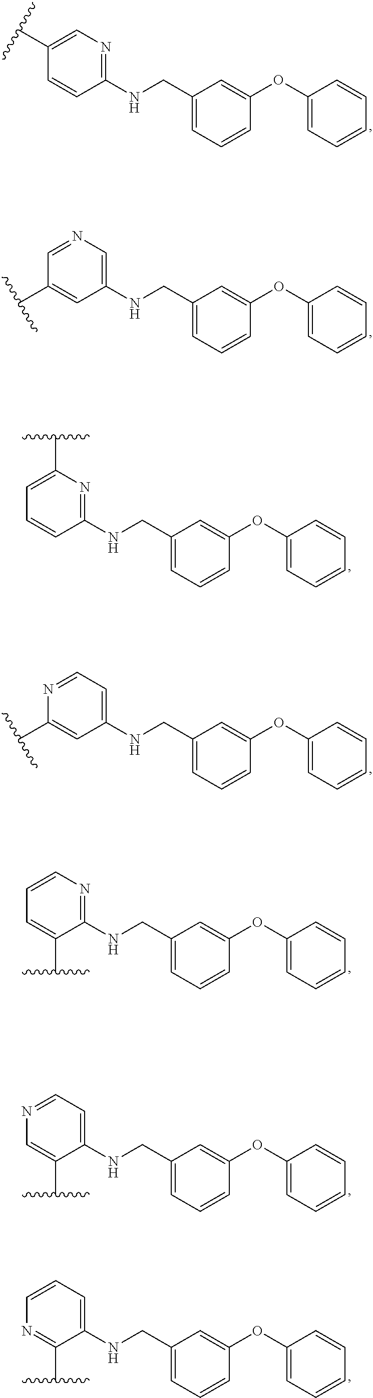

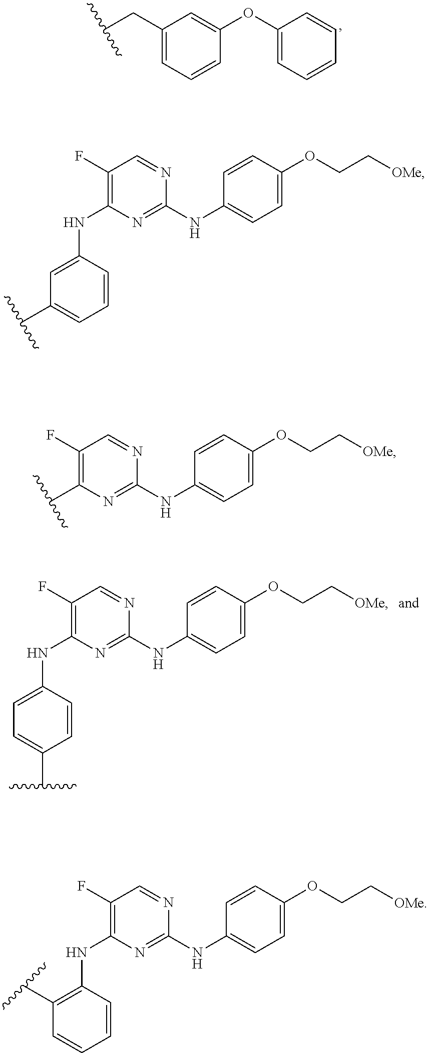 [SMILES: *Cc1cccc(Oc2ccccc2)c1 ; *c1cccc(Nc2nc(Nc3ccc(OCCOC)cc3)ncc2F)c1 ; *c1nc(Nc2ccc(OCCOC)cc2)ncc1F ; *c1ccc(Nc2nc(Nc3ccc(OCCOC)cc3)ncc2F)cc1 ; *c1ccccc1Nc1nc(Nc2ccc(OCCOC)cc2)ncc1F]

In some embodiments, G (or G¹) in Formula (I) is selected from:

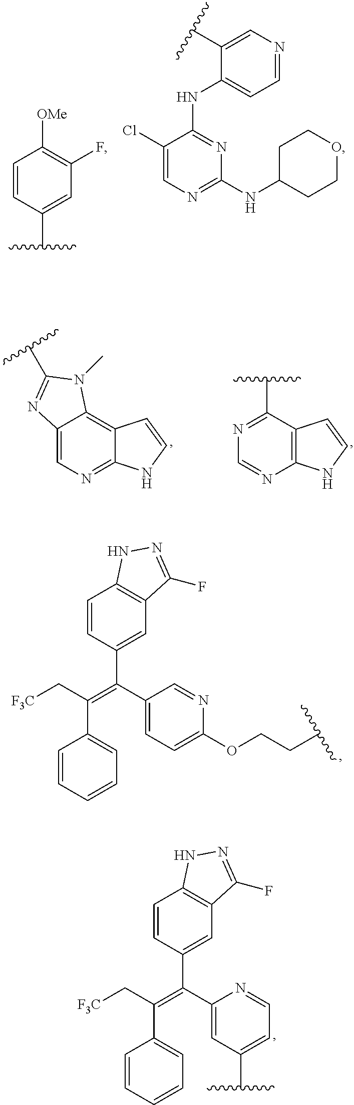

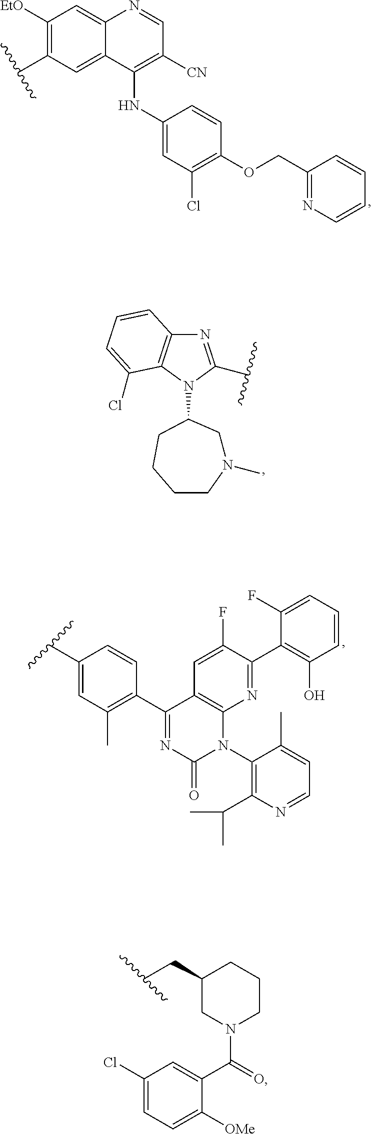 [SMILES: *c1cc2c(Nc3ccc(OCc4ccccn4)c(Cl)c3)c(C#N)cnc2cc1OCC ; *c1nc2cccc(Cl)c2n1[C@H]1CCCCN(C)C1 ; *c1ccc(-c2nc(=O)n(-c3c(C)ccnc3C(C)C)c3nc(-c4c(O)cccc4F)c(F)cc23)c(C)c1 ; *C[C@H]1CCCN(C(=O)c2cc(Cl)ccc2OC)C1]

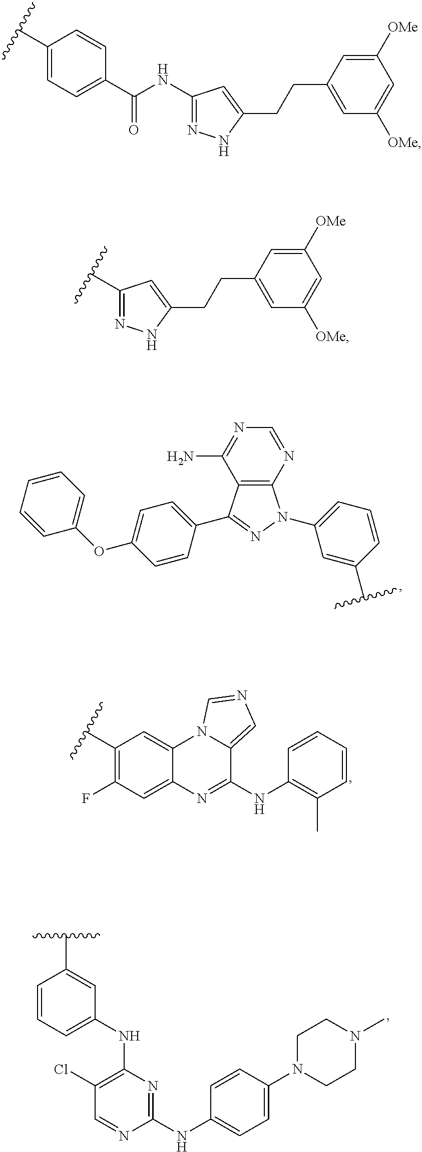 [SMILES: *c1ccc(C(=O)Nc2cc(CCc3cc(OC)cc(OC)c3)[nH]n2)cc1 ; *c1cc(CCc2cc(OC)cc(OC)c2)[nH]n1 ; *c1cccc(-n2nc(-c3ccc(Oc4ccccc4)cc3)c3c(N)ncnc32)c1 ; *c1cc2c(cc1F)nc(Nc1ccccc1C)c1cncn12 ; *c1cccc(Nc2nc(Nc3ccc(N4CCN(C)CC4)cc3)ncc2Cl)c1]

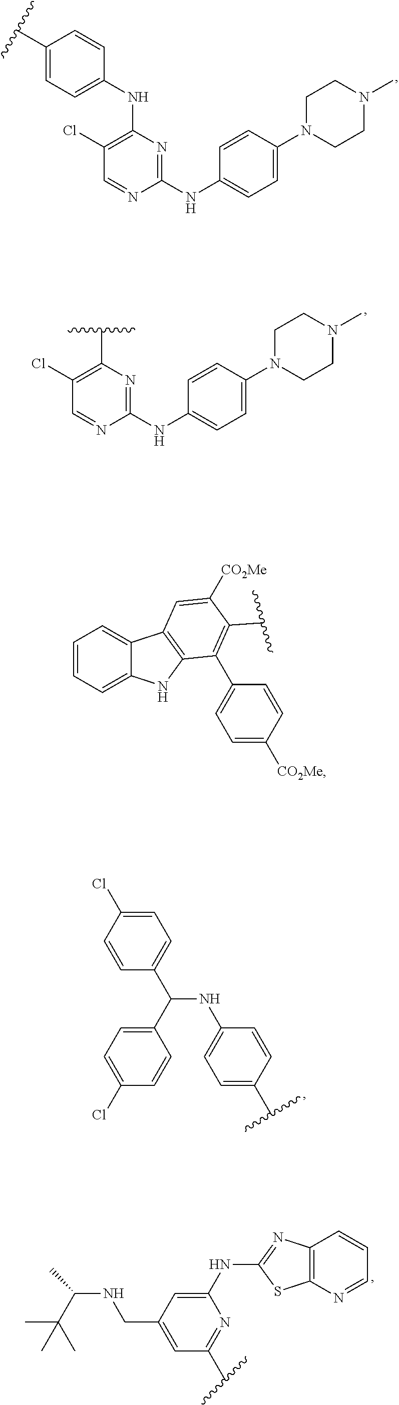

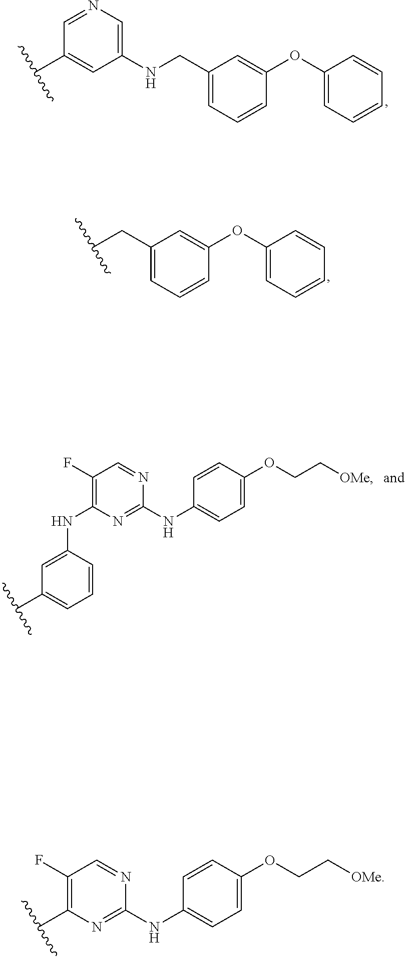 [SMILES: *c1cncc(NCc2cccc(Oc3ccccc3)c2)c1 ; *Cc1cccc(Oc2ccccc2)c1 ; *c1cccc(Nc2nc(Nc3ccc(OCCOC)cc3)ncc2F)c1 ; *c1nc(Nc2ccc(OCCOC)cc2)ncc1F]

In some embodiments,

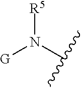

(G) (or G) in Formula (I) is selected from:

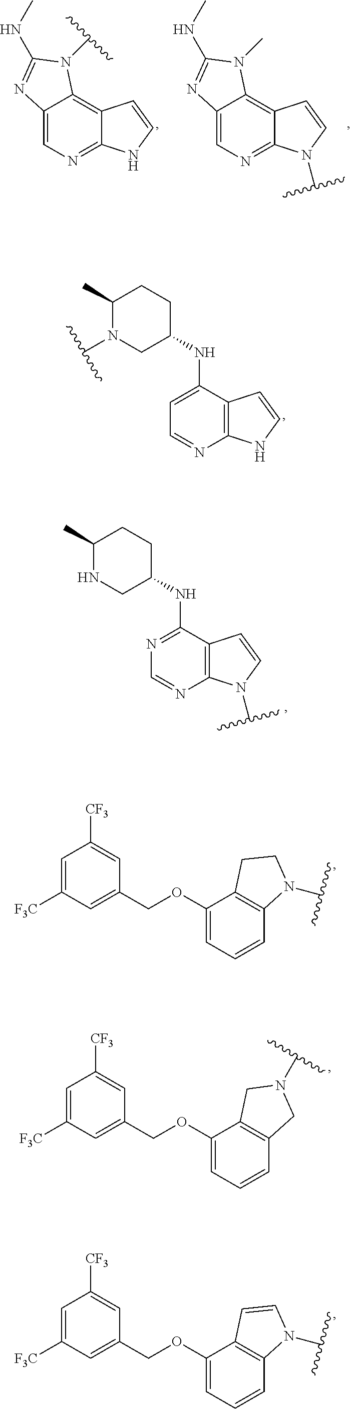

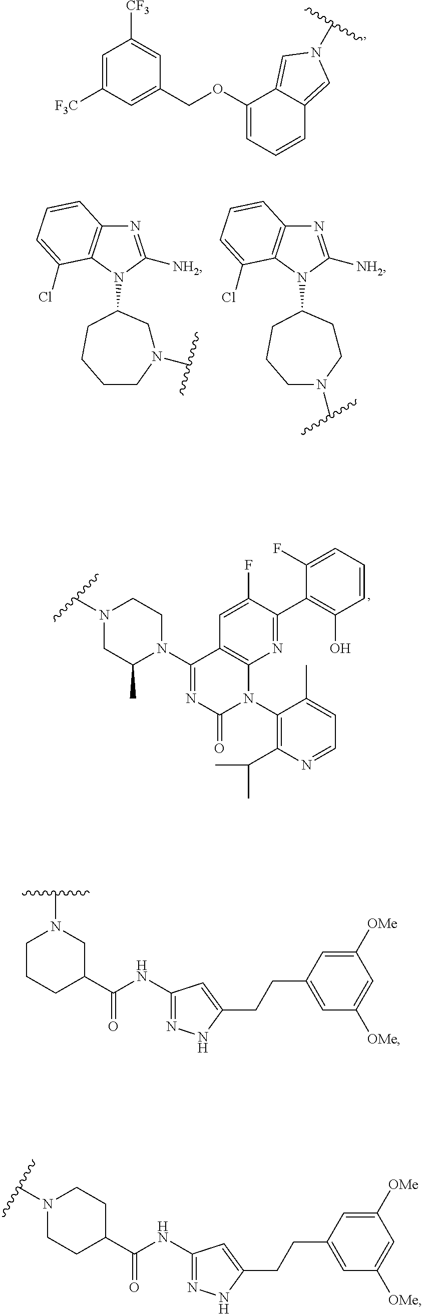 [SMILES: *n1cc2cccc(OCc3cc(C(F)(F)F)cc(C(F)(F)F)c3)c2c1 ; *N1CCCC[C@H](n2c(N)nc3cccc(Cl)c32)C1 ; *N1CCC[C@H](n2c(N)nc3cccc(Cl)c32)CC1 ; *N1CCN(c2nc(=O)n(-c3c(C)ccnc3C(C)C)c3nc(-c4c(O)cccc4F)c(F)cc23)[C@@H](C)C1 ; *N1CCCC(C(=O)Nc2cc(CCc3cc(OC)cc(OC)c3)[nH]n2)C1 ; *N1CCC(C(=O)Nc2cc(CCc3cc(OC)cc(OC)c3)[nH]n2)CC1]

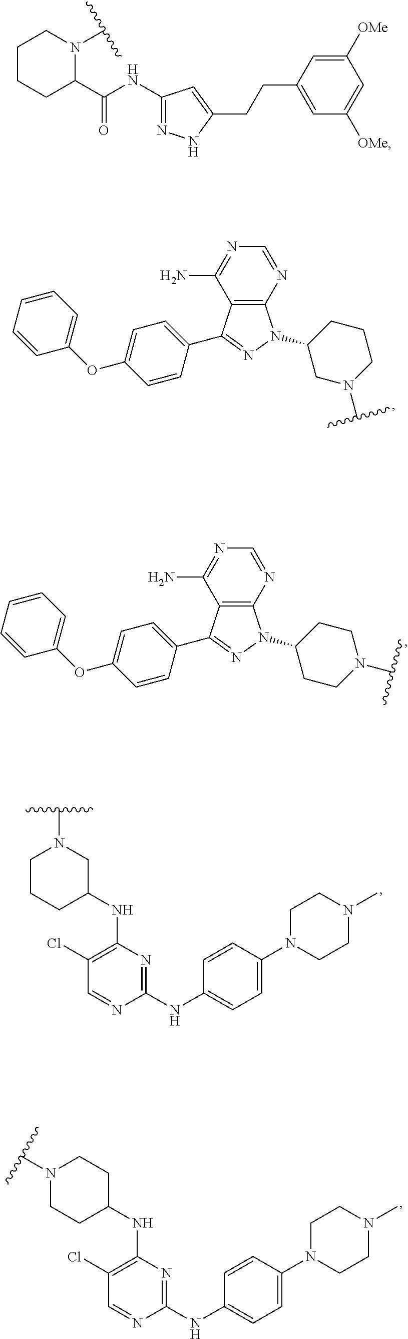

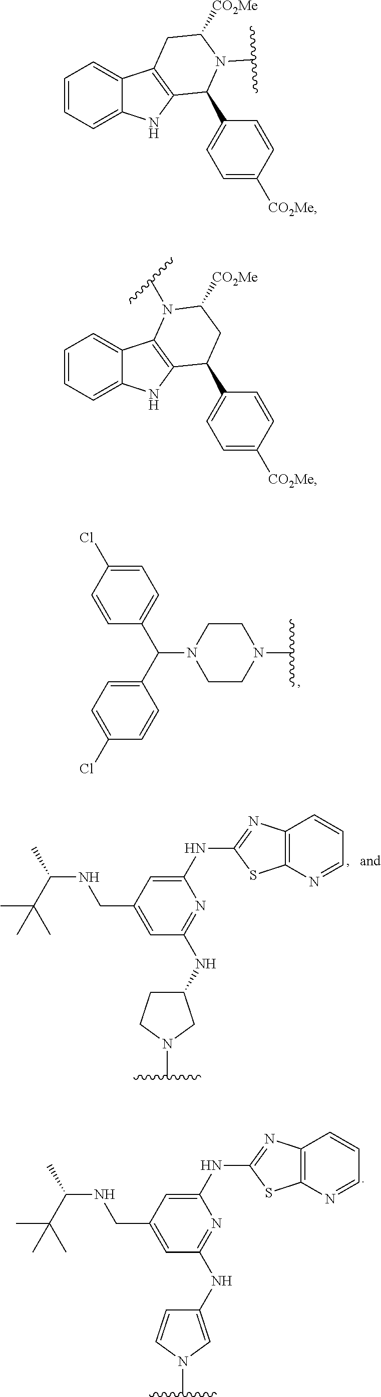 [SMILES: *N1[C@@H](c2ccc(C(=O)OC)cc2)c2[nH]c3ccccc3c2C[C@@H]1C(=O)OC ; *N1c2c([nH]c3ccccc23)[C@H](c2ccc(C(=O)OC)cc2)C[C@H]1C(=O)OC ; *N1CCN(C(c2ccc(Cl)cc2)c2ccc(Cl)cc2)CC1 ; *N1CC[C@H](Nc2cc(CN[C@@H](C)C(C)(C)C)cc(Nc3nc4cccnc4s3)n2)C1 ; *n1ccc(Nc2cc(CN[C@@H](C)C(C)(C)C)cc(Nc3nc4cccnc4s3)n2)c1]

In some embodiments

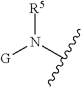

(G′) (or G) in Formula (I) is selected from:

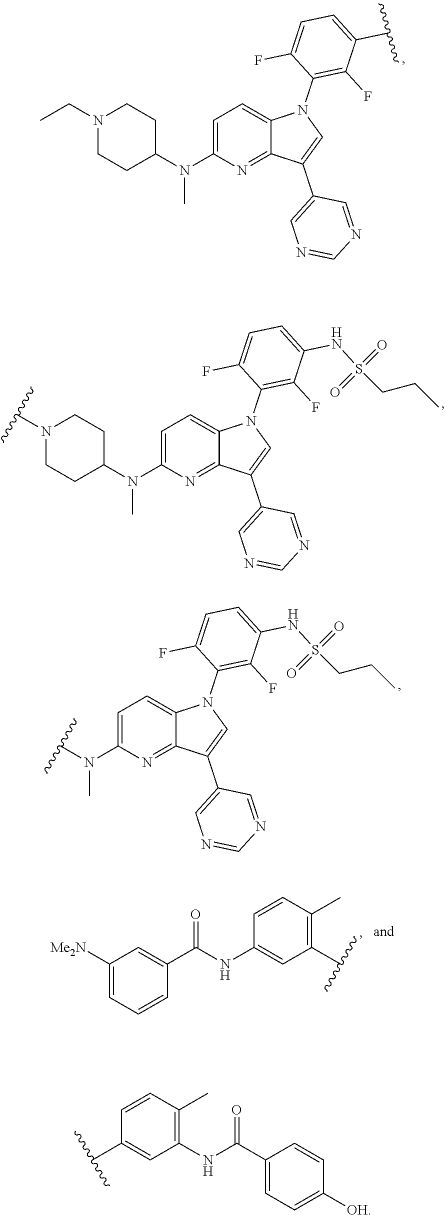

In some embodiments,

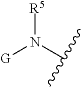

(G′) (or G) in Formula (I) is selected from:

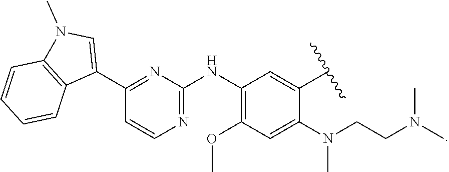 [SMILES: *c1cc(Nc2nccc(-c3cn(C)c4ccccc34)n2)c(OC)cc1N(C)CCN(C)C]

In some embodiments, G, G′, or G¹ in Formula (I) is substituted with 1, 2, or 3 substituents each independently selected from halogen, —CN, —OH, —CH₃, —CH₂CH₃, —CF₃, —OCH₃, —OCH₂CH₃, and —OCF₃.

In some embodiments, the compound of Formula (I) has a structure of Formula (Ia), or a salt or solvate thereof:

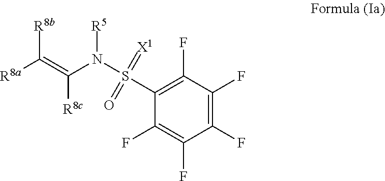

wherein,
each R^{8a}, R^{8b}, and R^{8c} is independently G¹ or R⁹, provided that only one of R^{8a}, R^{8b}, and R^{8c} is G¹; and
each R⁹ is independently hydrogen, substituted or unsubstituted alkyl, substituted or unsubstituted heteroalkyl, substituted or unsubstituted aryl, or substituted or unsubstituted heteroaryl.

In some embodiments, the compound of Formula (Ia) has a structure of Formula (Iaa), or a salt or solvate thereof:

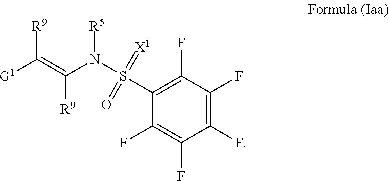

In some embodiments, the compound of Formula (I) has a structure of Formula (Ib), or a salt or solvate thereof:

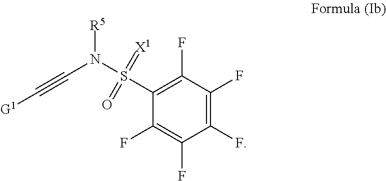

In some embodiments, R⁵ in Formula (I), Formula (Ia), Formula (Iaa), or Formula (Ib), is hydrogen, —CN, substituted or unsubstituted alkyl, or substituted or unsubstituted heteroalkyl.

In some embodiments, R⁵ in Formula (I), Formula (Ia), Formula (Iaa), or Formula (Ib), is hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl.

In some embodiments, R⁵ in Formula (I), Formula (Ia), Formula (Iaa), or Formula (Ib), is hydrogen, —CN, —CH₃, —CF₃, or cyclopropyl.

In some embodiments, R⁵ in Formula (I), Formula (Ia), Formula (Iaa), or Formula (Ib), is hydrogen.

In some embodiments, R⁵ in Formula (I), Formula (Ia), Formula (Iaa), or Formula (Ib), is independently hydrogen, substituted or unsubstituted alkyl, or substituted or unsubstituted heteroalkyl.

In some embodiments, R⁵ in Formula (I), Formula (Ia), Formula (Iaa), or Formula (Ib), is independently hydrogen, —OCH₂F, —OCHF₂, —OCF₃, —OCH₂CH₂F, —OCH₂CHF₂, —OCH₂CF₃, —NHCF₃, or —NHCH₂CF₃.

In some embodiments, R⁵ in Formula (I), Formula (Ia), Formula (Iaa), or Formula (Ib), is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy.

In some embodiments, R⁵ in Formula (I), Formula (Ia), Formula (Iaa), or Formula (Ib), is independently hydrogen, —CH₃, or —OCH₃.

In some embodiments, R⁵ in Formula (I), Formula (Ia), Formula (Iaa), or Formula (Ib), is independently hydrogen or —CH₃.

In some embodiments, R⁵ in Formula (I), Formula (Ia), Formula (Iaa), or Formula (Ib), is —CH₃.

In some embodiments, each R⁹ in Formula (Ia) or Formula (Iaa), is independently hydrogen, substituted or unsubstituted alkyl, or substituted or unsubstituted heteroalkyl.

In some embodiments, each R⁹ in Formula (Ia) or Formula (Iaa), is independently hydrogen, —OCH₂F, —OCHF₂, —OCF₃, —OCH₂CH₂F, —OCH₂CHF₂, —OCH₂CF₃, —NHCF₃, or —NHCH₂CF₃.

In some embodiments, each R⁹ in Formula (Ia) or Formula (Iaa), is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy.

In some embodiments, each R⁹ in Formula (Ia) or Formula (Iaa), is independently hydrogen, —CH₃, or —OCH₃.

In some embodiments, X¹ in Formula (I), Formula (Ia), Formula (Iaa), or Formula (Ib), is O, NH, or N (substituted or unsubstituted alkyl).

In some embodiments, X¹ in Formula (I), Formula (Ia), Formula (Iaa), or Formula (Ib), is O, NH, or N (unsubstituted alkyl).

In some embodiments, X¹ in Formula (I), Formula (Ia), Formula (Iaa), or Formula (Ib), is O, NH, or N(CH₃).

In some embodiments, X¹ in Formula (I), Formula (Ia), Formula (Iaa), or Formula (Ib), is O.

In some embodiments, X¹ in Formula (I), Formula (Ia), Formula (Iaa), or Formula (Ib), is NH or N(CH₃).

In some embodiments, provided herein is a compound of Formula (I), such as described herein, with the exception that the G of Formula (I) is R⁵ and the X¹ of Formula (I) is NG, wherein G and R⁵ are as described in Formula (I). In other words, in certain embodiments, —S(═O)(═X1)NR⁵G is substituted with —S(═O)(═NG) NR⁵₂.

In some embodiments, the pentafluorobenzenesulfonamide derivative compound described herein has a structure provided in Table 1.

TABLE 1

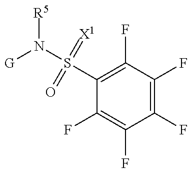

| # | 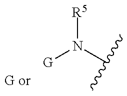 | R⁵ | X¹ |
|---|---|---|---|
| 1a | 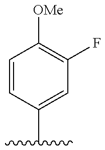 [SMILES: *c1ccc(OC)c(F)c1] | H | NH |
| 1b | 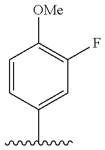 [SMILES: *c1ccc(OC)c(F)c1] | H | NMe |
| 1c | 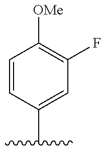 [SMILES: *c1ccc(OC)c(F)c1] | Me | NH |
| 1d | 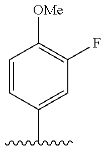 [SMILES: *c1ccc(OC)c(F)c1] | Me | NMe |
| 2a | 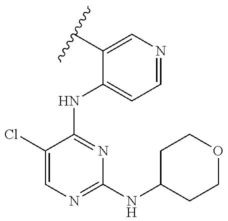 [SMILES: *c1cnccc1Nc1nc(NC2CCOCC2)ncc1Cl] | H | O |
| 2b | 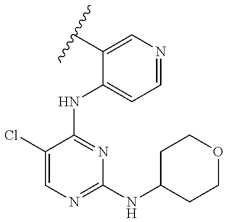 [SMILES: *c1cnccc1Nc1nc(NC2CCOCC2)ncc1Cl] | Me | O |
| 2c | 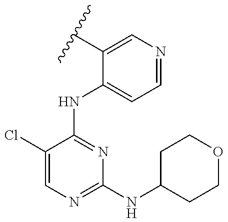 [SMILES: *c1cnccc1Nc1nc(NC2CCOCC2)ncc1Cl] | H | NH |
| 2d | 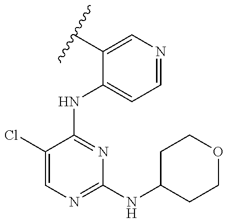 [SMILES: *c1cnccc1Nc1nc(NC2CCOCC2)ncc1Cl] | Me | NH |
| 2e | 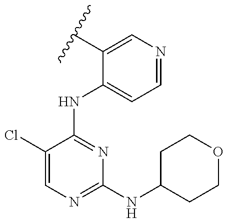 [SMILES: *c1cnccc1Nc1nc(NC2CCOCC2)ncc1Cl] | H | NMe |
| 2f | 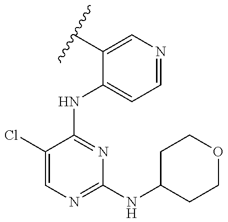 [SMILES: *c1cnccc1Nc1nc(NC2CCOCC2)ncc1Cl] | Me | NMe |
| 3a | 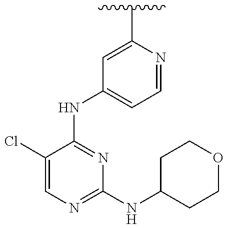 [SMILES: *c1cc(Nc2nc(NC3CCOCC3)ncc2Cl)ccn1] | H | O |
| 3b | 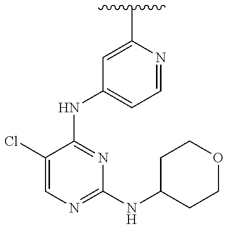 [SMILES: *c1cc(Nc2nc(NC3CCOCC3)ncc2Cl)ccn1] | Me | O |
| 3c | 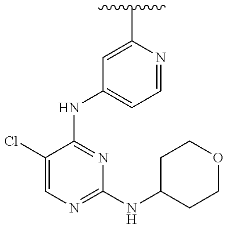 [SMILES: *c1cc(Nc2nc(NC3CCOCC3)ncc2Cl)ccn1] | H | NH |
| 3d | 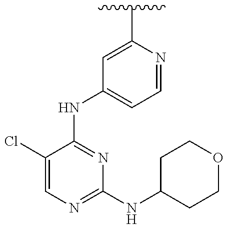 [SMILES: *c1cc(Nc2nc(NC3CCOCC3)ncc2Cl)ccn1] | Me | NH |
| 3e | 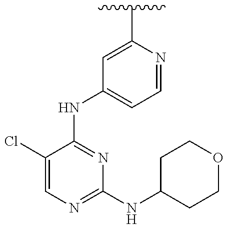 [SMILES: *c1cc(Nc2nc(NC3CCOCC3)ncc2Cl)ccn1] | H | NMe |
| 3f | 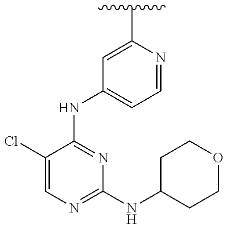 [SMILES: *c1cc(Nc2nc(NC3CCOCC3)ncc2Cl)ccn1] | Me | NMe |
| 4a | 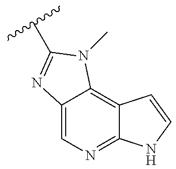 [SMILES: *c1nc2cnc3[nH]ccc3c2n1C] | H | O |
| 4b | 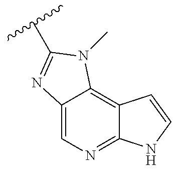 [SMILES: *c1nc2cnc3[nH]ccc3c2n1C] | Me | O |
| 4c | 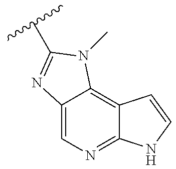 [SMILES: *c1nc2cnc3[nH]ccc3c2n1C] | H | NH |
| 4d | 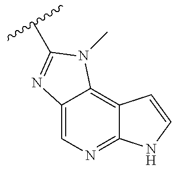 [SMILES: *c1nc2cnc3[nH]ccc3c2n1C] | Me | NH |
| 4e | 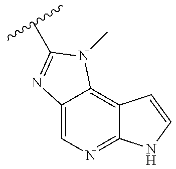 [SMILES: *c1nc2cnc3[nH]ccc3c2n1C] | H | NMe |
| 4f | 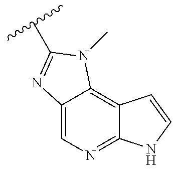 [SMILES: *c1nc2cnc3[nH]ccc3c2n1C] | Me | NMe |
| 5a | 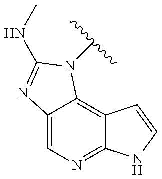 [SMILES: *n1c(NC)nc2cnc3[nH]ccc3c21] | N/A | O |
| 5b | 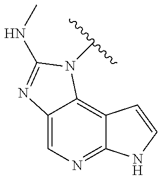 [SMILES: *n1c(NC)nc2cnc3[nH]ccc3c21] | N/A | NH |
| 5c | 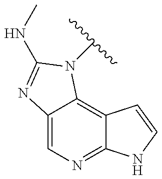 [SMILES: *n1c(NC)nc2cnc3[nH]ccc3c21] | N/A | NMe |
| 6a | 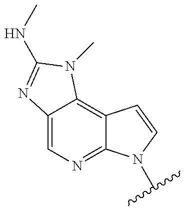 [SMILES: *n1ccc2c1ncc1nc(NC)n(C)c12] | N/A | O |
| 6b | 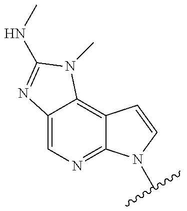 [SMILES: *n1ccc2c1ncc1nc(NC)n(C)c12] | N/A | NH |
| 6c | 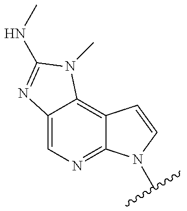 [SMILES: *n1ccc2c1ncc1nc(NC)n(C)c12] | N/A | NMe |
| 7a | 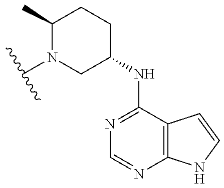 [SMILES: *N1C[C@@H](Nc2ncnc3[nH]ccc23)CC[C@@H]1C] | N/A | O |
| 7b | 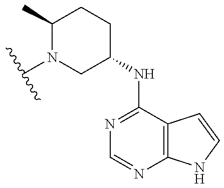 [SMILES: *N1C[C@@H](Nc2ncnc3[nH]ccc23)CC[C@@H]1C] | N/A | NH |
| 7c | 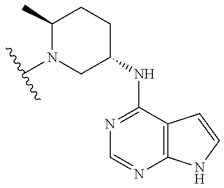 [SMILES: *N1C[C@@H](Nc2ncnc3[nH]ccc23)CC[C@@H]1C] | N/A | NMe |
| 8a | 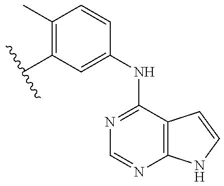 [SMILES: *c1cc(Nc2ncnc3[nH]ccc23)ccc1C] | H | O |
| 8b | 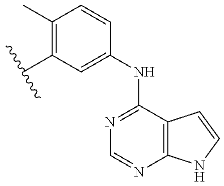 [SMILES: *c1cc(Nc2ncnc3[nH]ccc23)ccc1C] | Me | O |
| 8c | 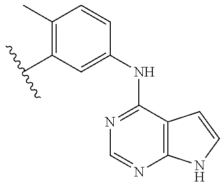 [SMILES: *c1cc(Nc2ncnc3[nH]ccc23)ccc1C] | H | NH |
| 8d | 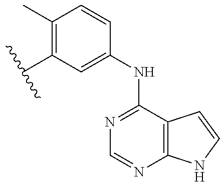 [SMILES: *c1cc(Nc2ncnc3[nH]ccc23)ccc1C] | Me | NH |
| 8e | 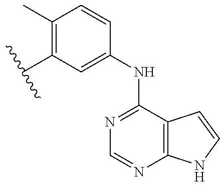 [SMILES: *c1cc(Nc2ncnc3[nH]ccc23)ccc1C] | H | NMe |
| 8f | 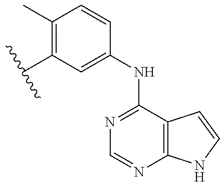 [SMILES: *c1cc(Nc2ncnc3[nH]ccc23)ccc1C] | Me | NMe |
| 9a | 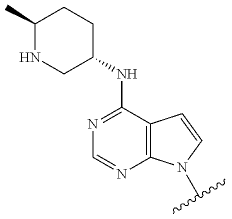 [SMILES: *n1ccc2c(N[C@H]3CC[C@H](C)NC3)ncnc21] | N/A | O |
| 9b | 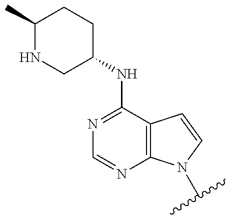 [SMILES: *n1ccc2c(N[C@H]3CC[C@H](C)NC3)ncnc21] | N/A | NH |
| 9c | 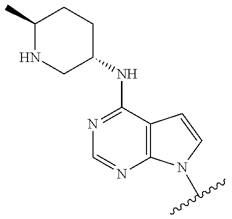 [SMILES: *n1ccc2c(N[C@H]3CC[C@H](C)NC3)ncnc21] | N/A | NMe |
| 10a | 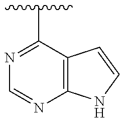 [SMILES: *c1ncnc2[nH]ccc12] | H | O |
| 10b | 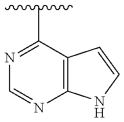 [SMILES: *c1ncnc2[nH]ccc12] | Me | O |
| 10c | 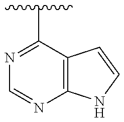 [SMILES: *c1ncnc2[nH]ccc12] | H | NH |
| 10d | 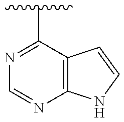 [SMILES: *c1ncnc2[nH]ccc12] | Me | NH |
| 10e | 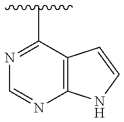 [SMILES: *c1ncnc2[nH]ccc12] | H | NMe |
| 10f | 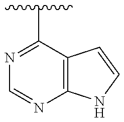 [SMILES: *c1ncnc2[nH]ccc12] | Me | NMe |
| 11a | 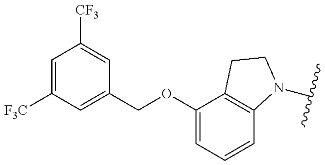 [SMILES: *N1CCc2c(OCc3cc(C(F)(F)F)cc(C(F)(F)F)c3)cccc21] | N/A | O |
| 11b | 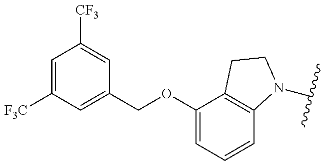 [SMILES: *N1CCc2c(OCc3cc(C(F)(F)F)cc(C(F)(F)F)c3)cccc21] | N/A | NH |
| 11c | 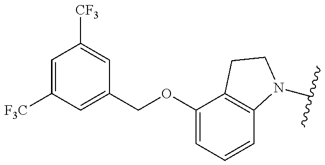 [SMILES: *N1CCc2c(OCc3cc(C(F)(F)F)cc(C(F)(F)F)c3)cccc21] | N/A | NMe |
| 12a | 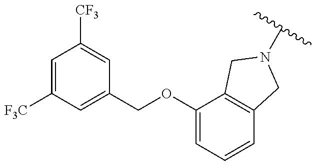 [SMILES: *N1Cc2cccc(OCc3cc(C(F)(F)F)cc(C(F)(F)F)c3)c2C1] | N/A | O |
| 12b | 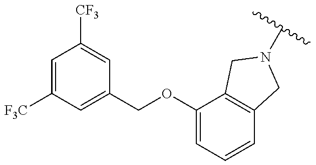 [SMILES: *N1Cc2cccc(OCc3cc(C(F)(F)F)cc(C(F)(F)F)c3)c2C1] | N/A | NH |
| 12c | 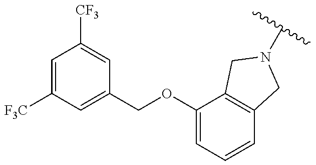 [SMILES: *N1Cc2cccc(OCc3cc(C(F)(F)F)cc(C(F)(F)F)c3)c2C1] | N/A | NMe |
| 13a | 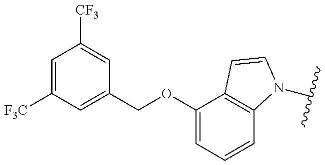 [SMILES: *n1ccc2c(OCc3cc(C(F)(F)F)cc(C(F)(F)F)c3)cccc21] | N/A | O |
| 13b | 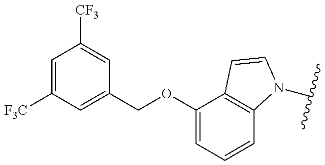 [SMILES: *n1ccc2c(OCc3cc(C(F)(F)F)cc(C(F)(F)F)c3)cccc21] | N/A | NH |
| 13c | 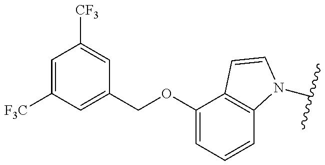 [SMILES: *n1ccc2c(OCc3cc(C(F)(F)F)cc(C(F)(F)F)c3)cccc21] | N/A | NMe |
| 14a | 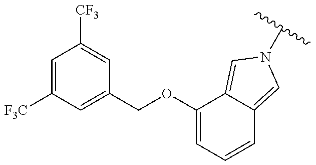 [SMILES: *n1cc2cccc(OCc3cc(C(F)(F)F)cc(C(F)(F)F)c3)c2c1] | N/A | O |
| 14b | 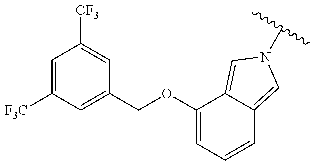 [SMILES: *n1cc2cccc(OCc3cc(C(F)(F)F)cc(C(F)(F)F)c3)c2c1] | N/A | NH |
| 14c | 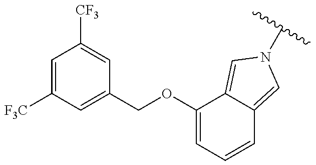 [SMILES: *n1cc2cccc(OCc3cc(C(F)(F)F)cc(C(F)(F)F)c3)c2c1] | N/A | NMe |
| 15a | 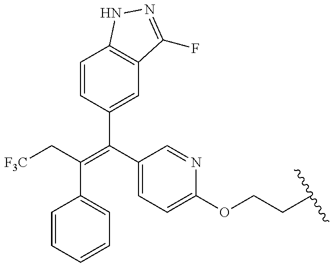 [SMILES: *CCOc1ccc(/C(=C(/CC(F)(F)F)c2ccccc2)c2ccc3[nH]nc(F)c3c2)cn1] | H | O |
| 15b | 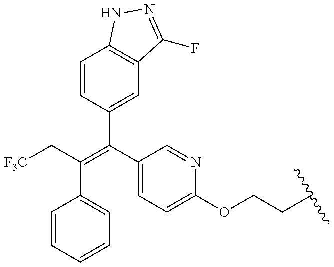 [SMILES: *CCOc1ccc(/C(=C(/CC(F)(F)F)c2ccccc2)c2ccc3[nH]nc(F)c3c2)cn1] | Me | O |
| 15c | 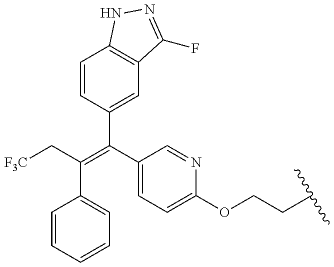 [SMILES: *CCOc1ccc(/C(=C(/CC(F)(F)F)c2ccccc2)c2ccc3[nH]nc(F)c3c2)cn1] | H | NH |
| 15d | 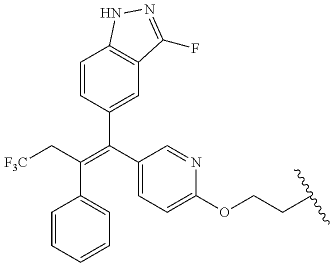 [SMILES: *CCOc1ccc(/C(=C(/CC(F)(F)F)c2ccccc2)c2ccc3[nH]nc(F)c3c2)cn1] | Me | NH |
| 15e | 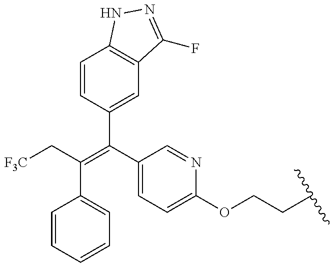 [SMILES: *CCOc1ccc(/C(=C(/CC(F)(F)F)c2ccccc2)c2ccc3[nH]nc(F)c3c2)cn1] | H | NMe |
| 15f | 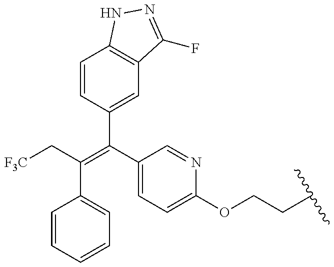 [SMILES: *CCOc1ccc(/C(=C(/CC(F)(F)F)c2ccccc2)c2ccc3[nH]nc(F)c3c2)cn1] | Me | NMe |
| 16a | 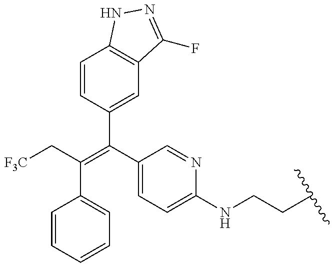 [SMILES: *CCNc1ccc(/C(=C(/CC(F)(F)F)c2ccccc2)c2ccc3[nH]nc(F)c3c2)cn1] | H | O |
| 16b | 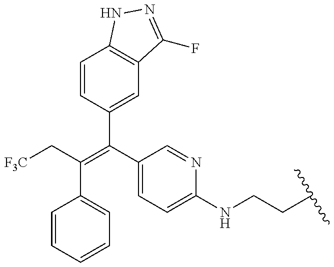 [SMILES: *CCNc1ccc(/C(=C(/CC(F)(F)F)c2ccccc2)c2ccc3[nH]nc(F)c3c2)cn1] | Me | O |
| 16c | 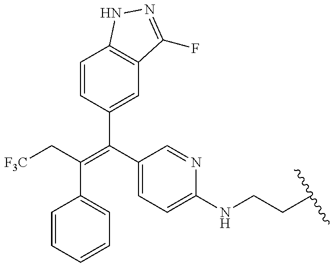 [SMILES: *CCNc1ccc(/C(=C(/CC(F)(F)F)c2ccccc2)c2ccc3[nH]nc(F)c3c2)cn1] | H | NH |
| 16d | 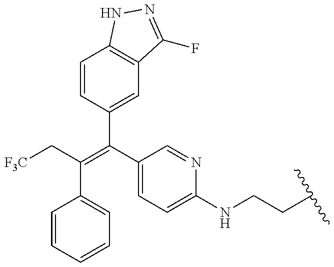 [SMILES: *CCNc1ccc(/C(=C(/CC(F)(F)F)c2ccccc2)c2ccc3[nH]nc(F)c3c2)cn1] | Me | NH |
| 16e | 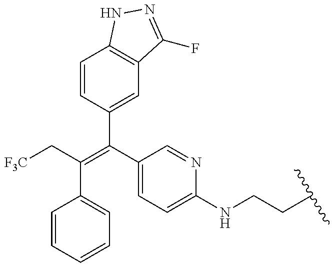 [SMILES: *CCNc1ccc(/C(=C(/CC(F)(F)F)c2ccccc2)c2ccc3[nH]nc(F)c3c2)cn1] | H | NMe |
| 16f | 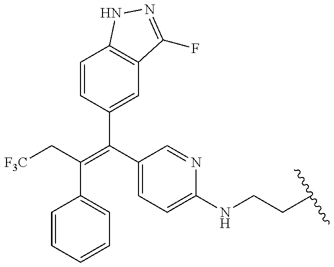 [SMILES: *CCNc1ccc(/C(=C(/CC(F)(F)F)c2ccccc2)c2ccc3[nH]nc(F)c3c2)cn1] | Me | NMe |
| 17a | 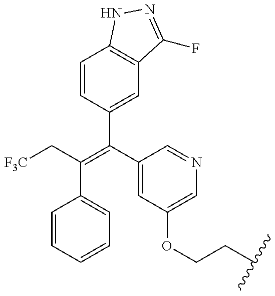 [SMILES: *CCOc1cncc(/C(=C(/CC(F)(F)F)c2ccccc2)c2ccc3[nH]nc(F)c3c2)c1] | H | O |
| 17b | 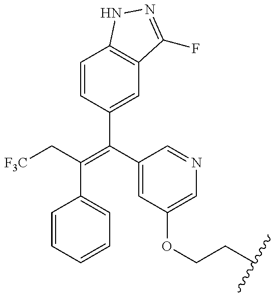 [SMILES: *CCOc1cncc(/C(=C(/CC(F)(F)F)c2ccccc2)c2ccc3[nH]nc(F)c3c2)c1] | Me | O |
| 17c | 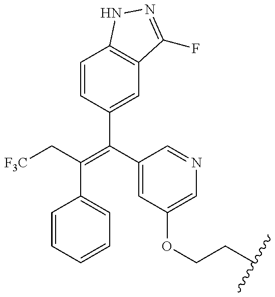 [SMILES: *CCOc1cncc(/C(=C(/CC(F)(F)F)c2ccccc2)c2ccc3[nH]nc(F)c3c2)c1] | H | NH |
| 17d | 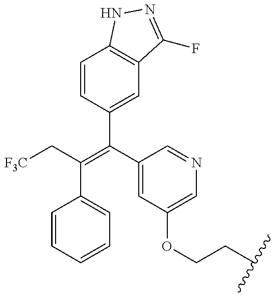 [SMILES: *CCOc1cncc(/C(=C(/CC(F)(F)F)c2ccccc2)c2ccc3[nH]nc(F)c3c2)c1] | Me | NH |
| 17e | 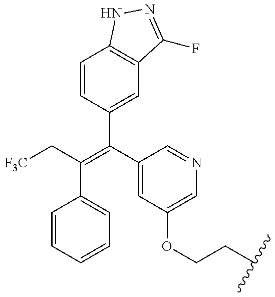 [SMILES: *CCOc1cncc(/C(=C(/CC(F)(F)F)c2ccccc2)c2ccc3[nH]nc(F)c3c2)c1] | H | NMe |
| 17f | 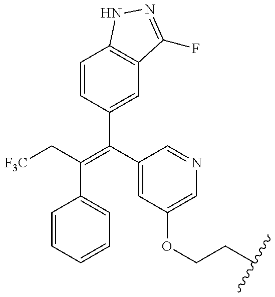 [SMILES: *CCOc1cncc(/C(=C(/CC(F)(F)F)c2ccccc2)c2ccc3[nH]nc(F)c3c2)c1] | Me | NMe |
| 18a | 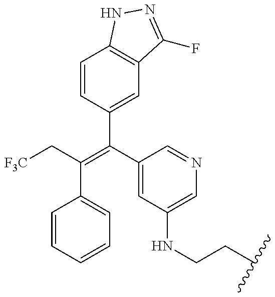 [SMILES: *CCNc1cncc(/C(=C(/CC(F)(F)F)c2ccccc2)c2ccc3[nH]nc(F)c3c2)c1] | H | O |
| 18b | 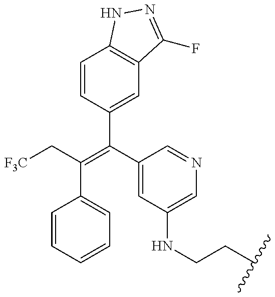 [SMILES: *CCNc1cncc(/C(=C(/CC(F)(F)F)c2ccccc2)c2ccc3[nH]nc(F)c3c2)c1] | Me | O |
| 18c | 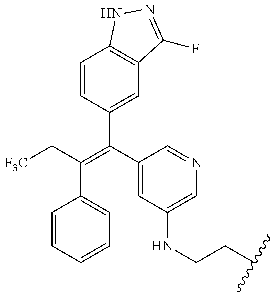 [SMILES: *CCNc1cncc(/C(=C(/CC(F)(F)F)c2ccccc2)c2ccc3[nH]nc(F)c3c2)c1] | H | NH |
| 18d | 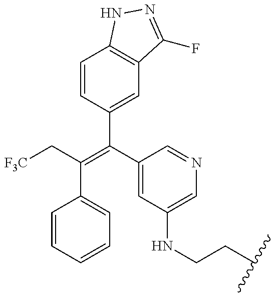 [SMILES: *CCNc1cncc(/C(=C(/CC(F)(F)F)c2ccccc2)c2ccc3[nH]nc(F)c3c2)c1] | Me | NH |
| 18e | 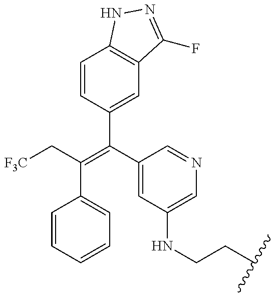 [SMILES: *CCNc1cncc(/C(=C(/CC(F)(F)F)c2ccccc2)c2ccc3[nH]nc(F)c3c2)c1] | H | NMe |
| 18f | 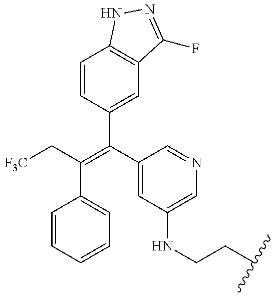 [SMILES: *CCNc1cncc(/C(=C(/CC(F)(F)F)c2ccccc2)c2ccc3[nH]nc(F)c3c2)c1] | Me | NMe |
| 19a | 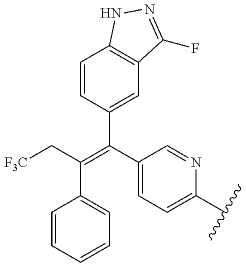 [SMILES: *c1ccc(/C(=C(/CC(F)(F)F)c2ccccc2)c2ccc3[nH]nc(F)c3c2)cn1] | H | O |
| 19b | 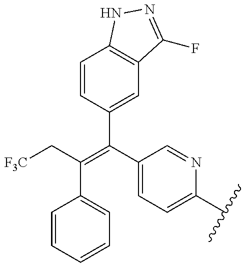 [SMILES: *c1ccc(/C(=C(/CC(F)(F)F)c2ccccc2)c2ccc3[nH]nc(F)c3c2)cn1] | Me | O |
| 19c | 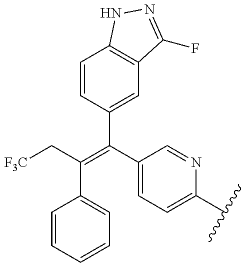 [SMILES: *c1ccc(/C(=C(/CC(F)(F)F)c2ccccc2)c2ccc3[nH]nc(F)c3c2)cn1] | H | NH |
| 19d | 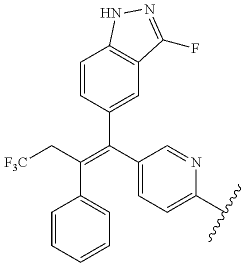 [SMILES: *c1ccc(/C(=C(/CC(F)(F)F)c2ccccc2)c2ccc3[nH]nc(F)c3c2)cn1] | Me | NH |
| 19e | 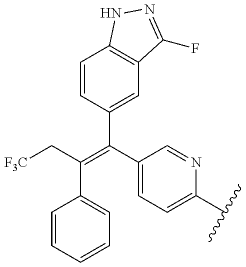 [SMILES: *c1ccc(/C(=C(/CC(F)(F)F)c2ccccc2)c2ccc3[nH]nc(F)c3c2)cn1] | H | NMe |
| 19f | 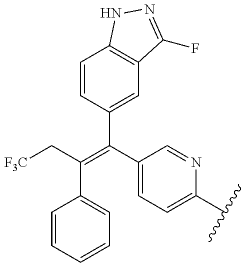 [SMILES: *c1ccc(/C(=C(/CC(F)(F)F)c2ccccc2)c2ccc3[nH]nc(F)c3c2)cn1] | Me | NMe |
| 20a | 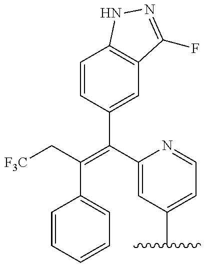 [SMILES: *c1ccnc(/C(=C(/CC(F)(F)F)c2ccccc2)c2ccc3[nH]nc(F)c3c2)c1] | H | O |
| 20b | 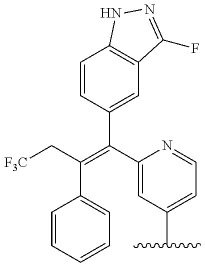 [SMILES: *c1ccnc(/C(=C(/CC(F)(F)F)c2ccccc2)c2ccc3[nH]nc(F)c3c2)c1] | Me | O |
| 20c | 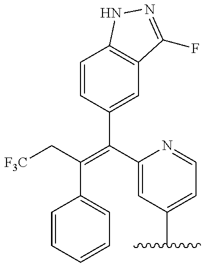 [SMILES: *c1ccnc(/C(=C(/CC(F)(F)F)c2ccccc2)c2ccc3[nH]nc(F)c3c2)c1] | H | NH |
| 20d | 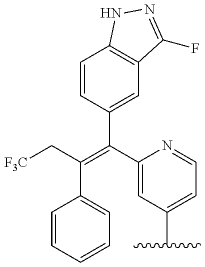 [SMILES: *c1ccnc(/C(=C(/CC(F)(F)F)c2ccccc2)c2ccc3[nH]nc(F)c3c2)c1] | Me | NH |
| 20e | 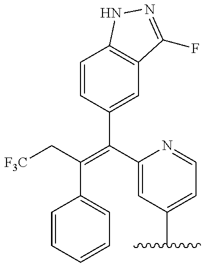 [SMILES: *c1ccnc(/C(=C(/CC(F)(F)F)c2ccccc2)c2ccc3[nH]nc(F)c3c2)c1] | H | NMe |
| 20f | 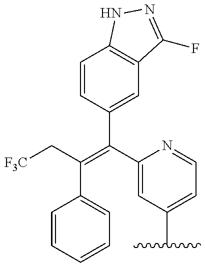 [SMILES: *c1ccnc(/C(=C(/CC(F)(F)F)c2ccccc2)c2ccc3[nH]nc(F)c3c2)c1] | Me | NMe |
| 21a | 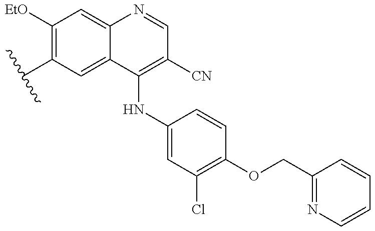 [SMILES: *c1cc2c(Nc3ccc(OCc4ccccn4)c(Cl)c3)c(C#N)cnc2cc1OCC] | H | O |
| 21b | 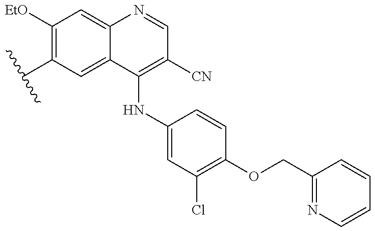 [SMILES: *c1cc2c(Nc3ccc(OCc4ccccn4)c(Cl)c3)c(C#N)cnc2cc1OCC] | Me | O |
| 21c | 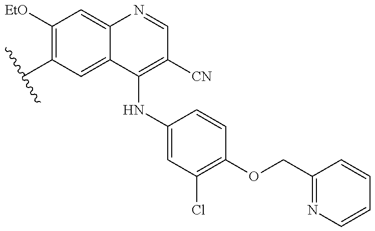 [SMILES: *c1cc2c(Nc3ccc(OCc4ccccn4)c(Cl)c3)c(C#N)cnc2cc1OCC] | H | NH |
| 21d | 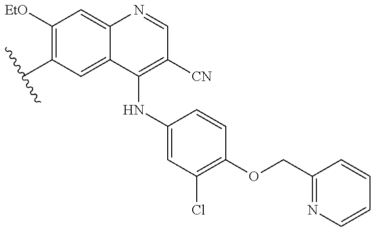 [SMILES: *c1cc2c(Nc3ccc(OCc4ccccn4)c(Cl)c3)c(C#N)cnc2cc1OCC] | Me | NH |
| 21e | 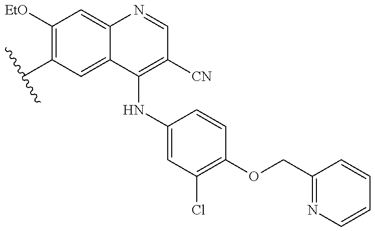 [SMILES: *c1cc2c(Nc3ccc(OCc4ccccn4)c(Cl)c3)c(C#N)cnc2cc1OCC] | H | NMe |
| 21f | 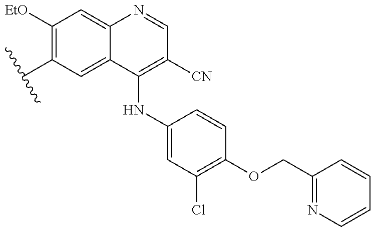 [SMILES: *c1cc2c(Nc3ccc(OCc4ccccn4)c(Cl)c3)c(C#N)cnc2cc1OCC] | Me | NMe |
| 22a | 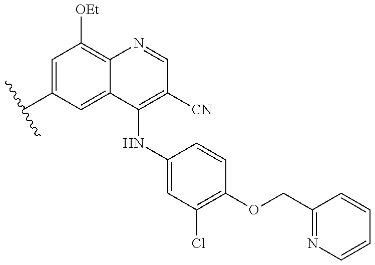 [SMILES: *c1cc(OCC)c2ncc(C#N)c(Nc3ccc(OCc4ccccn4)c(Cl)c3)c2c1] | H | O |
| 22b | 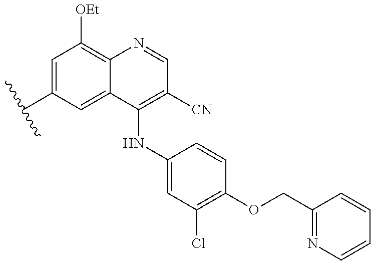 [SMILES: *c1cc(OCC)c2ncc(C#N)c(Nc3ccc(OCc4ccccn4)c(Cl)c3)c2c1] | Me | O |
| 22c | 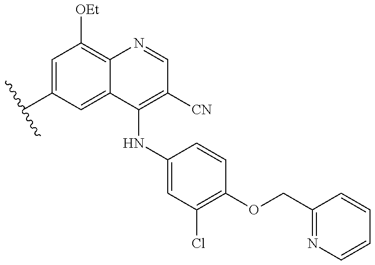 [SMILES: *c1cc(OCC)c2ncc(C#N)c(Nc3ccc(OCc4ccccn4)c(Cl)c3)c2c1] | H | NH |
| 22d | 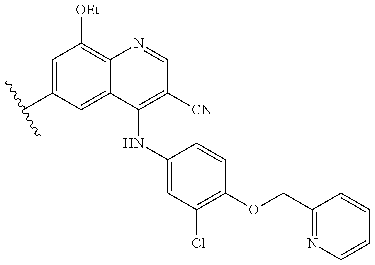 [SMILES: *c1cc(OCC)c2ncc(C#N)c(Nc3ccc(OCc4ccccn4)c(Cl)c3)c2c1] | Me | NH |
| 22e | 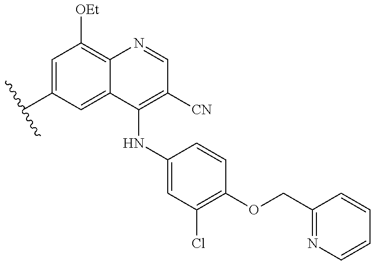 [SMILES: *c1cc(OCC)c2ncc(C#N)c(Nc3ccc(OCc4ccccn4)c(Cl)c3)c2c1] | H | NMe |
| 22f | 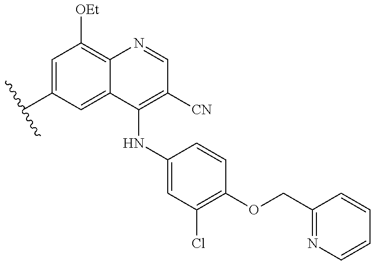 [SMILES: *c1cc(OCC)c2ncc(C#N)c(Nc3ccc(OCc4ccccn4)c(Cl)c3)c2c1] | Me | NMe |
| 23a | 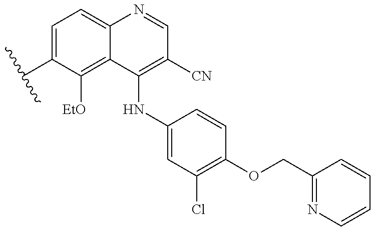 [SMILES: *c1ccc2ncc(C#N)c(Nc3ccc(OCc4ccccn4)c(Cl)c3)c2c1OCC] | H | O |
| 23b | 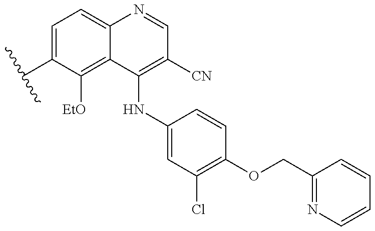 [SMILES: *c1ccc2ncc(C#N)c(Nc3ccc(OCc4ccccn4)c(Cl)c3)c2c1OCC] | Me | O |
| 23c | 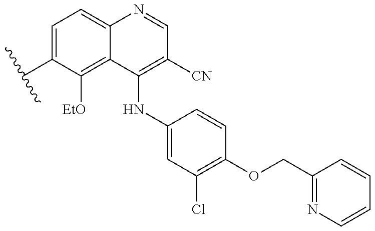 [SMILES: *c1ccc2ncc(C#N)c(Nc3ccc(OCc4ccccn4)c(Cl)c3)c2c1OCC] | H | NH |
| 23d | 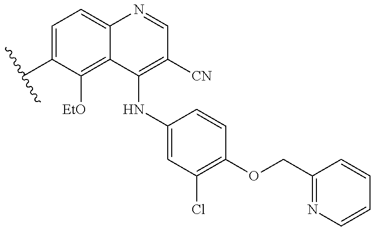 [SMILES: *c1ccc2ncc(C#N)c(Nc3ccc(OCc4ccccn4)c(Cl)c3)c2c1OCC] | Me | NH |
| 23e | 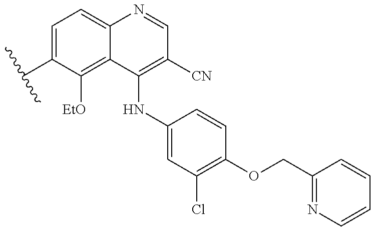 [SMILES: *c1ccc2ncc(C#N)c(Nc3ccc(OCc4ccccn4)c(Cl)c3)c2c1OCC] | H | NMe |
| 23f | 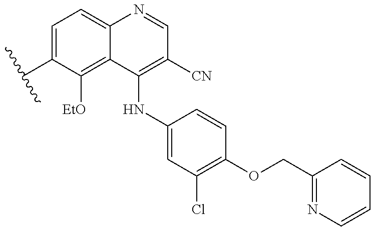 [SMILES: *c1ccc2ncc(C#N)c(Nc3ccc(OCc4ccccn4)c(Cl)c3)c2c1OCC] | Me | NMe |
| 24a | 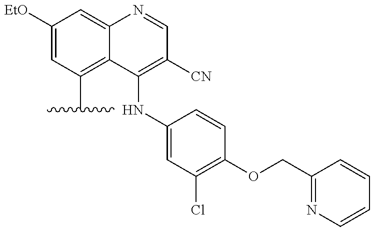 [SMILES: *c1cc(OCC)cc2ncc(C#N)c(Nc3ccc(OCc4ccccn4)c(Cl)c3)c12] | H | O |
| 24b | 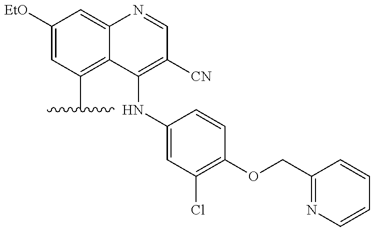 [SMILES: *c1cc(OCC)cc2ncc(C#N)c(Nc3ccc(OCc4ccccn4)c(Cl)c3)c12] | Me | O |
| 24c | 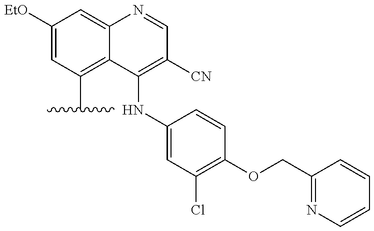 [SMILES: *c1cc(OCC)cc2ncc(C#N)c(Nc3ccc(OCc4ccccn4)c(Cl)c3)c12] | H | NH |
| 24d | 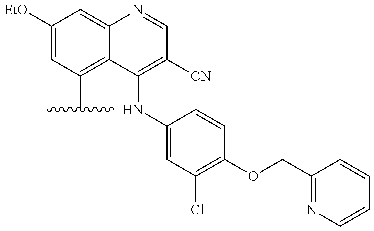 [SMILES: *c1cc(OCC)cc2ncc(C#N)c(Nc3ccc(OCc4ccccn4)c(Cl)c3)c12] | Me | NH |
| 24e | 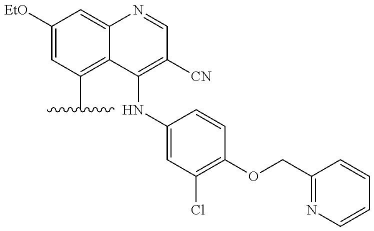 [SMILES: *c1cc(OCC)cc2ncc(C#N)c(Nc3ccc(OCc4ccccn4)c(Cl)c3)c12] | H | NMe |
| 24f | 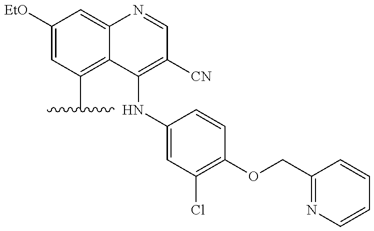 [SMILES: *c1cc(OCC)cc2ncc(C#N)c(Nc3ccc(OCc4ccccn4)c(Cl)c3)c12] | Me | NMe |
| 25a | 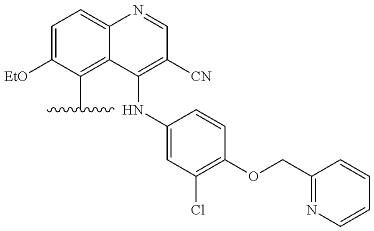 [SMILES: *c1c(OCC)ccc2ncc(C#N)c(Nc3ccc(OCc4ccccn4)c(Cl)c3)c12] | H | O |
| 25b | 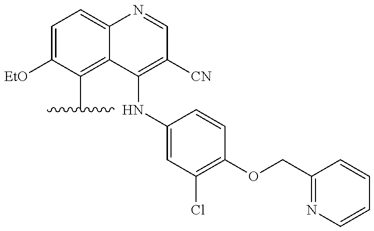 [SMILES: *c1c(OCC)ccc2ncc(C#N)c(Nc3ccc(OCc4ccccn4)c(Cl)c3)c12] | Me | O |
| 25c | 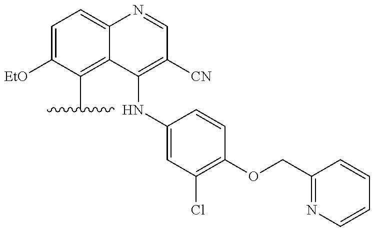 [SMILES: *c1c(OCC)ccc2ncc(C#N)c(Nc3ccc(OCc4ccccn4)c(Cl)c3)c12] | H | NH |
| 25d | 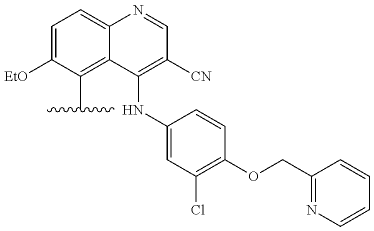 [SMILES: *c1c(OCC)ccc2ncc(C#N)c(Nc3ccc(OCc4ccccn4)c(Cl)c3)c12] | Me | NH |
| 25e | 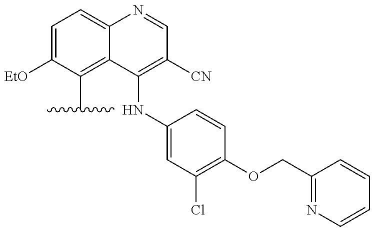 [SMILES: *c1c(OCC)ccc2ncc(C#N)c(Nc3ccc(OCc4ccccn4)c(Cl)c3)c12] | H | NMe |
| 25f | 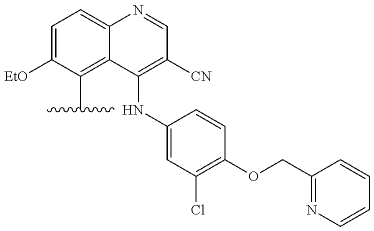 [SMILES: *c1c(OCC)ccc2ncc(C#N)c(Nc3ccc(OCc4ccccn4)c(Cl)c3)c12] | Me | NMe |
| 26a | 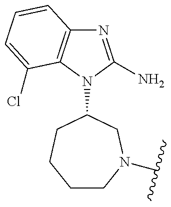 [SMILES: *N1CCCC[C@H](n2c(N)nc3cccc(Cl)c32)C1] | N/A | O |
| 26b | 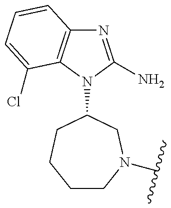 [SMILES: *N1CCCC[C@H](n2c(N)nc3cccc(Cl)c32)C1] | N/A | NH |
| 26c | 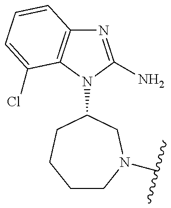 [SMILES: *N1CCCC[C@H](n2c(N)nc3cccc(Cl)c32)C1] | N/A | NMe |
| 27a | 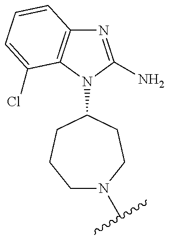 [SMILES: *N1CCC[C@H](n2c(N)nc3cccc(Cl)c32)CC1] | N/A | O |
| 27b | 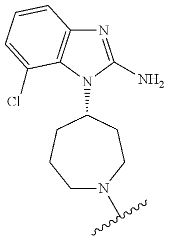 [SMILES: *N1CCC[C@H](n2c(N)nc3cccc(Cl)c32)CC1] | N/A | NH |
| 27c | 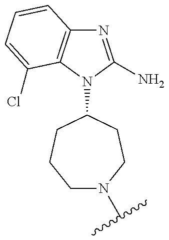 [SMILES: *N1CCC[C@H](n2c(N)nc3cccc(Cl)c32)CC1] | N/A | NMe |
| 28a | 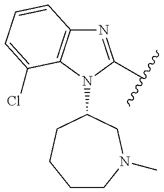 [SMILES: *c1nc2cccc(Cl)c2n1[C@H]1CCCCN(C)C1] | H | O |
| 28b | 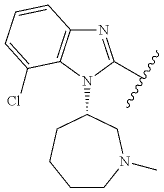 [SMILES: *c1nc2cccc(Cl)c2n1[C@H]1CCCCN(C)C1] | Me | O |
| 28c | 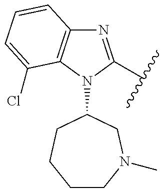 [SMILES: *c1nc2cccc(Cl)c2n1[C@H]1CCCCN(C)C1] | H | NH |
| 28d | 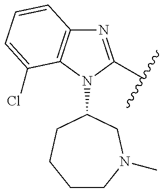 [SMILES: *c1nc2cccc(Cl)c2n1[C@H]1CCCCN(C)C1] | Me | NH |
| 28e | 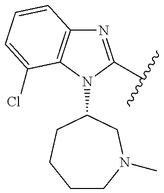 [SMILES: *c1nc2cccc(Cl)c2n1[C@H]1CCCCN(C)C1] | H | NMe |
| 28f | 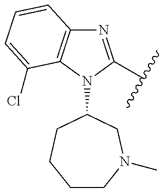 [SMILES: *c1nc2cccc(Cl)c2n1[C@H]1CCCCN(C)C1] | Me | NMe |
| 29a | 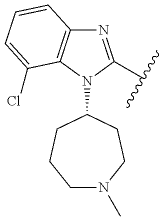 [SMILES: *c1nc2cccc(Cl)c2n1[C@H]1CCCN(C)CC1] | H | O |
| 29b | 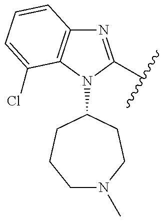 [SMILES: *c1nc2cccc(Cl)c2n1[C@H]1CCCN(C)CC1] | Me | O |
| 29c | 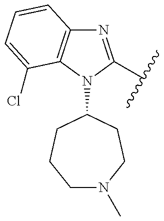 [SMILES: *c1nc2cccc(Cl)c2n1[C@H]1CCCN(C)CC1] | H | NH |
| 29d | 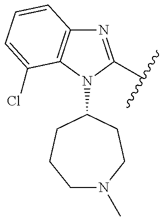 [SMILES: *c1nc2cccc(Cl)c2n1[C@H]1CCCN(C)CC1] | Me | NH |
| 29e | 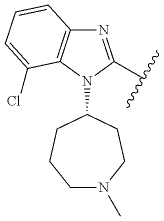 [SMILES: *c1nc2cccc(Cl)c2n1[C@H]1CCCN(C)CC1] | H | NMe |
| 29f | 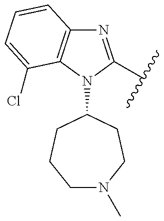 [SMILES: *c1nc2cccc(Cl)c2n1[C@H]1CCCN(C)CC1] | Me | NMe |
| 30a | 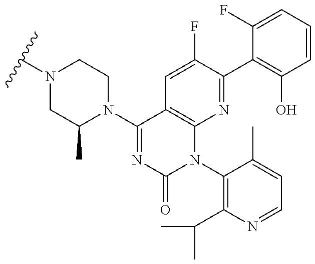 [SMILES: *N1CCN(c2nc(=O)n(-c3c(C)ccnc3C(C)C)c3nc(-c4c(O)cccc4F)c(F)cc23)[C@@H](C)C1] | N/A | O |
| 30b | 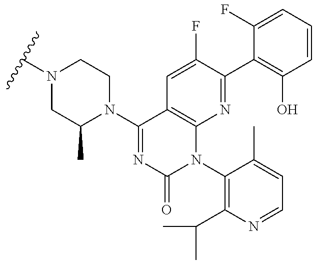 [SMILES: *N1CCN(c2nc(=O)n(-c3c(C)ccnc3C(C)C)c3nc(-c4c(O)cccc4F)c(F)cc23)[C@@H](C)C1] | N/A | NH |
| 30c | 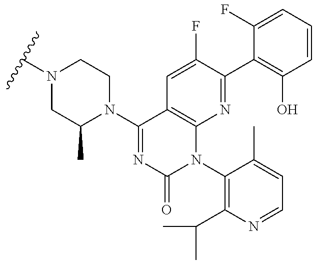 [SMILES: *N1CCN(c2nc(=O)n(-c3c(C)ccnc3C(C)C)c3nc(-c4c(O)cccc4F)c(F)cc23)[C@@H](C)C1] | N/A | NMe |
| 31a | 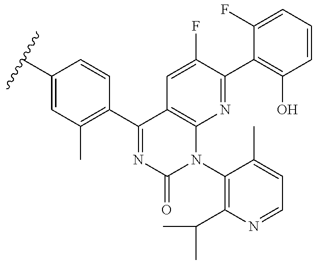 [SMILES: *c1ccc(-c2nc(=O)n(-c3c(C)ccnc3C(C)C)c3nc(-c4c(O)cccc4F)c(F)cc23)c(C)c1] | H | O |
| 31b | 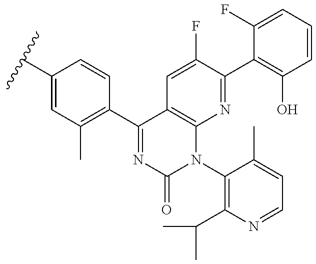 [SMILES: *c1ccc(-c2nc(=O)n(-c3c(C)ccnc3C(C)C)c3nc(-c4c(O)cccc4F)c(F)cc23)c(C)c1] | Me | O |
| 31c | 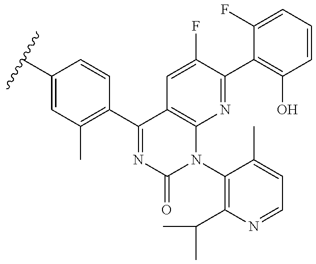 [SMILES: *c1ccc(-c2nc(=O)n(-c3c(C)ccnc3C(C)C)c3nc(-c4c(O)cccc4F)c(F)cc23)c(C)c1] | H | NH |
| 31d | 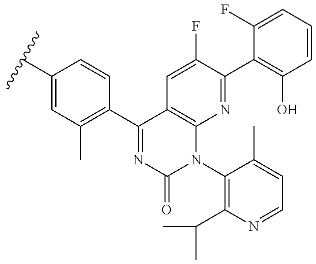 [SMILES: *c1ccc(-c2nc(=O)n(-c3c(C)ccnc3C(C)C)c3nc(-c4c(O)cccc4F)c(F)cc23)c(C)c1] | Me | NH |
| 31e | 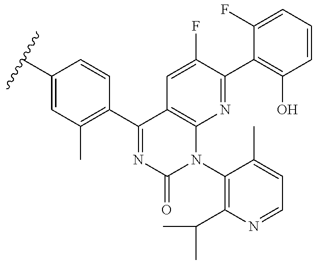 [SMILES: *c1ccc(-c2nc(=O)n(-c3c(C)ccnc3C(C)C)c3nc(-c4c(O)cccc4F)c(F)cc23)c(C)c1] | H | NMe |
| 31f | 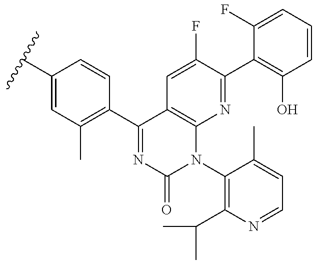 [SMILES: *c1ccc(-c2nc(=O)n(-c3c(C)ccnc3C(C)C)c3nc(-c4c(O)cccc4F)c(F)cc23)c(C)c1] | Me | NMe |
| 32a | 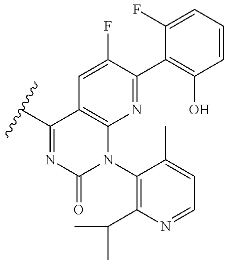 [SMILES: *c1nc(=O)n(-c2c(C)ccnc2C(C)C)c2nc(-c3c(O)cccc3F)c(F)cc12] | H | O |
| 32b | 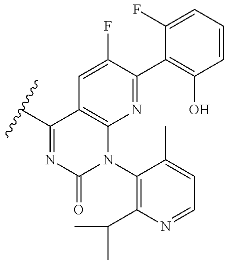 [SMILES: *c1nc(=O)n(-c2c(C)ccnc2C(C)C)c2nc(-c3c(O)cccc3F)c(F)cc12] | Me | O |
| 32c | 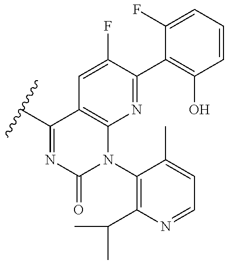 [SMILES: *c1nc(=O)n(-c2c(C)ccnc2C(C)C)c2nc(-c3c(O)cccc3F)c(F)cc12] | H | NH |
| 32d | 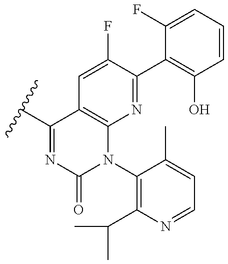 [SMILES: *c1nc(=O)n(-c2c(C)ccnc2C(C)C)c2nc(-c3c(O)cccc3F)c(F)cc12] | Me | NH |
| 32e | 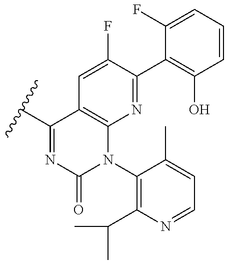 [SMILES: *c1nc(=O)n(-c2c(C)ccnc2C(C)C)c2nc(-c3c(O)cccc3F)c(F)cc12] | H | NMe |
| 32f | 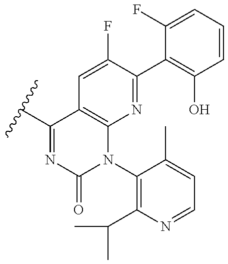 [SMILES: *c1nc(=O)n(-c2c(C)ccnc2C(C)C)c2nc(-c3c(O)cccc3F)c(F)cc12] | Me | NMe |
| 33a | 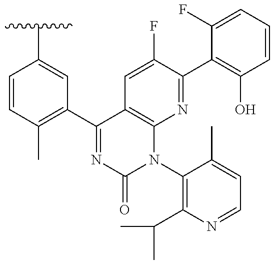 [SMILES: *c1ccc(C)c(-c2nc(=O)n(-c3c(C)ccnc3C(C)C)c3nc(-c4c(O)cccc4F)c(F)cc23)c1] | H | O |
| 33b | 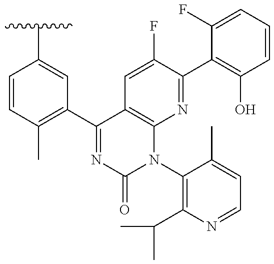 [SMILES: *c1ccc(C)c(-c2nc(=O)n(-c3c(C)ccnc3C(C)C)c3nc(-c4c(O)cccc4F)c(F)cc23)c1] | Me | O |
| 33c | 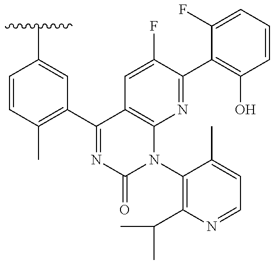 [SMILES: *c1ccc(C)c(-c2nc(=O)n(-c3c(C)ccnc3C(C)C)c3nc(-c4c(O)cccc4F)c(F)cc23)c1] | H | NH |
| 33d | 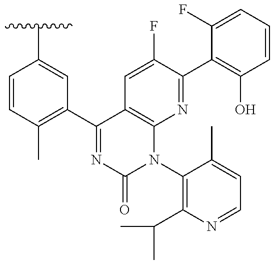 [SMILES: *c1ccc(C)c(-c2nc(=O)n(-c3c(C)ccnc3C(C)C)c3nc(-c4c(O)cccc4F)c(F)cc23)c1] | Me | NH |
| 33e | 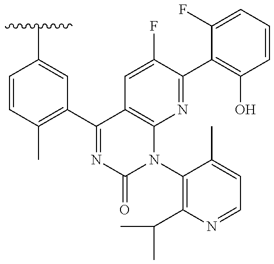 [SMILES: *c1ccc(C)c(-c2nc(=O)n(-c3c(C)ccnc3C(C)C)c3nc(-c4c(O)cccc4F)c(F)cc23)c1] | H | NMe |
| 33f | 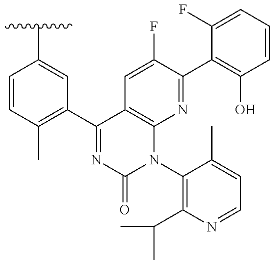 [SMILES: *c1ccc(C)c(-c2nc(=O)n(-c3c(C)ccnc3C(C)C)c3nc(-c4c(O)cccc4F)c(F)cc23)c1] | Me | NMe |
| 34a | 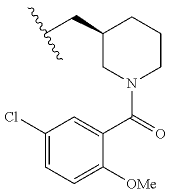 [SMILES: *C[C@H]1CCCN(C(=O)c2cc(Cl)ccc2OC)C1] | H | O |
| 34b | 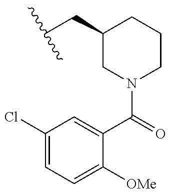 [SMILES: *C[C@H]1CCCN(C(=O)c2cc(Cl)ccc2OC)C1] | Me | O |
| 34c | 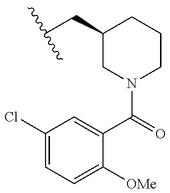 [SMILES: *C[C@H]1CCCN(C(=O)c2cc(Cl)ccc2OC)C1] | H | NH |
| 34d | 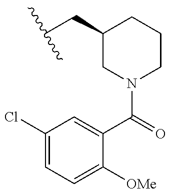 [SMILES: *C[C@H]1CCCN(C(=O)c2cc(Cl)ccc2OC)C1] | Me | NH |
| 34e | 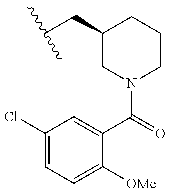 [SMILES: *C[C@H]1CCCN(C(=O)c2cc(Cl)ccc2OC)C1] | H | NMe |
| 34f | 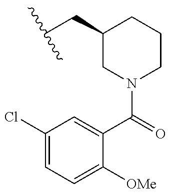 [SMILES: *C[C@H]1CCCN(C(=O)c2cc(Cl)ccc2OC)C1] | Me | NMe |
| 35a | 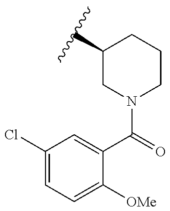 [SMILES: *[C@H]1CCCN(C(=O)c2cc(Cl)ccc2OC)C1] | H | O |
| 35b | 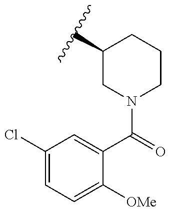 [SMILES: *[C@H]1CCCN(C(=O)c2cc(Cl)ccc2OC)C1] | Me | O |
| 35c | 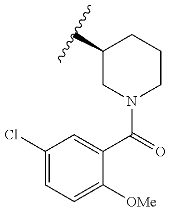 [SMILES: *[C@H]1CCCN(C(=O)c2cc(Cl)ccc2OC)C1] | H | NH |
| 35d | 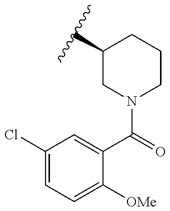 [SMILES: *[C@H]1CCCN(C(=O)c2cc(Cl)ccc2OC)C1] | Me | NH |
| 35e | 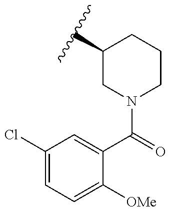 [SMILES: *[C@H]1CCCN(C(=O)c2cc(Cl)ccc2OC)C1] | H | NMe |
| 35f | 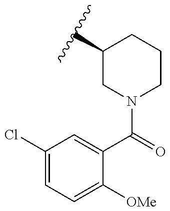 [SMILES: *[C@H]1CCCN(C(=O)c2cc(Cl)ccc2OC)C1] | Me | NMe |
| 36a | 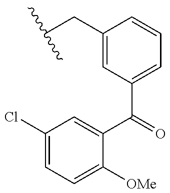 [SMILES: *Cc1cccc(C(=O)c2cc(Cl)ccc2OC)c1] | H | O |
| 36b | 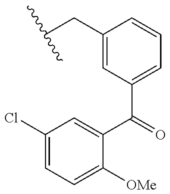 [SMILES: *Cc1cccc(C(=O)c2cc(Cl)ccc2OC)c1] | Me | O |
| 36c | 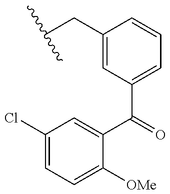 [SMILES: *Cc1cccc(C(=O)c2cc(Cl)ccc2OC)c1] | H | NH |
| 36d | 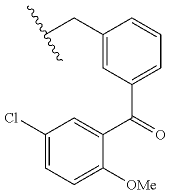 [SMILES: *Cc1cccc(C(=O)c2cc(Cl)ccc2OC)c1] | Me | NH |
| 36e | 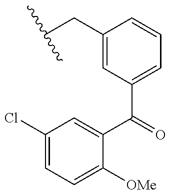 [SMILES: *Cc1cccc(C(=O)c2cc(Cl)ccc2OC)c1] | H | NMe |
| 36f | 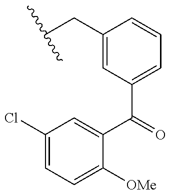 [SMILES: *Cc1cccc(C(=O)c2cc(Cl)ccc2OC)c1] | Me | NMe |
| 37a | 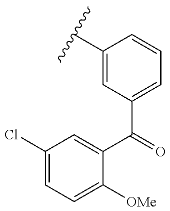 [SMILES: *c1cccc(C(=O)c2cc(Cl)ccc2OC)c1] | H | O |
| 37b | 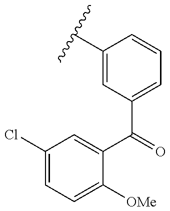 [SMILES: *c1cccc(C(=O)c2cc(Cl)ccc2OC)c1] | Me | O |
| 37c | 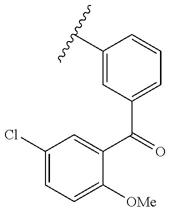 [SMILES: *c1cccc(C(=O)c2cc(Cl)ccc2OC)c1] | H | NH |
| 37d | 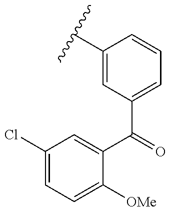 [SMILES: *c1cccc(C(=O)c2cc(Cl)ccc2OC)c1] | Me | NH |
| 37e | 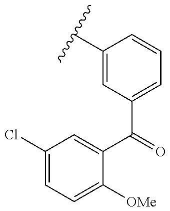 [SMILES: *c1cccc(C(=O)c2cc(Cl)ccc2OC)c1] | H | NMe |
| 37f | 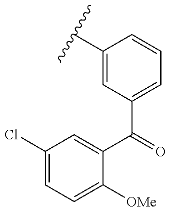 [SMILES: *c1cccc(C(=O)c2cc(Cl)ccc2OC)c1] | Me | NMe |
| 38a | 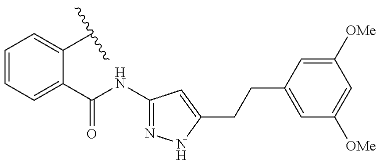 [SMILES: *c1ccccc1C(=O)Nc1cc(CCc2cc(OC)cc(OC)c2)[nH]n1] | H | O |
| 38b | 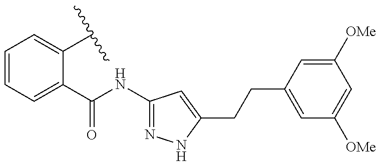 [SMILES: *c1ccccc1C(=O)Nc1cc(CCc2cc(OC)cc(OC)c2)[nH]n1] | Me | O |
| 38c | 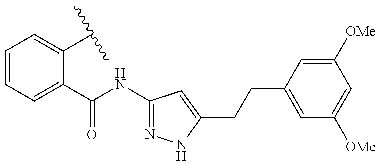 [SMILES: *c1ccccc1C(=O)Nc1cc(CCc2cc(OC)cc(OC)c2)[nH]n1] | H | NH |
| 38d | 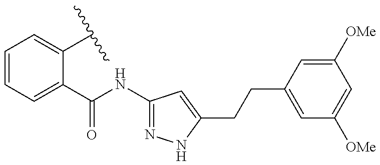 [SMILES: *c1ccccc1C(=O)Nc1cc(CCc2cc(OC)cc(OC)c2)[nH]n1] | Me | NH |
| 38e | 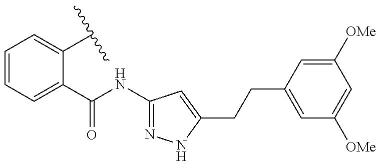 [SMILES: *c1ccccc1C(=O)Nc1cc(CCc2cc(OC)cc(OC)c2)[nH]n1] | H | NMe |
| 38f | 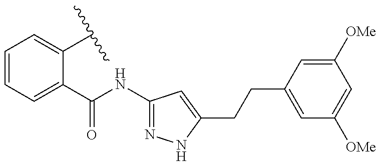 [SMILES: *c1ccccc1C(=O)Nc1cc(CCc2cc(OC)cc(OC)c2)[nH]n1] | Me | NMe |
| 39a | 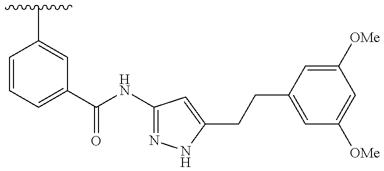 [SMILES: *c1cccc(C(=O)Nc2cc(CCc3cc(OC)cc(OC)c3)[nH]n2)c1] | H | O |
| 39b | 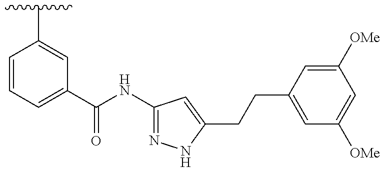 [SMILES: *c1cccc(C(=O)Nc2cc(CCc3cc(OC)cc(OC)c3)[nH]n2)c1] | Me | O |
| 39c | 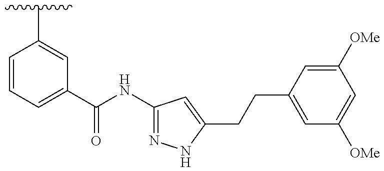 [SMILES: *c1cccc(C(=O)Nc2cc(CCc3cc(OC)cc(OC)c3)[nH]n2)c1] | H | NH |
| 39d | 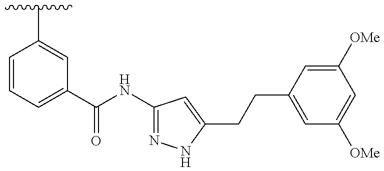 [SMILES: *c1cccc(C(=O)Nc2cc(CCc3cc(OC)cc(OC)c3)[nH]n2)c1] | Me | NH |
| 39e | 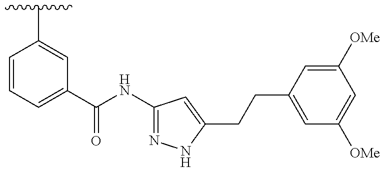 [SMILES: *c1cccc(C(=O)Nc2cc(CCc3cc(OC)cc(OC)c3)[nH]n2)c1] | H | NMe |
| 39f | 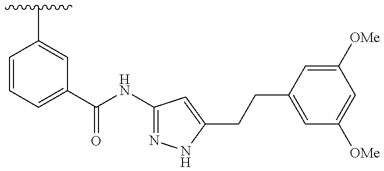 [SMILES: *c1cccc(C(=O)Nc2cc(CCc3cc(OC)cc(OC)c3)[nH]n2)c1] | Me | NMe |
| 40a | 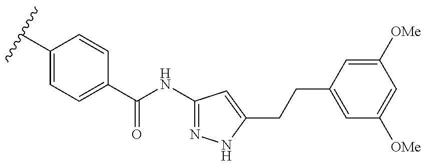 [SMILES: *c1ccc(C(=O)Nc2cc(CCc3cc(OC)cc(OC)c3)[nH]n2)cc1] | H | O |
| 40b | 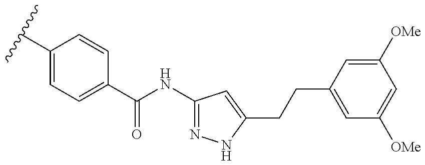 [SMILES: *c1ccc(C(=O)Nc2cc(CCc3cc(OC)cc(OC)c3)[nH]n2)cc1] | Me | O |
| 40c | 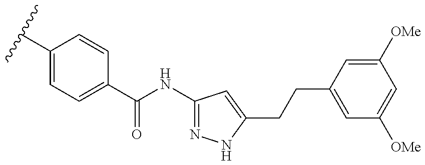 [SMILES: *c1ccc(C(=O)Nc2cc(CCc3cc(OC)cc(OC)c3)[nH]n2)cc1] | H | NH |
| 40d | 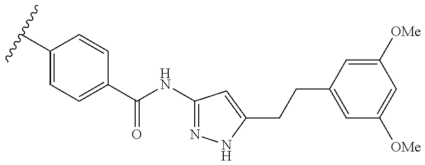 [SMILES: *c1ccc(C(=O)Nc2cc(CCc3cc(OC)cc(OC)c3)[nH]n2)cc1] | Me | NH |
| 40e | 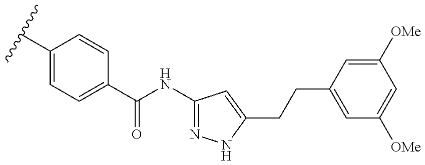 [SMILES: *c1ccc(C(=O)Nc2cc(CCc3cc(OC)cc(OC)c3)[nH]n2)cc1] | H | NMe |
| 40f | 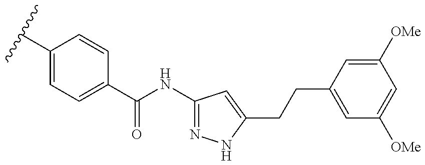 [SMILES: *c1ccc(C(=O)Nc2cc(CCc3cc(OC)cc(OC)c3)[nH]n2)cc1] | Me | NMe |
| 41a | 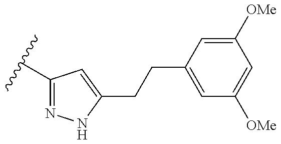 [SMILES: *c1cc(CCc2cc(OC)cc(OC)c2)[nH]n1] | H | O |
| 41b | 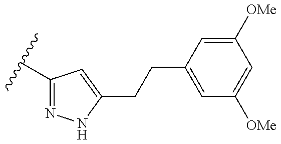 [SMILES: *c1cc(CCc2cc(OC)cc(OC)c2)[nH]n1] | Me | O |
| 41c | 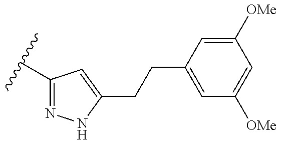 [SMILES: *c1cc(CCc2cc(OC)cc(OC)c2)[nH]n1] | H | NH |
| 41d | 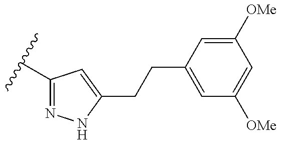 [SMILES: *c1cc(CCc2cc(OC)cc(OC)c2)[nH]n1] | Me | NH |
| 41e | 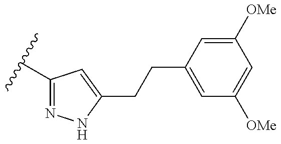 [SMILES: *c1cc(CCc2cc(OC)cc(OC)c2)[nH]n1] | H | NMe |
| 41f | 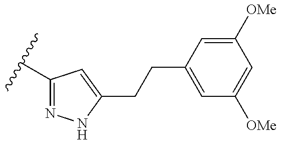 [SMILES: *c1cc(CCc2cc(OC)cc(OC)c2)[nH]n1] | Me | NMe |
| 42a | 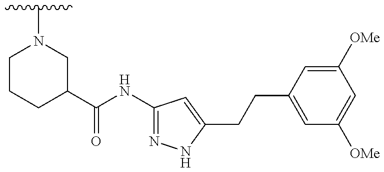 [SMILES: *N1CCCC(C(=O)Nc2cc(CCc3cc(OC)cc(OC)c3)[nH]n2)C1] | N/A | O |
| 42b | 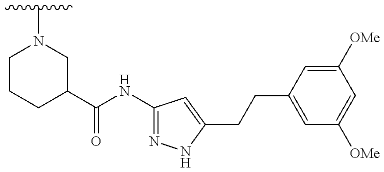 [SMILES: *N1CCCC(C(=O)Nc2cc(CCc3cc(OC)cc(OC)c3)[nH]n2)C1] | N/A | NH |
| 42c | 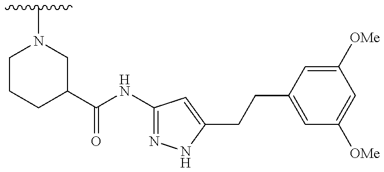 [SMILES: *N1CCCC(C(=O)Nc2cc(CCc3cc(OC)cc(OC)c3)[nH]n2)C1] | N/A | NMe |
| 43a | 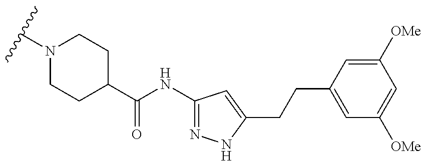 [SMILES: *N1CCC(C(=O)Nc2cc(CCc3cc(OC)cc(OC)c3)[nH]n2)CC1] | N/A | O |
| 43b | 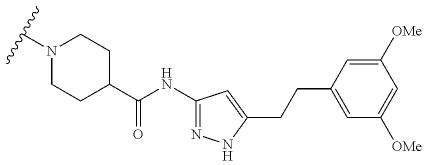 [SMILES: *N1CCC(C(=O)Nc2cc(CCc3cc(OC)cc(OC)c3)[nH]n2)CC1] | N/A | NH |
| 43c | 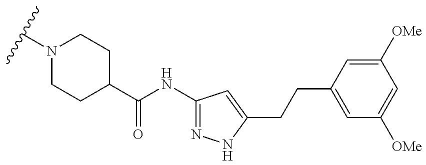 [SMILES: *N1CCC(C(=O)Nc2cc(CCc3cc(OC)cc(OC)c3)[nH]n2)CC1] | N/A | NMe |
| 44a | 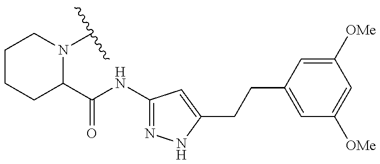 [SMILES: *N1CCCCC1C(=O)Nc1cc(CCc2cc(OC)cc(OC)c2)[nH]n1] | N/A | O |
| 44b | 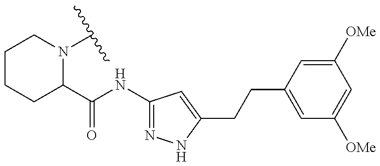 [SMILES: *N1CCCCC1C(=O)Nc1cc(CCc2cc(OC)cc(OC)c2)[nH]n1] | N/A | NH |
| 44c | 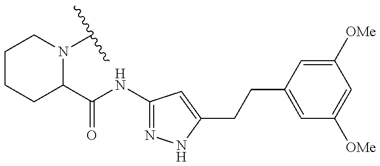 [SMILES: *N1CCCCC1C(=O)Nc1cc(CCc2cc(OC)cc(OC)c2)[nH]n1] | N/A | NMe |
| 45a | 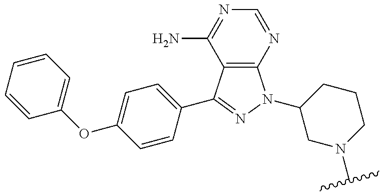 [SMILES: *N1CCCC(n2nc(-c3ccc(Oc4ccccc4)cc3)c3c(N)ncnc32)C1] | N/A | O |
| 45b | 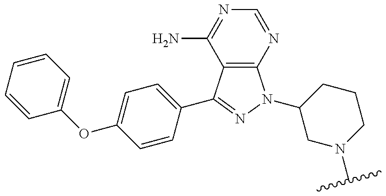 [SMILES: *N1CCCC(n2nc(-c3ccc(Oc4ccccc4)cc3)c3c(N)ncnc32)C1] | N/A | NH |
| 45c | 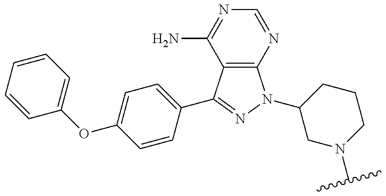 [SMILES: *N1CCCC(n2nc(-c3ccc(Oc4ccccc4)cc3)c3c(N)ncnc32)C1] | N/A | NMe |
| 46a | 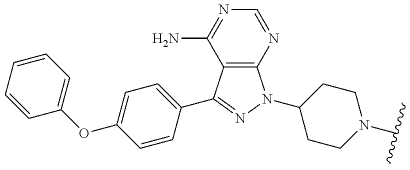 [SMILES: *N1CCC(n2nc(-c3ccc(Oc4ccccc4)cc3)c3c(N)ncnc32)CC1] | N/A | O |
| 46b | 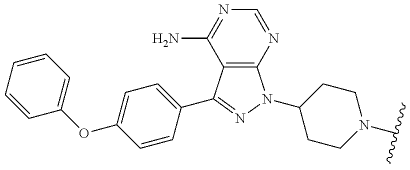 [SMILES: *N1CCC(n2nc(-c3ccc(Oc4ccccc4)cc3)c3c(N)ncnc32)CC1] | N/A | NH |
| 46c | 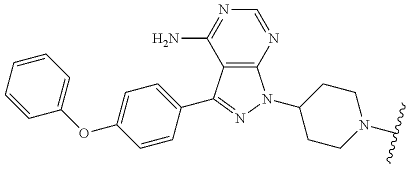 [SMILES: *N1CCC(n2nc(-c3ccc(Oc4ccccc4)cc3)c3c(N)ncnc32)CC1] | N/A | NMe |
| 47a | 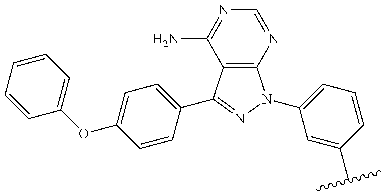 [SMILES: *c1cccc(-n2nc(-c3ccc(Oc4ccccc4)cc3)c3c(N)ncnc32)c1] | H | O |
| 47b | 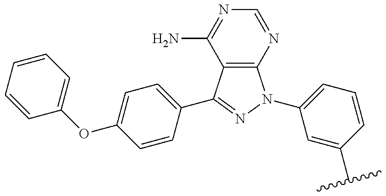 [SMILES: *c1cccc(-n2nc(-c3ccc(Oc4ccccc4)cc3)c3c(N)ncnc32)c1] | Me | O |
| 47c | 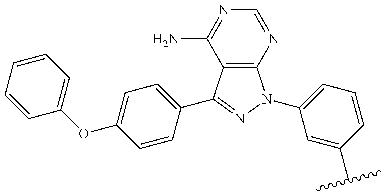 [SMILES: *c1cccc(-n2nc(-c3ccc(Oc4ccccc4)cc3)c3c(N)ncnc32)c1] | H | NH |
| 47d | 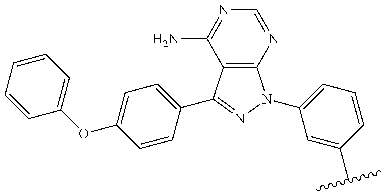 [SMILES: *c1cccc(-n2nc(-c3ccc(Oc4ccccc4)cc3)c3c(N)ncnc32)c1] | Me | NH |
| 47e | 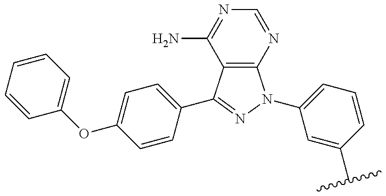 [SMILES: *c1cccc(-n2nc(-c3ccc(Oc4ccccc4)cc3)c3c(N)ncnc32)c1] | H | NMe |
| 47f | 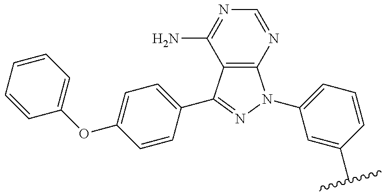 [SMILES: *c1cccc(-n2nc(-c3ccc(Oc4ccccc4)cc3)c3c(N)ncnc32)c1] | Me | NMe |
| 48a | 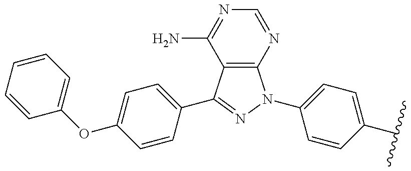 [SMILES: *c1ccc(-n2nc(-c3ccc(Oc4ccccc4)cc3)c3c(N)ncnc32)cc1] | H | O |
| 48b | 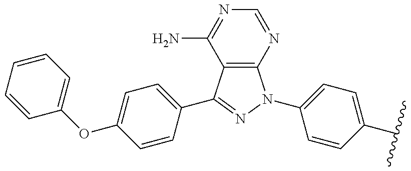 [SMILES: *c1ccc(-n2nc(-c3ccc(Oc4ccccc4)cc3)c3c(N)ncnc32)cc1] | Me | O |
| 48c | 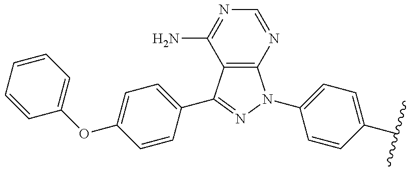 [SMILES: *c1ccc(-n2nc(-c3ccc(Oc4ccccc4)cc3)c3c(N)ncnc32)cc1] | H | NH |
| 48d | 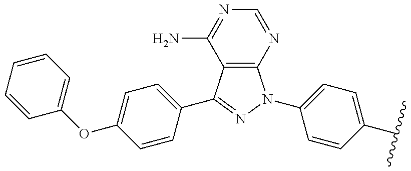 [SMILES: *c1ccc(-n2nc(-c3ccc(Oc4ccccc4)cc3)c3c(N)ncnc32)cc1] | Me | NH |
| 48e | 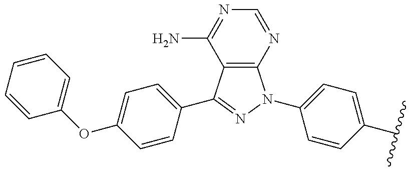 [SMILES: *c1ccc(-n2nc(-c3ccc(Oc4ccccc4)cc3)c3c(N)ncnc32)cc1] | H | NMe |
| 48f | 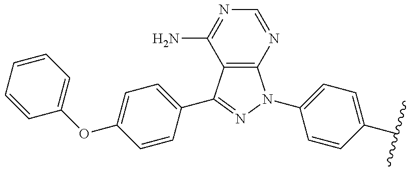 [SMILES: *c1ccc(-n2nc(-c3ccc(Oc4ccccc4)cc3)c3c(N)ncnc32)cc1] | Me | NMe |
| 49a | 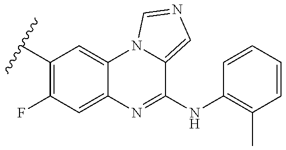 [SMILES: *c1cc2c(cc1F)nc(Nc1ccccc1C)c1cncn12] | H | O |
| 49b | 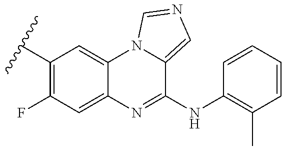 [SMILES: *c1cc2c(cc1F)nc(Nc1ccccc1C)c1cncn12] | Me | O |
| 49c | 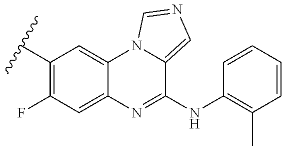 [SMILES: *c1cc2c(cc1F)nc(Nc1ccccc1C)c1cncn12] | H | NH |
| 49d | 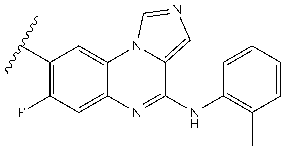 [SMILES: *c1cc2c(cc1F)nc(Nc1ccccc1C)c1cncn12] | Me | NH |
| 49e | 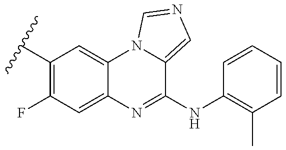 [SMILES: *c1cc2c(cc1F)nc(Nc1ccccc1C)c1cncn12] | H | NMe |
| 49f | 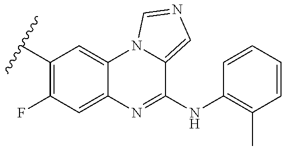 [SMILES: *c1cc2c(cc1F)nc(Nc1ccccc1C)c1cncn12] | Me | NMe |
| 50a | 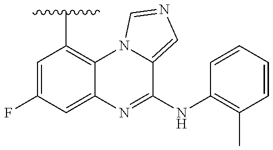 [SMILES: *c1cc(F)cc2nc(Nc3ccccc3C)c3cncn3c12] | H | O |
| 50b | 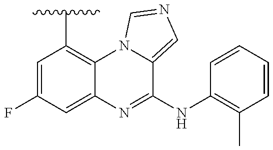 [SMILES: *c1cc(F)cc2nc(Nc3ccccc3C)c3cncn3c12] | Me | O |
| 50c | 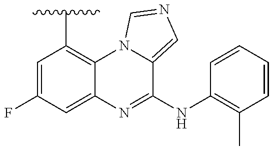 [SMILES: *c1cc(F)cc2nc(Nc3ccccc3C)c3cncn3c12] | H | NH |
| 50d | 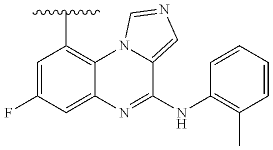 [SMILES: *c1cc(F)cc2nc(Nc3ccccc3C)c3cncn3c12] | Me | NH |
| 50e | 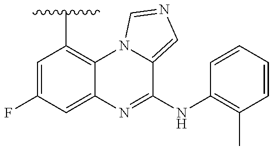 [SMILES: *c1cc(F)cc2nc(Nc3ccccc3C)c3cncn3c12] | H | NMe |
| 50f | 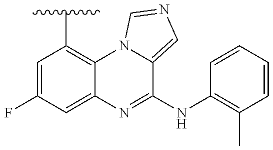 [SMILES: *c1cc(F)cc2nc(Nc3ccccc3C)c3cncn3c12] | Me | NMe |
| 51a | 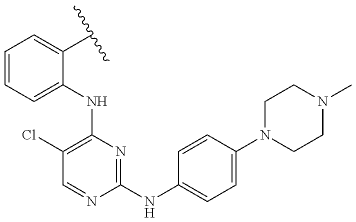 [SMILES: *c1ccccc1Nc1nc(Nc2ccc(N3CCN(C)CC3)cc2)ncc1Cl] | H | O |
| 51b | 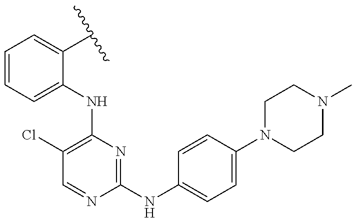 [SMILES: *c1ccccc1Nc1nc(Nc2ccc(N3CCN(C)CC3)cc2)ncc1Cl] | Me | O |
| 51c | 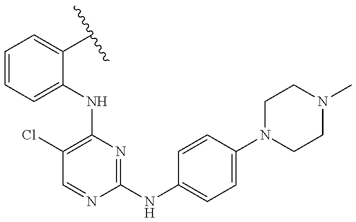 [SMILES: *c1ccccc1Nc1nc(Nc2ccc(N3CCN(C)CC3)cc2)ncc1Cl] | H | NH |
| 51d | 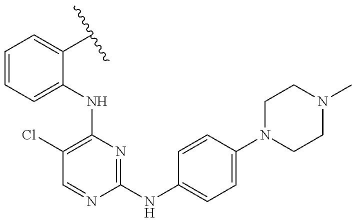 [SMILES: *c1ccccc1Nc1nc(Nc2ccc(N3CCN(C)CC3)cc2)ncc1Cl] | Me | NH |
| 51e | 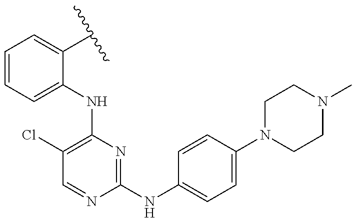 [SMILES: *c1ccccc1Nc1nc(Nc2ccc(N3CCN(C)CC3)cc2)ncc1Cl] | H | NMe |
| 51f | 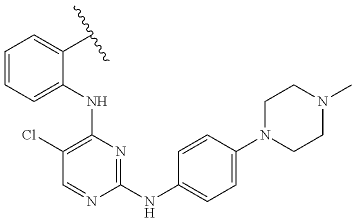 [SMILES: *c1ccccc1Nc1nc(Nc2ccc(N3CCN(C)CC3)cc2)ncc1Cl] | Me | NMe |
| 52a | 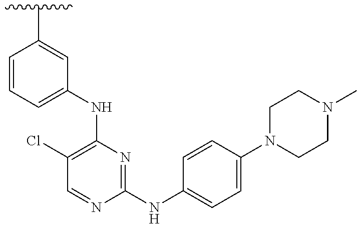 [SMILES: *c1cccc(Nc2nc(Nc3ccc(N4CCN(C)CC4)cc3)ncc2Cl)c1] | H | O |
| 52b | 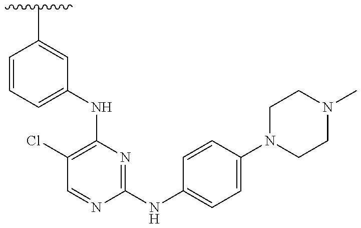 [SMILES: *c1cccc(Nc2nc(Nc3ccc(N4CCN(C)CC4)cc3)ncc2Cl)c1] | Me | O |
| 52c | 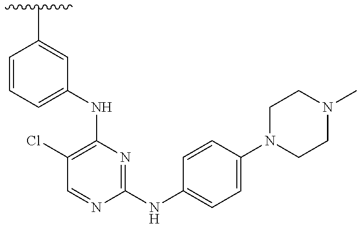 [SMILES: *c1cccc(Nc2nc(Nc3ccc(N4CCN(C)CC4)cc3)ncc2Cl)c1] | H | NH |
| 52d | 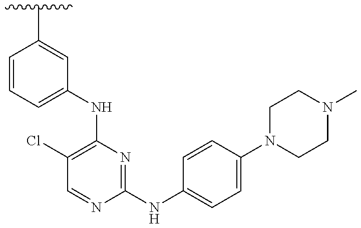 [SMILES: *c1cccc(Nc2nc(Nc3ccc(N4CCN(C)CC4)cc3)ncc2Cl)c1] | Me | NH |
| 52e | 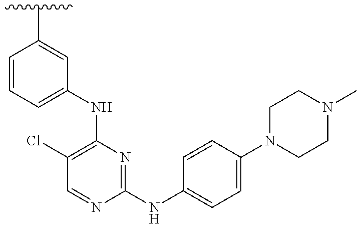 [SMILES: *c1cccc(Nc2nc(Nc3ccc(N4CCN(C)CC4)cc3)ncc2Cl)c1] | H | NMe |
| 52f | 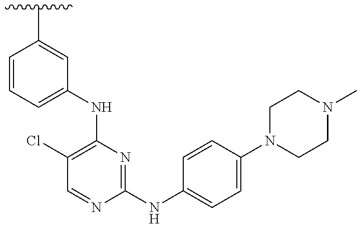 [SMILES: *c1cccc(Nc2nc(Nc3ccc(N4CCN(C)CC4)cc3)ncc2Cl)c1] | Me | NMe |
| 53a | 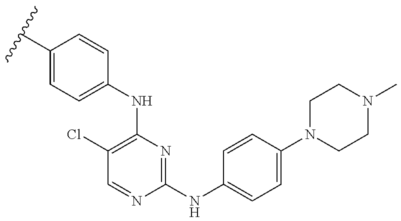 [SMILES: *c1ccc(Nc2nc(Nc3ccc(N4CCN(C)CC4)cc3)ncc2Cl)cc1] | H | O |
| 53b | 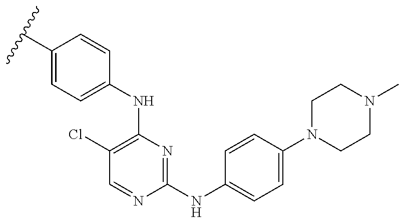 [SMILES: *c1ccc(Nc2nc(Nc3ccc(N4CCN(C)CC4)cc3)ncc2Cl)cc1] | Me | O |
| 53c | 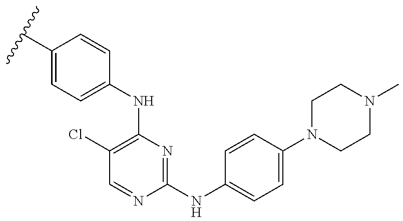 [SMILES: *c1ccc(Nc2nc(Nc3ccc(N4CCN(C)CC4)cc3)ncc2Cl)cc1] | H | NH |
| 53d | 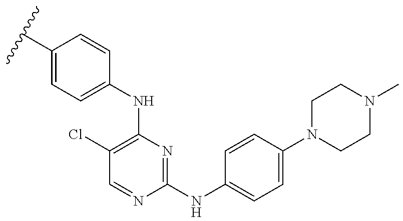 [SMILES: *c1ccc(Nc2nc(Nc3ccc(N4CCN(C)CC4)cc3)ncc2Cl)cc1] | Me | NH |
| 53e | 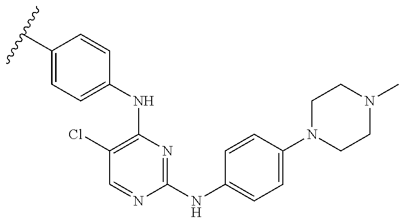 [SMILES: *c1ccc(Nc2nc(Nc3ccc(N4CCN(C)CC4)cc3)ncc2Cl)cc1] | H | NMe |
| 53f | 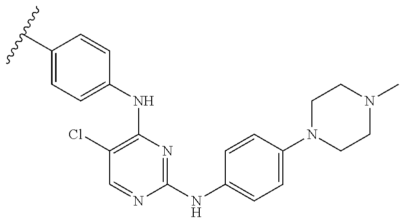 [SMILES: *c1ccc(Nc2nc(Nc3ccc(N4CCN(C)CC4)cc3)ncc2Cl)cc1] | Me | NMe |
| 54a | 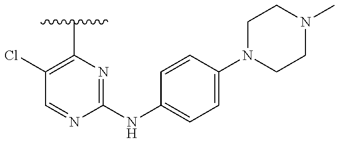 [SMILES: *c1nc(Nc2ccc(N3CCN(C)CC3)cc2)ncc1Cl] | H | O |
| 54b | 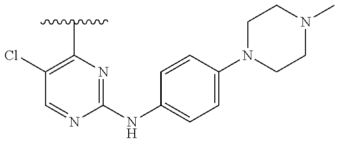 [SMILES: *c1nc(Nc2ccc(N3CCN(C)CC3)cc2)ncc1Cl] | Me | O |
| 54c | 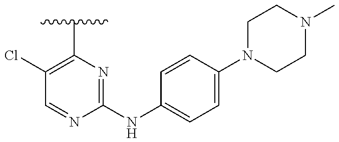 [SMILES: *c1nc(Nc2ccc(N3CCN(C)CC3)cc2)ncc1Cl] | H | NH |
| 54d | 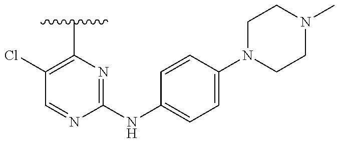 [SMILES: *c1nc(Nc2ccc(N3CCN(C)CC3)cc2)ncc1Cl] | Me | NH |
| 54e | 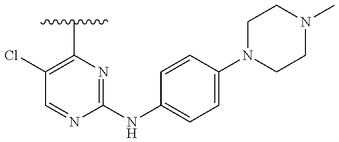 [SMILES: *c1nc(Nc2ccc(N3CCN(C)CC3)cc2)ncc1Cl] | H | NMe |
| 54f | 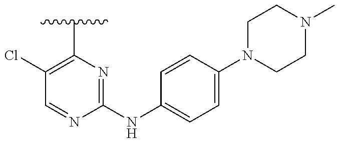 [SMILES: *c1nc(Nc2ccc(N3CCN(C)CC3)cc2)ncc1Cl] | Me | NMe |
| 55a | 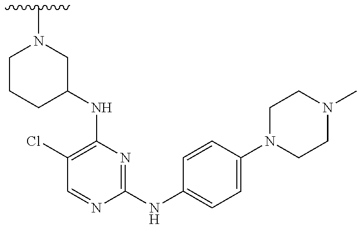 [SMILES: *N1CCCC(Nc2nc(Nc3ccc(N4CCN(C)CC4)cc3)ncc2Cl)C1] | N/A | O |
| 55b | 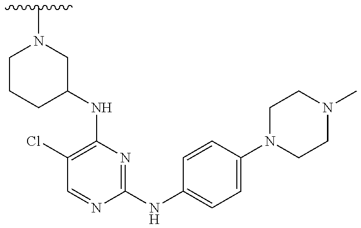 [SMILES: *N1CCCC(Nc2nc(Nc3ccc(N4CCN(C)CC4)cc3)ncc2Cl)C1] | N/A | NH |
| 55c | 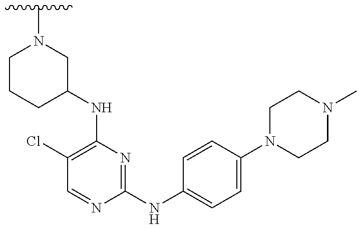 [SMILES: *N1CCCC(Nc2nc(Nc3ccc(N4CCN(C)CC4)cc3)ncc2Cl)C1] | N/A | NMe |
| 56a | 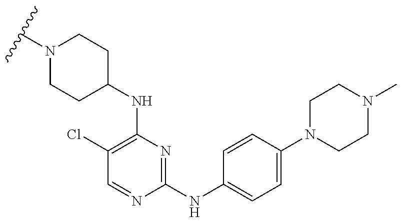 [SMILES: *N1CCC(Nc2nc(Nc3ccc(N4CCN(C)CC4)cc3)ncc2Cl)CC1] | N/A | O |
| 56b | 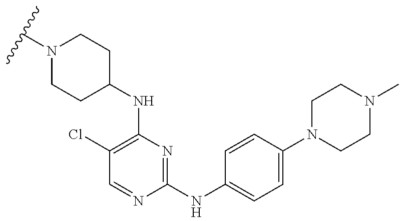 [SMILES: *N1CCC(Nc2nc(Nc3ccc(N4CCN(C)CC4)cc3)ncc2Cl)CC1] | N/A | NH |
| 56c | 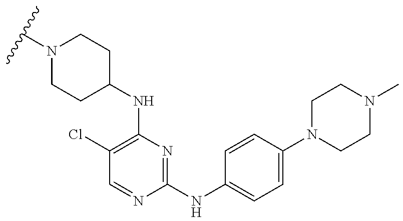 [SMILES: *N1CCC(Nc2nc(Nc3ccc(N4CCN(C)CC4)cc3)ncc2Cl)CC1] | N/A | NMe |
| 57a | 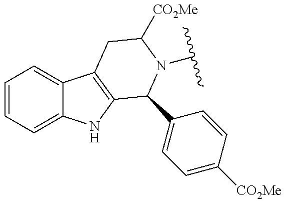 [SMILES: *N1C(C(=O)OC)Cc2c([nH]c3ccccc23)[C@@H]1c1ccc(C(=O)OC)cc1] | N/A | O |
| 57b | 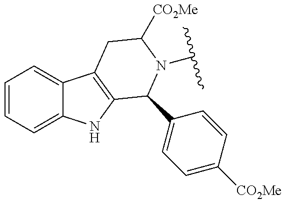 [SMILES: *N1C(C(=O)OC)Cc2c([nH]c3ccccc23)[C@@H]1c1ccc(C(=O)OC)cc1] | N/A | NH |
| 57c | 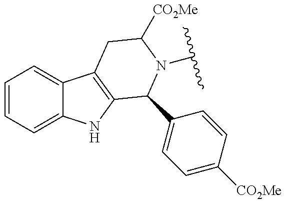 [SMILES: *N1C(C(=O)OC)Cc2c([nH]c3ccccc23)[C@@H]1c1ccc(C(=O)OC)cc1] | N/A | NMe |
| 58a | 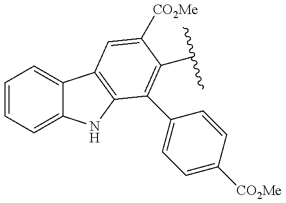 [SMILES: *c1c(C(=O)OC)cc2c([nH]c3ccccc32)c1-c1ccc(C(=O)OC)cc1] | H | O |
| 58b | 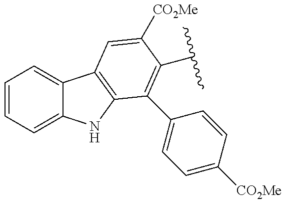 [SMILES: *c1c(C(=O)OC)cc2c([nH]c3ccccc32)c1-c1ccc(C(=O)OC)cc1] | Me | O |
| 58c | 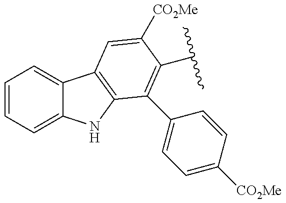 [SMILES: *c1c(C(=O)OC)cc2c([nH]c3ccccc32)c1-c1ccc(C(=O)OC)cc1] | H | NH |
| 58d | 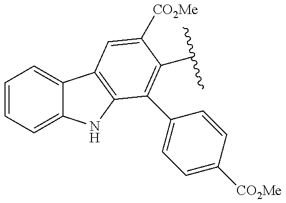 [SMILES: *c1c(C(=O)OC)cc2c([nH]c3ccccc32)c1-c1ccc(C(=O)OC)cc1] | Me | NH |
| 58e | 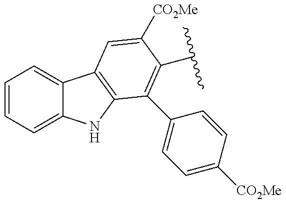 [SMILES: *c1c(C(=O)OC)cc2c([nH]c3ccccc32)c1-c1ccc(C(=O)OC)cc1] | H | NMe |
| 58f | 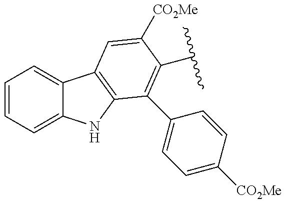 [SMILES: *c1c(C(=O)OC)cc2c([nH]c3ccccc32)c1-c1ccc(C(=O)OC)cc1] | Me | NMe |
| 59a | 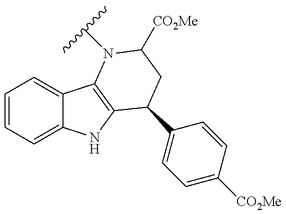 [SMILES: *N1c2c([nH]c3ccccc23)[C@H](c2ccc(C(=O)OC)cc2)CC1C(=O)OC] | N/A | O |
| 59b | 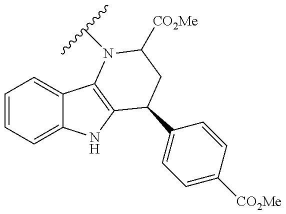 [SMILES: *N1c2c([nH]c3ccccc23)[C@H](c2ccc(C(=O)OC)cc2)CC1C(=O)OC] | N/A | NH |
| 59c | 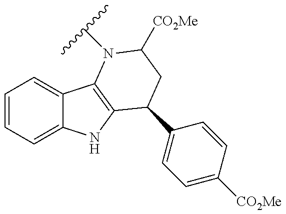 [SMILES: *N1c2c([nH]c3ccccc23)[C@H](c2ccc(C(=O)OC)cc2)CC1C(=O)OC] | N/A | NMe |
| 60a | 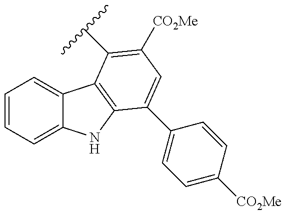 [SMILES: *c1c(C(=O)OC)cc(-c2ccc(C(=O)OC)cc2)c2[nH]c3ccccc3c12] | H | O |
| 60b | 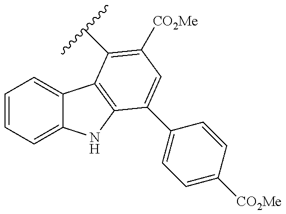 [SMILES: *c1c(C(=O)OC)cc(-c2ccc(C(=O)OC)cc2)c2[nH]c3ccccc3c12] | Me | O |
| 60c | 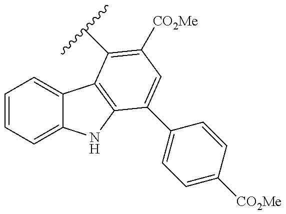 [SMILES: *c1c(C(=O)OC)cc(-c2ccc(C(=O)OC)cc2)c2[nH]c3ccccc3c12] | H | NH |
| 60d | 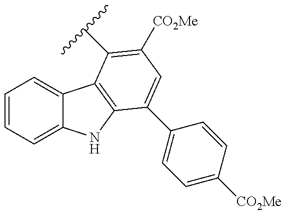 [SMILES: *c1c(C(=O)OC)cc(-c2ccc(C(=O)OC)cc2)c2[nH]c3ccccc3c12] | Me | NH |
| 60e | 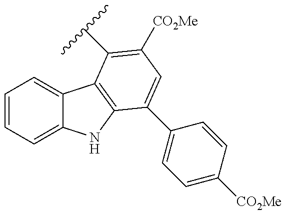 [SMILES: *c1c(C(=O)OC)cc(-c2ccc(C(=O)OC)cc2)c2[nH]c3ccccc3c12] | H | NMe |
| 60f | 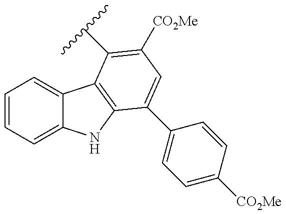 [SMILES: *c1c(C(=O)OC)cc(-c2ccc(C(=O)OC)cc2)c2[nH]c3ccccc3c12] | Me | NMe |
| 61a | 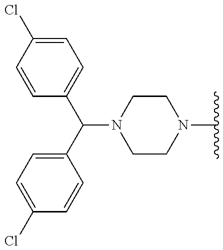 [SMILES: *N1CCN(C(c2ccc(Cl)cc2)c2ccc(Cl)cc2)CC1] | N/A | O |
| 61b | 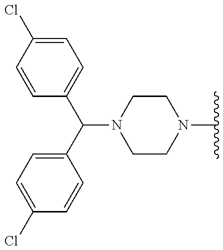 [SMILES: *N1CCN(C(c2ccc(Cl)cc2)c2ccc(Cl)cc2)CC1] | N/A | NH |
| 61c | 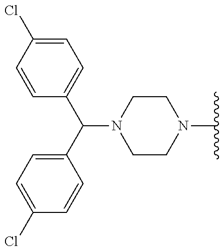 [SMILES: *N1CCN(C(c2ccc(Cl)cc2)c2ccc(Cl)cc2)CC1] | N/A | NMe |
| 62a | 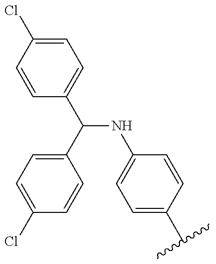 [SMILES: *c1ccc(NC(c2ccc(Cl)cc2)c2ccc(Cl)cc2)cc1] | H | O |
| 62b | 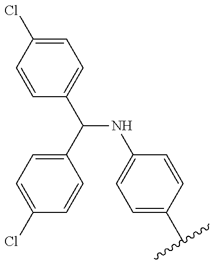 [SMILES: *c1ccc(NC(c2ccc(Cl)cc2)c2ccc(Cl)cc2)cc1] | Me | O |
| 62c | 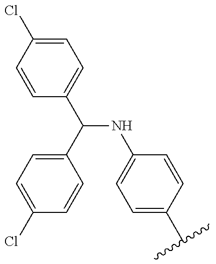 [SMILES: *c1ccc(NC(c2ccc(Cl)cc2)c2ccc(Cl)cc2)cc1] | H | NH |
| 62d | 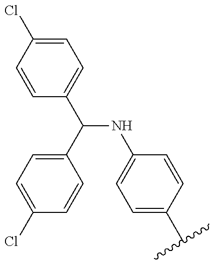 [SMILES: *c1ccc(NC(c2ccc(Cl)cc2)c2ccc(Cl)cc2)cc1] | Me | NH |
| 62e | 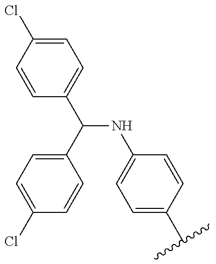 [SMILES: *c1ccc(NC(c2ccc(Cl)cc2)c2ccc(Cl)cc2)cc1] | H | NMe |
| 62f | 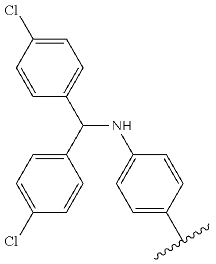 [SMILES: *c1ccc(NC(c2ccc(Cl)cc2)c2ccc(Cl)cc2)cc1] | Me | NMe |
| 63a | 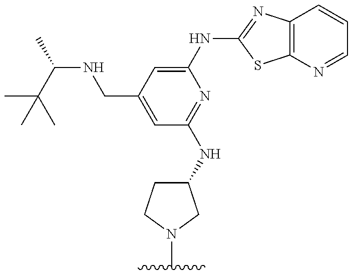 [SMILES: *N1CC[C@H](Nc2cc(CN[C@@H](C)C(C)(C)C)cc(Nc3nc4cccnc4s3)n2)C1] | N/A | O |
| 63b | 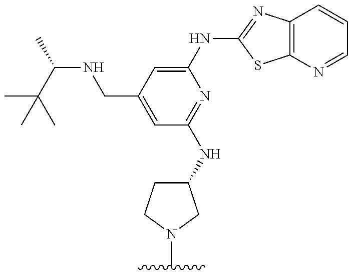 [SMILES: *N1CC[C@H](Nc2cc(CN[C@@H](C)C(C)(C)C)cc(Nc3nc4cccnc4s3)n2)C1] | N/A | NH |
| 63c | 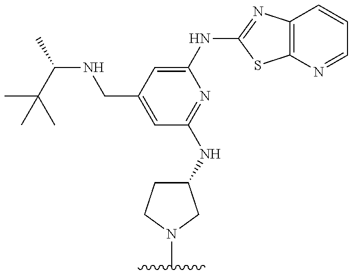 [SMILES: *N1CC[C@H](Nc2cc(CN[C@@H](C)C(C)(C)C)cc(Nc3nc4cccnc4s3)n2)C1] | N/A | NMe |
| 64a | 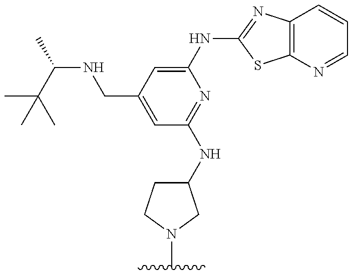 [SMILES: *N1CCC(Nc2cc(CN[C@@H](C)C(C)(C)C)cc(Nc3nc4cccnc4s3)n2)C1] | N/A | O |
| 64b | 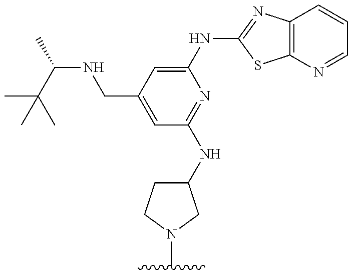 [SMILES: *N1CCC(Nc2cc(CN[C@@H](C)C(C)(C)C)cc(Nc3nc4cccnc4s3)n2)C1] | N/A | NH |
| 64c | 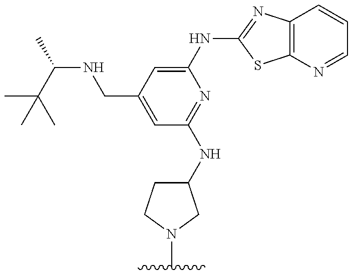 [SMILES: *N1CCC(Nc2cc(CN[C@@H](C)C(C)(C)C)cc(Nc3nc4cccnc4s3)n2)C1] | N/A | NMe |
| 65a | 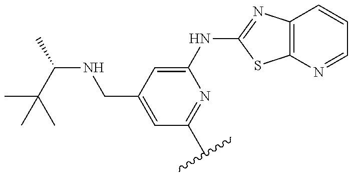 [SMILES: *c1cc(CN[C@@H](C)C(C)(C)C)cc(Nc2nc3cccnc3s2)n1] | H | O |
| 65b | 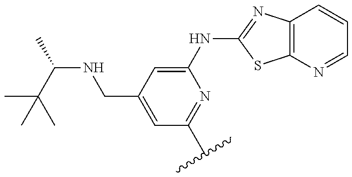 [SMILES: *c1cc(CN[C@@H](C)C(C)(C)C)cc(Nc2nc3cccnc3s2)n1] | Me | O |
| 65c | 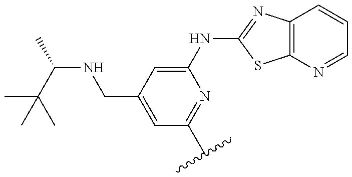 [SMILES: *c1cc(CN[C@@H](C)C(C)(C)C)cc(Nc2nc3cccnc3s2)n1] | H | NH |
| 65d | 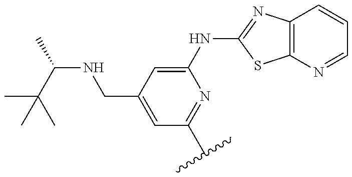 [SMILES: *c1cc(CN[C@@H](C)C(C)(C)C)cc(Nc2nc3cccnc3s2)n1] | Me | NH |
| 65e | 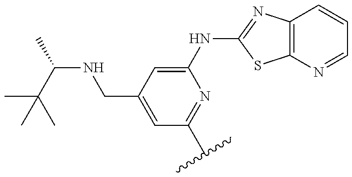 [SMILES: *c1cc(CN[C@@H](C)C(C)(C)C)cc(Nc2nc3cccnc3s2)n1] | H | NMe |
| 65f | 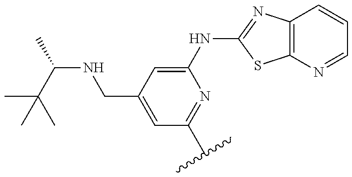 [SMILES: *c1cc(CN[C@@H](C)C(C)(C)C)cc(Nc2nc3cccnc3s2)n1] | Me | NMe |
| 66a | 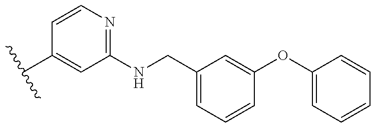 [SMILES: *c1ccnc(NCc2cccc(Oc3ccccc3)c2)c1] | H | O |
| 66b | 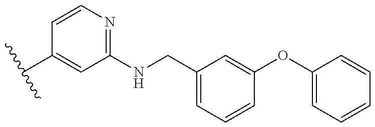 [SMILES: *c1ccnc(NCc2cccc(Oc3ccccc3)c2)c1] | Me | O |
| 66c | 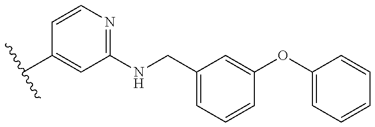 [SMILES: *c1ccnc(NCc2cccc(Oc3ccccc3)c2)c1] | H | NH |
| 66d | 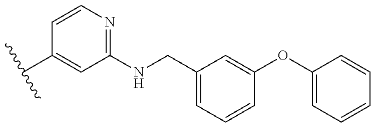 [SMILES: *c1ccnc(NCc2cccc(Oc3ccccc3)c2)c1] | Me | NH |
| 66e | 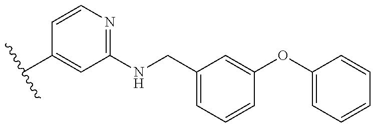 [SMILES: *c1ccnc(NCc2cccc(Oc3ccccc3)c2)c1] | H | NMe |
| 66f | 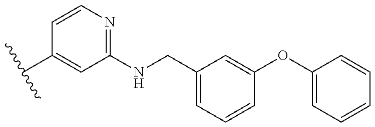 [SMILES: *c1ccnc(NCc2cccc(Oc3ccccc3)c2)c1] | Me | NMe |
| 67a | 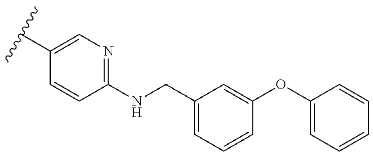 [SMILES: *c1ccc(NCc2cccc(Oc3ccccc3)c2)nc1] | H | O |
| 67b | 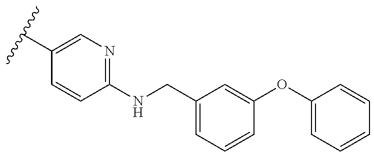 [SMILES: *c1ccc(NCc2cccc(Oc3ccccc3)c2)nc1] | Me | O |
| 67c | 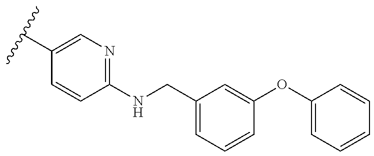 [SMILES: *c1ccc(NCc2cccc(Oc3ccccc3)c2)nc1] | H | NH |
| 67d | 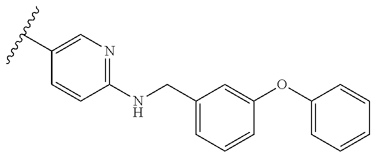 [SMILES: *c1ccc(NCc2cccc(Oc3ccccc3)c2)nc1] | Me | NH |
| 67e | 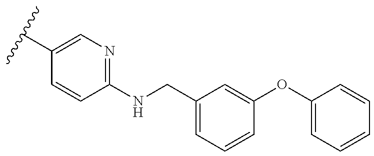 [SMILES: *c1ccc(NCc2cccc(Oc3ccccc3)c2)nc1] | H | NMe |
| 67f | 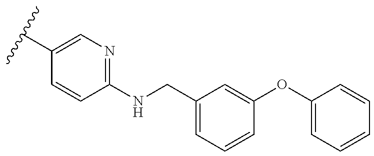 [SMILES: *c1ccc(NCc2cccc(Oc3ccccc3)c2)nc1] | Me | NMe |
| 68a | 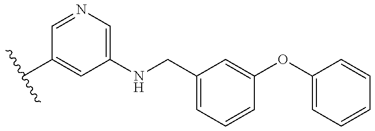 [SMILES: *c1cncc(NCc2cccc(Oc3ccccc3)c2)c1] | H | O |
| 68b | 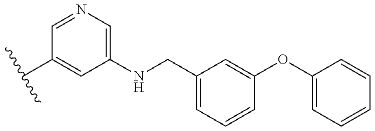 [SMILES: *c1cncc(NCc2cccc(Oc3ccccc3)c2)c1] | Me | O |
| 68c | 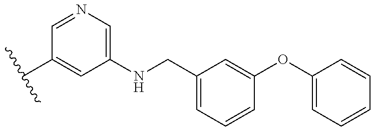 [SMILES: *c1cncc(NCc2cccc(Oc3ccccc3)c2)c1] | H | NH |
| 68d | 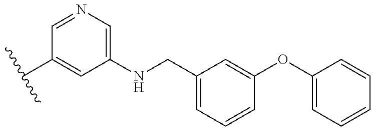 [SMILES: *c1cncc(NCc2cccc(Oc3ccccc3)c2)c1] | Me | NH |
| 68e | 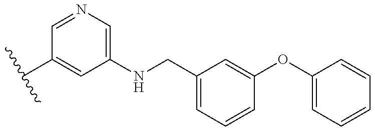 [SMILES: *c1cncc(NCc2cccc(Oc3ccccc3)c2)c1] | H | NMe |
| 68f | 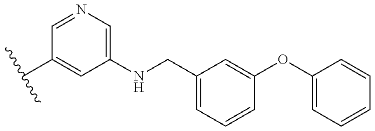 [SMILES: *c1cncc(NCc2cccc(Oc3ccccc3)c2)c1] | Me | NMe |
| 69a | 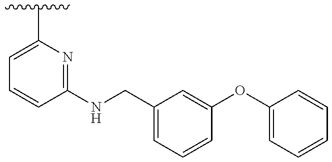 [SMILES: *c1cccc(NCc2cccc(Oc3ccccc3)c2)n1] | H | O |
| 69b | 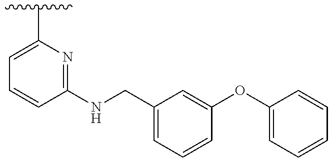 [SMILES: *c1cccc(NCc2cccc(Oc3ccccc3)c2)n1] | Me | O |
| 69c | 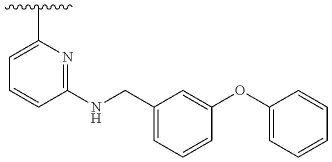 [SMILES: *c1cccc(NCc2cccc(Oc3ccccc3)c2)n1] | H | NH |
| 69d | 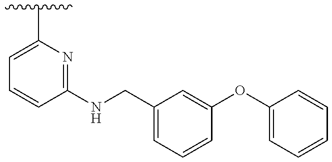 [SMILES: *c1cccc(NCc2cccc(Oc3ccccc3)c2)n1] | Me | NH |
| 69e | 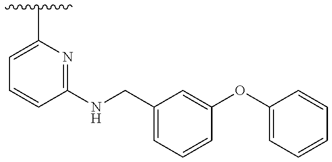 [SMILES: *c1cccc(NCc2cccc(Oc3ccccc3)c2)n1] | H | NMe |
| 69f | 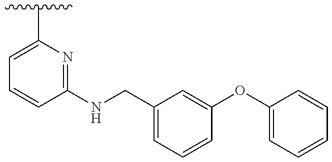 [SMILES: *c1cccc(NCc2cccc(Oc3ccccc3)c2)n1] | Me | NMe |
| 70a | 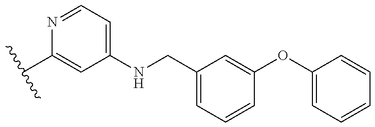 [SMILES: *c1cc(NCc2cccc(Oc3ccccc3)c2)ccn1] | H | O |
| 70b | 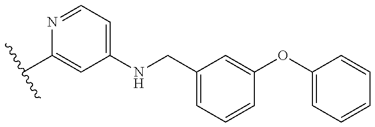 [SMILES: *c1cc(NCc2cccc(Oc3ccccc3)c2)ccn1] | Me | O |
| 70c | 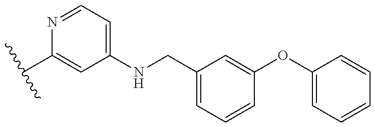 [SMILES: *c1cc(NCc2cccc(Oc3ccccc3)c2)ccn1] | H | NH |
| 70d | 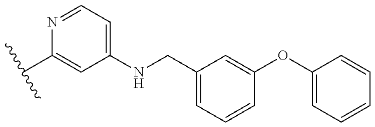 [SMILES: *c1cc(NCc2cccc(Oc3ccccc3)c2)ccn1] | Me | NH |
| 70e | 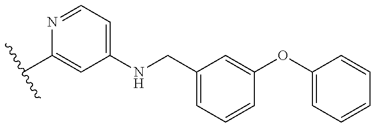 [SMILES: *c1cc(NCc2cccc(Oc3ccccc3)c2)ccn1] | H | NMe |
| 70f | 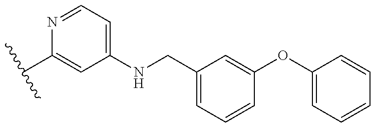 [SMILES: *c1cc(NCc2cccc(Oc3ccccc3)c2)ccn1] | Me | NMe |
| 71a | 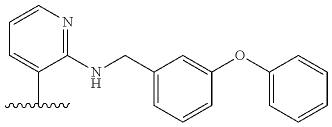 [SMILES: *c1cccnc1NCc1cccc(Oc2ccccc2)c1] | H | O |
| 71b | 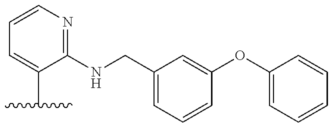 [SMILES: *c1cccnc1NCc1cccc(Oc2ccccc2)c1] | Me | O |
| 71c | 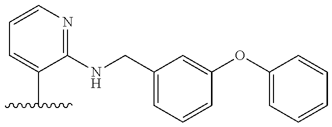 [SMILES: *c1cccnc1NCc1cccc(Oc2ccccc2)c1] | H | NH |
| 71d | 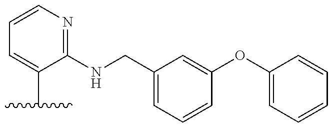 [SMILES: *c1cccnc1NCc1cccc(Oc2ccccc2)c1] | Me | NH |
| 71e | 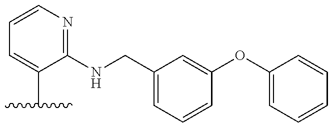 [SMILES: *c1cccnc1NCc1cccc(Oc2ccccc2)c1] | H | NMe |
| 71f | 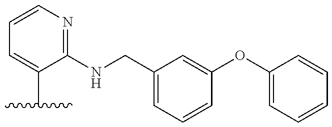 [SMILES: *c1cccnc1NCc1cccc(Oc2ccccc2)c1] | Me | NMe |
| 72a | 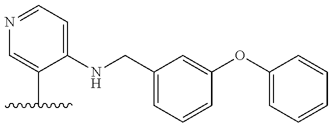 [SMILES: *c1cnccc1NCc1cccc(Oc2ccccc2)c1] | H | O |
| 72b | 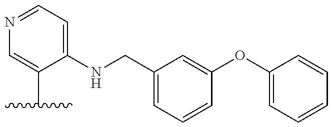 [SMILES: *c1cnccc1NCc1cccc(Oc2ccccc2)c1] | Me | O |
| 72c | 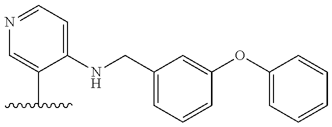 [SMILES: *c1cnccc1NCc1cccc(Oc2ccccc2)c1] | H | NH |
| 72d | 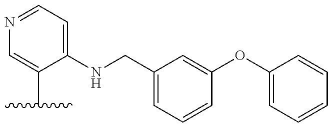 [SMILES: *c1cnccc1NCc1cccc(Oc2ccccc2)c1] | Me | NH |
| 72e | 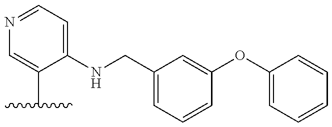 [SMILES: *c1cnccc1NCc1cccc(Oc2ccccc2)c1] | H | NMe |
| 72f | 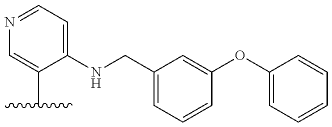 [SMILES: *c1cnccc1NCc1cccc(Oc2ccccc2)c1] | Me | NMe |
| 73a | 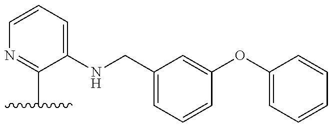 [SMILES: *c1ncccc1NCc1cccc(Oc2ccccc2)c1] | H | O |
| 73b | 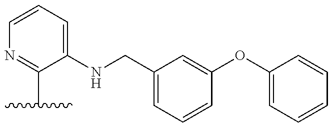 [SMILES: *c1ncccc1NCc1cccc(Oc2ccccc2)c1] | Me | O |
| 73c | 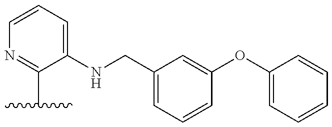 [SMILES: *c1ncccc1NCc1cccc(Oc2ccccc2)c1] | H | NH |
| 73d | 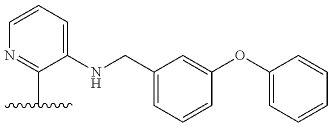 [SMILES: *c1ncccc1NCc1cccc(Oc2ccccc2)c1] | Me | NH |
| 73e | 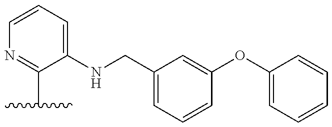 [SMILES: *c1ncccc1NCc1cccc(Oc2ccccc2)c1] | H | NMe |
| 73f | 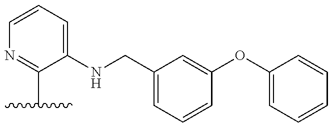 [SMILES: *c1ncccc1NCc1cccc(Oc2ccccc2)c1] | Me | NMe |
| 74a | 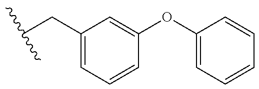 [SMILES: *Cc1cccc(Oc2ccccc2)c1] | H | O |
| 74b | 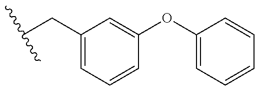 [SMILES: *Cc1cccc(Oc2ccccc2)c1] | Me | O |
| 74c | 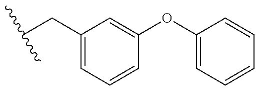 [SMILES: *Cc1cccc(Oc2ccccc2)c1] | H | NH |
| 74d | 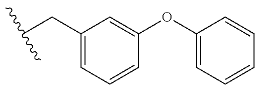 [SMILES: *Cc1cccc(Oc2ccccc2)c1] | Me | NH |
| 74e | 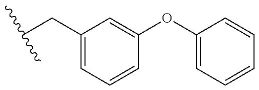 [SMILES: *Cc1cccc(Oc2ccccc2)c1] | H | NMe |
| 74f | 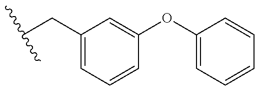 [SMILES: *Cc1cccc(Oc2ccccc2)c1] | Me | NMe |
| 75a | 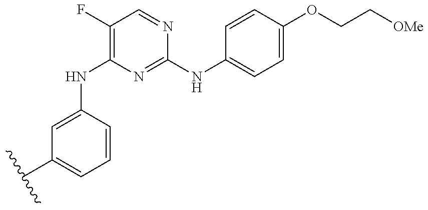 [SMILES: *c1cccc(Nc2nc(Nc3ccc(OCCOC)cc3)ncc2F)c1] | H | O |
| 75b | 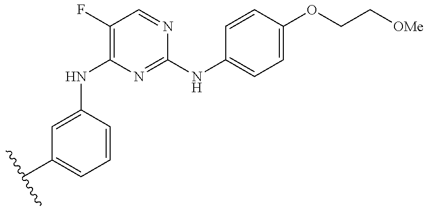 [SMILES: *c1cccc(Nc2nc(Nc3ccc(OCCOC)cc3)ncc2F)c1] | Me | O |
| 75c | 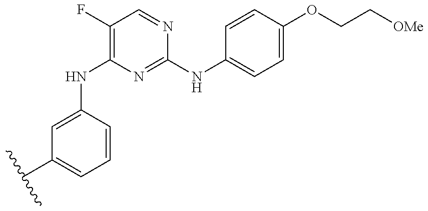 [SMILES: *c1cccc(Nc2nc(Nc3ccc(OCCOC)cc3)ncc2F)c1] | H | NH |
| 75d | 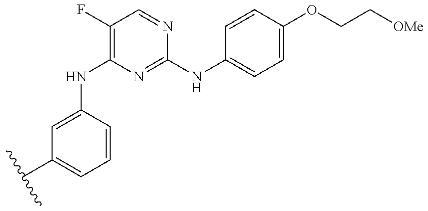 [SMILES: *c1cccc(Nc2nc(Nc3ccc(OCCOC)cc3)ncc2F)c1] | Me | NH |
| 75e | 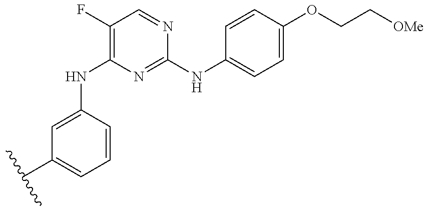 [SMILES: *c1cccc(Nc2nc(Nc3ccc(OCCOC)cc3)ncc2F)c1] | H | NMe |
| 75f | 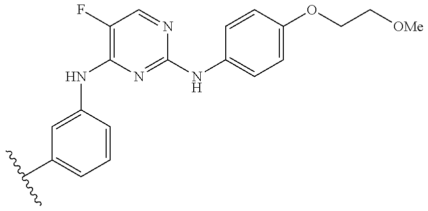 [SMILES: *c1cccc(Nc2nc(Nc3ccc(OCCOC)cc3)ncc2F)c1] | Me | NMe |
| 76a | 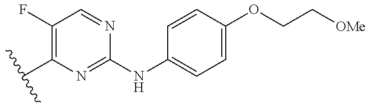 [SMILES: *c1nc(Nc2ccc(OCCOC)cc2)ncc1F] | H | O |
| 76b | 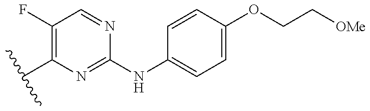 [SMILES: *c1nc(Nc2ccc(OCCOC)cc2)ncc1F] | Me | O |
| 76c | 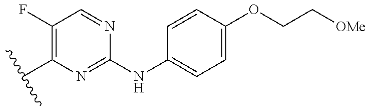 [SMILES: *c1nc(Nc2ccc(OCCOC)cc2)ncc1F] | H | NH |
| 76d | 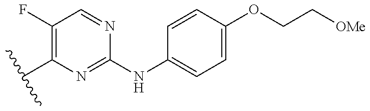 [SMILES: *c1nc(Nc2ccc(OCCOC)cc2)ncc1F] | Me | NH |
| 76e | 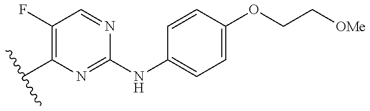 [SMILES: *c1nc(Nc2ccc(OCCOC)cc2)ncc1F] | H | NMe |
| 76f | 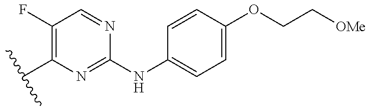 [SMILES: *c1nc(Nc2ccc(OCCOC)cc2)ncc1F] | Me | NMe |
| 77a | 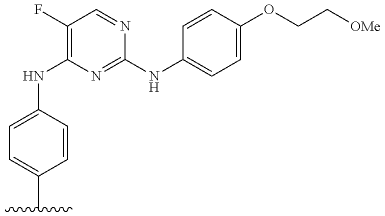 [SMILES: *c1ccc(Nc2nc(Nc3ccc(OCCOC)cc3)ncc2F)cc1] | H | O |
| 77b | 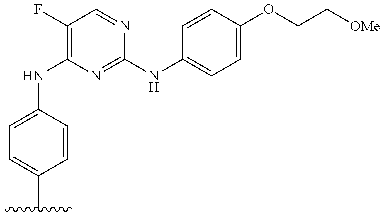 [SMILES: *c1ccc(Nc2nc(Nc3ccc(OCCOC)cc3)ncc2F)cc1] | Me | O |
| 77c | 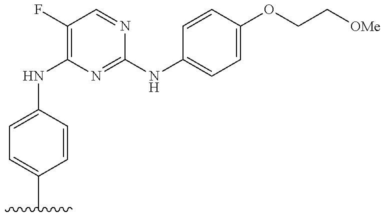 [SMILES: *c1ccc(Nc2nc(Nc3ccc(OCCOC)cc3)ncc2F)cc1] | H | NH |
| 77d | 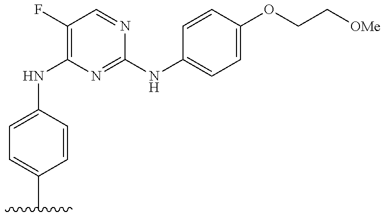 [SMILES: *c1ccc(Nc2nc(Nc3ccc(OCCOC)cc3)ncc2F)cc1] | Me | NH |
| 77e | 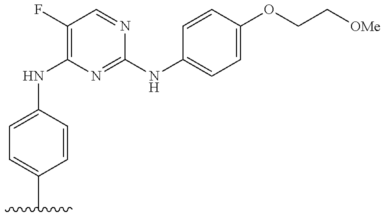 [SMILES: *c1ccc(Nc2nc(Nc3ccc(OCCOC)cc3)ncc2F)cc1] | H | NMe |
| 77f | 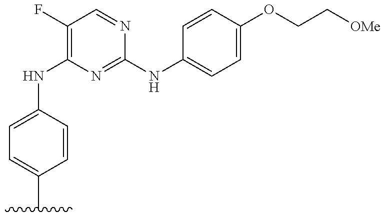 [SMILES: *c1ccc(Nc2nc(Nc3ccc(OCCOC)cc3)ncc2F)cc1] | Me | NMe |
| 78a | 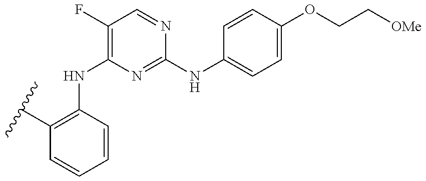 [SMILES: *c1ccccc1Nc1nc(Nc2ccc(OCCOC)cc2)ncc1F] | H | O |
| 78b | 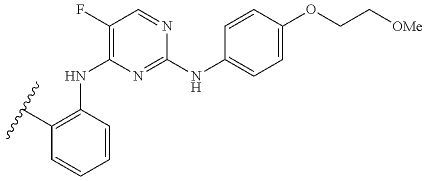 [SMILES: *c1ccccc1Nc1nc(Nc2ccc(OCCOC)cc2)ncc1F] | Me | O |
| 78c | 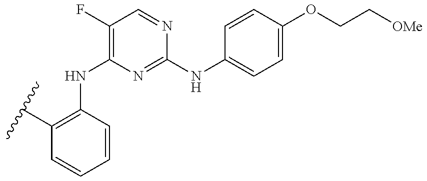 [SMILES: *c1ccccc1Nc1nc(Nc2ccc(OCCOC)cc2)ncc1F] | H | NH |
| 78d | 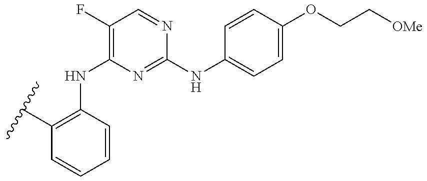 [SMILES: *c1ccccc1Nc1nc(Nc2ccc(OCCOC)cc2)ncc1F] | Me | NH |
| 78e | 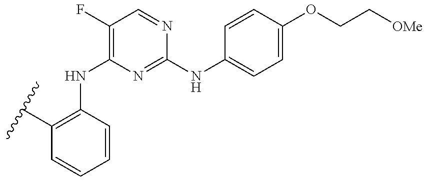 [SMILES: *c1ccccc1Nc1nc(Nc2ccc(OCCOC)cc2)ncc1F] | H | NMe |
| 78f | 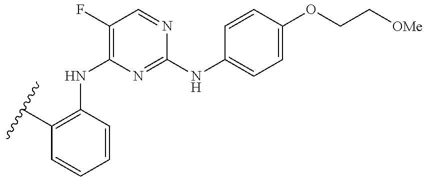 [SMILES: *c1ccccc1Nc1nc(Nc2ccc(OCCOC)cc2)ncc1F] | Me | NMe |
| 79a | 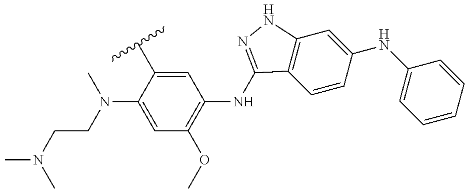 [SMILES: *c1cc(Nc2n[nH]c3cc(Nc4ccccc4)ccc23)c(OC)cc1N(C)CCN(C)C] | H | O |
| 79b | 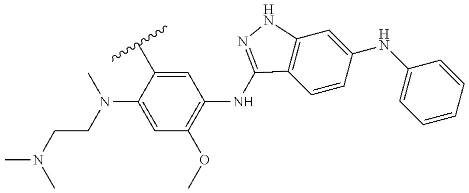 [SMILES: *c1cc(Nc2n[nH]c3cc(Nc4ccccc4)ccc23)c(OC)cc1N(C)CCN(C)C] | Me | O |
| 79c | 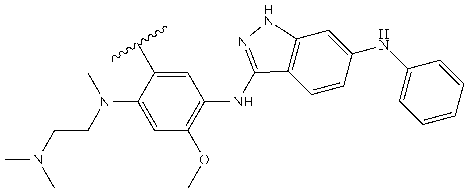 [SMILES: *c1cc(Nc2n[nH]c3cc(Nc4ccccc4)ccc23)c(OC)cc1N(C)CCN(C)C] | H | NH |
| 79d | 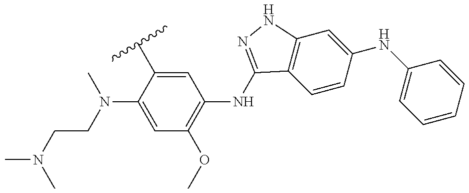 [SMILES: *c1cc(Nc2n[nH]c3cc(Nc4ccccc4)ccc23)c(OC)cc1N(C)CCN(C)C] | Me | NH |
| 79e | 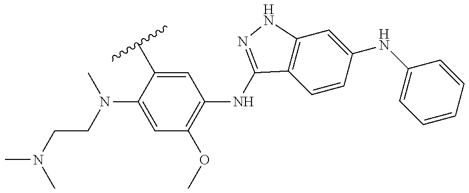 [SMILES: *c1cc(Nc2n[nH]c3cc(Nc4ccccc4)ccc23)c(OC)cc1N(C)CCN(C)C] | H | NMe |
| 79f | 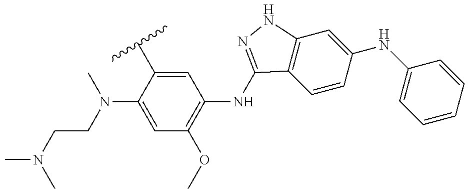 [SMILES: *c1cc(Nc2n[nH]c3cc(Nc4ccccc4)ccc23)c(OC)cc1N(C)CCN(C)C] | Me | NMe |
| 80a | 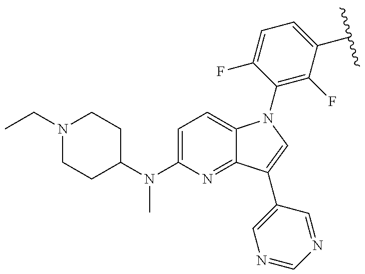 [SMILES: *c1ccc(F)c(-n2cc(-c3cncnc3)c3nc(N(C)C4CCN(CC)CC4)ccc32)c1F] | H | O |
| 80b | 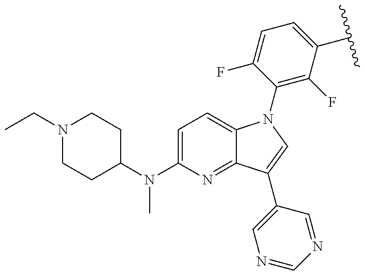 [SMILES: *c1ccc(F)c(-n2cc(-c3cncnc3)c3nc(N(C)C4CCN(CC)CC4)ccc32)c1F] | Me | O |
| 80c | 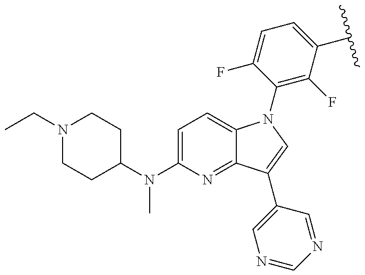 [SMILES: *c1ccc(F)c(-n2cc(-c3cncnc3)c3nc(N(C)C4CCN(CC)CC4)ccc32)c1F] | H | NH |
| 80d | 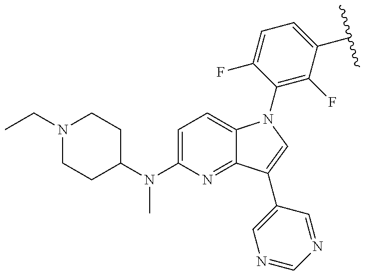 [SMILES: *c1ccc(F)c(-n2cc(-c3cncnc3)c3nc(N(C)C4CCN(CC)CC4)ccc32)c1F] | Me | NH |
| 80e | 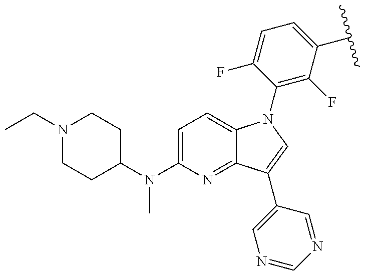 [SMILES: *c1ccc(F)c(-n2cc(-c3cncnc3)c3nc(N(C)C4CCN(CC)CC4)ccc32)c1F] | H | NMe |
| 80f | 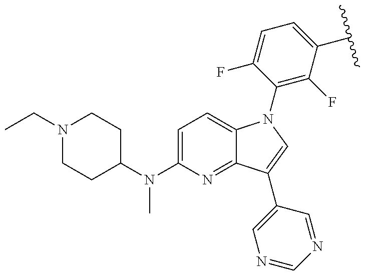 [SMILES: *c1ccc(F)c(-n2cc(-c3cncnc3)c3nc(N(C)C4CCN(CC)CC4)ccc32)c1F] | Me | NMe |
| 81a | 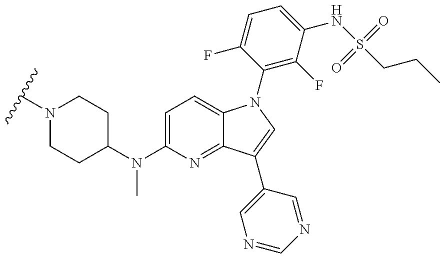 [SMILES: *N1CCC(N(C)c2ccc3c(n2)c(-c2cncnc2)cn3-c2c(F)ccc(NS(=O)(=O)CCC)c2F)CC1] | N/A | O |
| 81b | 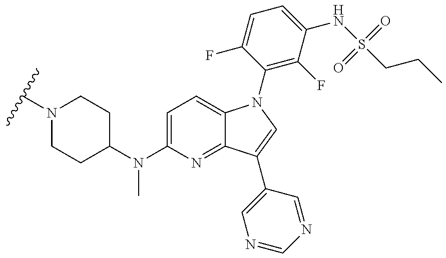 [SMILES: *N1CCC(N(C)c2ccc3c(n2)c(-c2cncnc2)cn3-c2c(F)ccc(NS(=O)(=O)CCC)c2F)CC1] | N/A | O |
| 81c | 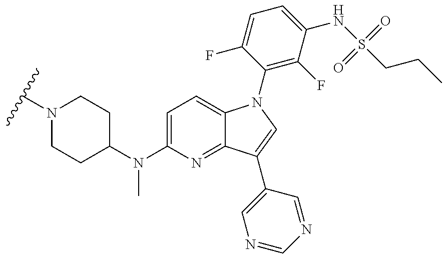 [SMILES: *N1CCC(N(C)c2ccc3c(n2)c(-c2cncnc2)cn3-c2c(F)ccc(NS(=O)(=O)CCC)c2F)CC1] | N/A | NH |
| 81d | 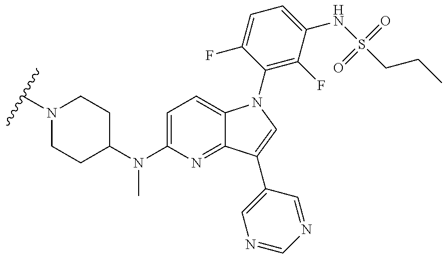 [SMILES: *N1CCC(N(C)c2ccc3c(n2)c(-c2cncnc2)cn3-c2c(F)ccc(NS(=O)(=O)CCC)c2F)CC1] | N/A | NH |
| 81e | 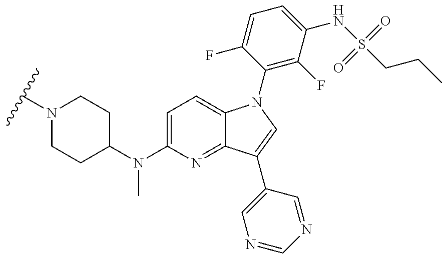 [SMILES: *N1CCC(N(C)c2ccc3c(n2)c(-c2cncnc2)cn3-c2c(F)ccc(NS(=O)(=O)CCC)c2F)CC1] | N/A | NMe |
| 81f | 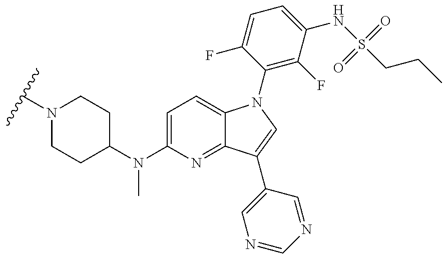 [SMILES: *N1CCC(N(C)c2ccc3c(n2)c(-c2cncnc2)cn3-c2c(F)ccc(NS(=O)(=O)CCC)c2F)CC1] | N/A | NMe |
| 82a | 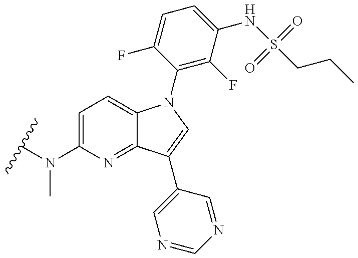 [SMILES: *N(C)c1ccc2c(n1)c(-c1cncnc1)cn2-c1c(F)ccc(NS(=O)(=O)CCC)c1F] | N/A | O |
| 82b | 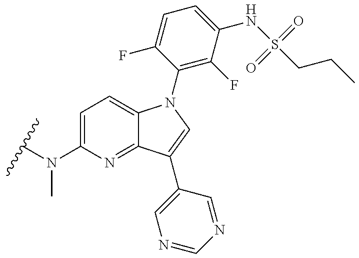 [SMILES: *N(C)c1ccc2c(n1)c(-c1cncnc1)cn2-c1c(F)ccc(NS(=O)(=O)CCC)c1F] | N/A | O |
| 82c | 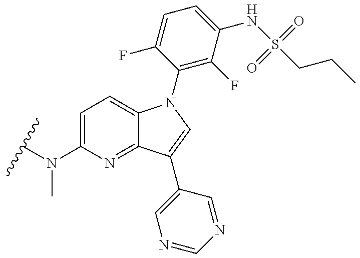 [SMILES: *N(C)c1ccc2c(n1)c(-c1cncnc1)cn2-c1c(F)ccc(NS(=O)(=O)CCC)c1F] | N/A | NH |
| 82d | 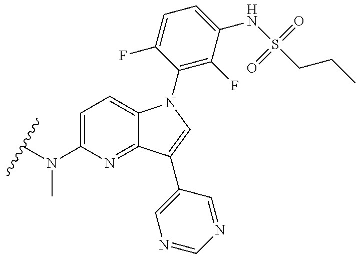 [SMILES: *N(C)c1ccc2c(n1)c(-c1cncnc1)cn2-c1c(F)ccc(NS(=O)(=O)CCC)c1F] | N/A | NH |
| 82e | 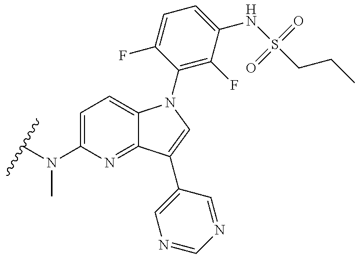 [SMILES: *N(C)c1ccc2c(n1)c(-c1cncnc1)cn2-c1c(F)ccc(NS(=O)(=O)CCC)c1F] | N/A | NMe |
| 82f | 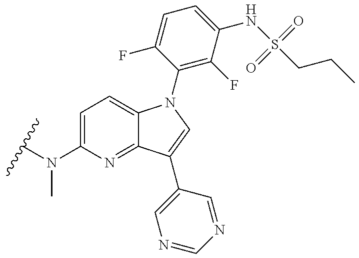 [SMILES: *N(C)c1ccc2c(n1)c(-c1cncnc1)cn2-c1c(F)ccc(NS(=O)(=O)CCC)c1F] | N/A | NMe |
| 83a | 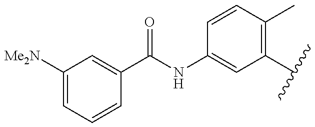 [SMILES: *c1cc(NC(=O)c2cccc(N(C)C)c2)ccc1C] | H | O |
| 83b | 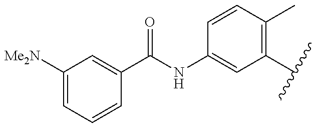 [SMILES: *c1cc(NC(=O)c2cccc(N(C)C)c2)ccc1C] | Me | O |
| 83c | 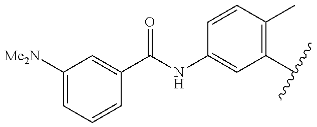 [SMILES: *c1cc(NC(=O)c2cccc(N(C)C)c2)ccc1C] | H | NH |
| 83d | 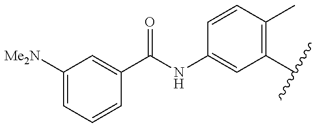 [SMILES: *c1cc(NC(=O)c2cccc(N(C)C)c2)ccc1C] | Me | NH |
| 83e | 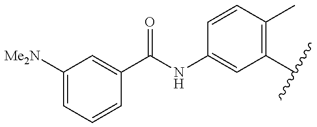 [SMILES: *c1cc(NC(=O)c2cccc(N(C)C)c2)ccc1C] | H | NMe |
| 83f | 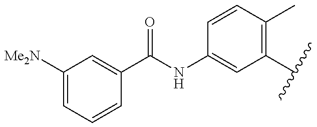 [SMILES: *c1cc(NC(=O)c2cccc(N(C)C)c2)ccc1C] | Me | NMe |
| 84a | 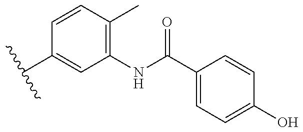 [SMILES: *c1ccc(C)c(NC(=O)c2ccc(O)cc2)c1] | H | O |
| 84b | 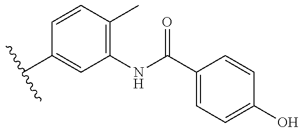 [SMILES: *c1ccc(C)c(NC(=O)c2ccc(O)cc2)c1] | Me | O |
| 84c | 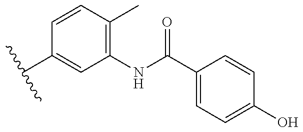 [SMILES: *c1ccc(C)c(NC(=O)c2ccc(O)cc2)c1] | H | NH |
| 84d | 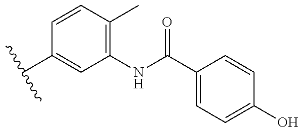 [SMILES: *c1ccc(C)c(NC(=O)c2ccc(O)cc2)c1] | Me | NH |
| 84e | 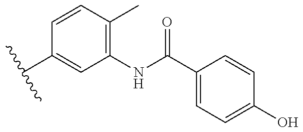 [SMILES: *c1ccc(C)c(NC(=O)c2ccc(O)cc2)c1] | H | NMe |
| 84f | 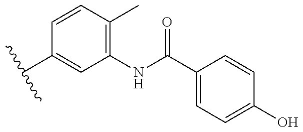 [SMILES: *c1ccc(C)c(NC(=O)c2ccc(O)cc2)c1] | Me | NMe |
| 85a | 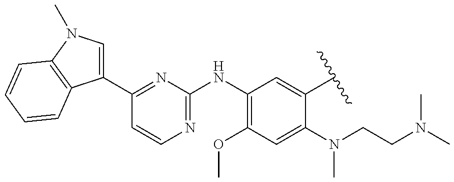 [SMILES: *c1cc(Nc2nccc(-c3cn(C)c4ccccc34)n2)c(OC)cc1N(C)CCN(C)C] | H | O |
| 85b | 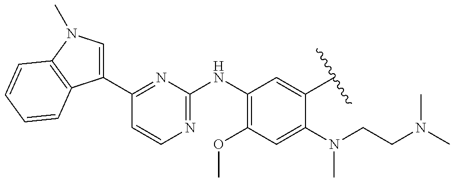 [SMILES: *c1cc(Nc2nccc(-c3cn(C)c4ccccc34)n2)c(OC)cc1N(C)CCN(C)C] | Me | O |
| 85c | 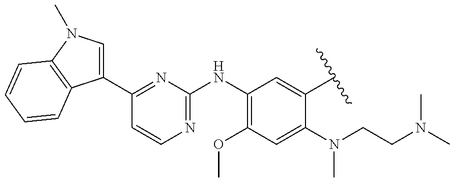 [SMILES: *c1cc(Nc2nccc(-c3cn(C)c4ccccc34)n2)c(OC)cc1N(C)CCN(C)C] | H | NH |
| 85d | 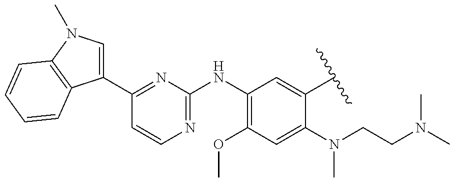 [SMILES: *c1cc(Nc2nccc(-c3cn(C)c4ccccc34)n2)c(OC)cc1N(C)CCN(C)C] | Me | NH |
| 85e | 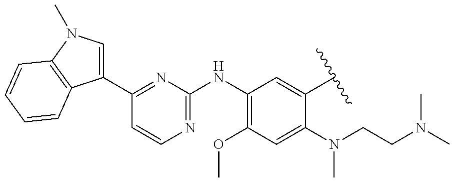 [SMILES: *c1cc(Nc2nccc(-c3cn(C)c4ccccc34)n2)c(OC)cc1N(C)CCN(C)C] | H | NMe |
| 85f | 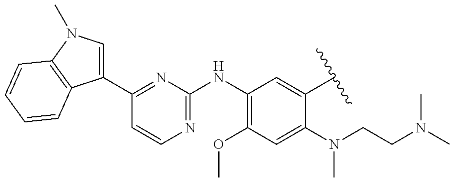 [SMILES: *c1cc(Nc2nccc(-c3cn(C)c4ccccc34)n2)c(OC)cc1N(C)CCN(C)C] | Me | NMe |

In some embodiments, disclosed herein is a pharmaceutically acceptable salt, solvate, or stereoisomer of a compound of Table 1.

In some embodiments, the pentafluorobenzenesulfonamide derivative compound described herein has a structure provided in Table 2.

TABLE 2

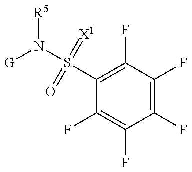

| # | 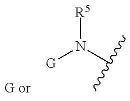 | R⁵ | X¹ |
|---|---|---|---|
| 1a | 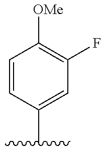 [SMILES: *c1ccc(OC)c(F)c1] | H | NH |
| 1b | 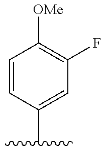 [SMILES: *c1ccc(OC)c(F)c1] | H | NMe |
| 2a | 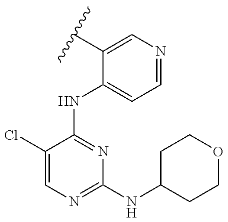 [SMILES: *c1cnccc1Nc1nc(NC2CCOCC2)ncc1Cl] | H | O |
| 3a | 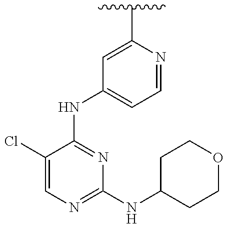 [SMILES: *c1cc(Nc2nc(NC3CCOCC3)ncc2Cl)ccn1] | H | O |
| 4a | 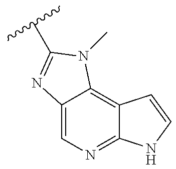 [SMILES: *c1nc2cnc3[nH]ccc3c2n1C] | H | O |
| 5a | 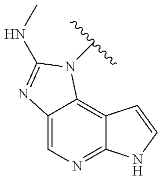 [SMILES: *n1c(NC)nc2cnc3[nH]ccc3c21] | N/A | O |
| 6a | 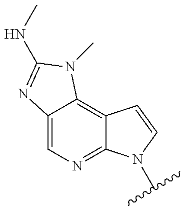 [SMILES: *n1ccc2c1ncc1nc(NC)n(C)c12] | N/A | O |
| 7a | 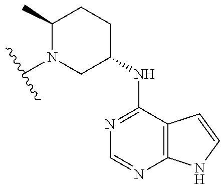 [SMILES: *N1C[C@@H](Nc2ncnc3[nH]ccc23)CC[C@@H]1C] | N/A | O |
| 8a | 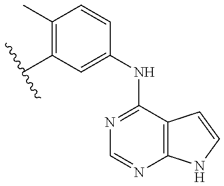 [SMILES: *c1cc(Nc2ncnc3[nH]ccc23)ccc1C] | H | O |
| 11a | 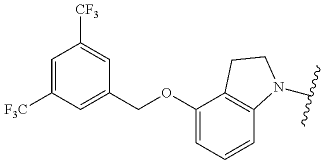 [SMILES: *N1CCc2c(OCc3cc(C(F)(F)F)cc(C(F)(F)F)c3)cccc21] | N/A | O |
| 13a | 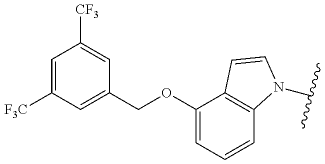 [SMILES: *n1ccc2c(OCc3cc(C(F)(F)F)cc(C(F)(F)F)c3)cccc21] | N/A | O |
| 15a | 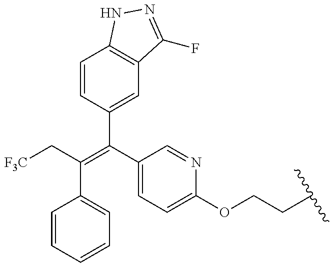 [SMILES: *CCOc1ccc(/C(=C(/CC(F)(F)F)c2ccccc2)c2ccc3[nH]nc(F)c3c2)cn1] | H | O |
| 21a | 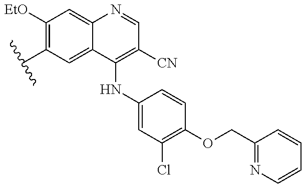 [SMILES: *c1cc2c(Nc3ccc(OCc4ccccn4)c(Cl)c3)c(C#N)cnc2cc1OCC] | H | O |
| 26a | 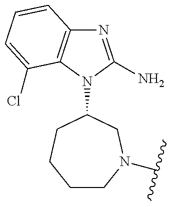 [SMILES: *N1CCCC[C@H](n2c(N)nc3cccc(Cl)c32)C1] | N/A | O |
| 30a | 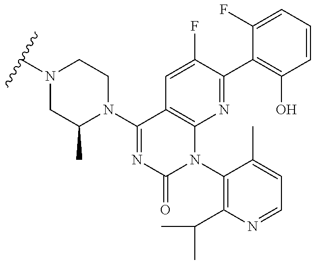 [SMILES: *N1CCN(c2nc(=O)n(-c3c(C)ccnc3C(C)C)c3nc(-c4c(O)cccc4F)c(F)cc23)[C@@H](C)C1] | N/A | O |
| 34a | 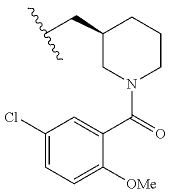 [SMILES: *C[C@H]1CCCN(C(=O)c2cc(Cl)ccc2OC)C1] | H | O |
| 38a | 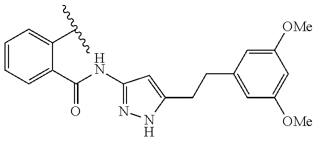 [SMILES: *c1ccccc1C(=O)Nc1cc(CCc2cc(OC)cc(OC)c2)[nH]n1] | H | O |
| 49a | 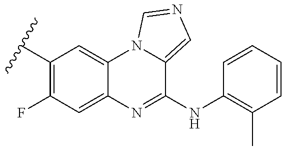 [SMILES: *c1cc2c(cc1F)nc(Nc1ccccc1C)c1cncn12] | H | O |
| 57a | 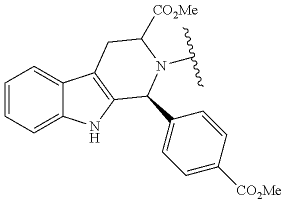 [SMILES: *N1C(C(=O)OC)Cc2c([nH]c3ccccc23)[C@@H]1c1ccc(C(=O)OC)cc1] | N/A | O |
| 61a | 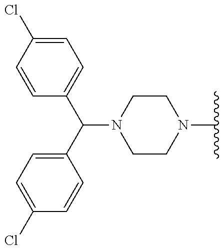 [SMILES: *N1CCN(C(c2ccc(Cl)cc2)c2ccc(Cl)cc2)CC1] | N/A | O |

In some embodiments, disclosed herein is a pharmaceutically acceptable salt, solvate, or stereoisomer of a compound of Table 2.

In some embodiments, the pentafluorobenzenesulfonamide derivative compound described herein has a structure provided in Table 3.

TABLE 3

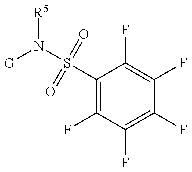

| # | 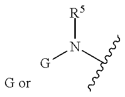 |
|---|---|
| 3-1 | 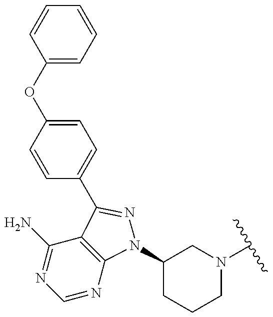 [SMILES: *N1CCC[C@@H](n2nc(-c3ccc(Oc4ccccc4)cc3)c3c(N)ncnc32)C1] |
| 3-2 | 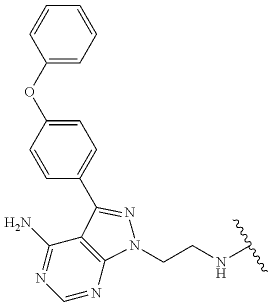 [SMILES: *NCCn1nc(-c2ccc(Oc3ccccc3)cc2)c2c(N)ncnc21] |
| 3-3 | 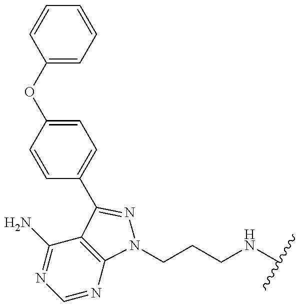 [SMILES: *NCCCn1nc(-c2ccc(Oc3ccccc3)cc2)c2c(N)ncnc21] |
| 3-4 | 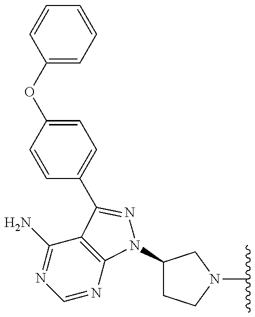 [SMILES: *N1CC[C@@H](n2nc(-c3ccc(Oc4ccccc4)cc3)c3c(N)ncnc32)C1] |
| 3-5 | 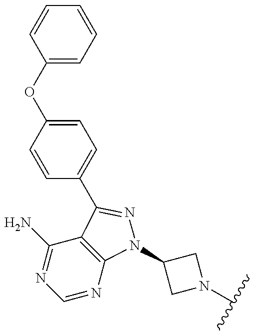 |
| 3-6 | 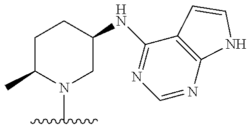 [SMILES: *N1C[C@H](Nc2ncnc3[nH]ccc23)CC[C@@H]1C] |
| 3-7 | 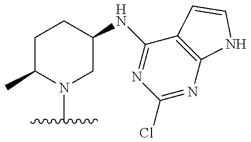 [SMILES: *N1C[C@H](Nc2nc(Cl)nc3[nH]ccc23)CC[C@@H]1C] |
| 3-8 | 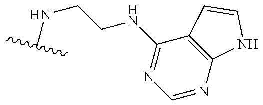 [SMILES: *NCCNc1ncnc2[nH]ccc12] |
| 3-9 | 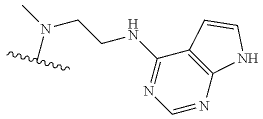 [SMILES: *N(C)CCNc1ncnc2[nH]ccc12] |
| 3-10 | 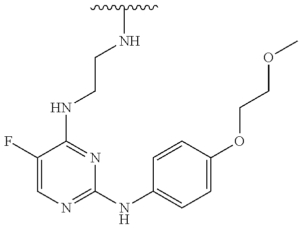 [SMILES: *NCCNc1nc(Nc2ccc(OCCOC)cc2)ncc1F] |
| 3-11 | 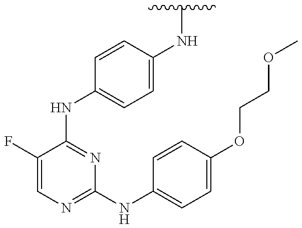 [SMILES: *Nc1ccc(Nc2nc(Nc3ccc(OCCOC)cc3)ncc2F)cc1] |
| 3-12 | 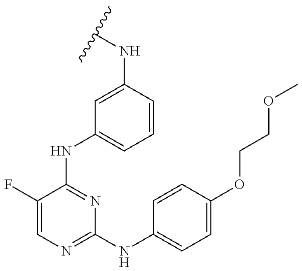 [SMILES: *Nc1cccc(Nc2nc(Nc3ccc(OCCOC)cc3)ncc2F)c1] |
| 3-13 | 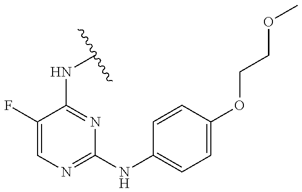 [SMILES: *Nc1nc(Nc2ccc(OCCOC)cc2)ncc1F] |
| 3-14 | 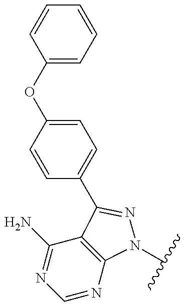 [SMILES: *n1nc(-c2ccc(Oc3ccccc3)cc2)c2c(N)ncnc21] |

In some embodiments, disclosed herein is a pharmaceutically acceptable salt, solvate, or stereoisomer of a compound of Table 3.

In some embodiments, the pentafluorobenzenesulfonamide derivative compound described herein is not compound 3-1 of Table 3.

In some embodiments, the pentafluorobenzenesulfonamide derivative compound described herein is not compound 3-2 of Table 3.

In some embodiments, the pentafluorobenzenesulfonamide derivative compound described herein is not compound 3-3 of Table 3.

In some embodiments, the pentafluorobenzenesulfonamide derivative compound described herein is not compound 3-4 of Table 3.

In some embodiments, the pentafluorobenzenesulfonamide derivative compound described herein is not compound 3-5 of Table 3.

In some embodiments, the pentafluorobenzenesulfonamide derivative compound described herein is not compound 3-6 of Table 3.

In some embodiments, the pentafluorobenzenesulfonamide derivative compound described herein is not compound 3-7 of Table 3.

In some embodiments, the pentafluorobenzenesulfonamide derivative compound described herein is not compound 3-8 of Table 3.

In some embodiments, the pentafluorobenzenesulfonamide derivative compound described herein is not compound 3-9 of Table 3.

In some embodiments, the pentafluorobenzenesulfonamide derivative compound described herein is not compound 3-10 of Table 3.

In some embodiments, the pentafluorobenzenesulfonamide derivative compound described herein is not compound 3-11 of Table 3.

In some embodiments, the pentafluorobenzenesulfonamide derivative compound described herein is not compound 3-12 of Table 3.

In some embodiments, the pentafluorobenzenesulfonamide derivative compound described herein is not compound 3-13 of Table 3.

In some embodiments, the pentafluorobenzenesulfonamide derivative compound described herein is not compound 3-14 of Table 3.

In some embodiments, the pentafluorobenzenesulfonamide derivative compound of Formula (I) is not compound of Table 3.

In some embodiments, the pentafluorobenzenesulfonamide derivative compound described herein has a structure provided in Table 4.

TABLE 4

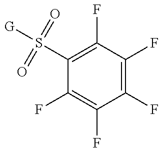

| | | |
|---|---|---|
| # | Name | G |

Batabulin analogues

| | | |
|---|---|---|
| 4-1 | 2,3,4,5,6-pentafluoro-N-(3-fluoro-4- (trifluoromethoxy)phenyl)benzenesulfonamide | 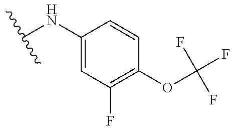 [SMILES: *Nc1ccc(OC(F)(F)F)c(F)c1] |
| 4-2 | 2,3,4,5,6-pentafluoro-N-(4- phenoxyphenyl)benzenesulfonamide | 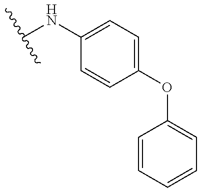 [SMILES: *Nc1ccc(Oc2ccccc2)cc1] |
| 4-3 | 2,3,4,5,6-pentafluoro-N-(3-fluoro-4- hydroxyphenyl)benzenesulfonamide | 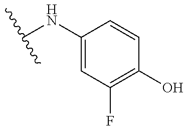 [SMILES: *Nc1ccc(O)c(F)c1] |
| 4-4 | N-(4-(difluoromethoxy)-3-fluorophenyl)-2,3,4,5,6- pentafluorobenzenesulfonamide | 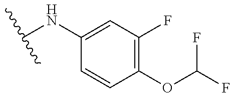 [SMILES: *Nc1ccc(OC(F)F)c(F)c1] |
| 4-5 | N-(4-cyano-3-fluorophenyl)-2,3,4,5,6- pentafluorobenzenesulfonamide | 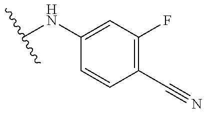 [SMILES: *Nc1ccc(C#N)c(F)c1] |
| 4-6 | 2,3,4,5,6-pentafluoro-N-(3-fluoro-4- (fluroomethoxy)phenyl)benzenesulfonamide | 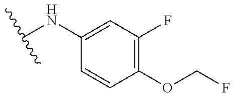 [SMILES: *Nc1ccc(OCF)c(F)c1] |
| 4-7 | N-(4-(cyclopropylmethyl)-3-fluorophenyl)-2,3,4,5,6- pentafluorobenzenesulfonamide | 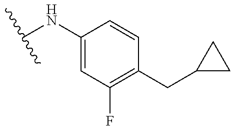 [SMILES: *Nc1ccc(CC2CC2)c(F)c1] |
| 4-8 | tert-butyl 4-((perfluorophenyl)sulfonyl)piperazine-1- carboxylate | 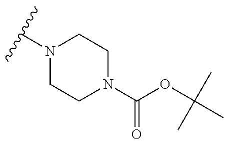 [SMILES: *N1CCN(C(=O)OC(C)(C)C)CC1] |
| 4-9 | 3-fluoro-4-methyl-1N- ((perfluorophenyl)sulfonyl)benzamide | 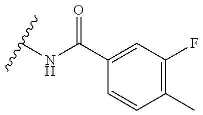 [SMILES: *NC(=O)c1ccc(C)c(F)c1] |
| 4-10 | N-(2,3-dihydrobenzo[b][1,4]dioxin-6-yl)-2,3,4,5,6- pentafluorobenzenesulfonamide | 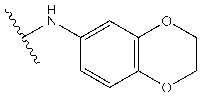 [SMILES: *Nc1ccc2c(c1)OCCO2] |
| 4-11 | 2,3,4,5,6-pentafluoro-N-(4-phenylthiazol-2- yl)benzenesulfonamide | 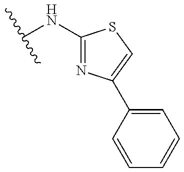 [SMILES: *Nc1nc(-c2ccccc2)cs1] |
| 4-12 | 2,3,4,5,6-pentafluoro-N-(3-fluoro-4-methoxyphenyl)-N- methylbenzenesulfonamide | 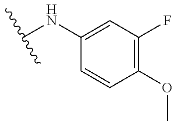 [SMILES: *Nc1ccc(OC)c(F)c1] |

In some embodiments, disclosed herein is a pharmaceutically acceptable salt, solvate, or stereoisomer of a compound of Table 4.

In some embodiments, the pentafluorobenzenesulfonamide derivative compound described herein has a structure provided in Table 5.

TABLE 5

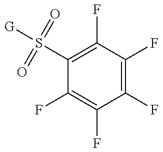

| | |
|---|---|
| # | G |

Ibrutinib - R¹ and R² variant

| | |
|---|---|
| 5-1 | 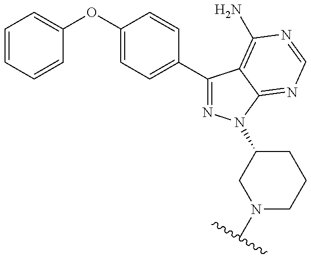 [SMILES: *N1CCC[C@@H](n2nc(-c3ccc(Oc4ccccc4)cc3)c3c(N)ncnc32)C1] |
| 5-2 | 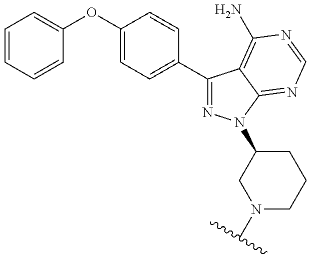 [SMILES: *N1CCC[C@H](n2nc(-c3ccc(Oc4ccccc4)cc3)c3c(N)ncnc32)C1] |
| 5-3 | 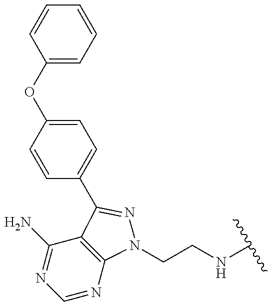 [SMILES: *NCCn1nc(-c2ccc(Oc3ccccc3)cc2)c2c(N)ncnc21] |
| 5-4 | 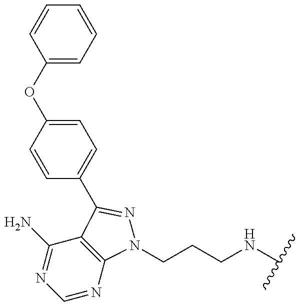 [SMILES: *NCCCn1nc(-c2ccc(Oc3ccccc3)cc2)c2c(N)ncnc21] |
| 5-5 | 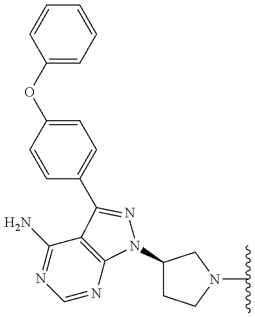 [SMILES: *N1CC[C@@H](n2nc(-c3ccc(Oc4ccccc4)cc3)c3c(N)ncnc32)C1] |
| 5-6 | 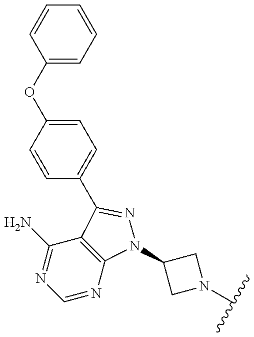 |

JAK3 - R¹ and R² variant

| | |
|---|---|
| 5-8 | 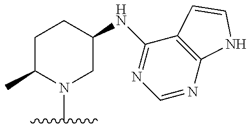 [SMILES: *N1C[C@H](Nc2ncnc3[nH]ccc23)CC[C@@H]1C] |
| 5-9 | 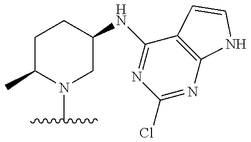 [SMILES: *N1C[C@H](Nc2nc(Cl)nc3[nH]ccc23)CC[C@@H]1C] |
| 5-10 | 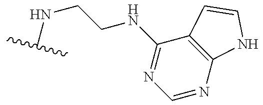 [SMILES: *NCCNc1ncnc2[nH]ccc12] |
| 5-11 | 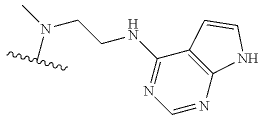 [SMILES: *N(C)CCNc1ncnc2[nH]ccc12] |
| 5-12 | 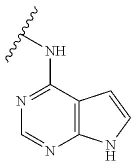 [SMILES: *Nc1ncnc2[nH]ccc12] |

Spebrutinib - R¹ and R² variant

| | |
|---|---|
| 5-7 | 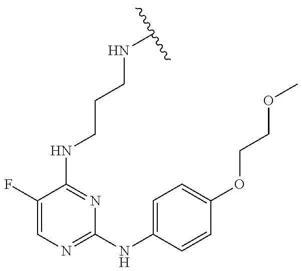 [SMILES: *NCCCNc1nc(Nc2ccc(OCCOC)cc2)ncc1F] |
| 5-13 | 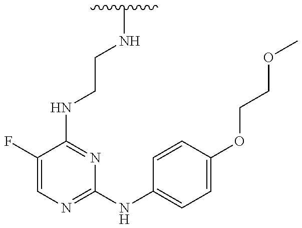 [SMILES: *NCCNc1nc(Nc2ccc(OCCOC)cc2)ncc1F] |
| 5-14 | 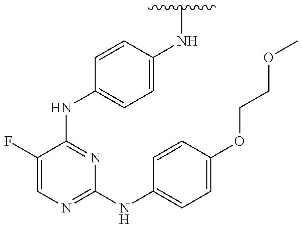 [SMILES: *Nc1ccc(Nc2nc(Nc3ccc(OCCOC)cc3)ncc2F)cc1] |
| 5-15 | 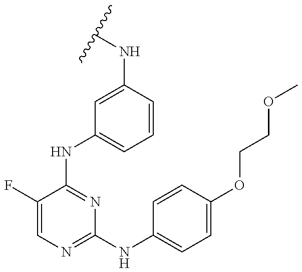 [SMILES: *Nc1cccc(Nc2nc(Nc3ccc(OCCOC)cc3)ncc2F)c1] |
| 5-16 | 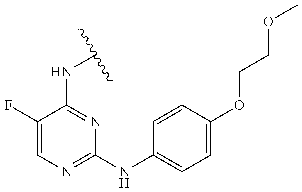 [SMILES: *Nc1nc(Nc2ccc(OCCOC)cc2)ncc1F] |
| 5-17 | 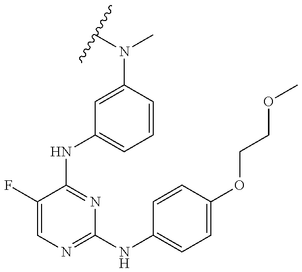 [SMILES: *N(C)c1cccc(Nc2nc(Nc3ccc(OCCOC)cc3)ncc2F)c1] |
| 5-18 | 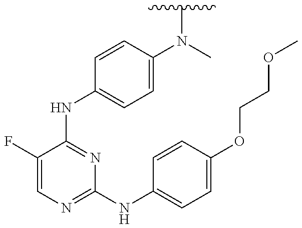 [SMILES: *N(C)c1ccc(Nc2nc(Nc3ccc(OCCOC)cc3)ncc2F)cc1] |

Direct Attachment to Hinge binder

| | |
|---|---|
| 5-19 | 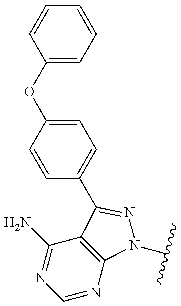 [SMILES: *n1nc(-c2ccc(Oc3ccccc3)cc2)c2c(N)ncnc21] |

EGFR

| | |
|---|---|
| 5-20 | 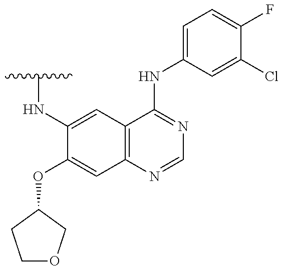 [SMILES: *Nc1cc2c(Nc3ccc(F)c(Cl)c3)ncnc2cc1O[C@H]1CCOC1] |
| 5-21 | 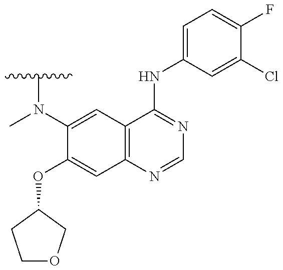 [SMILES: *N(C)c1cc2c(Nc3ccc(F)c(Cl)c3)ncnc2cc1O[C@H]1CCOC1] |

In some embodiments, disclosed herein is a pharmaceutically acceptable salt, solvate, or stereoisomer of a compound of Table 5.

In some instances, throughout specification same compounds might have different annotation; such distinct annotations do not indicate a difference in otherwise identical compounds.

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the pentafluorobenzenesulfonamide derivative compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the pentafluorobenzenesulfonamide derivative compound described herein is administered as a pure chemical. In other embodiments, the pentafluorobenzenesulfonamide derivative compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one pentafluorobenzenesulfonamide derivative compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), Formula (Ia), Formula (Iaa), or Formula (Ib), or a compound disclosed in Table 1, Table 2, Table 3, Table 4, or Table 5, or a pharmaceutically acceptable salt or solvate thereof.

One embodiment provides a method of preparing a pharmaceutical composition comprising mixing a compound of Formula (I), Formula (Ia), Formula (Iaa), or Formula (Ib), or a compound disclosed in Table 1, Table 2, Table 3, Table 4, or Table 5, or a pharmaceutically acceptable salt or solvate thereof, and a pharmaceutically acceptable carrier.

In certain embodiments, the pentafluorobenzenesulfonamide derivative compound as described by Formula (I), Formula (Ia), Formula (Iaa), or Formula (Ib), or a compound disclosed in Table 1, Table 2, Table 3, Table 4, or Table 5, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

In some embodiments, the pentafluorobenzenesulfonamide derivative compound as described by Formula (I), Formula (Ia), Formula (Iaa), or Formula (Ib), or a compound disclosed in Table 1, Table 2, Table 3, Table 4, or Table 5, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one pentafluorobenzenesulfonamide derivative compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), Formula (Ia), Formula (Iaa), or Formula (Ib), or a compound disclosed in Table 1, Table 2, Table 3, Table 4, or Table 5, or a pharmaceutically acceptable salt or solvate thereof, for use in a method of treatment of the human or animal body.

One embodiment provides a compound of Formula (I), Formula (Ia), Formula (Iaa), or Formula (Ib), or a compound disclosed in Table 1, Table 2, Table 3, Table 4, or Table 5, or a pharmaceutically acceptable salt or solvate thereof, for use in a method of treatment of cancer or neoplastic disease.

One embodiment provides a use of a compound of Formula (I), Formula (Ia), Formula (Iaa), or Formula (Ib), or a compound disclosed in Table 1, Table 2, Table 3, Table 4, or Table 5, or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), Formula (Ia), Formula (Iaa), or Formula (Ib), or a pharmaceutically acceptable salt or solvate thereof.

In some embodiments, described herein is a method of treating cancer in a patient in need thereof comprising administering to the patient a compound disclosed in Table 1, Table 2, Table 3, Table 4, or Table 5, or a pharmaceutically acceptable salt or solvate thereof.

In some embodiments, also described herein is a method of treating cancer in a patient in need thereof comprising administering to the patient a pharmaceutical composition comprising a compound of Formula (I), Formula (Ia), Formula (Iaa), or Formula (Ib), or a pharmaceutically acceptable salt or solvate thereof, and a pharmaceutically acceptable excipient.

In some embodiments, also described herein is a method of treating cancer in a patient in need thereof comprising administering to the patient a pharmaceutical composition comprising a compound disclosed in Table 1, Table 2, Table 3, Table 4, or Table 5, or a pharmaceutically acceptable salt or solvate thereof, and a pharmaceutically acceptable excipient.

In some embodiments, the cancer is selected from chronic and acute myeloid leukemia. In some embodiments, the cancer is selected from chronic lymphocytic leukemia and small lymphocytic lymphoma.

Provided herein is the method wherein the pharmaceutical composition is administered orally. Provided herein is the method wherein the pharmaceutical composition is administered by injection.

In some embodiments, a pentafluorobenzenesulfonamide derivative compound described herein comprises a protein binder or ligand. In some embodiments, G (or G¹) in Formula (I) is or comprises a protein binder or ligand. In some embodiments, the protein is selected ERK2, JAK3, mTORC1, HER2, EGFR, EGFR4, KRAS, FGFR4, BTK, TAK1, GPX4, ITK, and Tubulin. One embodiment provides a protein modified with a pentafluorobenzenesulfonamide derivative compound as described herein, wherein the compound forms a covalent bond with a sulfur atom of a cysteine residue of the protein. In some embodiments, the protein is selected from ERK2, JAK3, mTORC1, HER2, EGFR, EGFR4, KRAS, FGFR4, BTK, TAK1, GPX4, ITK, and Tubulin. In some embodiments, the protein is ERK2. In some embodiments, the protein is JAK3. mTORC1. In some embodiments, the protein is HER2. In some embodiments, the protein is EGFR. In some embodiments, the protein is EGFR4. In some embodiments, the protein is KRAS. In some embodiments, the protein is FGFR4. In some embodiments, the protein is BTK. In some embodiments, the protein is TAK1. In some embodiments, the protein is GPX4. In some embodiments, the protein is ITK. In some embodiments, the protein is Tubulin. In some embodiments, the protein is CRAF.

One embodiment provides a method of modifying (e.g., attaching to and/or degrading) a polypeptide with a pentafluorobenzenesulfonamide derivative compound as described herein, comprising contacting the polypeptide with the compound to form a covalent bond with a sulfur atom of a cysteine residue of the polypeptide.

One embodiment provides a method of binding a compound to a polypeptide, comprising contacting the polypeptide with a pentafluorobenzenesulfonamide derivative compound as described herein.

In some embodiments, the polypeptide is selected from ERK2, JAK3, mTORC1, HER2, EGFR, EGFR4, KRAS, FGFR4, BTK, TAK1, GPX4, ITK, and Tubulin. In some embodiments, the polypeptide is ERK2. In some embodiments, the polypeptide is JAK3. mTORC1. In some embodiments, the polypeptide is HER2. In some embodiments, the polypeptide is EGFR. In some embodiments, the polypeptide is EGFR4. In some embodiments, the polypeptide is KRAS. In some embodiments, the polypeptide is FGFR4. In some embodiments, the polypeptide is BTK. In some embodiments, the polypeptide is TAK1. In some embodiments, the polypeptide is GPX4. In some embodiments, the polypeptide is ITK. In some embodiments, the polypeptide is Tubulin. In some embodiments, the polypeptide is CRAF.

In some embodiments, a pentafluorobenzenesulfonamide derivative compound described herein comprises a protein binder or ligand. In some embodiments, pentafluorobenzenesulfonamide derivative compounds described herein are used for targets in Table 6.

TABLE 6

| Compound # | Potential Target |
|---|---|
| 1a-d | β-tubulin; PARP |
| 2a-f, 3a-f | ERK1/2 |
| 4a-f, 5a-c, 6a-c | JAK3 |
| 7a-c, 8a-f, 9a-c, 10a-f | JAK3, STAT, TYK2 |
| 11a-c, 12a-c, 13a-c, 14a-c | MTORC1 |
| 15a-f, 16a-f, 17a-f, 18a-f, 19a-f, | HER2, CDK 4/6 |
| 20a-f | |
| 21a-f, 22a-f, 23a-f, 24a-f, 25a-f | EGFR, HER2, CYP3A4 |
| 26a-c, 27a-c, 28a-f, 29a-f | EGFR, ERK, MEK |
| 30a-c, 31a-f, 32a-f, 33a-f | KRAS |
| 34a-f, 35a-f, 36a-f, 37a-f | KRAS |
| 38a-f, 39a-f, 40a-f, 41a-f, 42a-c, | FGFR |
| 43a-c, 44a-c | |
| 45a-c, 46a-c, 47a-f, 48a-f | BTK, PI3K, IRAK |
| 49a-f, 50a-f, | BTK, LCK, IRAK4 |
| 51a-f, 52a-f, 53a-f, 54a-f, 55a-c, | TAKI, ERK |
| 56a-c | |
| 57a-c, 58a-f, 59a-c, 60a-f | GPX4 |
| 61a-c, 62a-f | GPX4 |
| 63a-c, 64a-c, 65a-f | ITK |
| 66a-f, 67a-f, 68a-f, 69a-f, 70a-f, | P-tubulin |
| 71a-f, 72-a-f, 73a-f, 74a-f | |
| 75a-f, 76a-f, 77a-f, 78a-f | BTK, SYK, FLT3 |
| 79a-f, | EGFR |
| 80a-f, 81a-f, 82a-f, 83a-f, 84a-f | CRAF |

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the pentafluorobenzenesulfonamide derivative compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
T temperature
TFA trifluoroacetic acid
THE tetrahydrofuran

Exemplary compounds of the application are synthesized using the methods described herein, or other methods, which are known in the art. Unless otherwise noted, reagents and solvents are obtained from commercial suppliers

Anhydrous solvents, methanol, acetonitrile, dichloromethane, tetrahydrofuran and dimethylformamide, are purchased from Sigma Aldrich and used directly from Sure-Seal bottles. Reactions are performed under an atmosphere of dry nitrogen in oven-dried glassware and are monitored for completeness by thin-layer chromatography (TLC) using silica gel (visualized by UV light, or developed by treatment with KMnO₄ stain and ninhydrin stain). NMR spectra are recorded in Bruker Avance III spectrometer at 23° C., operating at 400 MHz for ¹H NMR and 100 MHz ¹³C NMR spectroscopy either in CDCl₃, CD₃OD or d₆-DMSO. Chemical shifts (d) are reported in parts per million (ppm) after calibration to residual isotopic solvent. Coupling constants (J) are reported in Hz. Mass spectrometry is performed with an AB/Sciex QStar mass spectrometer with an ESI source, MS/MS and accurate mass capabilities, associated with an Agilent 1100 capillary LC system. Before biological testing, inhibitor purity is evaluated by reversed-phase HPLC (rpHPLC). Analysis by rpHPLC is performed using a Phenomenex Luna 5 u C_{18 150} mm×4.6 mm column run at 1.2 mL/min, and using gradient mixtures. The linear gradient consisted of a changing solvent composition of either (I) 15% MeCN and 85% H₂O with 0.1% TFA (v/v) to 100% MeCN over 30 minutes and (II) 15% MeCN and 85% H₂O with 0.1% TFA (v/v) to 100% MeCN over 60 minutes, UV detection at 250 nm. For reporting HPLC data, percentage purity is given in parentheses after the retention time for each condition. All biologically evaluated compounds are >95% chemical purity as measured by HPLC. The HPLC traces for all tested compounds are provided in supporting information.

### General Procedure 1: Synthesis of Sulfonimidamides

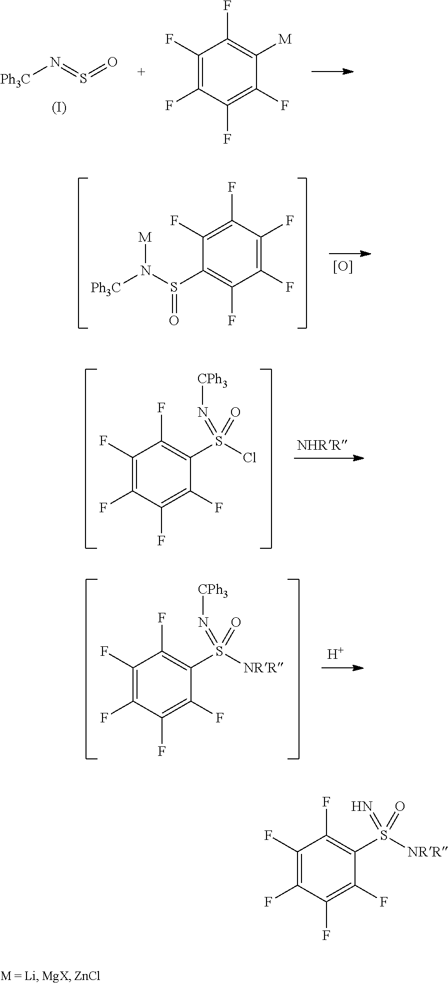

The starting material, compound (I), can be prepared according to the reported procedure (Angew. Chem. Int. Ed. 2017, 56, 14937).

An oven-dried flask charged with (I) (1.0 equiv.) and THE (0.1 M) is cooled to 0° C. Then the corresponding organometallic reagent (1.0 equiv.) can be added dropwise and stirred at 0° C. for 5 min. Next, in a dark fume hood, tert-butyl hypochlorite (1.05 equiv.) is added and the reaction mixture is allowed to stir for 15 min, followed by the addition of triethylamine (1.0 equiv.) and the corresponding amine (1.0-1.2 equiv.). The reaction mixture is left stirring at room temperature for 16 h. Finally, methanesulfonic acid (5.0 equiv.) is added, and the reaction stirred vigorously for 15 min at room temperature. The reaction is quenched by diluting it with DCM and the addition of a saturated aqueous solution of sodium bicarbonate. The two layers are partitioned and the aqueous layer is extracted with DCM (×3). Combined organic layers are dried over magnesium sulfate (MgSO₄), filtered and concentrated in vacuo. Crude samples can be purified by flash column chromatography on silica gel.

### Example 1: 2,3,4,5,6-pentafluoro-N-(3-fluoro-4-methoxyphenyl)benzenesulfonimidamide (Compound 1a

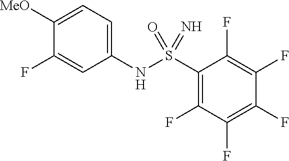 [SMILES: COc1ccc(NS(=N)(=O)c2c(F)c(F)c(F)c(F)c2F)cc1F]

Compound 1a can be prepared according to general procedure 1 using 3-fluoro-4-methoxyanaline.

### Example 2: 2,3,4,5,6-pentafluoro-N-(3-fluoro-4-methoxyphenyl)-N′-methylbenzenesulfonimidamide (Compound 1b

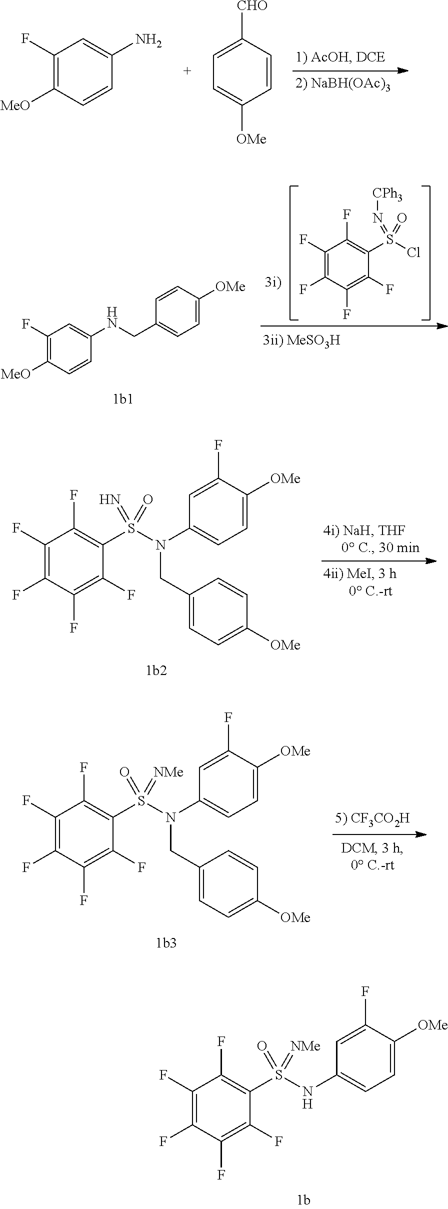

### Synthesis of 1b1

3-fluoro-4-methoxyanaline (1.1 equiv.) and AcOH (1.1 equiv.) are added to a suspension of 4-methoxybenzaldehyde (1.0) in DCE (0.5M). After stirring at room temperature for 20 min, NaBH(OAc)₃ (1.1 equiv.) is added in two portions and the reaction mixture is stirred at room temperature for 16 h. The reaction mixture is quenched with 0.1M NaOH, then the aqueous layer is extracted with DCM (×2) and the combined organic extracts are dried over MgSO₄. Crude sample can be purified by flash column chromatography on silica gel to afford 1b1.

### Synthesis of 1b2

1b2 can be prepared according to general procedure 1 using 1b1.

### Synthesis of 1b3

A suspension of NaH (1.1 equiv.) in THE (0.1M) is cooled 0° C. followed by the addition of 1b2 (1.0 equiv.). The resulting mixture is allowed to stir for 30 min, then Mel (1.1 equiv) is added dropwise at 0° C., and the reaction mixture allowed to gradually warm to room temperature. After stirring at room temperature for 3 h, the reaction is quenched with water and diluted with Et₂O. The two layers are partitioned and the aqueous layer is extracted with Et₂O (×2). Combined organic fractions are washed with a saturated solution of brine (×1) and then dried over MgSO₄. The crude sample is used directly in the next step.

### Synthesis of 1b

1b is prepared according a reported procedure. (J. Org. Chem. 2013, 78, 9396.)

### Example 3: N-(4-((5-chloro-2-((tetrahydro-2H-pyran-4-yl)amino)pyrimidin-4-yl)amino)pyridin-3-yl)-2,3,4,5,6-pentafluorobenzenesulfonamide (Compound 2a

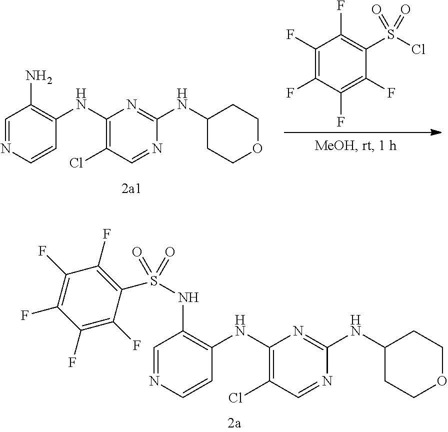

### Synthesis of 2a1

2a1 can be prepared according to the reported procedure by Ward (J. Med. Chem. 2015, 58, 4790).

### Synthesis of 2a

To a solution of pentafluorobenzenesulfonyl chloride (1.0 equiv) in anhydrous MeOH (0.3M) is added 2a1 (2.0 equiv), and the resulting mixture is stirred for 1 h. The reaction is then concentrated onto a small amount silica and purified by flash column chromatography.

### Example 4: N-(4-((5-chloro-2-((tetrahydro-2H-pyran-4-yl)amino)pyrimidin-4-yl)amino)pyridin-2-yl)-2,3,4,5,6-pentafluorobenzenesulfonamide (Compound 3a

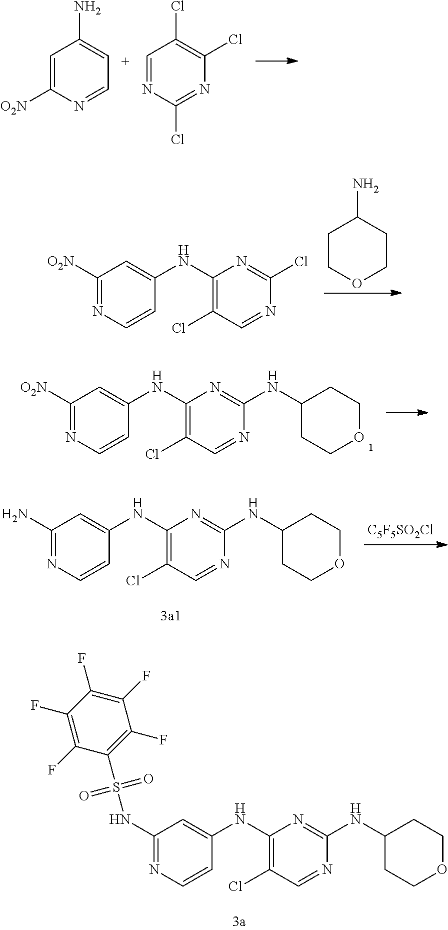

### Synthesis of 3a1

3a1 can be prepared according the general scheme above and described by Ward (J. Med. Chem. 2015, 58, 4790).

### Synthesis of 3a

3a can be prepared in an analogous manner as compound 2a using 3a1 as the amine.

### Example 5: 2,3,4,5,6-pentafluoro-N-(1-methyl-1,6-dihydroimidazo[4,5-d]pyrrolo[2,3-b]pyridin-2-yl)benzenesulfonamide (Compound 4a

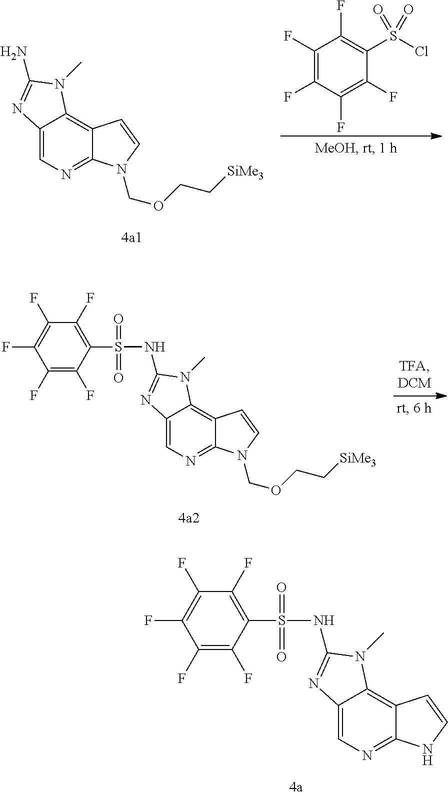 [SMILES: Cn1c(N)nc2cnc3c(ccn3COCC[Si](C)(C)C)c21 ; O=S(=O)(Cl)c1c(F)c(F)c(F)c(F)c1F ; Cn1c(NS(=O)(=O)c2c(F)c(F)c(F)c(F)c2F)nc2cnc3c(ccn3COCC[Si](C)(C)C)c21 ; Cn1c(NS(=O)(=O)c2c(F)c(F)c(F)c(F)c2F)nc2cnc3[nH]ccc3c21]

### Synthesis of 4a1

4a1 can be prepared according to the reported procedure by Goedken (J. Biol Chem. 2015, 290, 4573.)

### Synthesis of 4a2

4a2 can be prepared in an analogous manner as compound 2a using 4a2.

### Synthesis of 4a

Trifluoroacetic acid (3.0 equiv.) is added to a solution of 4a2 (1.0 equiv) and DCM (0.05 M) under N2 atmosphere. After stirring for 6 h at room temperature, the volatiles are removed under reduced pressure. The crude sample is then redissolved in a 10:1:15 mixture of THF:MeOH:H₂O and saturated aqueous NaHCO₃ and allowed to stir for 20 h. The organic volatiles are then removed under reduced pressure, and the residue is diluted with EtOAc. The two layers are partitioned and the aqueous layer is extracted with EtOAc (×2). Combined organic fractions are washed with a saturated solution of brine (×1) and then dried over MgSO₄. The crude sample is adsorbed onto a small amount of silica and purified by flash column chromatography.

### Example 6: N-methyl-1-((perfluorophenyl)sulfonyl)-1,6-dihydroimidazo[4,5-d]pyrrolo[2,3-b]pyridin-2-amine (Compound 5a

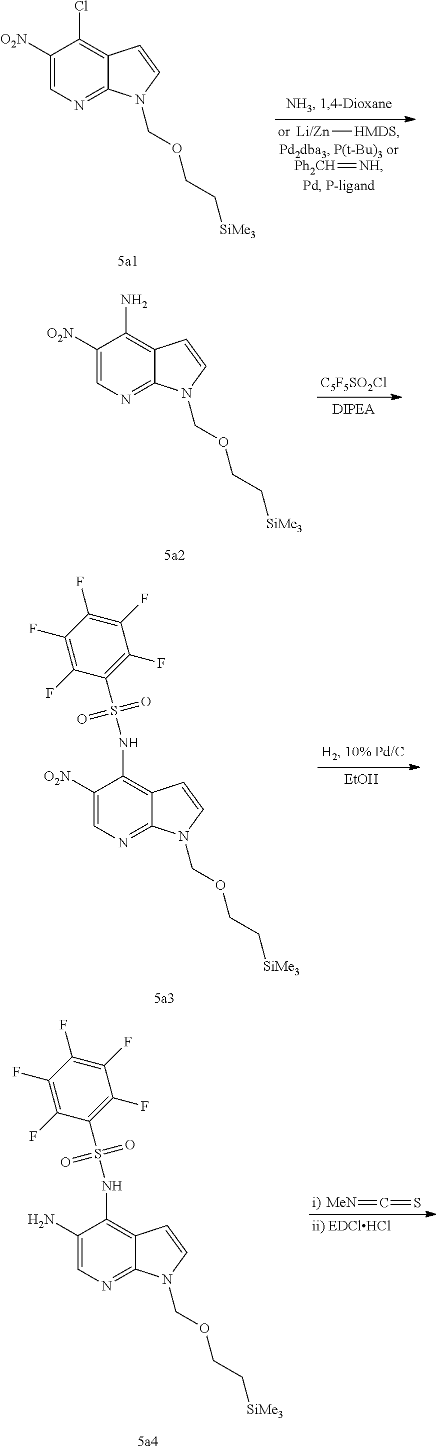

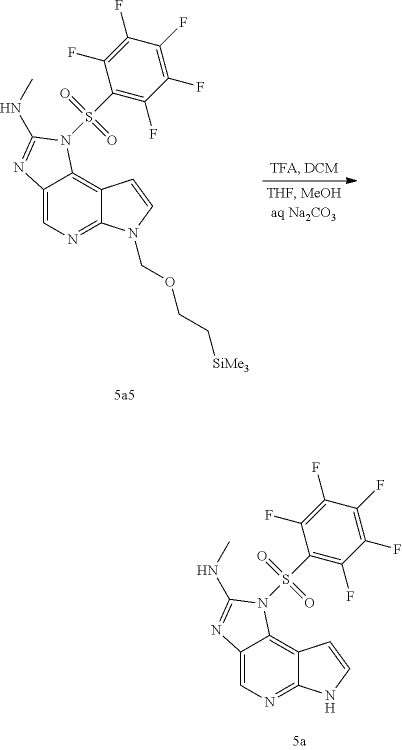 [SMILES: CNc1nc2cnc3c(ccn3COCC[Si](C)(C)C)c2n1S(=O)(=O)c1c(F)c(F)c(F)c(F)c1F ; CNc1nc2cnc3[nH]ccc3c2n1S(=O)(=O)c1c(F)c(F)c(F)c(F)c1F]

### Synthesis of 5a2

5a1 (prepared according to Goedken, Eric R. et al, Journal of Biological Chemistry (2015), 290(8), 4573-4589) can be aminated utilizing NH₃ in Dioxane at 80° C. (as per Liu, Bing; et al China, CN106336413 A) or with an ammonia surrogate such as Li or Zn HMDS catalyzed by a Palladium Phosphine complex (Pd2dba3/P(t-Bu)3, Toluene, rt-90° C.) (as per Lee, Sunwoo et al, Org. Lett. (2001), 3, 2729-2732) to give the desired amine 5a2.

### Synthesis of 5a3

A solution of the amine 5a2 (1 equiv.) and DIPEA (3 equiv.) are dissolved in anhydrous acetonitrile and cooled to 0° C. before 2,3,4,5,6-pentafluorobenzene-1-sulfonyl chloride (1.1 equiv.) is added dropwise. The resultant solution is allowed to stir overnight at R.T. The solvent is removed and the residue redissolved in CH₂C₂. The organics are then washed sequentially with 0.1 M HCl, saturated NaHCO₃ and brine. The organics are then dried over Na₂SO₄ and concentrated in vacuo to furnish the desired product 5a3 which is carried forward without any purification.

### Synthesis of 5a4

A mixture of 5a3, 10% Pd/C (wet, Deguessa type), and EtOH under N2 is evacuated under reduced pressure then back-filled with hydrogen using a balloon. After 2.5 h, the hydrogen atmosphere is evacuated then the mixture is filtered through Celite© rinsed with EtOH. The volatiles are removed under reduced pressure. The residue is purified on silica gel to afford the desired product 5a4.

### Synthesis of 5a5

Methyl isothiocyanate is added to a suspension of diamine 5a4 in THF. The mix is heated at reflux for 4 hours then a second aliquot of methyl isothiocyanate is added. The mixture is heated for a further 5 hour yielding the desired thiourea intermediate. The cooled mixture is treated with 1-(3-dimethylaminopropyl)-3-ethylcarbodiimide hydrochloride and stirred at ambient temperature. After 2 days a second aliquot of 1-(3-dimethylaminopropyl)-3-ethylcarbodiimide hydrochloride is added and stirring is continued for 3 days. The mixture is filtered, and the residue is washed with THF. The combined filtrate is concentrated under vacuum and purified by column chromatography on silica gel to give 5a5.

### Synthesis of 5a

Trifluoroacetic acid is added to a solution of 5a5 and CH₂Cl₂ under N2. After 6 h, the volatiles are removed under reduced pressure. THF, MeOH, water and saturated aqueous NaHCO₃ are added. After 20 h, the organic volatiles are removed under reduced pressure and the resulting solid is collected by filtration and rinsed with water. The residue is purified by silica gel chromatography to afford the desired product 5a.

### Example 7: N,1-dimethyl-6-((perfluorophenyl)sulfonyl)-1,6-dihydroimidazo[4,5-d]pyrrolo[2,3-b]pyridin-2-amine (Compound 6a

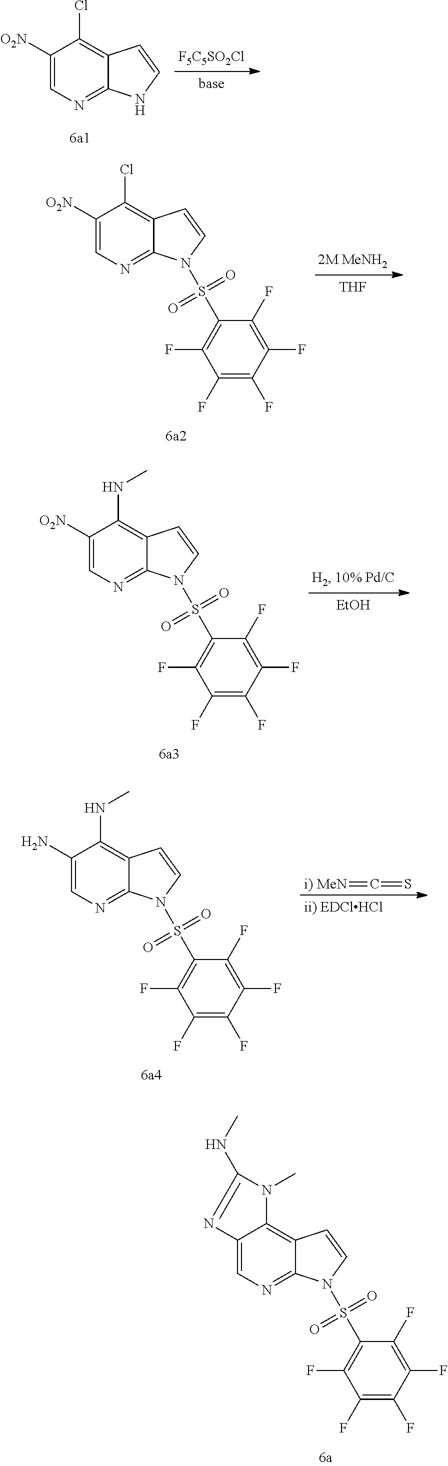

### Synthesis of 6a2

To a stirred solution of indole 6a1 in dimethylformamide at 25° C. is added pentafluorophenyl sulfonyl chloride followed by sodium carbonate, and the reaction mixture is stirred for 18 hours. The reaction mixture is diluted with ethyl acetate, washed with 20% ammonium chloride, and saturated sodium chloride. The organic layer is dried (sodium sulfite), and the ethyl acetate removed under vacuum. Column chromatography (3/1 ethyl acetate/hexane) yields the desired compound 6a2.

### Synthesis of 6a3

Methylamine (2 M solution in THF, 10.0 mL, 20.0 mmol) is added to 6a2 under air. The reaction vessel is sealed and left stirring for 1 h and saturated aqueous NaHCO₃ (25 mL) is added. The mixture is extracted with EtOAc (2×25 mL). The combined organics are dried over Na₂SO₄, filtered, and concentrated under reduced pressure to afford 6a3.

### Synthesis of 6a4

A mixture of 6a3, 10% Pd/C (wet, Deguessa type) (0.250 g), and EtOH (40 mL) under N₂ is evacuated under reduced pressure then back-filled with hydrogen using a balloon. After 2.5 h, the hydrogen atmosphere is evacuated then the mixture is filtered through Celite© and rinsed with EtOH (3×10 mL). The volatiles are removed under reduced pressure. The residue is purified on silica gel to afford 6a4.

### Synthesis of 6a

Cyanogen bromide (5 M solution in acetonitrile) is added to a solution of 6a4 and anhydrous EtOH. The reaction vessel is sealed and the solution is stirred for 2 h. After 15 min, the brown solution is warmed to 40° C. After 2 h, the reaction is allowed to cool to ambient temperature and the volatiles are removed under reduced pressure. Water (20 mL) and saturated aqueous NaHCO₃ (5 mL) are added. The aqueous phase is extracted with 5% MeOH in CH₂Cl₂ (25 mL). The combined organics is dried over Na₂SO₄, filtered, and concentrated under reduced pressure. The residue is purified on silica gel to afford 6a.

### Example 8: N-((3S,6S)-6-methyl-1-((perfluorophenyl)sulfonyl)piperidin-3-yl)-7H-pyrrolo[2,3-d]pyrimidin-4-amine (Compound 7a

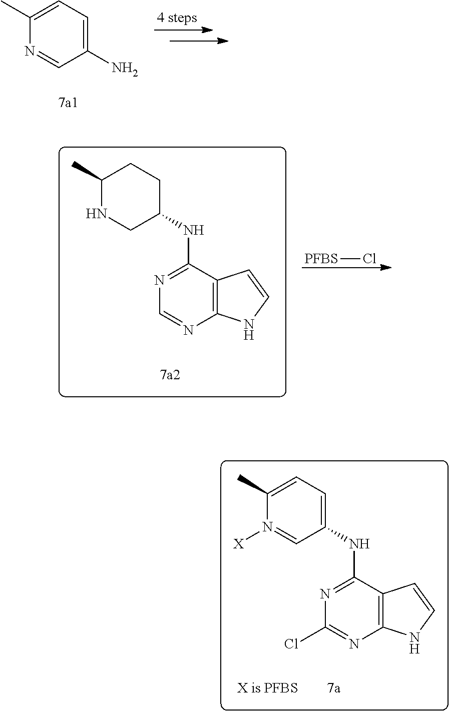

Compound 7a2 can be prepared from commercial starting material 7a1 using a reported procedure for its cis isomer (patent WO 2015/083028, example 5). Compound 7a2 is sulfonylated with PFBS-Cl analogously to the synthesis of 6a2 to afford compound 7a.

### Example 9: 2,3,4,5,6-pentafluoro-N-(2-methyl-5-(7H-pyrrolo[2,3-d]pyrimidine-4-carbonyl)phenyl)benzenesulfonamide (Compound 8a

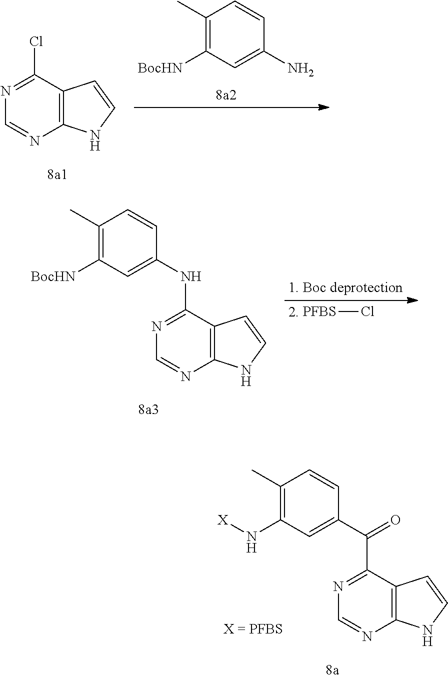

Commercially available compounds 8a1 and 8a2 are mixed in pyridine and heated to 85° C. (Ref: WO 98/23613, example 1) to afford 8a3 which is subsequently deprotected using a procedure analogous to that in the synthesis of 5a. The aniline is then sulfonylated with PFBS-Cl analogously to the synthesis of 6a2 to afford compound 8a.

### Example 10: 4-((3,5-bis(trifluoromethyl)benzyl)oxy)-1-((perfluorophenyl)sulfonyl)indoline (Compound 11a

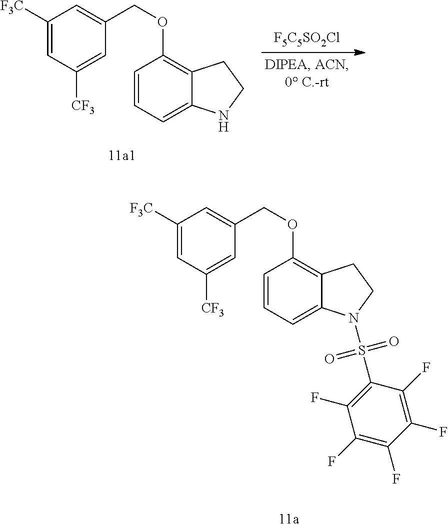

11a1 is prepared according to Nomura, Daniel K et al, WO2019075386.

A solution of the indoline 11a1 (1 equiv.) and DIPEA (3 equiv.) are dissolved in anhydrous acetonitrile and cooled to 0° C. before 2,3,4,5,6-pentafluorobenzene-1-sulfonyl chloride (1.1 equiv.) is added dropwise. The resultant solution is allowed to stir overnight at R.T. The solvent is removed, and the residue is purified by flash chromatography to yield 11a.

### Example 11: 4-((3,5-bis(trifluoromethyl)benzyl)oxy)-1-((perfluorophenyl)sulfonyl)-1H-indole (Compound 13a

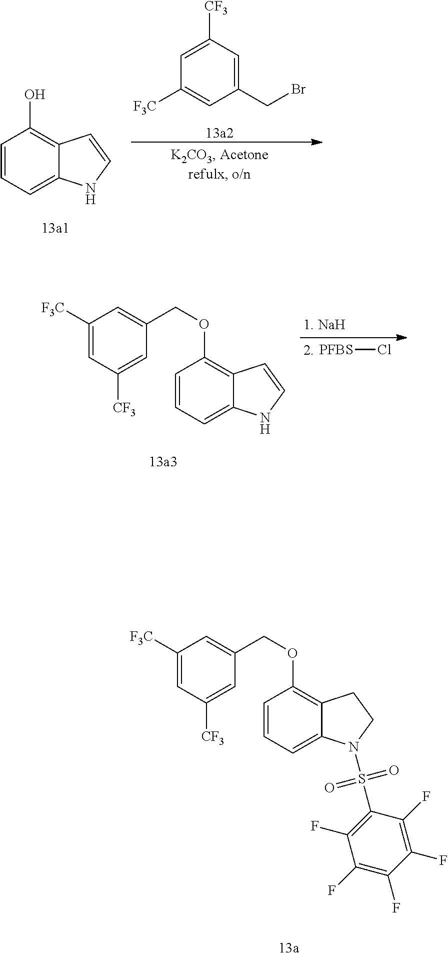

K₂CO₃ (1.56 g, 11.3 mmol) is added to indole 13a1 and benzyl bromide 13a2 in acetone (50 mL) is added. The solution is heated under reflux overnight. After the reaction, undissolved solid is filtered and the filtrate is evaporated under reduced pressure. The crude product is purified by column chromatography on silica gel using hexane/ethyl acetate (10:1, v/v) as eluent to yield 13a3.

To a stirred solution of indole 13a3 in dimethylformamide at 25° C. is added NaH and stirred for 10 minutes. Pentafluorophenyl sulfonyl chloride is added and the reaction mixture is stirred for 18 hours. The reaction mixture is diluted with ethyl acetate, washed with 20% ammonium chloride, and saturated sodium chloride. The organic layer is dried (sodium sulfite), and the ethyl acetate removed under vacuum. Column chromatography yields the desired compound 13a.

### Example 12: (Z)-2,3,4,5,6-pentafluoro-N-(2-((5-(4,4,4-trifluoro-1-(3-fluoro-1H-indazol-5-yl)-2-phenylbut-1-en-1-yl)pyridin-2-yl)oxy)ethyl)benzenesulfonamide (Compound 15a

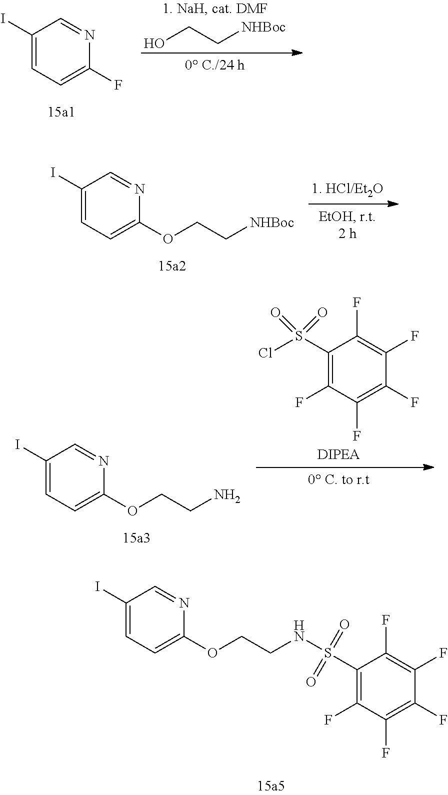 [SMILES: Fc1ccc(I)cn1 ; CC(C)(C)OC(=O)NCCO ; CC(C)(C)OC(=O)NCCOc1ccc(I)cn1 ; O=S(=O)(Cl)c1c(F)c(F)c(F)c(F)c1F ; NCCOc1ccc(I)cn1 ; O=S(=O)(NCCOc1ccc(I)cn1)c1c(F)c(F)c(F)c(F)c1F]

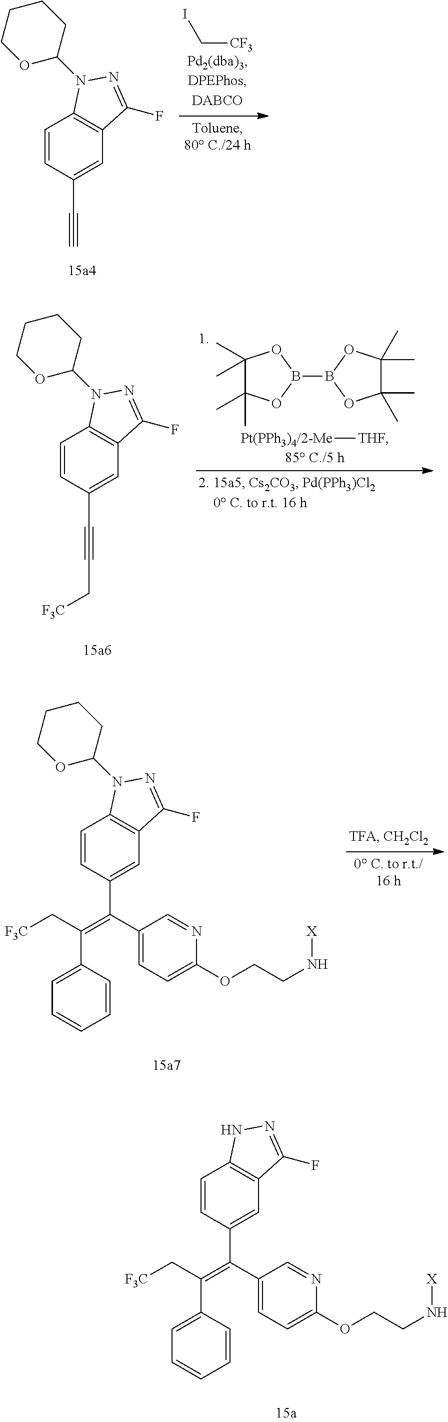

Compounds 15a3 and 15a4 can be prepared as described in US2016/347717.

15a3 can be converted to 15a5 by the following procedure: 2-((5-iodopyridin-2-yl)oxy)ethanamine 15a3 (1 equiv.) and DIPEA (3 equiv.) are dissolved in anhydrous acetonitrile and cooled to 0° C. before 2,3,4,5,6-pentafluorobenzene-1-sulfonyl chloride (1.1 equiv.). The resultant solution as allowed to stir overnight at R.T. The solvent is removed and the residue redissolved in CH₂Cl₂. The organics are then washed sequentially with 0.1 M HCl, saturated NaHCO₃ and brine. The organics are then dried over Na₂SO₄ and concentrated in vacuo to furnish sulfonamide 15a5.

15a4 can be converted to 15a using procedures analogous to those found in US2016/347717 by using compound 15a5 in place of the acrolein derivative.

### Example 13: N-(4-((3-chloro-4-(pyridin-3-ylmethoxy)phenyl)amino)-3-cyano-7-ethoxyquinolin-6-yl)-2,3,4,5,6-pentafluorobenzenesulfonamide (Compound 21a

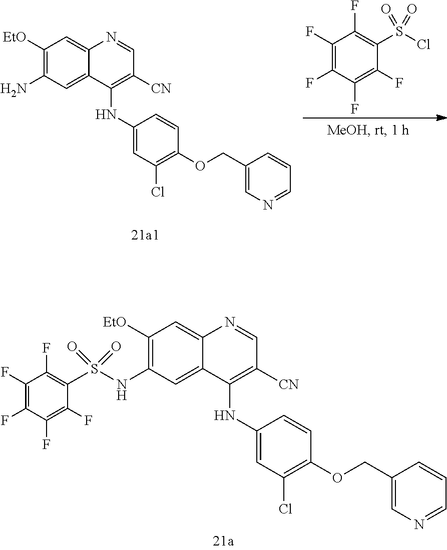 [SMILES: CCOc1cc2ncc(C#N)c(Nc3ccc(OCc4cccnc4)c(Cl)c3)c2cc1N ; O=S(=O)(Cl)c1c(F)c(F)c(F)c(F)c1F ; CCOc1cc2ncc(C#N)c(Nc3ccc(OCc4cccnc4)c(Cl)c3)c2cc1NS(=O)(=O)c1c(F)c(F)c(F)c(F)c1F]

### Synthesis of 21a1

21a1 can be prepared according the reported procedure by Ji. (Res. Chem. Intermed. 2013, 39, 3105.

### Synthesis of 21a

21a can be prepared in an analogous manner as compound 2a using 21a1.

### Example 14: (R)—N-(7-chloro-1-(1-((perfluorophenyl)sulfonyl)azepan-3-yl)-1H-benzo[d]imidazol-2-yl)-2-methylisonicotinamide (Compound 26a

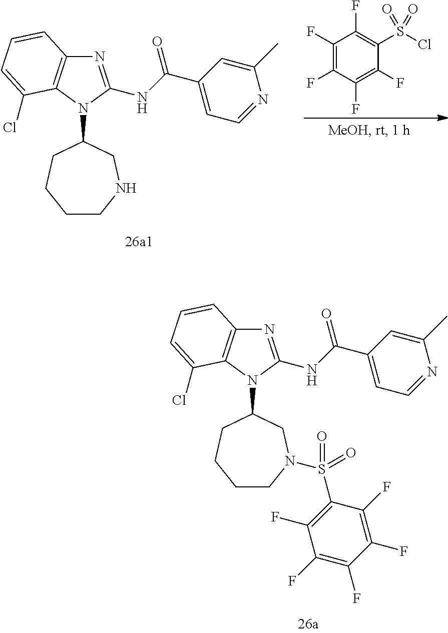

### Synthesis of 26a1

26a1 can be prepared according the reported procedure by Lelais. (J. Med. Chem. 2016, 59, 6671).

### Synthesis of 26a

26a can be prepared in an analogous manner as compound 2a using 26a1.

### Example 15: 6-fluoro-7-(2-fluoro-6-hydroxyphenyl)-1-(3-isopropyl-5-methylpyridin-4-yl)-4-((S)-2-methyl-4-((perfluorophenyl)sulfonyl)piperazin-1-yl)pyrido[2,3-d]pyrimidin-2(1H)-one (Compound 30a

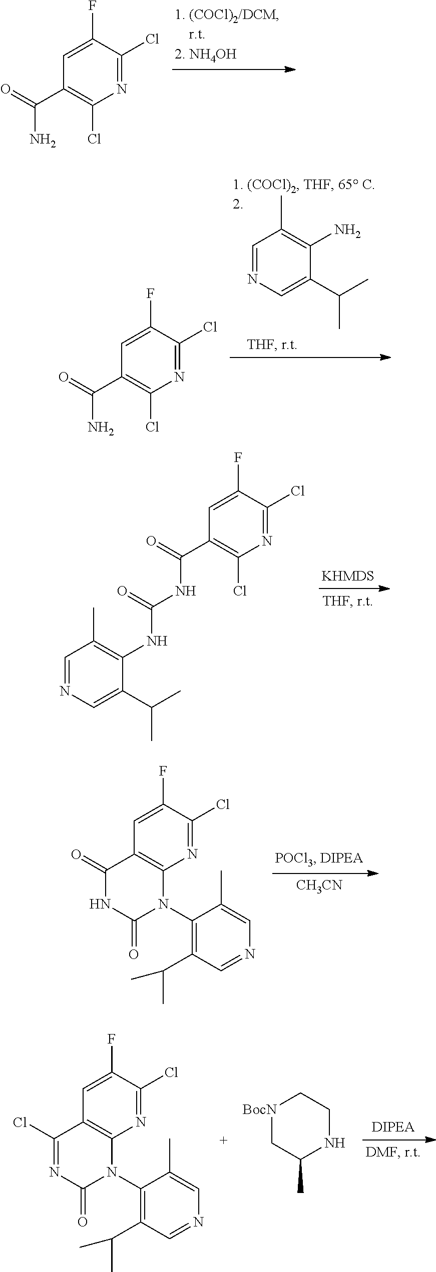

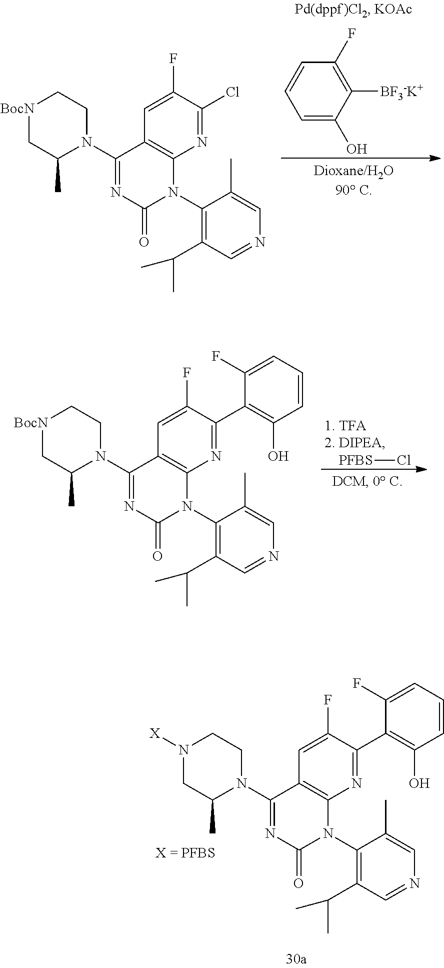

Compound 30a can be prepared from compound 30a1 (prepared as described in US2018/334454) by using a deprotection of the Boc protecting group using a procedure analogous to that in the synthesis of 5a followed by sulfonylation analogous the synthesis of 6a2. The mixture is then purified to afford 30a.

### Example 16: (S)—N-((1-(5-chloro-2-methoxybenzoyl)piperidin-3-yl)methyl)-2,3,4,5,6-pentafluorobenzenesulfonamide (Compound 34a

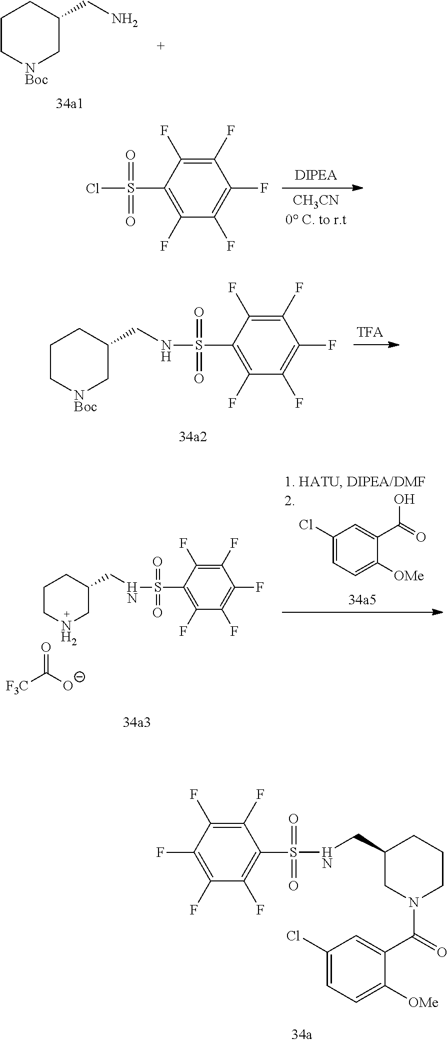

Compound 34a can be prepared using procedures adapted from WO2016179558A1. Compound 34a1 can be sulfonylated using a procedure analogous to the synthesis of 6a1. Deprotection of the boc protecting group can be accomplished using TFA in a step analogous to that in the synthesis of 5a. HATU mediated coupling of 34a3 with carboxylic acid 34a5 can be carried out using procedures analogous to those exemplified in WO2016179558A1.

### Example 17: N-(5-(3,5-dimethoxyphenethyl)-1H-pyrazol-3-yl)-2-((perfluorophenyl)sulfonamido)benzamide (Compound 38a

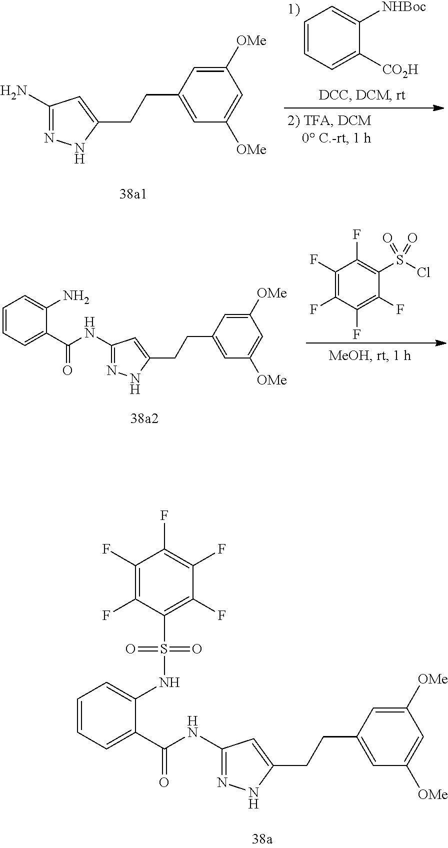 [SMILES: COc1cc(CCc2cc(N)n[nH]2)cc(OC)c1 ; CC(C)(C)OC(=O)Nc1ccccc1C(=O)O ; COc1cc(CCc2cc(NC(=O)c3ccccc3N)n[nH]2)cc(OC)c1 ; O=S(=O)(Cl)c1c(F)c(F)c(F)c(F)c1F ; COc1cc(CCc2cc(NC(=O)c3ccccc3NS(=O)(=O)c3c(F)c(F)c(F)c(F)c3F)n[nH]2)cc(OC)c1]

### Synthesis of 38a1

38a1 can be prepared according to reported procedure by Leach. (J. Med. Chem. 2012, 55, 5003.)

### Synthesis of 38a2

38a2 can be prepared according to the general scheme shown above using reported procedures.

### Synthesis of 38a

38a can be prepared in an analogous manner as compound 2a using 38a2.

### Example 18: 2,3,4,5,6-pentafluoro-N-(7-fluoro-4-(o-tolylamino)-3,3a-dihydroimidazo[1,5-a]quinoxalin-8-yl)benzenesulfonamide (Compound 49a

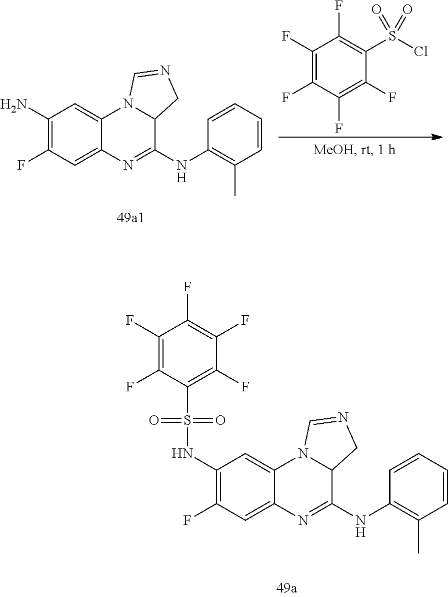

### Synthesis of 49a1

49a1 can be prepared according to reported procedure by Kim. (Bioorg. Med. Chem. Lett. 2011, 21, 6258.

### Synthesis of 49a

49a can be prepared in an analogous manner to compound 2a using 49a1.

### Example 19: methyl (1S,3R)-1-(4-(methoxycarbonyl)phenyl)-2-((perfluorophenyl)sulfonyl)-2,3,4,9-tetrahydro-1H-pyrido[3,4-b]indole-3-carboxylate (Compound 57a

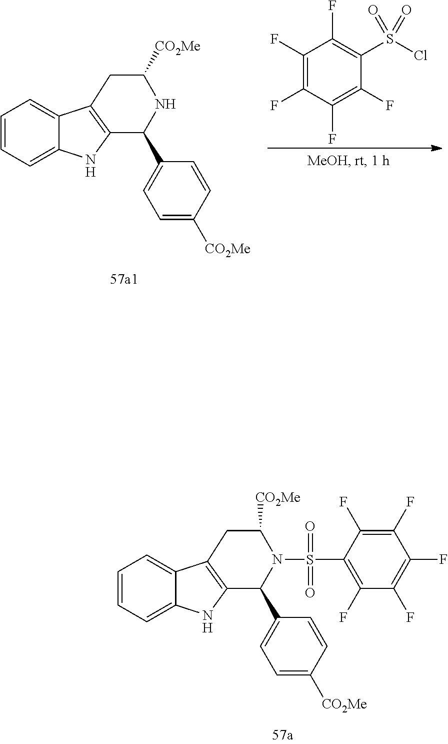 [SMILES: COC(=O)c1ccc([C@@H]2N[C@@H](C(=O)OC)Cc3c2[nH]c2ccccc32)cc1 ; O=S(=O)(Cl)c1c(F)c(F)c(F)c(F)c1F ; COC(=O)c1ccc([C@H]2c3[nH]c4ccccc4c3C[C@H](C(=O)OC)N2S(=O)(=O)c2c(F)c(F)c(F)c(F)c2F)cc1]

### Synthesis of 57a1

57a1 can be prepared according to reported procedure by Stockwell (US 20100081654A1).

### Synthesis of 57a

57a can be prepared in an analogous manner to compound 2a using 57a1.

### Example 20: 1-(bis(4-chlorophenyl)methyl)-4-((perfluorophenyl)sulfonyl)piperazine (Compound 61a

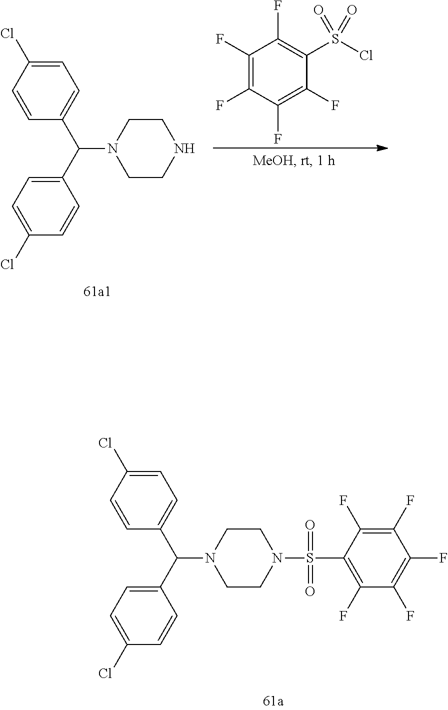 [SMILES: Clc1ccc(C(c2ccc(Cl)cc2)N2CCNCC2)cc1 ; O=S(=O)(Cl)c1c(F)c(F)c(F)c(F)c1F ; O=S(=O)(c1c(F)c(F)c(F)c(F)c1F)N1CCN(C(c2ccc(Cl)cc2)c2ccc(Cl)cc2)CC1]

### Synthesis of 61a1

61a1 can be prepared according to reported procedure by Munoz (Bioorg. Med. Chem. Lett. 2012, 22, 1822).

### Synthesis of 61a

61a can be prepared in an analogous manner to compound 2a using 61a1.

### General Procedure A—Synthesis of Batabulin Analogs

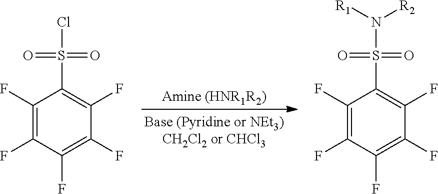

Pentafluorobenzenesulfonyl chloride (1 eq.) was added with dichloromethane or chloroform (0.3 M). The resulting solution was stirred at 0° C., followed by dropwise addition of appropriate starting amine (1.1 eq.) and base (3 eq.). The reaction was quenched with 0.1 M HCl and the aqueous phase was extracted thrice with dichloromethane. The combined organic layer was washed once with saturated sodium chloride solution, dried with sodium sulfate, and concentrated in vacuo. The crude sample was absorbed onto silica gel and purified using flash chromatography using a Hexane:Ethyl acetate gradient.

### General Procedure B—Synthesis of Batabulin Analogs

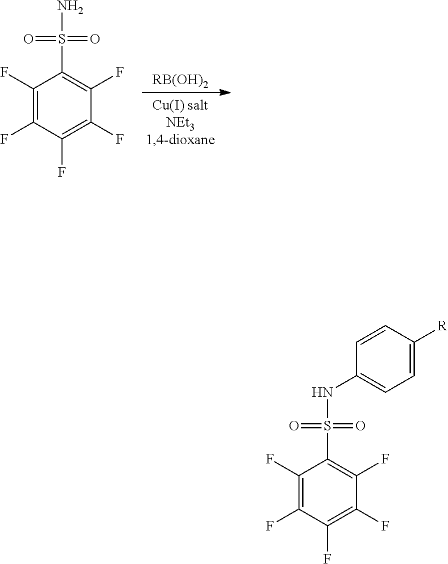

2,3,4,5,6-pentafluorobenzenesulfonamide (1 eq.) was dissolved in 1,4-dioxane (0.2 M) and the resulting solution was stirred at room temperature. After 10 minutes, the solution was added with an appropriate copper salt such as copper(I) iodide (0.4 eq.), triethylamine (1 eq.) and an appropriate boronic acid (1.5 eq.). The resulting mixture was stirred vigorously at room temperature. After 12 hours, the reaction mixture was filtered through a pad of Celite, and the collected organic layer was concentrated in vacuo. The crude sample was absorbed onto silica gel and purified using flash chromatography using a Hexane:Ethyl acetate gradient. The desired product was isolated as solid.

### Example 21: 2,3,4,5,6-pentafluoro-N-(3-fluoro-4-(trifluoromethoxy)phenyl)benzenesulfonamide (Compound 4-1

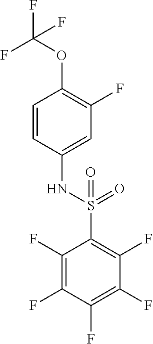 [SMILES: O=S(=O)(Nc1ccc(OC(F)(F)F)c(F)c1)c1c(F)c(F)c(F)c(F)c1F]

The title compound, 2,3,4,5,6-pentafluoro-N-(3-fluoro-4-(trifluoromethoxy)phenyl)benzenesulfonamide, was prepared via General Procedure A using pentafluorobenzenesulfonyl chloride (0.273 g, 1.03 mmol), anhydrous dichloromethane (0.25 M), 3-fluoro-4-(trifluoromethoxy)aniline (0.2 g, 1.03 mmol) and pyridine (0.365 g, 4.61 mmol). ¹H NMR (400 MHz, CDCl₃) δ 7.67 (s, 1H), 7.29 (t, 1H), 7.18 (dd, J=10.6, 2.7 Hz, 1H), 7.04-6.98 (m, 1H). ¹⁹F NMR (376 MHz, CDCl₃) δ −59.02 (d, J=4.8 Hz, 3F), −124.00-−124.13 (m, 1F), −136.13-−136.30 (m, 2F), −142.88-−143.05 (m, 1F), −157.18-−157.38 (m, 2F).

### Example 22: 2,3,4,5,6-pentafluoro-N-(4-phenoxyphenyl)benzenesulfonamide (Compound 4-2

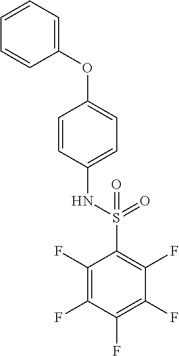 [SMILES: O=S(=O)(Nc1ccc(Oc2ccccc2)cc1)c1c(F)c(F)c(F)c(F)c1F]

The title compound, 2,3,4,5,6-pentafluoro-N-(4-phenoxyphenyl)benzenesulfonamide, was prepared via General Procedure B using 2,3,4,5,6-pentafluorobenzenesulfonamide (0.1 g, 0.405 mmol), anhydrous 1,4-dioxane (2.2 ml), CuI (0.031 g, 0.162 mmol), triethylamine (0.041 g, 0.405 mmol) and 4-phenoxyphenylboronic acid (0.13 g, 0.607 mmol). ¹H NMR (400 MHz, CDCl₃)) δ 7.40-7.35 (m, 2H), 7.20-7.14 (m, 3H), 7.03-6.99 (m, 2H), 6.99-6.94 (m, 2H), 6.91 (s, 1H). ¹⁹F NMR (376 MHz, CDCl₃) δ −135.99-−136.34 (m, 2F), −144.13-−144.45 (m, 1F), −157.84-−158.09 (m, 2F).

### Example 23: 2,3,4,5,6-pentafluoro-N-(3-fluoro-4-hydroxyphenyl)benzenesulfonamide (Compound 4-3

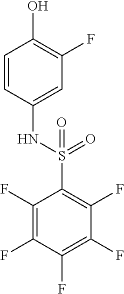 [SMILES: O=S(=O)(Nc1ccc(O)c(F)c1)c1c(F)c(F)c(F)c(F)c1F]

The intermediate, N-(4-(benzyloxy)-3-fluorophenyl)-2,3,4,5,6-pentafluorobenzenesulfonamide, was prepared via General Procedure B using 2,3,4,5,6-pentafluorobenzenesulfonamide (0.04 g, 0.162 mmol), anhydrous 1,4-dioxane (1.2 ml), copper(I) 2-thiophenecarboxylate (0.013 g, 0.0647 mmol), triethylamine (0.016 g, 0.162 mmol) and 4-benzyloxy-3-fluorophenylboronic acid (0.0796 g, 0.324 mmol). ¹H NMR (400 MHz, CDCl₃) δ 7.47-7.32 (m, 5H), 7.05 (dd, J=11.5, 2.6 Hz, 1H), 6.95 (t, J=8.7 Hz, 1H), 6.90-6.86 (m, 1H), 5.12 (s, 2H). ¹⁹F NMR (376 MHz, CDCl₃) δ −129.54 (dd, J=11.5, 8.5 Hz, 1F), −136.05-−136.20 (m, 2F), −144.01 (tt, J=21.1, 6.9 Hz, 1F), −157.84 (tt, J=21.2, 6.5 Hz, 2F).

N-(4-(benzyloxy)-3-fluorophenyl)-2,3,4,5,6-pentafluorobenzenesulfonamide (0.06 g, 0.134 mmol) was added with methanol (0.58 ml) and tetrahydrofuran (1.22 ml). The resulting solution was added with palladium 10% on carbon (2.14 mg) and stirred under hydrogen for 2 hours. The reaction mixture was filtered through a pad of Celite, and the collected organic layer was concentrated in vacuo. The title compound was isolated as beige solid and was lyophilized from water/acetonitrile to afford a white powder (100 mg, 100%). ¹H NMR (400 MHz, CD₃CN) δ 7.01 (dd, J=11.8, 2.5 Hz, 1H), 6.92 (t, J=8.9 Hz, 1H), 6.86 (ddd, J=8.7, 2.5, 1.0 Hz, 1H). ¹⁹F NMR (376 MHz, CD₃CN) 6-135.81-−135.96 (m, 1F), −138.18-−138.39 (m, 2F), −148.13-−148.33 (m, 1F), −161.11-−161.32 (m, 2F).

### Example 24: N-(4-(difluoromethoxy)-3-fluorophenyl)-2,3,4,5,6-pentafluorobenzenesulfonamide (Compound 4-4

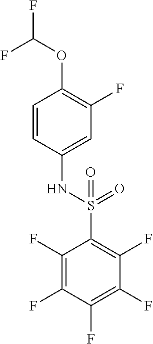 [SMILES: O=S(=O)(Nc1ccc(OC(F)F)c(F)c1)c1c(F)c(F)c(F)c(F)c1F]

To a 100 ml round-bottom flask equipped with a stir bar, was added 2-fluoro-4-nitrophenol (0.543 g, 3.42 mmol) and potassium hydroxide (3.838 g, 68.4 mmol) in a mixture of acetonitrile (17 ml) and H₂O (17 ml). While stirring at −50° C., the reaction mixture was added with diethyl (bromodifluoromethyl)phosphonate (1.826 g, 6.84 mmol). The reaction mixture was stirred for 2 hours while gradually warming upto room temperature. The mixture was then quenched with water and extracted three times with ethyl acetate. The collected organic layer was washed with saturated sodium chloride, dried over sodium sulfate, filtered and evaporated under reduced pressure to afford the intermediate, 1-(difluoromethoxy)-2-fluoro-4-nitrobenzene. ¹H NMR (400 MHz, CDCl₃) δ 8.11-7.99 (m, 2H), 7.49-7.40 (m, 1H), 6.74 (t, J=72.0 Hz, 1H). ¹⁹F NMR (376 MHz, CDCl₃) δ −82.45 (dd, J=72.0, 3.8 Hz, 2F), −124.73 (tt, J=8.8, 3.8 Hz, 1F).

To a 50 ml round-bottom flask equipped with a stir bar and purged with argon, was added 1-(difluoromethoxy)-2-fluoro-4-nitrobenzene (0.341 g, 1.65 mmol) and ethanol. The solution was then added with a saturated solution of ammonium chloride (0.440 g, 8.23 mmol) in a dropwise manner. To this solution was added iron (0.276 g, 4.94 mmol), and the resulting mixture was stirred at 75° C. for 3 hours. The reaction mixture was then filtered through a pad of Celite and then concentrated under reduced pressure. The mixture was diluted in water and extracted three times with DCM. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient to afford the intermediate, 4-(difluoromethoxy)-3-fluoroaniline. ¹H NMR (400 MHz, CDCl₃) δ 7.02 (t, J=8.7 Hz, 1H), 6.66-6.23 (m, 3H), 3.77 (s, 2H).

The title compound, N-(4-(difluoromethoxy)-3-fluorophenyl)-2,3,4,5,6-pentafluorobenzenesulfonamide, was prepared via General Procedure A using pentafluorobenzenesulfonyl chloride (0.075 g, 0.282 mmol), anhydrous dichloromethane (0.25 M), 4-(difluoromethoxy)-3-fluoroaniline (0.05 g, 0.282 mmol) and pyridine (0.022 g, 0.282 mmol). ¹H NMR (400 MHz, CDCl₃) δ 7.24 (t, J=8.6 Hz, 1H), 7.16 (dd, J=10.9, 2.7 Hz, 1H), 7.11 (s, 1H), 6.95 (ddd, J=8.8, 2.7, 1.6 Hz, 1H), 6.54 (t, J=72.9 Hz, 1H). ¹⁹F NMR (377 MHz, CDCl₃) δ −82.00 (dd, J=72.8, 4.5 Hz, 2F), −125.00-−125.13 (m, 1F), −136.16-−136.34 (m, 2F), −143.21 (tt, J=21.0, 7.1 Hz, 1F), −157.21-−157.44 (m, 2F).

### Example 25: N-(4-cyano-3-fluorophenyl)-2,3,4,5,6-pentafluorobenzenesulfonamide (Compound 4-5

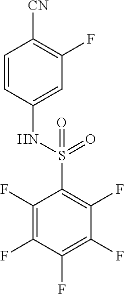 [SMILES: N#Cc1ccc(NS(=O)(=O)c2c(F)c(F)c(F)c(F)c2F)cc1F]

The title compound, N-(4-cyano-3-fluorophenyl)-2,3,4,5,6-pentafluorobenzenesulfonamide, was prepared via General Procedure A using pentafluorobenzenesulfonyl chloride (0.196 g, 0.735 mmol), anhydrous dichloromethane (0.25 M), 4-amino-2-fluorobenzonitrile (0.1 g, 0.735 mmol) and pyridine (0.058 g, 0.735 mmol). ¹H NMR (400 MHz, CDCl₃) δ 7.62 (dd, J=8.5, 7.0 Hz, 1H), 7.47 (s, 1H), 7.17 (dd, J=9.9, 2.2 Hz, 1H), 7.05 (dd, J=8.3, 2.2 Hz, 1H). ¹⁹F NMR (376 MHz, CDCl₃) δ −101.78 (dd, J=10.0, 7.0 Hz, 1F), −135.94-−136.10 (m, 2F), −141.88-−142.10 (m, 1F), −156.59-−156.79 (m, 2F).

### Example 26: 2,3,4,5,6-pentafluoro-N-(3-fluoro-4-(fluoromethoxy)phenyl)benzenesulfonamide (Compound 4-6

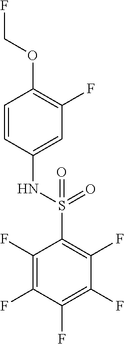 [SMILES: O=S(=O)(Nc1ccc(OCF)c(F)c1)c1c(F)c(F)c(F)c(F)c1F]

To a 100 ml round-bottom flask equipped with a stir bar, was added 2-fluoro-4-nitrophenol (0.148 g, 0.931 mmol) and cesium carbonate (0.393 g, 1.21 mmol) in anhydrous acetonitrile (9.3 ml). While stirring at 25° C., the reaction mixture was added with (fluoromethyl)(phenyl)(2,3,4,5-tetramethylphenyl)sulfonium tetrafluoroborate (0.370 g, 1.02 mmol). The reaction mixture was stirred for 2 hours while gradually warming upto room temperature. The mixture was then quenched with water and extracted three times with DCM. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient to afford the intermediate, 2-fluoro-1-(fluoromethoxy)-4-nitrobenzene. ¹H NMR (400 MHz, CDCl₃) δ 8.14-8.05 (m, 1H), 7.36 (t, J=8.3 Hz, 1H), 7.31-7.28 (m, 1H), 5.85 (d, J=52.9 Hz, 2H). ¹⁹F NMR (376 MHz, CDCl₃) δ −128.05 (t, J=8.9 Hz, 1F), −150.99 (t, J=52.9 Hz, 1F).

2-fluoro-1-(fluoromethoxy)-4-nitrobenzene (0.05 g, 0.264 mmol) was added with methanol (1.15 ml) and tetrahydrofuran (2.24 ml). The resulting solution was added with palladium 10% on carbon (28.1 mg) and stirred under hydrogen for 2 hours. The reaction mixture was filtered through a pad of Celite, and the collected organic layer was concentrated in vacuo to afford the desired intermediate, 3-fluoro-4-(fluoromethoxy)aniline. ¹H NMR (400 MHz, CD₃Cl) δ 7.03 (t, J=8.8 Hz, 1H), 6.47 (dd, J=12.3, 2.7 Hz, 1H), 6.39 (ddd, J=8.7, 2.8, 1.4 Hz, 1H), 5.61 (d, J=54.8 Hz, 2H), 3.67 (s, 2H). ¹⁹F NMR (376 MHz, CD₃Cl) δ −131.71-−131.89 (m, 1F), −147.75 (td, J=54.5, 3.0 Hz, 1F).

The title compound, 2,3,4,5,6-pentafluoro-N-(3-fluoro-4-(fluoromethoxy)phenyl)benzenesulfonamide, was prepared via General Procedure A using pentafluorobenzenesulfonyl chloride (0.061 g, 0.229 mmol), anhydrous dichloromethane (0.25 M), 3-fluoro-4-(fluoromethoxy)aniline (0.037 g, 0.229 mmol) and pyridine (0.018 g, 0.229 mmol). ¹H NMR (400 MHz, CDCl₃) δ 7.22-7.15 (m, 1H), 7.13 (dd, J=11.2, 2.6 Hz, 1H), 7.00-6.91 (m, 1H), 5.69 (d, J=53.8 Hz, 2H). ¹⁹F NMR (376 MHz, CDCl₃) δ −128.42 (t, J=9.9 Hz, 1F), −136.19 (ddt, J=19.9, 13.8, 7.8 Hz, 2F), −143.59 (tt, J=20.7, 6.9 Hz, 1F), −149.33 (t, J=53.9 Hz, 1F), −157.61 (tt, J=21.1, 6.6 Hz, 2F).

### Example 27: N-(4-(cyclopropylmethyl)-3-fluorophenyl)-2,3,4,5,6-pentafluorobenzenesulfonamide (Compound 4-7

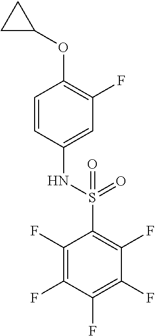 [SMILES: O=S(=O)(Nc1ccc(OC2CC2)c(F)c1)c1c(F)c(F)c(F)c(F)c1F]

To a 25 ml round-bottom flask equipped with a stir bar and purged with argon, was added pentafluorobenzenesulfonyl chloride (0.117 g, 0.439 mmol) in anhydrous DCM (0.67 M). While stirring at 0° C., the reaction mixture was added with 4-cyclopropoxy-3-fluoroaniline (0.068 g, 0.407 mmol) and 1,4-diazabicyclo[2.2.2]octane (0.059 g, 0.526 mmol). The reaction mixture was stirred for 2 hours while gradually warming upto room temperature. The mixture was then quenched with water and extracted three times with ethyl acetate. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient to afford the title compound, N-(4-cyclopropoxy-3-fluorophenyl)-2,3,4,5,6-pentafluorobenzenesulfonamide. ¹H NMR (400 MHz, CDCl₃) δ 7.24 (t, J=8.8 Hz, 1H), 7.01 (dd, J=11.4, 2.7 Hz, 1H), 6.97-6.91 (m, 2H), 3.79 (tt, J=5.9, 3.3 Hz, 1H), 0.85-0.80 (m, 4H). ¹⁹F NMR (376 MHz, CDCl₃) δ −130.83-−130.96 (m, 1F), −136.07-−136.31 (m, 2F), −144.08 (tt, J=20.9, 6.9 Hz, 1F), −157.72-−157.92 (m, 2F).

### Example 28: tert-butyl 4-((perfluorophenyl)sulfonyl)piperazine-1-carboxylate (Compound 4-8

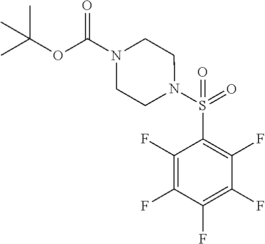 [SMILES: CC(C)(C)OC(=O)N1CCN(S(=O)(=O)c2c(F)c(F)c(F)c(F)c2F)CC1]

The title compound, tert-butyl 4-((perfluorophenyl)sulfonyl)piperazine-1-carboxylate, was prepared via General Procedure A using pentafluorobenzenesulfonyl chloride (2 g, 7.5 mmol), anhydrous dichloromethane (0.25 M), 1-Boc-piperazine (1.4 g, 7.5 mmol) and triethylamine (1.53 g, 15 mmol). ¹H NMR (400 MHz, CDCl₃) δ 3.62-3.57 (m, 4H), 3.28 (t, J=5.1 Hz, 4H), 1.48 (s, 9H). ¹⁹F NMR (376 MHz, CDCl₃) δ −134.10 (m, 2F), −144.68 (m, 1F), −157.62-−158.10 (m, 2F).

### Example 29: 3-fluoro-4-methyl-N-((perfluorophenyl)sulfonyl)benzamide (Compound 4-9

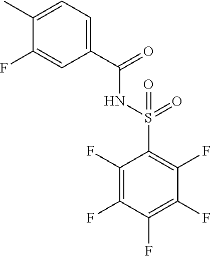 [SMILES: Cc1ccc(C(=O)NS(=O)(=O)c2c(F)c(F)c(F)c(F)c2F)cc1F]

To an oven-dried 50 ml round-bottom flask equipped with a stir bar and purged with argon, was added 2,3,4,5,6-pentafluorobenzenesulfonamide (0.1 g, 0.405 mmol) and 3-fluoro-4-methylbenzoic acid (0.0624 g, 0.405 mmol) in anhydrous DCM (4.05 ml). While stirring at room temperature, the reaction mixture was added with N-(3-Dimethylaminopropyl)-N-ethylcarbodiimide hydrochloride (0.116 g, 0.607 mmol) followed by 4-(Dimethylamino)pyridine (0.0494 g, 0.405 mmol). The resulting mixture was stirred at room temperature for 6 hours. The mixture was then quenched with 1 M HCl and extracted three times with DCM. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient. ¹H NMR (400 MHz, CH₃CN) δ 7.60 (dd, J=7.9, 1.9 Hz, 1H), 7.54 (dd, J=10.3, 1.8 Hz, 1H), 7.41 (td, J=7.7, 0.9 Hz, 1H), 2.35 (d, J=2.0 Hz, 3H). ¹⁹F NMR (376 MHz, CH₃CN) δ −117.41 (ddd, J=10.3, 7.7, 2.3 Hz, 1F), −137.23-−137.46 (m, 2F), −146.93 (tt, J=20.5, 7.7 Hz, 1F), −161.38-−161.59 (m, 2F).

### Example 30: N-(2,3-dihydrobenzo[b][1,4]dioxin-6-yl)-2,3,4,5,6-pentafluorobenzenesulfonamide (Compound 4-10

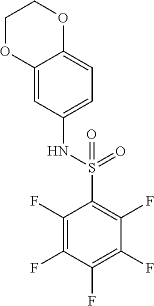 [SMILES: O=S(=O)(Nc1ccc2c(c1)OCCO2)c1c(F)c(F)c(F)c(F)c1F]

To an oven-dried 5 ml microwave vial equipped with a stir bar and purged with argon, was added Pentafluorobenzenesulfonyl chloride (0.1 g, 0.375 mmol) in anhydrous DCM (1.88 ml). While stirring at 0° C., the reaction mixture was added with 3,4-ethylenedioxyaniline (017 g, 1.13 mmol). The resulting mixture was stirred for 12 hours while gradually warming upto room temperature. The mixture was then quenched with 1 M 1-1 (and extracted three times with DCM. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient to afford the title compound. ¹H NMR (400 MHz, CDCl₃) δ 6.81 (d, J=8.6 Hz, 1H), 6.76 (d, J=2.6 Hz, 1H), 6.66 (dd, J=8.7, 2.6 Hz, 1H), 4.25 (s, 4H). ¹⁹F NMR (376 MHz, CDCl₃) δ −136.12-−136.28 (m, 2F), −144.62 (tt, J=21.4, 6.8 Hz, 1F), −158.02-−158.23 (m, 2F).

### Example 31: 2,3,4,5,6-pentafluoro-N-(4-phenylthiazol-2-yl)benzenesulfonamide (Compound 4-11

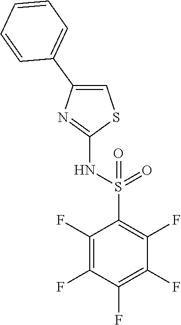 [SMILES: O=S(=O)(Nc1nc(-c2ccccc2)cs1)c1c(F)c(F)c(F)c(F)c1F]

The title compound, 2,3,4,5,6-pentafluoro-N-(4-phenylthiazol-2-yl)benzenesulfonamide, was prepared via General Procedure A using pentafluorobenzenesulfonyl chloride (0.166 g, 0.624 mmol), anhydrous dichloromethane (0.1 M), 2-amino-4-phenylthiazole (0.1 g, 0.567 mmol) and triethylamine (0.183 g, 1.42 mmol). ¹H NMR (400 MHz, CH₃CN) δ 7.70-7.63 (m, 2H), 7.53-7.42 (m, 3H), 6.93 (s, 1H). ¹⁹F NMR (376 MHz, CH₃CN) 6-138.58 (dt, J=21.2, 5.6 Hz, 2F), −151.97-−152.15 (m, 1F), −162.67 (tt, J=20.5, 5.7 Hz, 2F).

### Example 32: 2,3,4,5,6-pentafluoro-N-(3-fluoro-4-methoxyphenyl)-N-methylbenzenesulfonamide (Compound 4-12

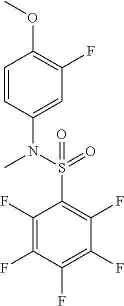 [SMILES: COc1ccc(N(C)S(=O)(=O)c2c(F)c(F)c(F)c(F)c2F)cc1F]

To a 50 ml round-bottom flask equipped with a stir bar and purged with argon, was added pentafluorobenzenesulfonyl chloride (0.726 g, 2.73 mmol) in anhydrous DCM (0.3 M). While stirring at 0° C., the reaction mixture was added with 3-fluoro-4-methoxy-aniline (0.5 g, 3.54 mmol) and 1,4-diazabicyclo[2.2.2]octane (0.306 g, 2.73 mmol). The reaction mixture was stirred for 2 hours while gradually warming upto room temperature. The mixture was then quenched with water and extracted three times with ethyl acetate. The collected organic layer was washed with saturated sodium chloride, dried over sodium sulfate, filtered and evaporated under reduced pressure. The crude sample was absorbed onto silica gel and purified using flash chromatography using a Hexane:Ethyl acetate gradient to afford the intermediate, 2,3,4,5,6-pentafluoro-N-(3-fluoro-4-methoxy-phenyl)benzenesulfonamide. ¹H NMR (400 MHz, CDCl₃) δ 7.22 (s, 1H), 7.03 (dd, J=11.6, 2.4 Hz, 1H), 6.98-6.87 (m, 2H), 3.88 (s, 3H).

To a 25 ml round-bottom flask equipped with a stir bar and purged with argon, was added 2,3,4,5,6-pentafluoro-N-(3-fluoro-4-methoxy-phenyl)benzenesulfonamide (0.2 g, 0.538 mmol), potassium carbonate (0.223 g, 1.62 mmol) and DMF (1.5 ml). The reaction mixture was then added with iodomethane (0.153 g, 1.08 mmol) at room temperature and stirred for 4 hours. The mixture was then quenched with water and extracted with EtOAc. The collected organic layer was washed once with saturated sodium chloride, dried with sodium sulfate, filtered and evaporated under reduced pressure. The crude sample was absorbed onto silica gel and purified using flash chromatography using a Hexane:Ethyl acetate gradient to afford the title compound, 2,3,4,5,6-pentafluoro-N-(3-fluoro-4-methoxy-phenyl)-N-methyl-benzenesulfonamide (0.160 g, 415.28 umol, 77.09% yield). ¹H NMR (400 MHz, CDCl₃) δ 7.04-6.97 (m, 2H), 6.94 (t, J=9.0 Hz, 1H), 3.92 (s, 3H), 3.40 (t, J=1.4 Hz, 3H). ¹⁹F NMR (376 MHz, CDCl₃) δ −131.56 (dd, J=11.8, 8.4 Hz, 1F), −133.82 (dt, J=20.7, 6.0 Hz, 2F), −144.73 (tt, J=21.0, 6.8 Hz, 1F), −158.04-−158.23 (m, 2F). Purity by HPLC: 99.5% @ 254 nm.

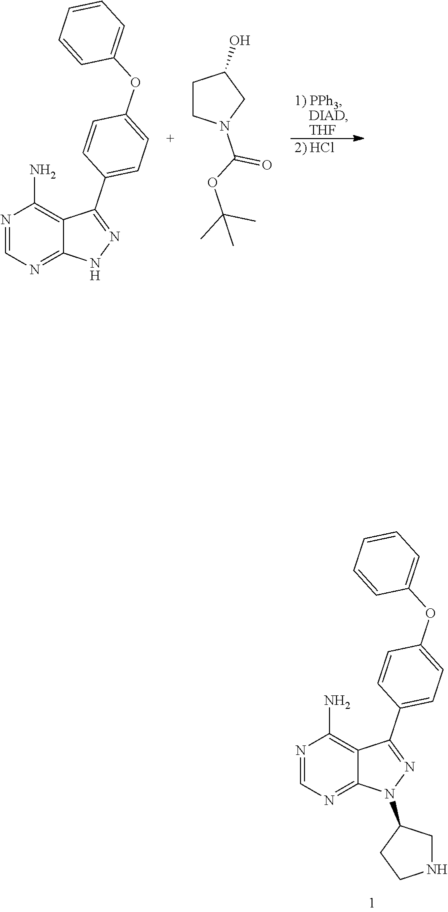

### Synthesis of (1S)-3-(4-phenoxyphenyl)-1-pyrrolidin-3-yl-pyrazolo[3,4-d]pyrimidin-4-amine (1)

To a solution of 3-(4-phenoxyphenyl)-1H-pyrazolo[3,4-d]pyrimidin-4-amine (2 g, 6.59 mmol) in anhydrous tetrahydrofuran (100 mL) were added tert-butyl (3S)-3-hydroxypyrrolidine-1-carboxylate (2.47 g, 13.19 mmol) and triphenylphosphine (3.46 g, 13.19 mmol) at room temperature. The mixture was cooled down to 0° C. in an ice bath and a solution of diisopropyl azodicarboxylate (2.67 g, 13.19 mmol, 2.59 mL) in 20 mL of anhydrous THE was added dropwise over 2 hours. The mixture was allowed to warm gradually to room temperature and stirred overnight. After 16 hours, concentrated HCl (13 mL) was added to the mixture and it was stirred at 50° C. for 3 hrs (bubbling was observed during the first 1.5 hours) and then cooled down to room temperature. THE was evaporated, 10 mL of water was added and the aqueous phase was extracted with DCM to remove all undesired organic materials (monitored by TLC, Ethyl Acetate/DCM 1:1). 10% NaOH was added to the aqueous phase dropwise until a pH of 9 was achieved. The aqueous solution was extracted with 10% MeOH/DCM. The combined organic layers were dried over sodium sulfate and evaporated, providing the crude product as brown semi-solid (0.89 g). The crude was purified by column chromatography on silica gel eluting with 0-25% MeOH/DCM providing the anticipated product as yellowish solid (0.53 g, 21% yield). ¹H NMR (400 MHz, DMSO-d₆) δ 9.69 (s, 2H), 8.28 (s, 1H), 7.72 (d, J=8.7 Hz, 2H), 7.44 (dd, J=8.6, 7.3 Hz, 2H), 7.26-7.10 (m, 5H), 5.60 (tt, J=7.2, 4.7 Hz, 1H), 3.82-3.30 (m, 5H), 2.49-2.27 (m, 2H). ESI-MS: measured m/z 372.90 [M+H]⁺.

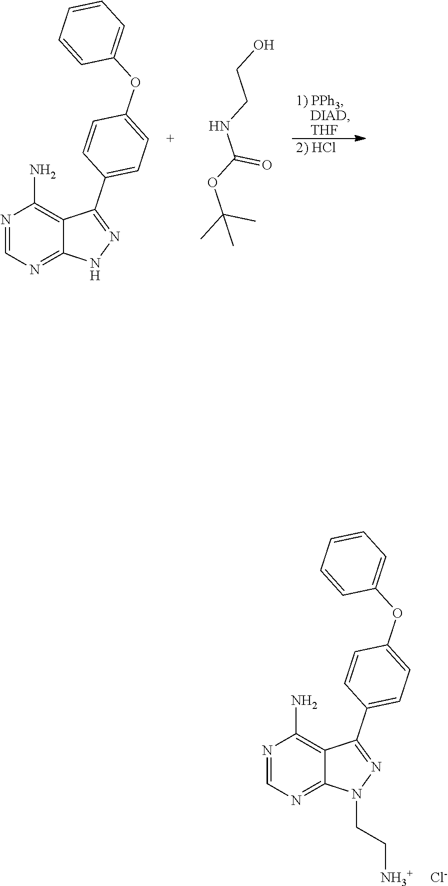

### Synthesis of 1-(2-aminoethyl)-3-(4-phenoxyphenyl)pyrazolo[3,4-d]pyrimidin-4-amine hydrochloride

To a solution of 3-(4-phenoxyphenyl)-1H-pyrazolo[3,4-d]pyrimidin-4-amine (20 g, 65.94 mmol) in tetrahydrofuran, anhydrous (900 mL) were added tert-butyl N-(2-hydroxyethyl)carbamate (21.26 g, 131.88 mmol, 20.44 mL) and triphenylphosphine (34.59 g, 131.88 mmol). The mixture was cooled down to 0° C. in an ice bath and a solution of diisopropyl azodicarboxylate (26.67 g, 131.88 mmol, 25.89 mL) in 100 mL of anhydrous THE was added dropwise over 5 hours. The solution was allowed to warm slowly in the ice bath and stirred overnight at room temperature. Concentrated HCl (130 mL) was added to the reaction mixture slowly and it was stirred at 50° C. for 4 hrs (gas evolving during the first 2.5 hours observed) and then cooled down to 0° C. in an ice bath. After 1 hour at 0° C., a beige solid precipitated from the solution. It was filtered off, washed with THF, and dried under vacuum to afford the anticipated product as a beige solid (14.79 g, 56.82% yield). ¹H NMR (400 MHz, DMSO-d₆) δ 8.61 (s, 1H), 8.38 (s, 3H), 7.70 (d, J=8.7 Hz, 2H), 7.46 (dd, J=8.6, 7.3 Hz, 2H), 7.27-7.06 (m, 5H), 4.70 (t, J=6.1 Hz, 2H), 3.36 (q, J=6.0 Hz, 2H). ESI-MS: measured m/z 346.90 [M+H]⁺.

### Synthesis of Intermediate D

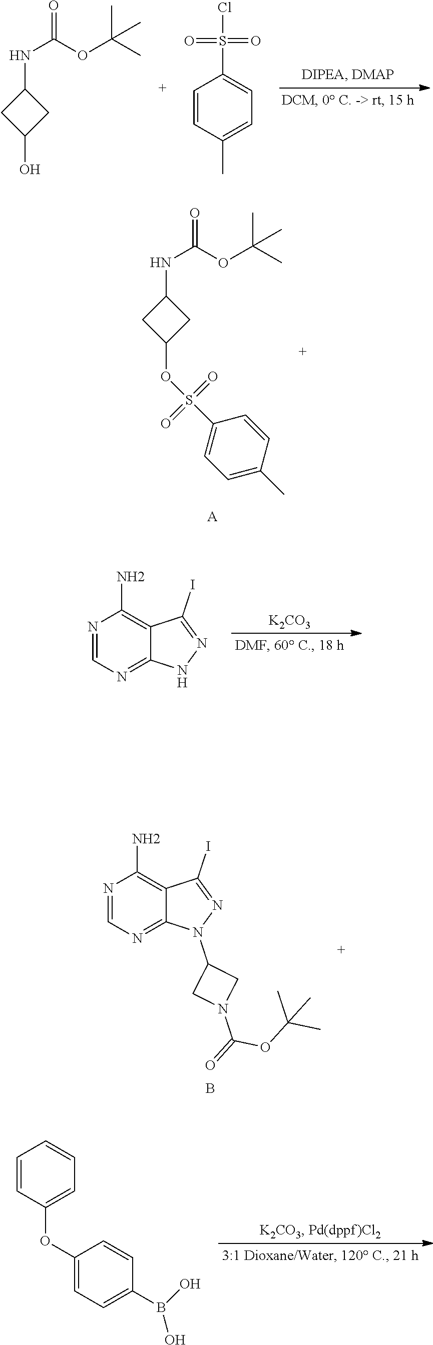

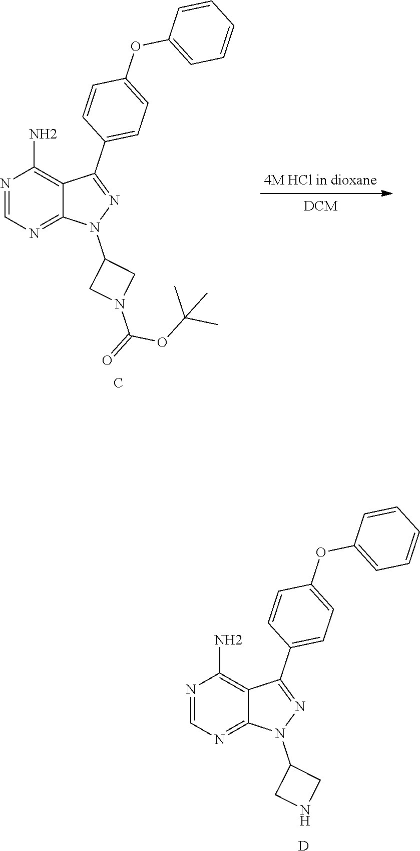

Intermediate A: 3-((tert-butoxycarbonyl)amino)cyclobutyl 4-methylbenzenesulfonate. To a oven dried microwave vial was added 1-(tert-Butoxycarbonyl)-3-hydroxyazetidine (500 mg, 2.89 mmol) in Dichloromethane (28.9 mL, 0.1M). The solution was cooled to 0° C. and N,N-Diisopropylethylamine (1.5 mL, 8.66 mmol) and 4-(Dimethylamino)pyridine (35.3 mg, 0.289 mmol) were added and the solution was stirred at 0° C. for 10 min. p-Toluenesulfonyl Chloride (248 mg, 1.3 mmol) was then added to the mixture which was left to stir at room temperature for 15 h. The reaction was subsequently quenched on ice with 1M HCl, and washed 3× with water. Combined organic layers were dried over Mg₂SO₄ and concentrated in vacuo to yield tert-butyl 3-(tosyloxy)azetidine-1-carboxylate as a white solid (283 mg, 99%). Product used as crude in the next step. ¹H NMR (400 MHz, Chloroform-d) δ 7.77-7.72 (m, 2H), 7.34 (d, J=8.0 Hz, 2H), 4.97 (tt, J=6.7, 4.3 Hz, 1H), 4.07 (dd, J=10.1, 6.7 Hz, 2H), 3.89 (dd, J=10.6, 4.2 Hz, 2H), 2.43 (s, 3H), 1.38 (s, 9H).

Intermediate B: tert-butyl 3-(4-amino-3-iodo-1H-pyrazolo[3,4-d]pyrimidin-1-yl)azetidine-1-carboxylate To an oven dried round bottom flask was added 3-iodo-1H-pyrazolo[3,4-d]pyrimidin-4-amine (631 mg, 2.42 mmol) (intermediate A). The vial was subsequently purged once with nitrogen followed by the addition of DMF (12.1 mL, 0.2 M). The reaction was cooled to 0° C. and potassium carbonate (668 mg, 4.84 mmol) was then added on ice followed by the addition of tert-butyl 3-(tosyloxy)azetidine-1-carboxylate (950 mg, 2.9 mmol). The reaction was then heated to 60° C. for 18 h and subsequently quenched over 1M HCl, extracted thrice with ethyl acetate and washed four times with brine. Combined organic layers were dried over Mg₂SO₄, and evaporated. Crude product purified on a Biotage Isolera using a 100 g cartridge and a 1-5% MeOH/DCM gradient to obtain tert-butyl 3-(4-amino-3-iodo-1H-pyrazolo[3,4-d]pyrimidin-1-yl)azetidine-1-carboxylate as an off-white solid (232 mg, 20%). ¹H NMR (400 MHz, Chloroform-d) δ 8.32 (s, 1H), 6.22 (s, 2H), 5.61 (tt, J=8.1, 5.7 Hz, 1H), 4.16 (ddd, J=9.6, 6.7, 1.2 Hz, 2H), 3.82 (ddd, J=9.5, 4.4, 1.1 Hz, 2H). MS (ESI, [M+H]⁺) m/z 417.23.

Intermediate C: tert-butyl 3-(4-amino-3-(4-phenoxyphenyl)-1H-pyrazolo[3,4-d]pyrimidin-1-yl)azetidine-1-carboxylate. A sealed microwave vial containing intermediate B (232 mg, 0.557 mmol), 4-methoxyphenylboronic acid (167 mg, 0.78 mmol), potassium carbonate (154 mg, 1.11 mmol) in 3:1 1,4-dioxane:H₂O (0.05 M) was purged under nitrogen for 15 min. [1,1′-Bis(diphenylphosphino)ferrocene]dichloropalladium(II) (20.4 mg, 0.0279 mmol) was subsequently added and nitrogen was bubbled through the solution for 10 min. The reaction mixture was then heated to 120° C. and left to stir for 21 h. The solution was filtered through celite and the filtrate concentrated. Filtrate was re-dissolved in ethyl acetate and extracted three times with water and the combined organic layers were dried over Mg₂SO₄ and concentrated. Silica gel chromatography (1-4% MeOH/DCM) yielded the desired tert-butyl 3-(4-amino-3-(4-phenoxyphenyl)-1H-pyrazolo[3,4-d]pyrimidin-1-yl)azetidine-1-carboxylate product as an off-white solid (254 mg, 98%). ¹H NMR (400 MHz, Chloroform-d) δ 8.30 (s, 1H), 7.70-7.64 (m, 2H), 7.41-7.34 (m, 2H), 7.19-7.11 (m, 3H), 7.10-7.04 (m, 2H), 5.69 (tt, J=8.1, 5.7 Hz, 1H), 4.61-4.50 (m, 2H), 4.40 (t, J=8.6 Hz, 2H), 1.46 (s, 9H). MS (ESI, [M+H]⁺) m/z 459.3.

Intermediate D: 1-(azetidin-3-yl)-3-(4-phenoxyphenyl)-1H-pyrazolo[3,4-d]pyrimidin-4-amine. In a 25 mL round bottom was dissolved intermediate C in DCM and treated with 4M HCl in dioxane (total concentration 0.07 M, 1:1 4M HCl:DCM). The reaction mixture was stirred at room temperature for 12 h. The solvent was subsequently evaporated off under vacuum and co-distilled twice using chloroform to afford 1-(azetidin-3-yl)-3-(4-phenoxyphenyl)-1H-pyrazolo[3,4-d]pyrimidin-4-amine as a yellow, off-white solid which was used without further purification. MS (ESI, [M+H]⁺) m/z 359.6.

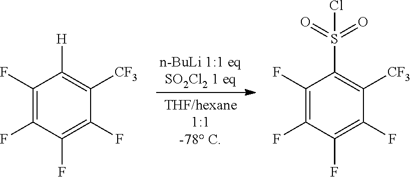 [SMILES: Fc1cc(C(F)(F)F)c(F)c(F)c1F ; O=S(=O)(Cl)c1c(F)c(F)c(F)c(F)c1C(F)(F)F]

### Synthesis of 2,3,4,5-tetrafluoro-6-(trifluoromethyl)benzenesulfonyl chloride

To a solution of 1,2,3,4-tetrafluoro-5-(trifluoromethyl)benzene (1.87 g, 8.58 mmol) in anhydrous THE cooled to −78° C. was added n-butyllithium (2.5 M in hexane, 3.77 mL) dropwise over a period of 10 min under argon. The resulting dark violet solution was slowly added to a hexane (5 mL) solution of sulfuryl chloride (1.16 g, 8.58 mmol, 693.05 uL) at −78° C. via cannula. After stirring for 3 h, the mixture was quenched with 5 mL of water at −78° C. and the bath was removed. The mixture was partitioned between ethyl acetate and cold water and the organic phase was separated. The organic phase was washed with cold water twice, dried over sodium sulfate and concentrated in vacuo to afford the anticipated product as brown oil (1.43 g, 52.68% yield). ¹⁹F NMR (376 MHz, CDCl₃) δ −50.67 (d, J=37.7 Hz), −122.70 (ddd, J=23.5, 14.6, 8.8 Hz), −129.90 (ddt, J=37.7, 20.4, 9.6 Hz), −138.15 (td, J=20.6, 13.9 Hz), −142.22 (ddd, J=22.9, 19.8, 10.7 Hz).

### General Procedure A-1

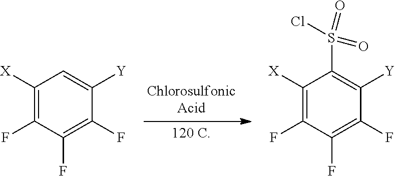

A substituted fluoro-arene (1 eq) was added to a cold solution of chlorosulfonic acid cooled to 0° C. The reaction vessel was outfitted with a water jacketed reflux condenser and subsequently heated to 120° C. using a sand bath for 1-16 hrs. Once starting material was consumed, the reaction was cooled to room temperature then poured slowly over crushed ice. The resulting mixture was partitioned between DCM and 1M HCl and the organic phase separated. The remaining aqueous phase was extracted twice more with DCM. The combined organic phases were washed with brine, dried over sodium sulfate, and concentrated in vacuo to afford the desired arylsulfonyl chloride.

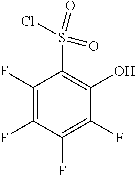 [SMILES: O=S(=O)(Cl)c1c(O)c(F)c(F)c(F)c1F]

### 2,3,4,5-tetrafluoro-6-hydroxybenzene-1-sulfonyl chloride

Using potassium 2,3,4,5-tetrafluorophenoxide as a starting material, 2,3,4,5-tetrafluoro-6-hydroxybenzene-1-sulfonyl chloride was prepared according to the protocol described in general procedure A-1. (red oil, 52-60% yield). ¹⁹F NMR (376 MHz, CDCl₃) δ −134.49-−134.89 (m), −140.19-−140.39 (m), −156.03-−156.45 (m), −164.26-−164.38 (m).

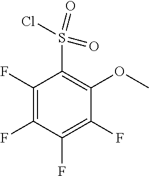 [SMILES: COc1c(F)c(F)c(F)c(F)c1S(=O)(=O)Cl]

### 2,3,4,5-tetrafluoro-6-methoxybenzene-1-sulfonyl chloride

Using 1,2,3,4-tetrafluoro-5-methoxybenzene as a starting material, 2,3,4,5-tetrafluoro-6-methoxybenzene-1-sulfonyl chloride was prepared according to the protocol described in general procedure A-1. (red oil, 40% yield). ¹⁹F NMR (376 MHz, CDCl₃) δ −134.21-−134.81 (m), −141.73-−141.93 (m), −151.49-−151.89 (m), −159.14-−159.54 (m).

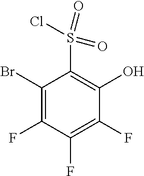 [SMILES: O=S(=O)(Cl)c1c(O)c(F)c(F)c(F)c1Br]

### 2-bromo-3,4,5-trifluoro-6-hydroxybenzene-1-sulfonyl chloride

Using 5-bromo-2,3,4-trifluorophenol as a starting material, 2-bromo-3,4,5-trifluoro-6-hydroxybenzene-1-sulfonyl chloride was prepared according to the protocol described in general procedure A-1. (white solid, 63% yield). ¹⁹F NMR (376 MHz, CDCl₃) δ −137.08-−137.15 (m), −155.80-−155.92 (m), −156.48-−156.53 (m).

### General Procedure B-1

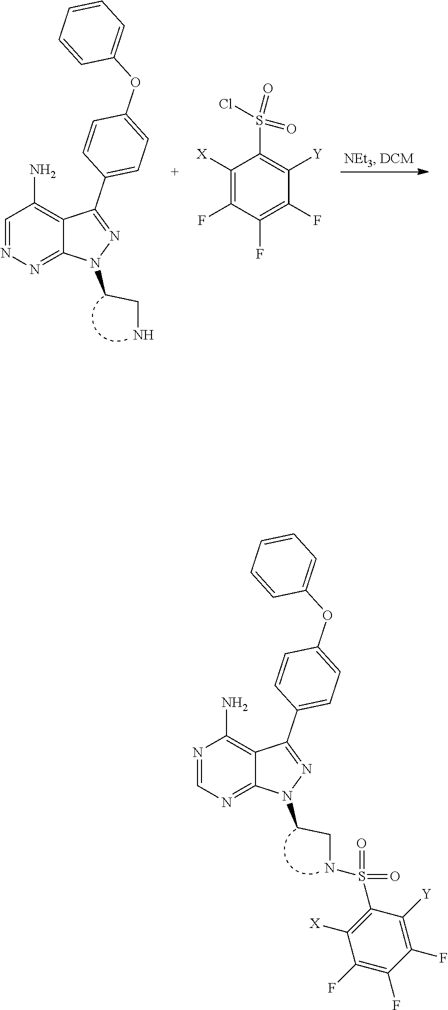

An appropriate sulfonyl chloride (0.9-1.2 eq) was incubated with its corresponding pyrazololopyrimidine (1 eq) in anhydrous DCM (0.1 M-0.25 M) under an atmosphere of argon. The resulting mixture was cooled to 0° C. and stirred for 15 minutes. Neat triethylamine (3-5 eq) was slowly added to the mixture and it was stirred at 0° C. for a further 3-16 hrs. The reaction quenched with 0.1M HCl (aq) and vigorously stirred for 10-15 min, after which the organic layer was separated. The aqueous layer was extracted with DCM one further time. The combined organic layers were dried over sodium sulfate, filtered, and evaporated. The crude material was purified by either flash column chromatography, eluting with a solvent system comprised of ethyl acetate/DCM, or reverse-phase chromatography employing a solvent system comprised of ACN/Water containing 0.1% formic acid. The isolated material was lyophilized from ACN and water to afford the desired product as a free flowing off-white solid.

### Example 33: N-[2-[4-amino-3-(4-phenoxyphenyl)pyrazolo[3,4-d]pyrimidin-1-yl]ethyl]-2,3,4,5,6-pentafluoro-benzenesulfonamide (Compound 5-3

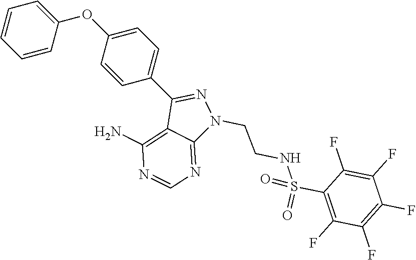 [SMILES: Nc1ncnc2c1c(-c1ccc(Oc3ccccc3)cc1)nn2CCNS(=O)(=O)c1c(F)c(F)c(F)c(F)c1F]

The title compound N-[2-[4-amino-3-(4-phenoxyphenyl)pyrazolo[3,4-d]pyrimidin-1-yl]ethyl]-2,3,4,5,6-pentafluoro-benzenesulfonamide, was prepared using the protocol described in general procedure B-1 (1.68 g, 74% yield). ¹H NMR (400 MHz, CDCl₃) δ 8.26 (s, 1H), 7.69 (s, 1H), 7.60-7.51 (m, 2H), 7.40 (dd, J=8.6, 7.4 Hz, 2H), 7.23-7.02 (m, 5H), 5.78 (s, 2H), 4.66-4.52 (m, 2H), 3.89-3.75 (m, 2H). ¹⁹F NMR (376 MHz, CDCl₃) δ −136.77 (d, J=20.7 Hz), −146.13-−146.66 (m), −158.33-−158.77 (m). ESI-MS: measured m/z 576.70 [M+H]⁺. Purity by HPLC: 98.5% @ 254 nm

### Example 34: (R)-1-(1-((perfluorophenyl)sulfonyl)piperidin-3-yl)-3-(4-phenoxyphenyl)-1H-pyrazolo[3,4-d]pyrimidin-4-amine (Compound 5-1

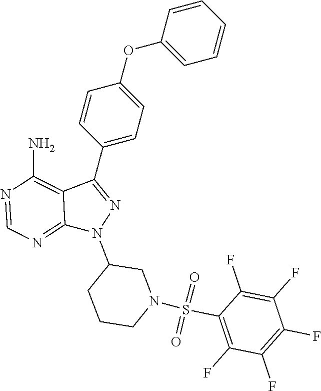 [SMILES: Nc1ncnc2c1c(-c1ccc(Oc3ccccc3)cc1)nn2C1CCCN(S(=O)(=O)c2c(F)c(F)c(F)c(F)c2F)C1]

The title compound (R)-1-(1-((perfluorophenyl)sulfonyl)piperidin-3-yl)-3-(4-phenoxyphenyl)-1H-pyrazolo[3,4-d]pyrimidin-4-amine, was prepared using the protocol described in general procedure B-1 (0.029 g, 13% yield). ¹H NMR (400 MHz, CDCl₃) δ 8.39 (s, 1H), 7.69-7.60 (m, 2H), 7.46-7.37 (m, 2H), 7.24-7.15 (m, 3H), 7.15-7.08 (m, 2H), 5.70 (s, 2H), 5.05 (tt, J=10.4, 5.0 Hz, 1H), 4.19 (dd, J=12.1, 4.5 Hz, 1H), 4.05 (d, J=12.4 Hz, 1H), 3.40 (t, J=11.3 Hz, 1H), 2.86 (t, J=11.6 Hz, 1H), 2.33-2.23 (m, 2H), 2.17-2.04 (m, 2H), 2.04-1.90 (m, 1H). ¹⁹F NMR (376 MHz, Chloroform-d) 6-134.36 (qd, J=13.8, 7.8 Hz), −145.32 (tt, J=21.0, 6.3 Hz), −158.14 (tt, J=21.1, 6.7 Hz). ESI-MS: measured m/z 616.7 [M+H]⁺. Purity by HPLC: 98.3% @ 254 nm.

### Example 35: (S)-1-(1-((perfluorophenyl)sulfonyl)piperidin-3-yl)-3-(4-phenoxyphenyl)-1H-pyrazolo[3,4-d]pyrimidin-4-amine (Compound 5-2

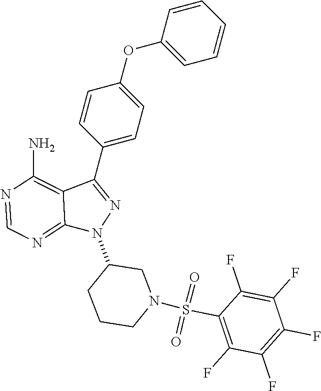

The title compound (S)-1-(1-((perfluorophenyl)sulfonyl)piperidin-3-yl)-3-(4-phenoxyphenyl)-1H-pyrazolo[3,4-d]pyrimidin-4-amine, was prepared using the protocol described in general procedure B-1 (0.020 g, 40% yield). ¹H NMR (400 MHz, CDCl₃) δ 8.39 (s, 1H), 7.69-7.61 (m, 2H), 7.46-7.35 (m, 2H), 7.24-7.16 (m, 3H), 7.16-7.08 (m, 2H), 5.80 (s, 2H), 5.05 (tt, J=10.4, 4.8 Hz, 1H), 4.17 (td, J=11.5, 10.8, 5.8 Hz, 1H), 4.04 (d, J=12.6 Hz, 1H), 3.40 (t, J=11.3 Hz, 1H), 2.91-2.79 (m, 1H), 2.32-2.22 (m, 2H), 2.12-2.03 (m, 1H), 2.03-1.89 (m, 1H). ¹⁹F NMR (376 MHz, Chloroform-d) 6-134.41 (dt, J=21.2, 6.0 Hz), −145.39 (tt, J=21.1, 6.5 Hz), −157.98-−158.31 (m). ESI-MS: measured m/z 616.7 [M+H]⁺. Purity by HPLC: 99.9% @ 254 nm.

### Example 36: 1-((perfluorophenyl)sulfonyl)-3-(4-phenoxyphenyl)-1H-pyrazolo[3,4-d]pyrimidin-4-amine (Compound 5-19

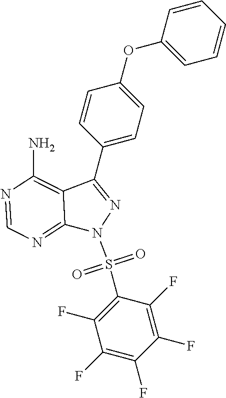 [SMILES: Nc1ncnc2c1c(-c1ccc(Oc3ccccc3)cc1)nn2S(=O)(=O)c1c(F)c(F)c(F)c(F)c1F]

The title compound 1-((perfluorophenyl)sulfonyl)-3-(4-phenoxyphenyl)-1H-pyrazolo[3,4-d]pyrimidin-4-amine, was prepared using the protocol described in general procedure B-1 (0.012 g, 10% yield). ¹H NMR (400 MHz, CDCl₃) δ 8.58 (s, 1H), 7.65 (d, J=8.3 Hz, 2H), 7.45 (t, J=7.8 Hz, 2H), 7.26 (s, 1H), 7.20 (d, J=8.1 Hz, 2H), 7.13 (d, J=8.0 Hz, 2H). ¹⁹F NMR (376 MHz, CDCl₃) δ −132.39 (td, J=16.4, 15.5, 9.6 Hz), −139.15 (q, J=18.8, 15.7 Hz), −156.32-−156.64 (m). ESI-MS: measured m/z 533.7 [M+H]⁺. Purity by HPLC: 99.8% @254 nm.

### Example 37: N-(3-(4-amino-3-(4-phenoxyphenyl)-1H-pyrazolo[3,4-d]pyrimidin-1-yl)propyl)-2,3,4,5,6-pentafluorobenzenesulfonamide (Compound 5-4

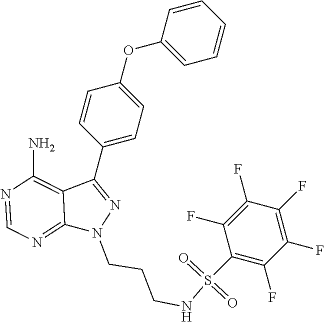 [SMILES: Nc1ncnc2c1c(-c1ccc(Oc3ccccc3)cc1)nn2CCCNS(=O)(=O)c1c(F)c(F)c(F)c(F)c1F]

The title compound N-(3-(4-amino-3-(4-phenoxyphenyl)-1H-pyrazolo[3,4-d]pyrimidin-1-yl)propyl)-2,3,4,5,6-pentafluorobenzenesulfonamide, was prepared using the protocol described in general procedure B-1 (0.011 g, 17% yield). ¹H NMR (400 MHz, CD₃CN) δ 8.29 (s, 1H), 7.72-7.63 (m, 2H), 7.49-7.41 (m, 2H), 7.27-7.12 (m, 5H), 5.94 (s, 2H), 4.45 (t, J=6.4 Hz, 2H), 3.11 (t, J=6.7 Hz, 2H), 2.12 (p, J=6.6 Hz, 2H). ¹⁹F NMR (376 MHz, CDCl₃) δ −138.96-−139.33 (m), −149.75-−149.98 (m), −161.32-−161.75 (m). ESI-MS: measured m/z 590.7 [M+H]⁺. Purity by HPLC: 99.3% @ 254 nm.

### Example 38: 1-(1-((perfluorophenyl)sulfonyl)azetidin-3-yl)-3-(4-phenoxyphenyl)-1H-pyrazolo[3,4-d]pyrimidin-4-amine (Compound 5-6

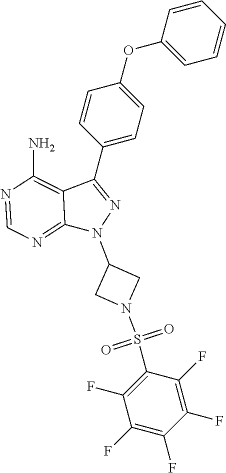 [SMILES: Nc1ncnc2c1c(-c1ccc(Oc3ccccc3)cc1)nn2C1CN(S(=O)(=O)c2c(F)c(F)c(F)c(F)c2F)C1]

The title compound 1-(1-((perfluorophenyl)sulfonyl)azetidin-3-yl)-3-(4-phenoxyphenyl)-1H-pyrazolo[3,4-d]pyrimidin-4-amine, was prepared using the protocol described in general procedure B-1 (0.068 g, 18% yield). ¹H NMR (400 MHz, DMSO-d₆) δ 8.20 (s, 1H), 7.56-7.44 (m, 5H), 7.23 (td, J=7.4, 1.2 Hz, 1H), 7.20-7.13 (m, 4H), 5.81-5.68 (m, 1H), 4.58 (t, J=8.6 Hz, 2H), 4.46 (dd, J=9.2, 6.0 Hz, 2H). ¹⁹F NMR (376 MHz, DMSO-d₆) δ −133.55-−134.01 (m), −145.23-−145.69 (m), −159.10 (tt, J=22.5, 6.2 Hz). ESI-MS: measured m/z 588.7 [M+H]⁺. Purity by HPLC: 98.8% @ 254 nm.

### Example 39: (R)-1-(1-((perfluorophenyl)sulfonyl)pyrrolidin-3-yl)-3-(4-phenoxyphenyl)-1H-pyrazolo[3,4-d]pyrimidin-4-amine (Compound 5-5

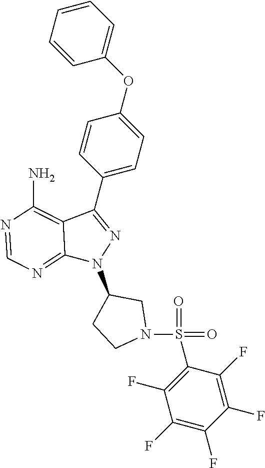 [SMILES: Nc1ncnc2c1c(-c1ccc(Oc3ccccc3)cc1)nn2[C@@H]1CCN(S(=O)(=O)c2c(F)c(F)c(F)c(F)c2F)C1]

The title compound (R)-1-(1-((perfluorophenyl)sulfonyl)pyrrolidin-3-yl)-3-(4-phenoxyphenyl)-1H-pyrazolo[3,4-d]pyrimidin-4-amine, was prepared using the protocol described in general procedure B-1 (0.51 g, 63% yield). ¹H NMR (400 MHz, CDCl₃) δ 8.30 (s, 1H), 7.44-7.41 (m, 4H), 7.26-7.21 (m, 1H), 7.16-7.12 (m, 4H), 5.69-5.51 (m, 3H), 4.03-3.96 (m, 2H), 3.90-3.79 (m, 2H), 2.63-2.49 (m, 2H). ¹⁹F NMR (376 MHz, CDCl₃) δ −133.80-−133.95 (m, 2F), −145.29-−145.43 (m, 1H), −158.83-−158.95 (m, 2F). ESI-MS: measured m/z 602.7 [M+H]⁺. Purity by HPLC: 99.8% @ 254 nm.

### General Procedure D

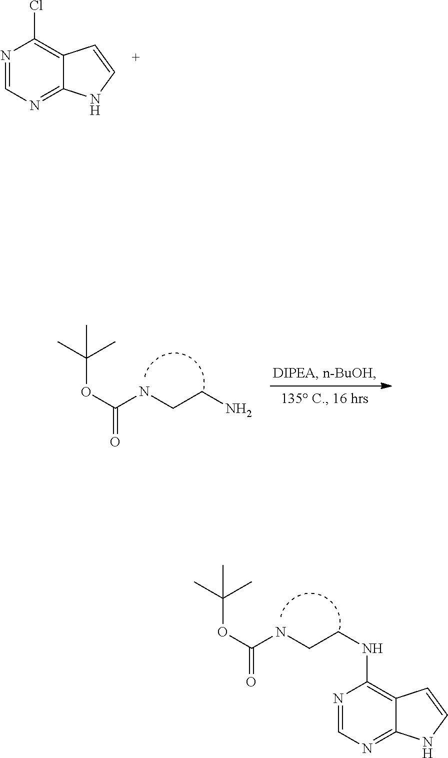

Neat diisopropylethylamine (DIPEA) was added to a mixture of 4-Chloro-7H-pyrrolo[2,3-d]pyrimidine (1 eq), aminocarbamate (1 eq) and n-butanol (0.5-1 M). The reaction vessel was equipped with a water jacketed condenser and the apparatus heated at 135° C. in an oil bath overnight. After 16 hours, the reaction was cooled to room temperature and partitioned between ethyl acetate and brine. The aqueous phase was separated and the organic phase washed with water, dilute HCl (done 4 times), and brine. The organic phase was dried over anhydrous sodium sulfate, filtered, concentrated and purified using flash column chromatography techniques (DCM/MeOH mobile phase) to afford the desired product.

### General Procedure E

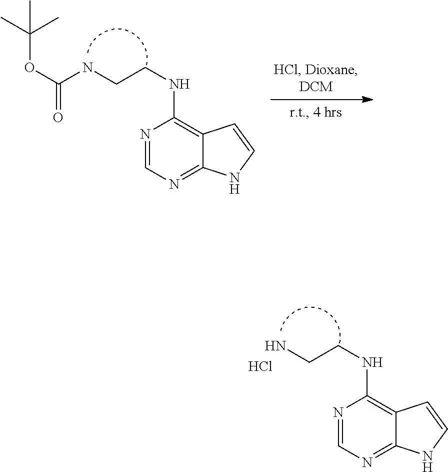 [SMILES: CC(C)(C)OC(=O)NCCNc1ncnc2[nH]ccc12 ; Cl ; NCCNc1ncnc2[nH]ccc12]

To a mixture of boc-protected pyrrolopyrimidine (1 eq.) in DCM was added a solution of HCl in dioxane (4 M, 4 eq.). The resulting mixture was stirred at room temperature until consumption of the starting material was observed by LC/MS. Once the reaction was complete, excess solvent was removed using a rotary evaporator and the remaining residue dried in vacuo to afford the anticipated product.

### General Procedure F

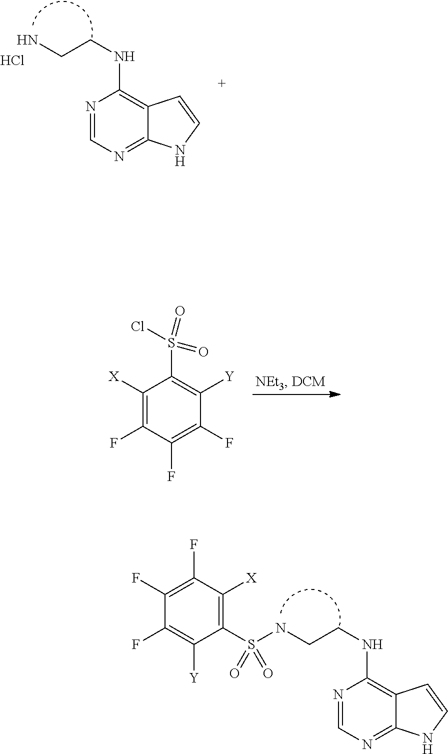

An appropriate sulfonyl chloride (0.9-1.2 eq) was incubated with its corresponding pyrrolopyrimidine (1 eq) in anhydrous DCM (0.1 M-0.25 M) under an atmosphere of argon. The resulting mixture was cooled to 0° C. and stirred for 15 minutes. Neat triethylamine (3-5 eq) was slowly added to the mixture and it was stirred at 0° C. for a further 3-16 hrs. The reaction quenched with 0.1M HCl (aq) and vigorously stirred for 10-15 min, after which the organic layer was separated. The aqueous layer was extracted with DCM one further time. The combined organic layers were dried over sodium sulfate, filtered, and evaporated. The crude material was purified by either flash column chromatography, eluting with a solvent system comprised of MeOH/DCM, or reverse-phase chromatography, employing a solvent system comprised of ACN/Water containing 0.1% formic acid. The isolated material was lyophilized from ACN and water to afford the desired product.

### Synthesis of tert-butyl (2-((7H-pyrrolo[2,3-d]pyrimidin-4-yl)amino)ethyl)carbamate

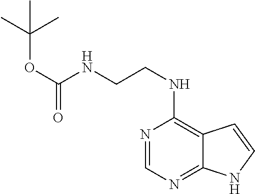 [SMILES: CC(C)(C)OC(=O)NCCNc1ncnc2[nH]ccc12]

Tert-butyl (2-((7H-pyrrolo[2,3-d]pyrimidin-4-yl)amino)ethyl)carbamate was prepared according to the protocol described in general procedure D and isolated as an off-white powdery solid (63% yield). ¹H NMR (400 MHz, DMSO-d6) δ 12.23 (br s, 1H), 9.03 (br s, 1H), 8.26 (s, 1H), 7.32 (br s, 1H), 6.99 (s, 1H), 6.87 (s, 1H), 3.58 (q, J=8.0 Hz, 2H), 3.25 (q, J=8.0 Hz, 2H), 1.34 (s, 9H) ESI-MS: measured m/z 277.9 [M+H]⁺.

### Synthesis of tert-butyl (2-((7H-pyrrolo[2,3-d]pyrimidin-4-yl)amino)ethyl)(methyl)carbamate

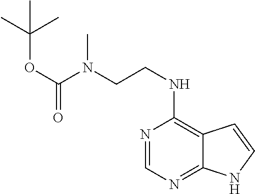 [SMILES: CN(CCNc1ncnc2[nH]ccc12)C(=O)OC(C)(C)C]

Tert-butyl (2-((7H-pyrrolo[2,3-d]pyrimidin-4-yl)amino)ethyl)(methyl)carbamate was prepared according to the protocol described in general procedure D and isolated as a white powdery solid (1.48 g, 78% yield). ¹H NMR (400 MHz, DMSO-d₆) δ 11.44 (brs, 1H), 8.08 (s, 1H), 7.53-7.38 (m, 1H), 7.08-7.01 (m, 1H), 6.53-6.44 (m, 1H), 3.61-3.48 (m, 2H), 3.46-3.33 (m, 2H), 2.80 (s, 3H), 1.41-1.07 (9H). ESI-MS: measured m/z 292.0 [M+H]⁺.

### Synthesis of N¹-(7H-pyrrolo[2,3-d]pyrimidin-4-yl)ethane-1,2-diamine hydrochloride

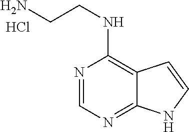 [SMILES: Cl ; NCCNc1ncnc2[nH]ccc12]

N¹-(7H-pyrrolo[2,3-d]pyrimidin-4-yl)ethane-1,2-diamine hydrochloride was prepared according to the protocol described in general procedure E and isolated as a beige solid (2.1 g, 97% yield). ¹H NMR (400 MHz, DMSO-d₆) δ 12.75 (br s, 1H), 10.09 (br s, 1H), 8.35 (s, 1H), 7.43 (s, 1H), 7.13 (s, 1H), 3.89 (d, J=8.0 Hz, 2H), 3.18 (d, J=8.0 Hz, 2H). ESI-MS: measured m/z 178.0 [M+H]⁺.

### Synthesis of N¹-methyl-N²-(7H-pyrrolo[2,3-d]pyrimidin-4-yl)ethane-1,2-diamine hydrochloride

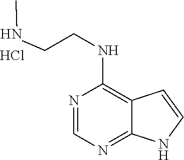

N¹-methyl-N²-(7H-pyrrolo[2,3-d]pyrimidin-4-yl)ethane-1,2-diamine hydrochloride was prepared according to the protocol described in general procedure E and isolated as a beige solid (1.34 g, 100% yield). ¹H NMR (400 MHz, DMSO-d₆+D₂O) δ 8.34 (s, 1H), 7.41-7.34 (m, 1H), 6.80-6.83 (m, 1H), 3.86-3.79 (m, 2H), 3.26-3.19 (m, 2H), 2.60 (s, 3H). ESI-MS: measured m/z 192.0 [M+H]⁺.

### Example 40: N-(2-((7H-pyrrolo[2,3-d]pyrimidin-4-yl)amino)ethyl)-2,3,4,5,6-pentafluorobenzene sulfonamide (Compound 5-10

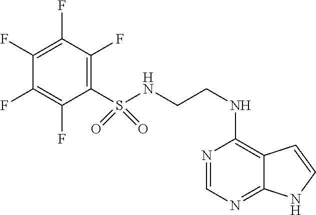 [SMILES: O=S(=O)(NCCNc1ncnc2[nH]ccc12)c1c(F)c(F)c(F)c(F)c1F]

The title compound N-(2-((7H-pyrrolo[2,3-d]pyrimidin-4-yl)amino)ethyl)-2,3,4,5,6-pentafluorobenzene sulfonamide, was prepared according to the protocol described in general procedure F and isolated as a yellow powder (13% yield). ¹H NMR (400 MHz, DMSO-d₆) δ 11.62 (br s, 1H), 8.81 (br s, 1H), 8.05 (s, 1H), 7.35 (t, J=8.0 Hz, 1H), 7.06 (dd, J=4.0, 8.0 Hz, 1H), 6.35 (dd, J=4.0, 8.0 Hz, 1H), 3.51 (q, J=8.0 Hz, 2H), 3.42 (q, J=8.0 Hz, 2H). ¹⁹F NMR (376 MHz, DMSO-d₆) δ −138.22-−138.39 (m), −148.56-−148.47 (m), −160.06-−160.28 (m). ESI-MS: measured m/z 407.8 [M+H]⁺. Purity by HPLC: 96.1% @ 254 nm.

### Example 41: N-(2-((7H-pyrrolo[2,3-d]pyrimidin-4-yl)amino)ethyl)-2,3,4,5,6-pentafluoro-N-methylbenzenesulfonamide (Compound 5-11

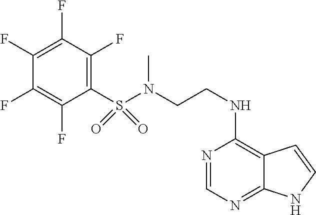 [SMILES: CN(CCNc1ncnc2[nH]ccc12)S(=O)(=O)c1c(F)c(F)c(F)c(F)c1F]

The title compound N-(2-((7H-pyrrolo[2,3-d]pyrimidin-4-yl)amino)ethyl)-2,3,4,5,6-pentafluoro-N-methylbenzenesulfonamide, was prepared according to the protocol described in general procedure F and isolated as a yellow powder (0.075 g, 35% yield). ¹H NMR (400 MHz, DMSO-d₆) δ 11.51 (brs, 1H), 8.05 (s, 1H), 7.45-7.38 (brs, 1H), 7.07-7.02 (m, 1H), 6.33-6.28 (m, 1H), 3.65-3.56 (m, 4H), 3.10 (s, 3H). ¹⁹F NMR (376 MHz, DMSO-d₆) δ −136.8 (m), −147.8 (m), −159.7 (m). ESI-MS: measured m/z 421.7 [M+H]⁺. Purity by HPLC: 99.1% @254 nm.

### Example 42: N-((3R,6S)-6-methyl-1-((perfluorophenyl)sulfonyl)piperidin-3-yl)-7H-pyrrolo[2,3-d]pyrimidin-4-amine (Compound 5-8

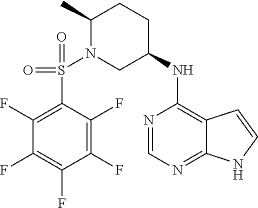 [SMILES: C[C@H]1CC[C@@H](Nc2ncnc3[nH]ccc23)CN1S(=O)(=O)c1c(F)c(F)c(F)c(F)c1F]

The title compound N-((3R,6S)-6-methyl-1-((perfluorophenyl)sulfonyl)piperidin-3-yl)-7H-pyrrolo[2,3-d]pyrimidin-4-amine, was prepared according to the protocol described in general procedure F and isolated as a pale yellow solid (0.14 g, 31% yield). ¹H NMR (400 MHz, DMSO-d6) δ 11.54 (brs, 1H), 8.06 (s, 1H), 7.24 (d, J=8 Hz, 1H), 7.11-7.07 (m, 1H), 6.53-6.49 (m, 1H), 4.34-4.24 (m, 1H), 4.01-3.88 (m, 2H), 3.01-2.91 (m, 1H), 1.81-1.60 (m, 4H), 1.24-1.13 (m, 3H).

¹⁹F NMR (376 MHz, DMSO-d₆) δ 136.8 (m), −147.3 (m), −159.4 (m). ESI-MS: measured m/z 461.7 [M+H]⁺. Purity by HPLC: 97% @ 254 nm.

### General Procedure G

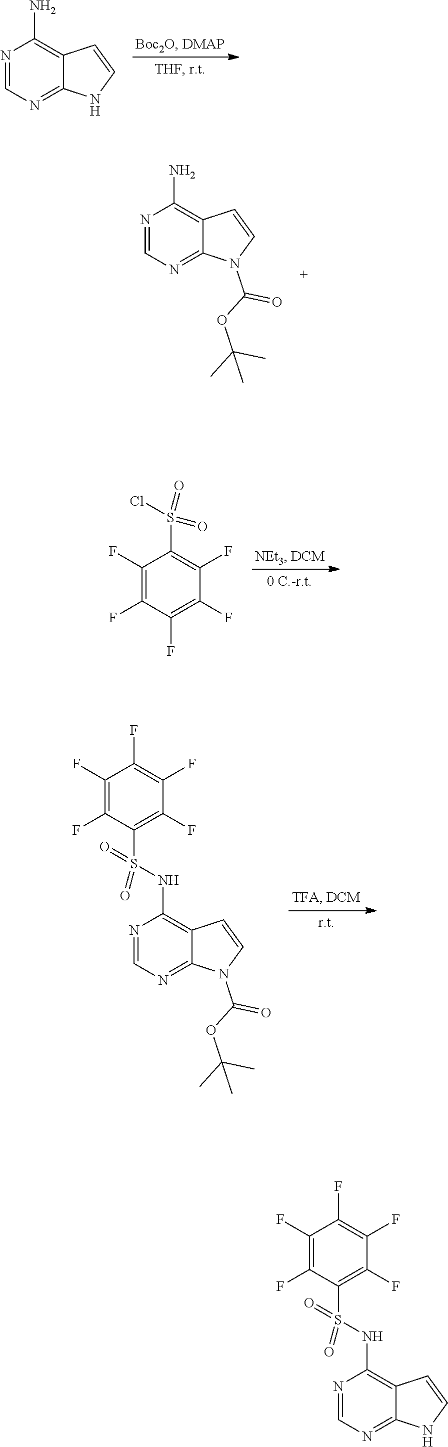

### Synthesis of tert-butyl 4-amino-7H-pyrrolo[2,3-d]pyrimidine-7-carboxylate

To a stirred suspension of 7H-pyrrolo[2,3-d]pyrimidin-4-amine (5 g, 37.27 mmol) and DMAP (455.38 mg, 3.73 mmol) in THF (20 mL) at room temperature under argon was added di-tert-butyl dicarbonate (8.95 g, 41.00 mmol, 9.41 mL) slowly as a solution in THE (20 mL). The mixture was stirred for at room temperature for 3 h. Once the reaction was finished, the mixture was diluted with water/brine and extracted with ethyl acetate (3×). The combined organic phases were washed with brine (2×), dried over anhydrous sodium sulfate, filtered and concentrated in vacuo. The product of interest was isolated via flash column chromatography (50-100% EtOAc in DCM) and subsequently triturated in a solution of diethyl ether and hexanes. The mixture was filtered to collect the desired product as a white solid (4.5 g, 19.21 mmol, 51.54% yield). ¹H NMR (400 MHz, DMSO-d₆) δ 8.18 (s, 1H), 7.44 (d, J=4 Hz, 1H), 7.22 brs, 2H), 6.74 (d, J=4 Hz, 1H), 1.60 (s, 9H). ESI-MS: measured m/z 234.9 [M+H]⁺.

### Synthesis of tert-butyl 4-((perfluorophenyl)sulfonamido)-7H-pyrrolo[2,3-d]pyrimidine-7-carboxylate

To a solution of tert-butyl 4-aminopyrrolo[2,3-d]pyrimidine-7-carboxylate (0.1 g, 426.89 umol, 1 eq.) in chloroform (0.1 M) (4.2 mL) cooled to 0° C. was added neat 2,3,4,5,6-pentafluoro benzene sulfonyl chloride (113.80 mg, 426.89 umol, 63.22 uL, 1 eq.) under inert conditions (nitrogen atmosphere). The resulting solution was stirred at 0° C. for 5 minutes before neat N,N-diethylethanamine (64.80 mg, 640.33 umol, 89.25 uL, 1.5 eq.) was added slowly over a period of 2 minutes. The mixture was stirred for 5 hours while slowly warming to room temperature. Water was added to quench the reaction and the resulting mixture extracted three times with DCM. The collected organic layers were washed with brine, dried over anhydrous sodium sulfate, filtered, and evaporated under reduced pressure using a rotary evaporator. The resulting residue was separated on a pad of silica eluting with a gradient of 20% to 30% ethyl acetate in hexanes to afford the desired product (0.08 g, 172.2 umol, 40% yield). ¹H NMR (400 MHz, CDCl₃) δ 12.46 (s, 1H), 8.25 (s, 1H), 7.56 (d, J=3.9 Hz, 1H), 6.78 (d, J=3.9 Hz, 1H). ¹⁹F NMR (376 MHz, CDCl₃) δ −136.95 (m), −147.48 (m), −159.22-−159.41 (m).

### Example 43: 2,3,4,5,6-pentafluoro-N-(7H-pyrrolo[2,3-d]pyrimidin-4-yl)benzene sulfonamide (Compound 5-12

To a stirred solution of tert-butyl 4-[(2,3,4,5,6-pentafluorophenyl)sulfonylamino]pyrrolo[2,3-d]pyrimidine-7-carboxylate (0.05 g, 0.107 mmol) in DCM (0.1 M) (1 mL) was added TFA (0.1 M) (1 mL) in a dropwise manner. The resulting mixture was stirred at room temperature for 1 hour. Upon complete consumption of the starting material based on TLC (H:E=2:1), the mixture was concentrated under reduced pressure using rotary evaporator. The mixture was diluted with EtOAc (40 mL) and washed three times with a saturated aqueous solution of sodium bicarbonate (30 mL×3). The collected organic layers were dried with anhydrous sodium sulfate, filtered, and evaporated under reduced pressure to yield the crude product. The crude residue was purified by Prep-HPLC, running a mobile phase of 90% to 0% H₂O (0.1% FA) in ACN (0.1% FA) over 60 minutes to afford the desired product. ¹H NMR (400 MHz, CD₃CN) δ 11.90 (s, 1H), 10.38 (s, 1H), 8.20 (s, 1H), 7.27 (dd, J=3.6, 2.2 Hz, 1H), 6.72 (dd, J=3.6, 1.9 Hz, 1H). ¹⁹F NMR (376 MHz, CD₃CN) δ −139.51 (dt, J=20.8, 5.4 Hz, 2F), −151.68-−151.85 (m, 1F), −162.26-−162.44 (m, 2F). 2-chloro-N-((3R,6S)-6-methylpiperidin-3-yl)-7H-pyrrolo[2,3-d]pyrimidin-4-amine

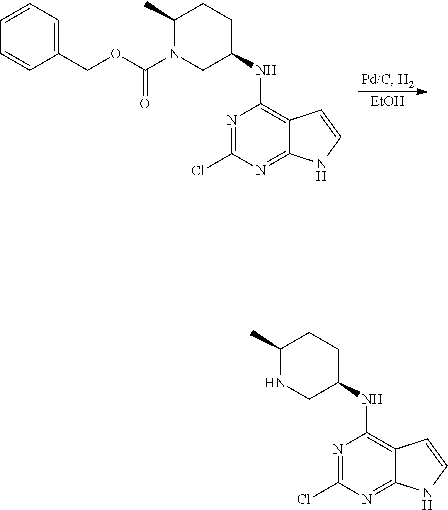 [SMILES: "EtOH" ; C[C@H]1CC[C@@H](Nc2nc(Cl)nc3[nH]ccc23)CN1C(=O)OCc1ccccc1 ; C[C@H]1CC[C@@H](Nc2nc(Cl)nc3[nH]ccc23)CN1]

Benzyl (2S,5R)-5-[(2-chloro-7H-pyrrolo[2,3-d]pyrimidin-4-yl)amino]-2-methyl-piperidine-1-carboxylate (200 mg, 500.1 μmol) was charged to a flask fitted with a stirbar and purged with argon. Palladium, 10 wt % on activated carbon (40 mg) was added with ethanol (3 mL). The mixture was purged with argon then with hydrogen and stirred under an atmosphere of hydrogen (balloon pressure) overnight. After the overnight period, the reaction was filtered through a pad of celite, washing with methanol and DCM. The filtrate was diluted with water, acidified with HCl and extracted with DCM to remove non-polar components. The aqueous phase was basified with NaOH to pH ˜11 and extracted with DCM (3×). The combined organics (from basic aqueous phase) were dried over magnesium sulfate, filtered and concentrated in vacuo to afford an off-white solid which was used directly in the subsequent reaction without further purification. ¹H NMR (d₆-DMSO) δ 11.64 (brs, 1H), 7.36-7.30 (m, 1H), 7.09-7.03 (m, 1H), 6.73-6.65 (m, 1H), 4.14-4.03 (m, brs, 1H), 3.21-3.12 (m, 1H), 2.99-2.90 (m, 1H), 2.83-2.75 (m, 1H), 2.67-2.55 (m, 1H), 1.96-1.82 (m, 1H), 1.69-1.55 (m, 1H), 1.49-1.38 (m, 1H), 1.38-1.22 (m, 1H), 1.03 (d, J=8 Hz, 3H). ESI-MS: measured m/z 265.9 [M+H]^{m}.

### Example 44: 2-chloro-N-((3R,6S)-6-methyl-1-((perfluorophenyl)sulfonyl)piperidin-3-yl)-7H-pyrrolo[2,3-d]pyrimidin-4-amine (Compound 5-9

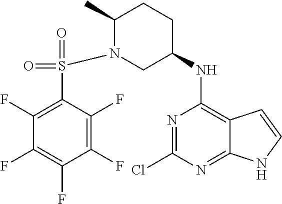 [SMILES: C[C@H]1CC[C@@H](Nc2nc(Cl)nc3[nH]ccc23)CN1S(=O)(=O)c1c(F)c(F)c(F)c(F)c1F]

The title compound 2-chloro-N-((3R,6S)-6-methyl-1-((perfluorophenyl)sulfonyl)piperidin-3-yl)-7H-pyrrolo[2,3-d]pyrimidin-4-amine, was prepared according to the protocol described in general procedure F and isolated as a pale yellow solid (0.040 g, 33% yield). δ 10.42 (brs, 1H), 7.13-7.05 (m, 1H), 6.42-6.32 (m, 1H), 5.03 (brs, 1H), 4.56-4.46 (m, 1H), 4.27-4.17 (m, 1H), 4.12-3.98 (m, 1H), 3.04-2.93 (1H), 2.06-1.91 (m, 2H), 1.81-1.69 (m, 2H), 1.31 (d, J=8.0 Hz, 3H). ¹⁹F NMR (376 MHz, CDCl₃) δ −137.01-−137.13 (m, 2F), −147.22-−147.35 (m, 1F), −159.11-−159.34 (m, 2F). ESI-MS: measured m/z 495.7 [M+H]⁺. Purity by HPLC: 97% @ 254 nm.

### General Procedure H

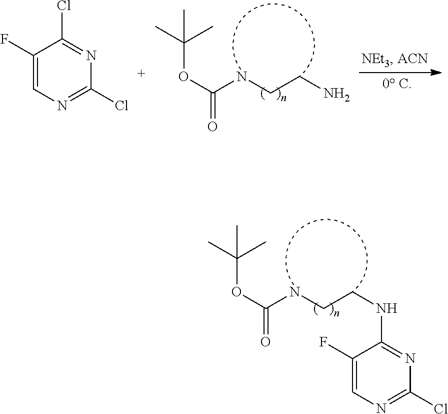

Neat triethylamine (1.6 eq.) was added dropwise to a cold solution (0° C.) of 2,4-dichloro-5-fluoro-pyrimidine (1 eq.), aminocarbamate (1.6 eq.) and acetonitrile (0.5 M). After 2-16 hours of stirring, the reaction was partitioned between water and ethyl acetate. The organic phase was removed and the remaining aqueous phase extracted a further two times with ethyl acetate. The organic phases were combined and washed with brine, dried over anhydrous magnesium sulfate, filtered, and concentrated under reduced pressure to afford the desired product.

### General Procedure I

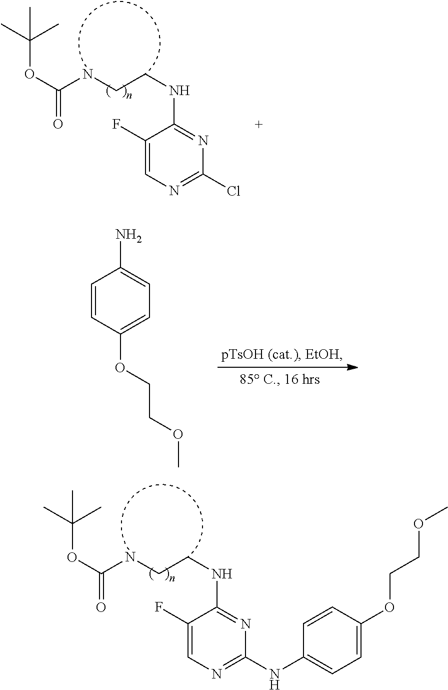

To a solution of boc-protected pyrimidine (1 eq.), 4-(2-methoxyethoxy)aniline (2 eq.), and ethanol (0.1-0.5 M) was added 4-methylbenzenesulfonic acid hydrate (0.05 eq.). The reaction vessel was fitted with a water jacketed reflux condenser and the apparatus heated at 85° C. for 18 hours. The reaction mixture was allowed to cool to room temperature and the solvent removed under reduced pressure. The compound of interest was isolated using flash column chromatography techniques employing a mobile phase of hexanes and ethyl acetate.

### General Procedure J

To a stirred solution of boc-protected pyrimidine (1 eq.) in DCM (0.1 M) was added TFA (3 eq.). The resulting solution was stirred at room temperature for 30 minutes and then quenched with saturated a saturated aqueous solution of sodium bicarbonate, followed by extraction with ethyl acetate (3 times). The combined organic phases were washed with brine, dried over anhydrous sodium sulfate, filtered, and evaporated under reduced pressure to afford the desired product.

### General Procedure K

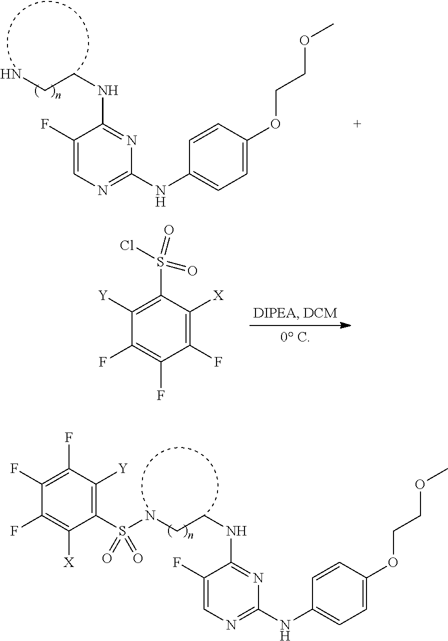

An appropriate sulfonyl chloride (0.9-1.2 eq) was incubated with its corresponding pyrimidine (1 eq) in anhydrous DCM (0.05 M-0.1 M) under an atmosphere of argon. The resulting mixture was cooled to 0° C. and stirred for 15 minutes. Neat diisopropylethylamine (3-5 eq) was slowly added to the mixture and it was stirred at 0° C. for a further 3-16 hrs. The reaction was quenched with water and extracted with ethyl acetate (3 times). The combined organic layers were washed with brine, dried over anhydrous sodium sulfate, filtered, and evaporated to dryness. The crude material was purified by either flash column chromatography, eluting with a solvent system comprised of MeOH/DCM, or reverse-phase chromatography, employing a solvent system comprised of ACN/Water containing 0.1% formic acid. The isolated material was lyophilized from ACN and water to afford the desired product.

### Synthesis of 2-chloro-5-fluoro-N-(3-nitrophenyl)pyrimidin-4-amine

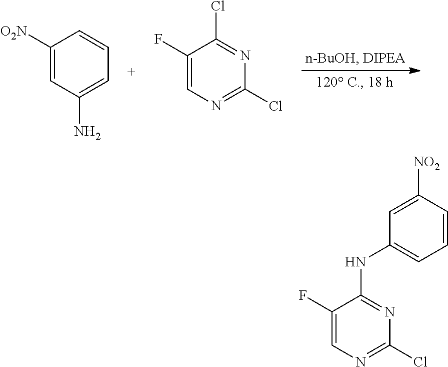 [SMILES: Nc1cccc([N+](=O)[O-])c1 ; Fc1cnc(Cl)nc1Cl ; O=[N+]([O-])c1cccc(Nc2nc(Cl)ncc2F)c1]

In an oven-dried m round bottom as purge with nitrogen, 3-nitroaniline (3 g, 21.72 mmol, 1 eq.), 2,4-dichloro-5-fluoro-pyrimidine (5.44 g, 32.58 mmol, 1.5 eq.) and N-ethyl-N-isopropyl-propan-2-amine (7.57 ml, 43.44 mmol, 2 eq.) were dissolved in n-butanol (0.27M) (80 mL). The reaction mixture was heated to reflux at 120° C. for 18 h. Reaction was monitored by TLC. After completion, the reaction mixture was allowed to cool to room temperature. The reaction was poured into water and extracted with ethyl acetate (3×25 mL). The combined ethyl acetate layers were washed with brine, dried over anhydrous sodium sulfate and concentrated under reduced pressure. The resulting residue was triturated with diethyl ether three times to afford the desired product (3.7 g, 13.77 mmol, 63%). ¹H NMR (400 MHz, DMSO-d₆) δ 10.43 (s, 1H), 8.75 (t, J=2.2 Hz, 1H), 8.45 (d, J=3.3 Hz, 1H), 8.17 (ddd, J=8.2, 2.3, 0.9 Hz, 1H), 8.02-7.97 (m, 1H), 7.70 (t, J=8.2 Hz, 1H). ¹⁹F NMR (376 MHz, DMSO-d₆) δ −152.69 (d, J=3.4 Hz, 1F).

### Synthesis of 5-fluoro-N²-(4-(2-methoxyethoxy)phenyl)-N⁴-(3-nitrophenyl)pyrimidine-2,4-diamine

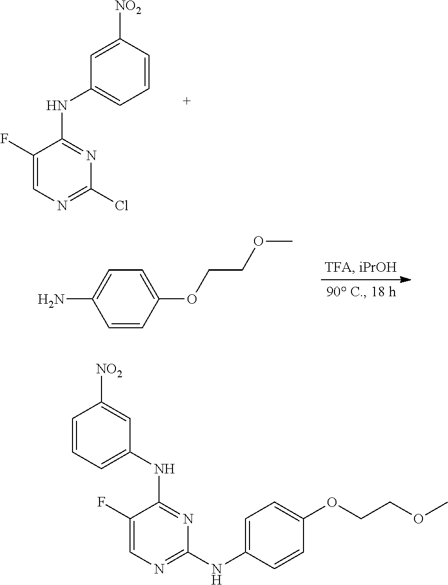

2-chloro-5-fluoro-N-(3-nitrophenyl)pyrimidin-4-amine (400 mg, 1.49 mmol) and 4-(2-methoxyethoxy)aniline (273.87 mg, 1.64 mmol) were dissolved in isopropanol (9 mL) under an inert atmosphere of argon. Neat trifluoroacetic acid (339.57 mg, 2.98 mmol, 227.90 uL) was introduced dropwise and the resulting solution was heated at 90° C. for 18 h. The reaction was permitted to cool to room temperature. Upon cooling, a solid started to precipitate from the solution. The solid was collected by vacuum filtration and triturated with ether to afford the desired product (0.386 g, 65% yield). ¹H NMR (400 MHz, DMSO-d₆) δ 10.49 (s, 1H), 9.86 (s, 1H), 8.53 (t, J=2.0 Hz, 1H), 8.29 (s, 1H), 8.23 (s, 1H), 7.99 (d, J=8.2 Hz, 1H), 7.64 (t, J=8.2 Hz, 1H), 7.46-7.33 (m, 2H), 6.86 (d, J=8.9 Hz, 2H), 4.14-3.91 (m, 2H), 3.66 (dd, J=5.4, 3.8 Hz, 2H), 3.32 (s, 3H).

### Synthesis of N⁴-(3-aminophenyl)-5-fluoro-N²-(4-(2-methoxyethoxy)phenyl)pyrimidine-2,4-diamine

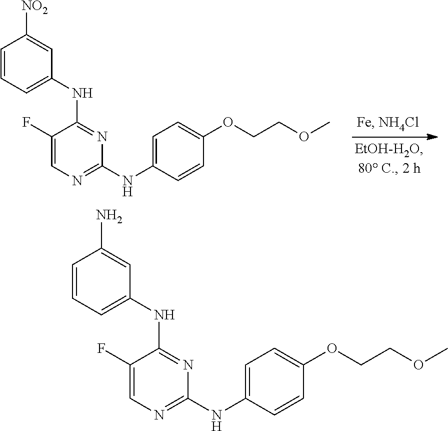

5-fluoro-N²-[4-(2-methoxyethoxy)phenyl]-N4-(3-nitrophenyl)pyrimidine-2,4-diamine (364.70 mg, 913.18 umol) was dissolved in ethanol (5.5 mL). Iron (254.98 mg, 4.57 mmol), ammonium chloride (73.27 mg, 1.37 mmol), and water (0.9 mL) were added and the resulting mixture heated to reflux. After 2 hours, the reaction mixture was allowed to cool to room temperature, filtered, and residue washed with ethyl acetate. The filtrate was partitioned between water and ethyl acetate. The organic phase was removed and the remaining aqueous phase extracted a further two times with ethyl acetate. The combined organic phases were washed with brine, dried over anhydrous sodium sulfate, filtered, and concentrated to dryness to afford the desired product (0.197 g, 58% yield) as a crude mixture of materials that was used without further purification. ¹H NMR (400 MHz, DMSO-d₆) δ 9.00 (s, 1H), 8.90 (s, 1H), 8.01 (d, J=3.8 Hz, 1H), 7.60-7.53 (m, 2H), 7.02-6.88 (m, 3H), 6.87-6.78 (m, 2H), 6.33 (d, J=7.8 Hz, 1H), 4.98 (s, 2H), 4.06-3.99 (m, 2H), 3.67-3.61 (m, 2H), 3.31 (s, 3H).

### Synthesis of 2-chloro-5-fluoro-N-(4-nitrophenyl)pyrimidin-4-amine

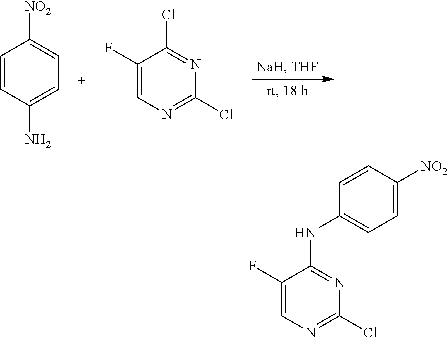 [SMILES: Nc1ccc([N+](=O)[O-])cc1 ; Fc1cnc(Cl)nc1Cl ; O=[N+]([O-])c1ccc(Nc2nc(Cl)ncc2F)cc1]

2,4-dichloro-5-fluoro-pyrimidine (1 g, 5.99 mmol) and 4-nitroaniline (992.70 mg, 7.19 mmol, 689.37 uL) were dissolved in THE (50 mL) and cooled to 0° C. by using an ice bath. A mineral dispersion of sodium hydride (287.45 mg, 7.19 mmol, 60% in mineral oil) was added in three portions to the cold solution. The reaction mixture was allowed to warm slowly to room temperature and stir for 18 hours. The reaction mixture was quenched with water and the bi-phasic mixture extracted three times with ethyl acetate. The combined organic layers were washed with brine, dried over anhydrous magnesium sulfate, and concentrated to dryness to afford the desired product (0.298 g, 19% yield). ¹H NMR (400 MHz, DMSO-d6) δ 10.55 (s, 1H), 8.51 (d, J=3.3 Hz, 1H), 8.34-8.23 (m, 2H), 8.11-7.97 (m, 2H).

### Synthesis of 5-fluoro-N²-(4-(2-methoxyethoxy)phenyl)-N⁴-(4-nitrophenyl)pyrimidine-2,4-diamine

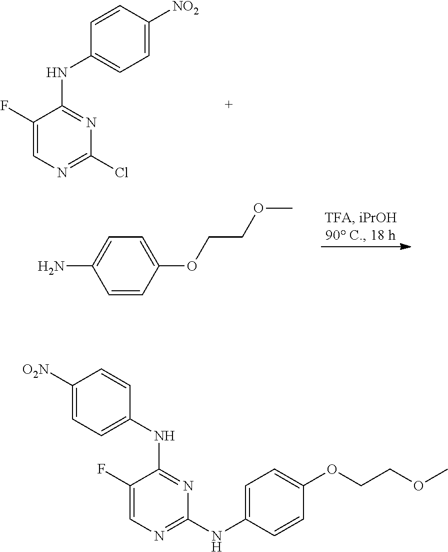 [SMILES: O=[N+]([O-])c1ccc(Nc2nc(Cl)ncc2F)cc1 ; COCCOc1ccc(N)cc1 ; COCCOc1ccc(Nc2ncc(F)c(Nc3ccc([N+](=O)[O-])cc3)n2)cc1]

2-chloro-5-fluoro-N-(4-nitrophenyl)pyrimidin-4-amine (386.1 mg, 1.44 mmol), and 4-(2-methoxyethoxy)aniline (240.3 mg, 1.44 mmol) were dissolved in isopropanol (9 mL) under an inert atmosphere of argon. Neat trifluoroacetic acid (327.7 mg, 2.87 mmol, 221.5 uL) was added dropwise and the resulting solution heated at 90° C. for 18 h. After cooling to room temperature, a solid began to precipitate from the solution. The solid was collect via vacuum filtration and rinsed with ether to afford the desired product (0.328 g, 57% yield). ¹H NMR (400 MHz, DMSO-d₆) δ 10.55 (s, 1H), 9.89 (s, 1H), 8.34 (d, J=4.0 Hz, 1H), 8.18 (d, J=8.3 Hz, 2H), 8.08 (d, J=8.5 Hz, 2H), 7.44 (d, J=8.5 Hz, 2H), 6.98 (d, J=7.6 Hz, 2H), 4.10 (d, J=3.4 Hz, 2H), 3.67 (d, J=3.3 Hz, 2H), 3.32 (d, J=3.9 Hz, 3H).

### Synthesis of N⁴-(4-aminophenyl)-5-fluoro-N²-(4-(2-methoxyethoxy)phenyl)pyrimidine-2,4-diamine

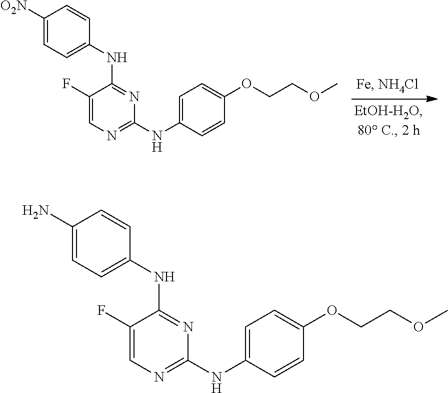 [SMILES: COCCOc1ccc(Nc2ncc(F)c(Nc3ccc([N+](=O)[O-])cc3)n2)cc1 ; COCCOc1ccc(Nc2ncc(F)c(Nc3ccc(N)cc3)n2)cc1]

5-fluoro-N²-[4-(2-methoxyethoxy)phenyl]-N4-(4-nitrophenyl)pyrimidine-2,4-diamine (328 mg, 821.28 umol) was dissolved in ethanol (4.9 mL). Iron (229.34 mg, 4.11 mmol, 29.18 uL), ammonium chloride (65.90 mg, 1.23 mmol), and water (0.8 mL) were added and the resulting mixture heated to reflux. After 2 hours, the reaction mixture was cooled to room temperature, filtered, and the residue washed with ethyl acetate. The filtrate was partitioned between water and ethyl acetate. The organic phase was removed and the remaining aqueous phase extracted a further two times with ethyl acetate. The combined organic phases were washed with brine, dried over anhydrous sodium sulfate, filtered, and concentrated to dryness to afford the desired product (0.208 g, 69% yield) as a crude mixture of materials that was used without further purification. ¹H NMR (400 MHz, DMSO-d6) δ 8.95 (d, J=37.2 Hz, 2H), 7.97 (s, 1H), 7.46 (dd, J=62.1, 7.2 Hz, 4H), 6.72 (dd, J=58.1, 7.5 Hz, 4H), 4.03 (s, 2H), 3.53 (s, 2H), 3.32 (s, 3H).

### Synthesis of N¹ tert-Butyl (2-((2-chloro-5-fluoropyrimidin-4-yl)amino)ethyl)carbamate

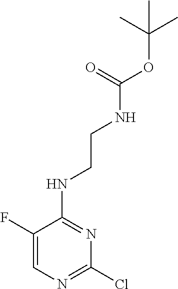 [SMILES: CC(C)(C)OC(=O)NCCNc1nc(Cl)ncc1F]

tert-Butyl (2-((2-chloro-5-fluoropyrimidin-4-yl)amino)ethyl)carbamate was prepared according to the protocol described in general procedure H (8.46 g, 97% yield). ¹H NMR (400 MHz, CDCl₃) δ 7.89 (s, 1H), 6.31 (s, 1H), 4.94 (s, 1H), 3.63 (dd, J=10.9, 5.3 Hz, 2H), 3.45 (dd, J=11.0, 5.8 Hz, 2H), 1.46 (s, 9H).

### Synthesis of tert-butyl (2-((5-fluoro-2-((4-(2-methoxyethoxy)phenyl)amino)pyrimidin-4-yl)amino)ethyl)carbamate

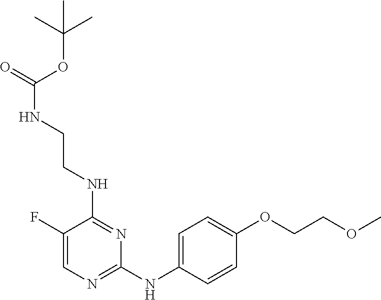 [SMILES: COCCOc1ccc(Nc2ncc(F)c(NCCNC(=O)OC(C)(C)C)n2)cc1]

tert-butyl (2-((5-fluoro-2-((4-(2-methoxyethoxy)phenyl)amino)pyrimidin-4-yl)amino)ethyl) carbamate was prepared according to the protocol described in general procedure I (0.486 g, 67% yield). ¹H NMR (400 MHz, CDCl₃) δ 7.78 (d, J=3.2 Hz, 1H), 7.47-7.40 (m, 2H), 6.95-6.88 (m, 2H), 5.50 (s, 1H), 4.92 (s, 1H), 4.13 (dd, J=5.5, 4.0 Hz, 2H), 3.80-3.74 (m, 2H), 3.60 (dd, J=11.2, 5.6 Hz, 2H), 3.48 (s, 3H), 3.42 (d, J=5.4 Hz, 2H), 1.46 (s, 9H).

### Synthesis of N⁴-(2-aminoethyl)-5-fluoro-N²-(4-(2 methoxyethoxy)phenyl)pyrimidine-2,4-diamine

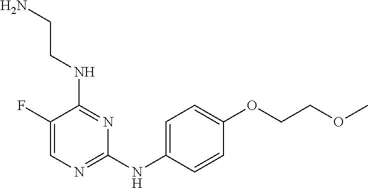 [SMILES: COCCOc1ccc(Nc2ncc(F)c(NCCN)n2)cc1]

N⁴-(2-aminoethyl)-5-fluoro-N²-(4-(2 methoxyethoxy)phenyl)pyrimidine-2,4-diamine was prepared according to the protocol described in general procedure J. ¹H NMR (400 MHz, DMSO-d₆) δ 8.86 (s, 1H), 7.87 (d, J=3.6 Hz, 1H), 7.57 (d, J=9.1 Hz, 2H), 7.39 (t, J=5.6 Hz, 1H), 6.85 (d, J=9.1 Hz, 2H), 6.16 (s, 2H), 4.05-4.01 (m, 2H), 3.66-3.62 (m, 2H), 3.50 (q, J=6.0 Hz, 2H), 3.31 (s, 3H), 2.98 (t, J=6.1 Hz, 2H). ¹⁹F NMR (376 MHz, DMSO-d₆) δ −75.69.

### Synthesis of tert-butyl (3-((2-chloro-5-fluoropyrimidin-4-yl)amino)propyl)carbamate

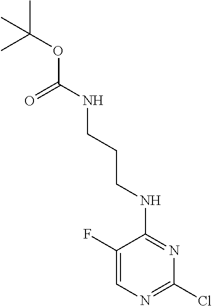 [SMILES: CC(C)(C)OC(=O)NCCCNc1nc(Cl)ncc1F]

tert-butyl (3-((2-chloro-5-fluoropyrimidin-4-yl)amino)propyl)carbamate was prepared according to the protocol described in general procedure H (8.5 g, 93% yield). ¹H NMR (400 MHz, CDCl₃) δ 7.86 (d, J=7.0 Hz, 1H), 6.31 (s, 1H), 4.94 (d, J=25.3 Hz, 1H), 3.66-3.53 (m, 2H), 3.24 (s, 2H), 1.77 (s, 2H), 1.46 (s, 9H). ¹⁹F NMR (376 MHz, CDCl₃) δ −159.52 (d, J=23.6 Hz).

### Synthesis of tert-butyl (3-((5-fluoro-2-((4-(2-methoxyethoxy)phenyl)amino)pyrimidin-4-yl)amino)propyl)carbamate

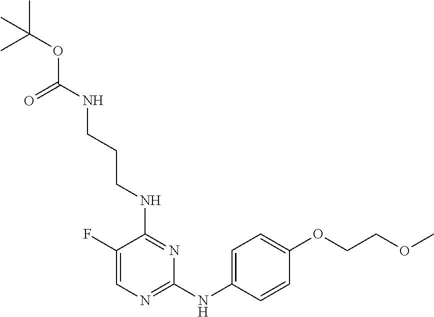 [SMILES: COCCOc1ccc(Nc2ncc(F)c(NCCCNC(=O)OC(C)(C)C)n2)cc1]

tert-butyl (3-((5-fluoro-2-((4-(2-methoxyethoxy)phenyl)amino)pyrimidin-4-yl)amino)propyl) carbamate was prepared according to the protocol described in general procedure I (4 g, 93% yield). ¹H NMR (400 MHz, DMSO-d₆) δ 10.35 (s, 1H), 9.10 (s, 1H), 8.12 (d, J=5.2 Hz, 1H), 7.46 (dd, J=21.9, 8.4 Hz, 2H), 7.03 (dd, J=24.0, 8.8 Hz, 2H), 6.87 (s, 1H), 4.19-3.89 (m, 2H), 3.68-3.53 (m, 2H), 3.41 (dd, J=12.8, 6.5 Hz, 2H), 3.32 (s, 3H), 3.14-2.76 (m, 2H), 1.71 (p, J=7.0 Hz, 2H), 1.37 (s, 9H). ¹⁹F NMR (376 MHz, DMSO-d₆) δ −163.25 (s).

### Synthesis of N⁴-(3-aminopropyl)-5-fluoro-N²-(4-(2-methoxyethoxy)phenyl)pyrimidine-2,4-diamine

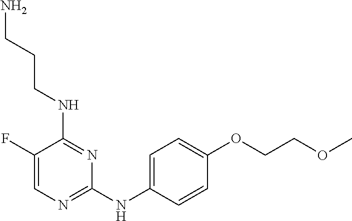 [SMILES: COCCOc1ccc(Nc2ncc(F)c(NCCCN)n2)cc1]

N⁴-(3-aminopropyl)-5-fluoro-N²-(4-(2-methoxyethoxy)phenyl)pyrimidine-2,4-diamine was prepared according to the protocol described in general procedure J (0.192 g, 50% yield). ¹H NMR (400 MHz, CD₃CN) δ 7.74 (d, J=3.7 Hz, 1H), 7.58-7.48 (m, 2H), 7.36 (s, 1H), 6.93-6.81 (m, 2H), 6.38 (s, 1H), 4.14-4.03 (m, 2H), 3.68 (ddd, J=17.8, 7.0, 3.8 Hz, 2H), 3.58-3.47 (m, 2H), 3.38 (s, 3H), 2.78 (t, J=6.4 Hz, 2H), 1.81-1.63 (m, 2H). ¹⁹F NMR (376 MHz, CD₃CN) 6-170.97 (d, J=3.4 Hz).

### Synthesis of 5-fluoro-N²-(4-(2-methoxyethoxy)phenyl)pyrimidine-2,4-diamine

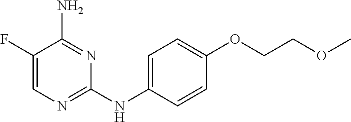 [SMILES: COCCOc1ccc(Nc2ncc(F)c(N)n2)cc1]

5-fluoro-N²-(4-(2-methoxyethoxy)phenyl)pyrimidine-2,4-diamine was prepared according to the protocol described in general procedure K (1.45 g, 77% yield). ¹H NMR (400 MHz, CD₃CN) δ 7.83 (d, J=3.4 Hz, 1H), 7.62-7.44 (m, 2H), 7.29 (s, 1H), 6.97-6.75 (m, 2H), 5.74-5.41 (m, 2H), 4.08 (dd, J=5.4, 3.8 Hz, 2H), 3.67 (ddd, J=16.9, 5.4, 3.8 Hz, 2H), 3.38 (s, 3H). ¹⁹F NMR (376 MHz, CD₃CN) 6-169.77 (d, J=3.4 Hz).

### Example 45: 2,3,4,5,6-pentafluoro-N-(3-((5-fluoro-2-((4-(2-methoxyethoxy)phenyl) amino)pyrimidin-4-yl)amino)phenyl)benzenesulfonamide (Compound 5-15

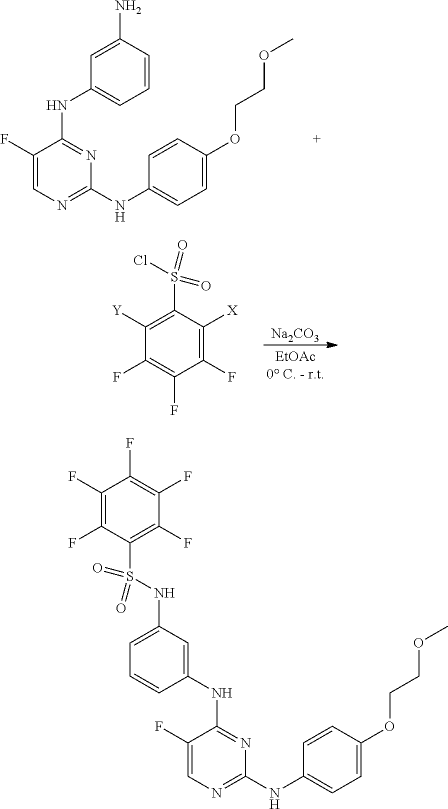

To a solution of N⁴-(3-aminophenyl)-5-fluoro-N²-[4-(2-methoxyethoxy)phenyl]pyrimidine-2,4-iamine (0.05 g, 135.36 umol, 1 eq.) in ethyl acetate (0.1 M, 1.35 mL) was added sodium carbonate (14.35 mg, 135.36 umol, 1 eq.) at 0° C. under a nitrogen atmosphere. 2,3,4,5,6 pentafluorobenzenesulfonyl chloride (21.05 uL, 142.13 umol, 1.05 eq.) was added dropwise to the stirring mixture and the reaction permitted to warm to room temperature over 2 hours. The reaction was quenched with water and extracted three times with ethyl acetate. The combined organic phases were washed with brine, dried over anhydrous sodium sulfate, filtered, and concentrated in vacuo. The crude material was purified on a Biotage Isolera equipped with a 10 g silica cartridge running a solvent gradient of 20 to 30% EtOAc in Hexanes to afford the desired product (0.052 g, 0.088 mmol, 64%). ¹H NMR (400 MHz, CDCl₃) δ 7.94 (s, 1H), 7.81 (s, 1H), 7.36 (d, J=8.9 Hz, 2H), 7.32-7.28 (m, 1H), 7.22 (t, J=8.1 Hz, 1H), 7.20 (s, 1H), 6.96 (d, J=3.0 Hz, 2H), 6.94-6.89 (m, 1H), 6.86 (d, J=8.9 Hz, 2H), 4.15-4.11 (m, 2H), 3.82-3.78 (m, 2H), 3.49 (s, 3H). ¹⁹F NMR (376 MHz, CDCl₃) δ −136.37 (qd, J=13.2, 7.5 Hz), −144.32 (tt, J=21.1, 6.9 Hz), −157.96 (tt, J=21.1, 6.3 Hz), −167.58 (d, J=3.5 Hz). ESI-MS: measured m/z 597.6 [M+H]⁺.

### Example 46: 2,3,4,5,6-pentafluoro-N-(3-((5-fluoro-2-((4-(2-methoxyethoxy)phenyl)amino) pyrimidin-4-yl)amino)phenyl)-N-methylbenzenesulfonamide (Compound 5-17

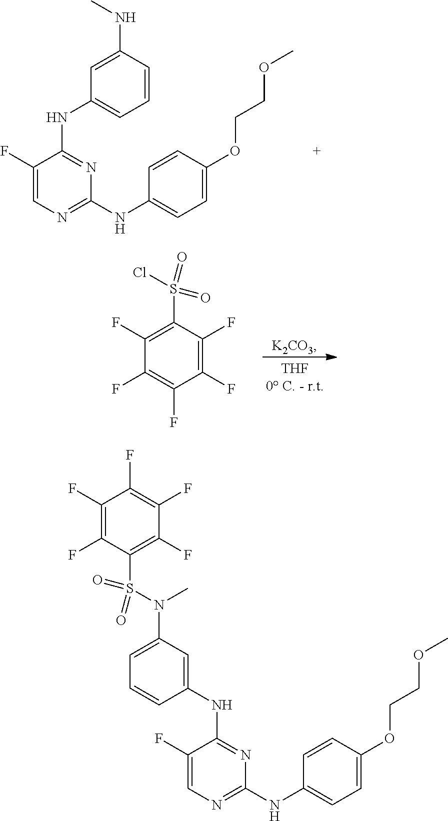

To a solution of 5-fluoro-N²-(4-(2-methoxyethoxy)phenyl)-N4-(3-(methylamino)phenyl)pyrimidine-2,4-diamine (0.06 g, 156 umol, 1 eq.) in tetrahydofuran (0.1 M, 1.5 mL) was added sodium carbonate (43 mg, 313 umol, 2 eq.) at 0° C. under a nitrogen atmosphere. 2,3,4,5,6 pentafluorobenzenesulfonyl chloride (35 uL, 267 umol, 1.5 eq.) was added dropwise to the stirring mixture and the reaction permitted to warm to room temperature over 2 hours. After 16 hours, the reaction was quenched with water and extracted three times with ethyl acetate. The combined organic phases were washed with brine, dried over anhydrous sodium sulfate, filtered, and concentrated in vacuo. The crude material was purified on a Biotage Isolera equipped with a 12 g reverse-phase cartridge running a solvent gradient of 30% to 80% ACN (0.1% FA v/v) in water (0.1% FA v/v). The isolated product was lyophilized from ACN and water to afford the desired product as a white powder (0.052 g, 0.088 mmol, 64%). ¹H NMR (400 MHz, CDCl₃) δ 7.96 (br, 1H), 7.76 (t, 1H), 7.55 (dd, 1H), 7.40-7.36 (m, 2H), 7.28 (t, 1H), 6.95 (br, 1H), 6.92-6.88 (m, 2H), 6.83 (dd, 2H), 4.13-4.11 (m, 2H), 3.77-3.74 (m, 2H), 3.45 (s, 3H), 3.39 (t, 3H). ¹⁹F NMR (376 MHz, CDCl₃) δ −133.9, −144.8, —158.2, −167.5 ESI-MS: measured m/z 614.2 [M+H]⁺. Purity by HPLC: 97.8% @ 254 nm.

### Example 47: 2,3,4,5,6-pentafluoro-N-(4-((5-fluoro-2-((4-(2-methoxyethoxy)phenyl)amino) pyrimidin-4-yl)amino)phenyl)benzenesulfonamide (Compound 5-14

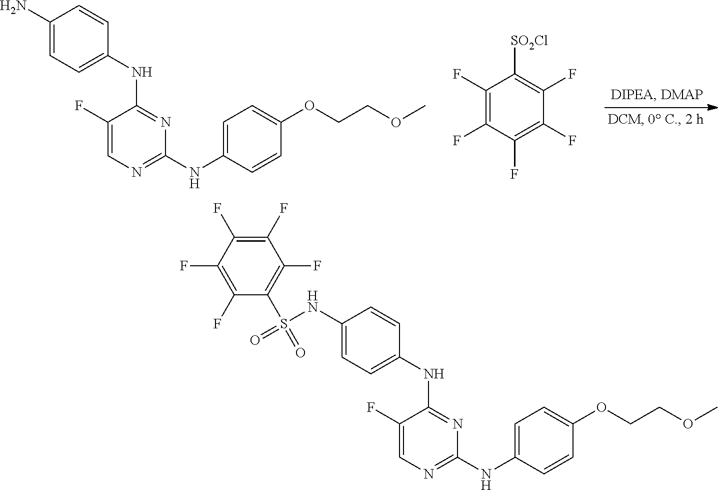

A mixture of N⁴-(4-aminophenyl)-5-fluoro-N²-[4-(2-methoxyethoxy)phenyl]pyrimidine-2,4-diamine (50.00 mg, 135.36 umol), N,N-dimethylpyridin-4-amine (3.31 mg, 27.07 umol), and DIPEA (26.24 mg, 203.04 umol, 35.37 uL) in anhydrous dichloromethane (1.25 mL) was cooled to 0° C. under a nitrogen atmosphere. Once cold, a solution of 2,3,4,5,6-pentafluorobenzenesulfonyl chloride (32.47 mg, 121.82 umol, 18.04 uL) in anhydrous dichloromethane (1.25 mL) was added dropwise and the resulting mixture stirred for 2 hours. The reaction was quenched with water and extracted 3 times with ethyl acetate. The combined organic phases were washed with brine, dried over anhydrous sodium sulfate, filtered, and concentrated to dryness. The crude residue was purified by Prep-HPLC running a mobile phase of 90% to 0% H₂O (0.1% FA) in ACN (0.1% FA) over 60 minutes to afford the desired product (0.023 g, 28% yield, 98.6% purity). ¹H NMR (400 MHz, CDCl₃) δ 7.95 (d, J=3.0 Hz, 1H), 7.57 (d, J=8.9 Hz, 2H), 7.37 (d, J=8.9 Hz, 2H), 7.15 (d, J=8.8 Hz, 2H), 6.98-6.89 (m, 3H), 6.79 (s, 1H), 4.20-4.13 (m, 2H), 3.85-3.79 (m, 2H), 3.53 (s, 3H). ¹⁹F NMR (376 MHz, CDCl₃) δ −136.00-−136.62 (m), −144.09-−144.75 (m), −157.86-−158.33 (m), −167.80 (s). ESI-MS: measured m/z 599.1 [M+H]⁺. Purity by HPLC: 98.6% @ 254 nm.

### Example 48: 2,3,4,5,6-pentafluoro-N-(2-((5-fluoro-2-((4-(2-methoxyethoxy)phenyl)amino) pyrimidin-4-yl)amino)ethyl)benzenesulfonamide (Compound 5-13

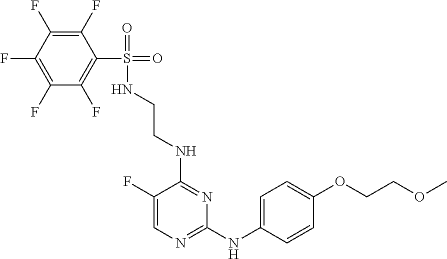 [SMILES: COCCOc1ccc(Nc2ncc(F)c(NCCNS(=O)(=O)c3c(F)c(F)c(F)c(F)c3F)n2)cc1]

The title compound 2,3,4,5,6-pentafluoro-N-(2-((5-fluoro-2-((4-(2-methoxyethoxy) phenyl)amino)pyrimidin-4-yl)amino)ethyl)benzenesulfonamide, was prepared according to the protocol described in general procedure K and isolated as a white powder (0.037 g, 20% yield). ¹H NMR (400 MHz, CD₃CN) δ 7.75 (s, 1H), 7.53 (s, 1H), 7.48 (d, J=8.7 Hz, 2H), 6.88 (d, J=8.5 Hz, 2H), 6.62 (s, 1H), 6.01 (s, 1H), 4.08 (s, 2H), 3.70 (d, J=3.0 Hz, 2H), 3.54 (d, J=5.0 Hz, 2H), 3.43 (d, J=4.8 Hz, 2H), 3.38 (s, 3H). ¹⁹F NMR (376 MHz, CD₃CN) δ −139.04-−139.26 (m), −149.80 (t, J=20.3 Hz), −161.48 (t, J=18.0 Hz), −170.68 (s). ESI-MS: measured m/z 551.7 [M+H]⁺. Purity by HPLC: 99.1% @ 254 nm.

### Example 49: 2,3,4,5,6-pentafluoro-N-(3-((5-fluoro-2-((4-(2-methoxyethoxy)phenyl)amino) pyrimidin-4-yl)amino)propyl)benzenesulfonamide (Compound 5-7

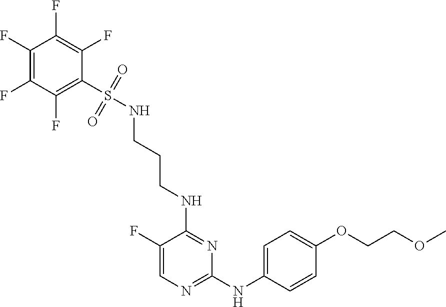 [SMILES: COCCOc1ccc(Nc2ncc(F)c(NCCCNS(=O)(=O)c3c(F)c(F)c(F)c(F)c3F)n2)cc1]

The title compound 2,3,4,5,6-pentafluoro-N-(3-((5-fluoro-2-((4-(2-methoxyethoxy)phenyl) amino)pyrimidin-4-yl)amino)propyl)benzenesulfonamide, was prepared according to the protocol described in general procedure K and isolated as a white powder. ¹H NMR (400 MHz, CD₃CN) δ 8.07 (s, 1H), 7.76 (d, J=3.7 Hz, 1H), 7.56-7.48 (m, 2H), 6.93-6.85 (m, 2H), 6.66 (s, 1H), 5.98 (s, 1H), 4.08 (dd, J=5.4, 3.8 Hz, 2H), 3.70 (dd, J=5.4, 3.8 Hz, 2H), 3.50 (q, J=6.4 Hz, 2H), 3.39 (s, 3H), 3.18 (q, J=6.6 Hz, 2H), 1.86 (p, J=6.7 Hz, 2H). ¹⁹F NMR (376 MHz, CD₃CN) δ −139.12, −139.24 (m), −149.59-−149.73 (m), −161.44-−161.61 (m). ESI-MS: measured m/z 566.5 [M+H]⁺.

### Example 50: 2,3,4,5,6-pentafluoro-N-(5-fluoro-2-((4-(2-methoxyethoxy)phenyl)amino)pyrimidin-4-yl)benzenesulfonamide (Compound 5-16

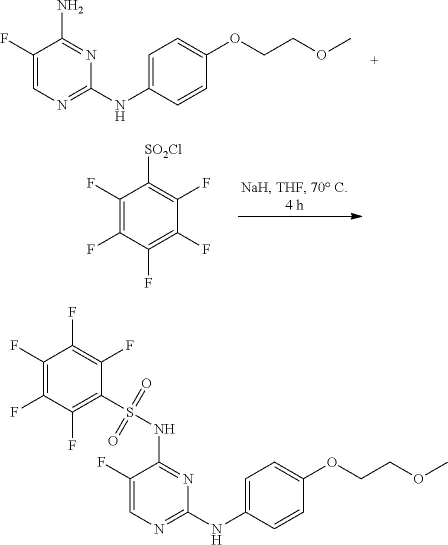 [SMILES: COCCOc1ccc(Nc2ncc(F)c(N)n2)cc1 ; O=S(=O)(Cl)c1c(F)c(F)c(F)c(F)c1F ; COCCOc1ccc(Nc2ncc(F)c(NS(=O)(=O)c3c(F)c(F)c(F)c(F)c3F)n2)cc1]

A mixture of 5-fluoro-N2-[4-(2-methoxyethoxy)phenyl]pyrimidine-2,4-diamine (49.79 mg, 178.90 umol) and a mineral dispersion of sodium hydride (7.87 mg, 196.79 umol, 60% in mineral oil) were dissolved in THE (1.8 mL) and cooled to 0° C. using an ice bath. Once sufficiently cold, a solution of 2,3,4,5,6-pentafluorobenzenesulfonyl chloride (42.92 mg, 161.01 umol, 23.85 uL) in THE (1.8 mL) was added dropwise. The resulting mixture was allowed to warm to room temperature and then refluxed for 4 h. Once the reaction was finished, excess solvent was removed under reduced pressure and the residue reconstituted in ethyl acetate. The crude material was washed with a saturated aqueous solution of sodium bicarbonate, brine, dried over anhydrous sodium sulfate, and concentrated to dryness. The crude residue was purified by Prep-HPLC running a mobile phase of 90% to 0% H₂O (0.1% FA) in ACN (0.1% FA) over 60 minutes to afford the desired product. ¹H NMR (400 MHz, CD₃CN) δ 11.90 (s, 1H), 10.38 (s, 1H), 8.20 (s, 1H), 7.27 (dd, J=3.6, 2.2 Hz, 1H), 6.72 (dd, J=3.6, 1.9 Hz, 1H); ¹⁹F NMR (376 MHz, CD3CN) 6-139.51 (dt, J=20.8, 5.4 Hz, 2F), −151.68-−151.85 (m, 1F), −162.26-−162.44 (m, 2F); ESI-MS: measured m/z 509.3 [M+H]⁺.

### Example 51: (S)—N-(4-((3-chloro-4-fluorophenyl)amino)-7-((tetrahydrofuran-3-yl)oxy)quinazolin-6-yl)-2,3,4,5,6-pentafluorobenzenesulfonamide (Compound 5-20

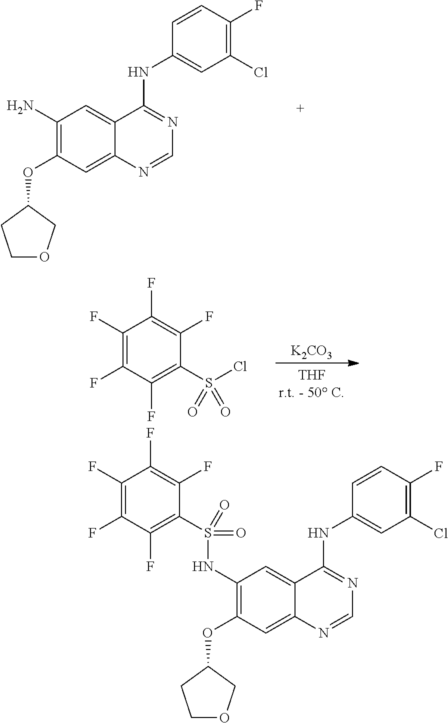

To an oven dried microwave vial (2-5 mL) equipped with a stir bar and purged with argon, was added N-4-(3-chloro-4-fluoro-phenyl)-7-[(3S)-tetrahydrofuran-3-yl]oxy-quinazoline-4,6-diamine (150 mg, 400.22 μmol) and potassium carbonate (110.63 mg, 800.44 μmol). THE (1 mL) was added and the resulting suspension stirred at room temperature for 5 minutes. 2,3,4,5,6-pentafluorobenzenesulfonyl chloride (117.36 mg, 440.24 μmol, 65.20 μL) was added dropwise and the reaction stirred at room temperature for 1 h, then for 30 min at 50° C. Upon observation of a new product by TLC, the mixture was cooled to room temperature and partitioned between ethyl acetate and brine/water (1:1 v/v). The organic phase was separated and washed with a saturated aqueous solution of sodium bicarbonate, brine, dried over anhydrous magnesium sulfate, filtered and concentrated in vacuo. The concentrate was chromatographed on silica gel employing a mobile phase of 0-100% EtOAc in Hexanes. The product containing fractions were concentrated in vacuo to afford N-[4-(3-chloro-4-fluoro-anilino)-7-[(3S)-tetrahydrofuran-3-yl]oxy-quinazolin-6-yl]-2,3,4,5,6-pentafluoro-benzenesulfonamide (51 mg, 84.31 μmol, 21% yield) as a light yellow solid. ¹H NMR (d6-DMSO) δ 11.03 (brs, 1H), 9.97 (s, 1H), 8.61-8.55 (m, 2H), 8.19-8.15 (m, 1H), 7.86-7.80 (m, 1H), 7.49-7.42 (m, 1H), 7.13 (s, 1H), 5.12-5.06 (m, 1H), 3.79-3.72 (m, 1H), 3.69-3.55 (m, 2H), 3.51-3.44 (m, 1H), 2.35-2.23 (m, 1H), 1.79-1.68 (m, 1H). ¹⁹F NMR (d6-DMSO) δ −122.6 (1F), −137.1 (2F), −147.5 (1F), −160.0 (2F). ESI-MS: measured m/z 603.1 [M−H]⁻. Purity by HPLC: 97.5% @ 254 nm.

### Example 52: (S)—N-(4-((3-chloro-4-fluorophenyl)amino)-7-((tetrahydrofuran-3-yl)oxy)quinazolin-6-yl)-2,3,4,5,6-pentafluoro-N-methylbenzenesulfonamide (Compound 5-21

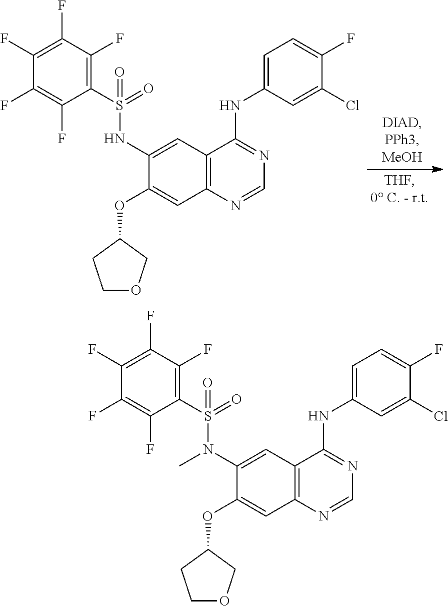

An oven-dried 10 mL microwave vial was charged with N-[4-(3-chloro-4-fluoro-anilino)-7-[(3S)-tetrahydrofuran-3-yl]oxy-quinazolin-6-yl]-2,3,4,5,6-pentafluoro-benzenesulfonamide (1 eq., 138 mg, 228 μmol), triphenylphosphine (1.2 eq., 72 mg, 274 μmol), methanol (1.2 eq., 9 mg, 274 μmol, 11.09 μL), and THE (6 mL). The vial was cooled to 0° C. Once at temperature, neat diisopropyl azodicarboxylate (DIAD, 1.2 eq., 55.36 mg, 274 μmol, 53.74 uL) was added dropwise and the reaction permitted to warm to room temperature overnight. After 16 hours, the reaction was quenched with a saturated aqueous solution of ammonium chloride and the mixture was extracted with ethyl acetate (twice). The combined organic phases were washed with brine, dried over anhydrous sodium sulfate, filtered, and concentrated under reduced pressure. The crude material was purified by reverse phase chromatography using a mixture of ACN (0.1% FA)/H₂O (0.1% FA) (30% to 0% gradient) and the product containing fractions were concentrated to afford (S)—N-(4-((3-chloro-4-fluorophenyl)amino)-7-((tetrahydrofuran-3-yl)oxy)quinazolin-6-yl)-2,3,4,5,6-pentafluoro-N-methylbenzenesulfonamide (29 mg, 46.85 umol, 20.54% yield) as a white solid. ¹H NMR (400 MHz, CD₃CN) δ 8.61 (s, 1H), 8.42-8.38 (bs, 1H), 8.37 (s, 1H), 8.12 (dd, J=6.8, 2.7 Hz, 1H), 7.70 (ddd, J=9.0, 4.3, 2.7 Hz, 1H), 7.30 (t, J=9.0 Hz, 1H), 7.20 (s, 1H), 5.12-5.06 (m, 1H), 3.84-3.78 (m, 2H), 3.66 (q, J=7.9 Hz, 2H), 3.44 (s, 3H), 2.47-2.33 (m, 2H). ¹⁹F NMR (376 MHz, CD3CN) δ −123.2 (1F), −136.7 (2F), −148.6 (1F), −161.1 (2F). ESI-MS: measured m/z 619.2 [M+H]⁺. Purity by HPLC: 98.3% @ 254 nm.

### II. Biological Evaluation

### Examples B1: In Vitro Cell Viability Studies

Anti-cancer efficacy of exemplary compounds of this application is assessed in vitro in different cancer cell lines. Cell viability is examined following treatment at various concentration of inhibitor (0.097656-50 μM) using a cell Titer-Blue cell viability assay. 1×104 cells (NHF cells)/well are plated in 96-well assay plates in culture medium. All cells are grown in DMEM, IMDM and RPMI-1640 supplemented with 10% FBS. After 24 hrs, test compounds and vehicle controls are added to appropriate wells so the final volume is 100p in each well. The cells are cultured for the desired test exposure period (72 hrs) at 37° C. and 5% CO2. The assay plates are removed from 37° C. incubator and 20 μl/well of CellTiter-Blue® Reagent is added. The plates are incubated using standard cell culture conditions for 1-4 hours and the plates are shaken for 10 seconds and record fluorescence at 560/590 nm.

### Examples B2 Reactivity Profiling with Glutathione

The experiment is started by placing 1 μL of 1 mM stocking solution of the test compound in DMSO in 199 μL of PBS buffer at pH 7.4 with 5 mM GSH to reach a final concentration of 5 μM. The final DMSO concentration is 0.5%. The solution is then incubated at 25° C. at 600 rpm, and is quenched with 600 μL solution of acetonitrile at 0, 30, 60 and 120 minutes. The quenched solution is vortexed for 10 minutes and centrifuged for 40 minutes at 3,220 g. An aliquot of 100 μL of the supernatant is diluted by 100 μL ultra-pure water, and the mixture is used for LC/MS/MS analysis. The data is processed and analyzed using Microsoft Excel.

### Examples B3 Parallel Artificial Membrane Permeability Assay (PAMPA

The stock solutions of positive controls are prepared in DMSO at the concentration of 10 mM. Testosterone and methotrexate are used as control compounds in this assay. Prepare a stock solution of compounds in DMSO at the concentration of 10 mM, and further dilute with PBS (pH 7.4). The final concentration of the test compound is 10 μM.

Assay Procedures. 1) Prepare a 1.8% solution (w/v) of lecithin in dodecane, and sonicate the mixture to ensure a complete dissolution. 2)Carefully pipette 5 μL of the lecithin/dodecane mixture into each acceptor plate well (top compartment), avoiding pipette tip contact with the membrane. 3) Immediately after the application of the artificial membrane (within 10 minutes), add 300 μL of PBS (pH 7.4) solution to each well of the acceptor plate. Add 300 μL of drug-containing solutions to each well of the donor plate (bottom compartment) in triplicate. 4) Slowly and carefully place the acceptor plate into the donor plate, making sure the underside of the membrane is in contact with the drug-containing solutions in all wells. 5) Replace the plate lid and incubate at 25° C., 60 rpm for 16 hours. 6) After incubation, aliquots of 50 μL from each well of acceptor and donor plate are transferred into a 96-well plate. Add 200 L of methanol (containing IS: 100 nM Alprazolam, 200 nM Labetalol and 2 μM Ketoprofen) into each well. 7) Cover with plate lid. Vortex at 750 rpm for 100 seconds. Samples are centrifuged at 3,220 g for 20 minutes. Determine the compound concentrations by LC/MS/MS.

### Examples B4 Kinase Assay

A 96-well half-area clear flat-bottom microplate (Corning© #3697) is pre-heated in a plate reader (Cytation 3, BioTek) at 37° C. for 15 minutes prior to the start of each assay. Kinase buffer (80 mM PIPES pH 6.9, 2 mM MgCl₂, 0.5 mM EGTA, 15% glycerol, 1 mM GTP) is prepared from stock solutions and placed on ice. Inhibitors of this application are prepared to 10 μM concentrations in buffer (80 mM PIPES pH 6.9, 2 mM MgCl₂, 0.5 mM EGTA, 5% DMSO) from DMSO stock solutions. After the assay plate is pre-warmed, 10 μL of inhibitor or buffer control is added to selected wells. Every assay contained a kinase only negative control for normalization of data, and a known compound positive control. The assay plate is incubated at 37° C. for 3 minutes. During this time, a frozen aliquot of the kinase (10 mg/mL) in buffer (80 mM PIPES pH 6.9, 2 mM MgCl₂, 0.5 mM EGTA) is defrosted by placing in a room temperature water bath. Once thawed, 200 μL of the kinase is mixed with 420 μL of the ice-cold kinase buffer (3 mg/mL kinase in 80 mM PIPES, pH 6.9, 2 mM MgCl₂, 0.5 mM EGTA, 1 mM GTP, 10.2% glycerol). To a 96-well plate on ice, aliquots of 100 μL kinase is added to each well. From this plate, 90 μL of the kinase is immediately pipetted into all sample wells of the warmed assay plate using a multi-channel pipette. The assay plate is immediately put in the reader at 37° C. and shook for 5 s with orbital shaking at medium speed. The reader records the absorbance at 340 nm every 15 s for 30 min.

The resulting absorbance curves are normalized by subtracting each data point by the absorbance at time 0. The slope of the initial linear portion (“Vₘₐₓ”) is determined in mOD/min, and normalized to the Vₘₐₓ value of the kinase only control, using the following equation, resulting in comparable % inhibition values:

$$\% inhibition = ( 1 - \frac{{V}_{max ( kinase + i n h i b itor )}}{{V}_{max ( kinase )}} ) \times 100$$

### Examples E1 Target Engagement and Mechanism of Action Studies

### Target Engagement

IC50 Values (nM) for the Inhibition of BTK Activity

In vitro BTK and JAK3 inhibition studies are shown in Table 7 and Table 8 (no preincubation).

TABLE 7

| Compound | BTK (IC₅₀, nM) |
|---|---|
| Ibrutinib | B |
| Compound 5-1 | C |
| Compound 5-3 | B |
| Compound 5-4 | B |
| Compound 5-5 | B |
| Compound 5-6 | B |

Note:
Biochemical assay IC₅₀ data are designated within the following ranges
A: ≤0.1 nM
B: >0.1 nM to ≤ 10 nM
C: >10 nM to ≤ 100 nM
D: >100 nM to ≤ 1000 nN

TABLE 8

| Compound | BTK (IC₅₀, nM) | JAK3 (IC₅₀, nM) |
|---|---|---|
| Spebrutinib | C | — |
| Compound 5-3 | B | — |
| Compound 5-15 | D | — |
| Compound 5-8 | — | E |
| Compound 5-10 | B | E |
| Compound 5-11 | D | E |

Note:
Biochemical assay IC₅₀ data are designated within the following ranges
A: ≤0.1 nM
B: >0.1 nM to ≤ 10 nM
C: >10 nM to ≤ 100 nM
D: >100 nM to ≤ 1000 nM
E: >1000 nM to ≤ 0,000 nM

In vitro kinase inhibition studies are shown in Table 9.

TABLE 9

| | BMX | FGFR1 | FGFR4 |
|---|---|---|---|
| | (IC₅₀, | (IC₅₀, | (IC₅₀, |
| Compound | nM) | nM) | nM) |
| 5-2 | B | | |
| 5-15 | — | B | C |
| 5-14 | — | C | C |
| 5-1 | B | C | C |
| 5-7 | — | D | D |
| 5-13 | — | E | E |
| 5-17 | B | — | — |
| 5-3 | B | — | — |
| 5-19 | C | — | — |

Note:
Biochemical assay IC₅₀ data are designated within the following ranges
A: ≤0.1 nM
B: >0.1 nM to ≤ 10 nM
C: >10 nM to ≤ 300 nM
D: >300 nM to ≤ 2 μM
E: >2 μM

In vitro irreversible inhibition studies are shown in Table 10.

TABLE 10

| Inhibitor | k_{inact}/Kᵢ (μM⁻¹s⁻¹)* |
|---|---|
| Compound 5-4 | B |
| Compound 5-5 | B |
| Compound 5-6 | B |
| Compound 5-1 | A |

*Based on the following two-step kinetic scheme:

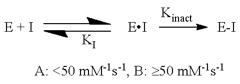

In Vitro Efficacy Studies

In vitro IC₅₀ against human cancer cells and normal human cells are shown in Table 11.

TABLE 11
Human cancer cell lines (IC₅₀, μM)

| Compound | MV-4-11 | MOLM-13 | K562 |
|---|---|---|---|
| Batabulin | A | A | A |
| 4-1 | C | C | C |
| 4-2 | C | C | N/A |
| 4-3 | B | N/A | N/A |
| 4-4 | B | B | N/A |
| 4-5 | C | N/A | N/A |
| 4-6 | B | B | B |
| 4-7 | B | — | B |
| 4-12 | N/A | N/A | B |

Note:
Biochemical assay IC₅₀ data are designated within the following ranges
A: ≤0.1 μM
B: >0.1 μM to ≤ 10 μM
C: >10 μM to ≤ 100 μM
D: >100 μM to ≤ 1000 μM

TABLE 12
In vitro % inhibition of tubulin polymerization are shown in Table

| Compound | CLASS | % inhibition |
|---|---|---|
| Batabulin | — | C |
| 4-1 | Batabulin | B |
| 4-3 | Batabulin | A |
| 4-4 | Batabulin | B |
| 4-6 | Batabulin | B |
| 4-7 | Batabulin | C |

Inhibition:
A: < 10%,
B: 10-20%,
C > 20%

In vitro kinase inhibition studies are shown in Table 13.

TABLE 13

| Kinase (IC₅₀, nM) | Ibrutinib | 5-1 |
|---|---|---|
| BLK | B | D |
| BMX | B | B |
| FGFR2 | D | D |
| JAK3 | D | D |
| MAP2K7 | C | C |
| CSK | C | D |
| EGFR | B | D |
| EGFR L850R | D | D |
| EGFR L850R, T790M | D | D |
| ERBB2 | C | D |
| ERBB4 | B | D |
| FGFR | B | D |
| SRC | B | D |

Note:
Biochemical assay IC₅₀ data are designated within the following ranges
A: ≤0.1 nM
B: >0.1 nM to ≤ 10 nM
C: >10 nM to ≤ 100 nM
D: >100 nM to ≤ 1000 nN

### General Definitions:

Methods for identifying if a compound is acting as an irreversible inhibitor are known to one of ordinary skill in the art. Such methods include, but are not limited to, the use of mass spectrometry of the protein drug target modified in the presence of the inhibitor compound, enzyme kinetic analysis of the inhibition profile of the compound with the target protein, and discontinuous exposure, also known as “washout,” experiments, as well as other methods known to one of skill in the art.

One of ordinary skill in the art will recognize that certain reactive functional groups can act as “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified), present in or near the binding pocket of a target protein, thereby irreversibly inhibiting the protein. It will be appreciated that in some embodiments the Linker-Warhead group (L-WH), as defined and described herein, provides such warhead groups for covalently, and irreversibly, inhibiting the protein.

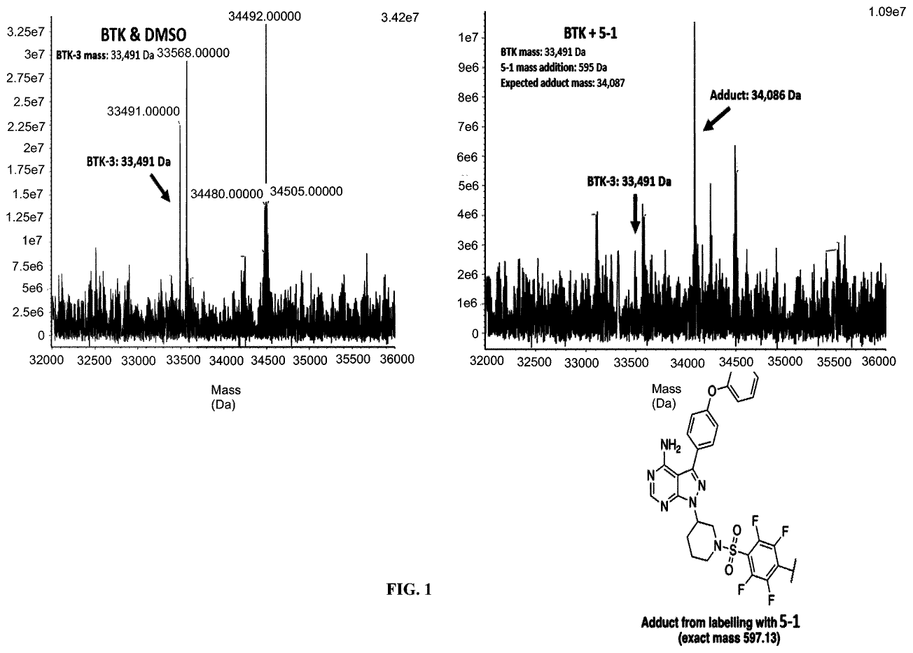

In one example, covalent modification of the enzyme BTK with Compound 5-1 has been demonstrated by mass spectrometry. After incubation of 10 μM BTK in the presence of 50 μM Compound 5-1 for 2 hours at 30° C., the mass of the modified protein was determined by mass spectrometry. As shown in FIG. 1, the mass of the parent ion of the His-tagged protein prior to incubation was the third most intense peak, in this experiment corresponding to 33491 Da. After incubation with Compound 5-1, the intensity of this had decreased by an order of magnitude, and the most intense peak is consistent with the protein whose mass was increased by 595 Da, corresponding closely to the expected mass of the inhibitor Compound 5-1 (less one fluoride from inhibitor, and one proton from protein, 596 Da). One of ordinary skill in the art will recognize that this is consistent with the covalent modification of BTK after irreversible inhibition by Compound 5-1.

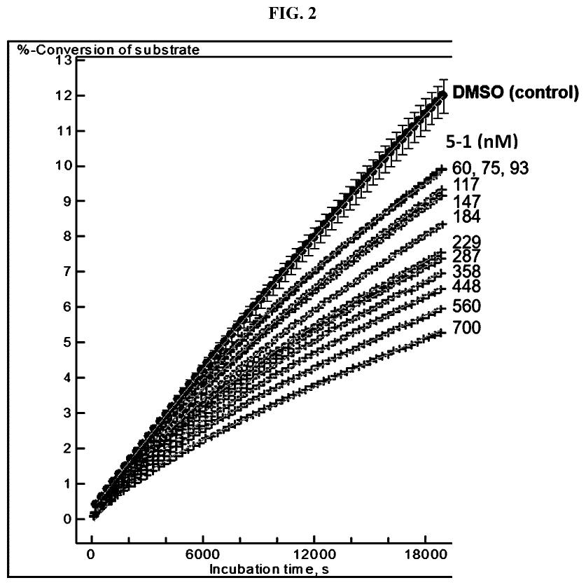

In another example, covalent modification of the enzyme BTK with Compound 5-1 has been demonstrated by enzyme kinetic analysis of the inhibition profile of Compound 5-1. The reaction of 0.1 nM BTK with 500 μM of its substrate ATP in the presence of 60-700 nM Compound 5-1 was shown to exhibit time-dependent inhibition corresponding to mono-exponential time courses (FIG. 2). Further, the rate constant of this time-dependent inhibition was shown to increase in a dose-dependent manner on the concentration of Compound 5-1. One of ordinary skill in the art will recognize that this is consistent with the irreversible inhibition of BTK by Compound 5-1.

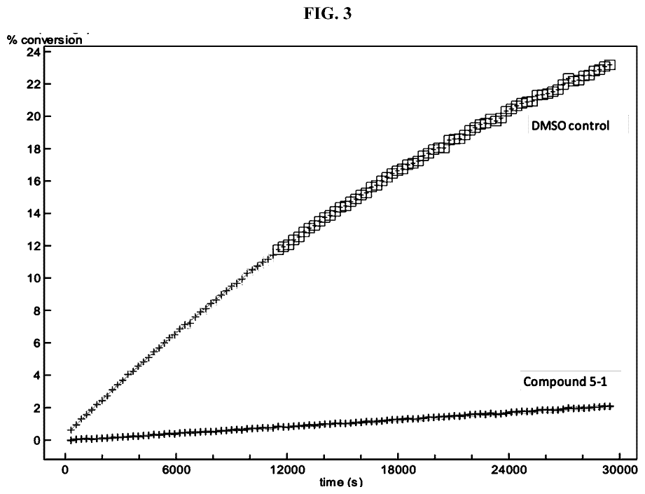

In another example, covalent modification of the enzyme BTK with Compound 5-1 has been demonstrated by a “washout” experiment. After a 3-h incubation of 40 nM BTK in the presence of 296 nM of compound Compound 5-1, a 400-fold dilution was performed to remove excess inhibitor. As shown in FIG. 3, the residual activity of the enzyme was observed to be 7% of that of the enzyme incubated with DMSO alone (positive control), indicating to one of ordinary skill in the art that >90% of the enzyme was inhibited irreversibly during the incubation period.

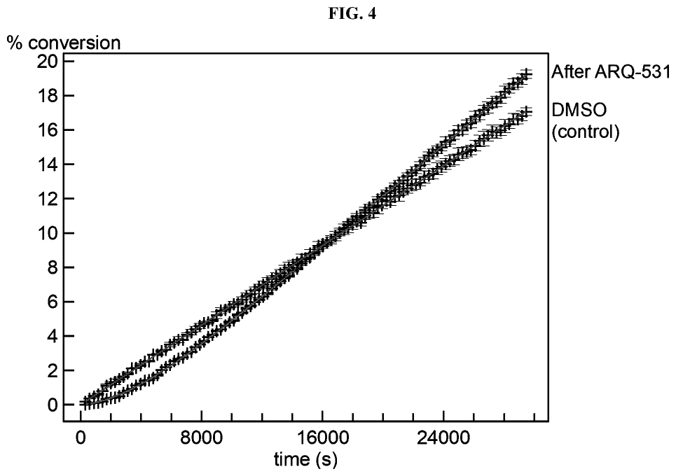

By way of contrast, a “washout” experiment performed with ARQ-531 gives a very different result, because this compound lacks a warhead and is known to inhibit BTK reversibly. After a 6-h incubation of 50 nM BTK in the presence of 177 nM of ARQ-531, a 400-fold dilution was performed to remove excess inhibitor. As shown in FIG. 4, after a brief period during which reversibly bound inhibitor was released into solution, the enzyme recovered all of its activity relative to that of the enzyme incubated with DMSO alone (positive control). This indicates to one of ordinary skill in the art that the enzyme was not inhibited irreversibly during the incubation with ARQ-531. The comparison of this result obtained with the known reversible inhibitor ARQ-531 (FIG. 4) to the results of the “washout” experiments performed with Compound 5-1 (FIG. 3) indicates to one of ordinary skill in the art that the enzyme was inhibited irreversibly during incubation with Compound 5-1.

Mass Spectral Analysis

A protein kinase that is inhibited by compound and/or pharmaceutically acceptable salt of the present disclosure may be subjected to mass spectral analysis to assess the formation of permanent, irreversible covalent adducts. Suitable analytical methods to examine peptide fragments generated upon tryptic cleavage of a protein are generally known in the art. Such methods identify permanent, irreversible covalent protein adducts by observing a mass peak that corresponds to the mass of a control sample plus the mass of an irreversible adduct.

Method:

His-tagged BTK kinase domain was prepared from pET-28b(+)-BTK (402-655) vector, transformed into E. coli BL21 (DE3) RILP. The starter cultures were incubated with shaking at 37° C. until cloudy and used to inoculate 1 L of Super broth containing same concentrations of kanamycin and chloramphenicol, 10 mM MgSO₄, 5 mM CaCl₂), and 0.1% (w/v) glucose. The cultures were incubated under similar conditions to an OD₆₀₀ of 0.6-0.8. Before induction with 0.5 mM IPTG, the temperature was reduced to 16° C. The cells were harvested by centrifugation following approximately 18 h and frozen in a −80° C. freezer until use.

Cells were thawed in lysis buffer (50 mM HEPES [pH 7.6], 300 mM NaCl, 10 mM MgCl₂, 10% [v/v] glycerol, 0.1 mg/mL lysozyme, 50 μg/mL DNase I, and protease inhibitors). Cells were sonicated (30 s on, 60 s off) for 3 repeating cycles. The cell lysates were cleared by centrifugation at 4° C. The supernatant was filtered through a 0.45 μm syringe filter and loaded into a Nickel affinity resin (GE Healthcare) with the target protein eluted with 50 mM HEPES buffer containing 10% glucose, 150 mM NaCl, and 500 mM imidazole. This eluant was cleaved with Ulp1 protease and loaded onto a S200 Superdex FPLC column (GE healthcare) pre-equilibrated with 20 mM HEPES pH 7.4, 150 mM NaCl, and 10% [v/v] glycerol. Pure fractions of His-tagged BTK kinase domain, as judged by SDS-PAGE, were pooled and concentrated using Amicon 10 kDa cutoff concentrators and after aliquoting stored at −80 C until use.

His-tagged BTK kinase domain (10 μM) was incubated for 45 min in 20 mM HEPES (pH 8.0) containing 10 μM compound with final DMSO concentration of 1%. After the incubation time, the reactions were quenched by acetone precipitation and the pellet was re-dissolved in 8 M Urea. The protein was then reduced (5 mM DTT), alkylated (15 mM iodoacetamide) and digested (1 μg trypsin at 37° C. for 4 hours). The digest was then stopped by addition of trifluoroacetic acid, and peptides were removed from gel band by sonicating with increasing amounts of acetonitrile (0%, 30%, & 60%). Peptides were then purified using C₁₈ ziptips, spotted on the MALDI target plate with a-cyano-4-hydroxycinnamic acid as the desorption matrix (10 mg/ml in 0.1% TFA: Acetonitrile 50:50), and analyzed in reflectron mode.

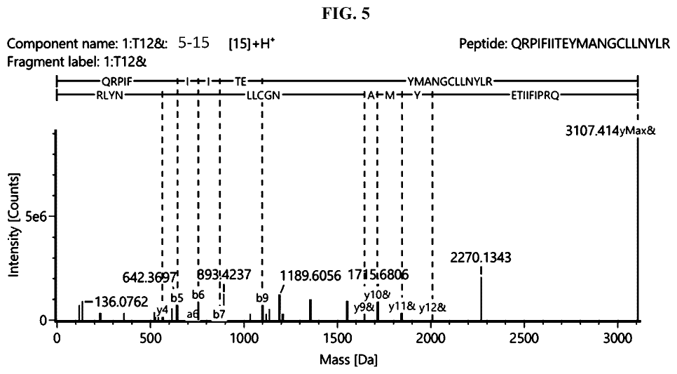

FIG. 5 shows the MSMS spectrum of peptide 467QRPIFIITEYMANGCLLNYLR487 from example Compound 5-15 treated His-BTK KD digest where the Cys is modified by one equivalent of Compound 5-15 (observed mass 3107.4 Da). The alignment of b and y ions confirms that Cys-481 is the amino acid that is modified by Compound 5-15.

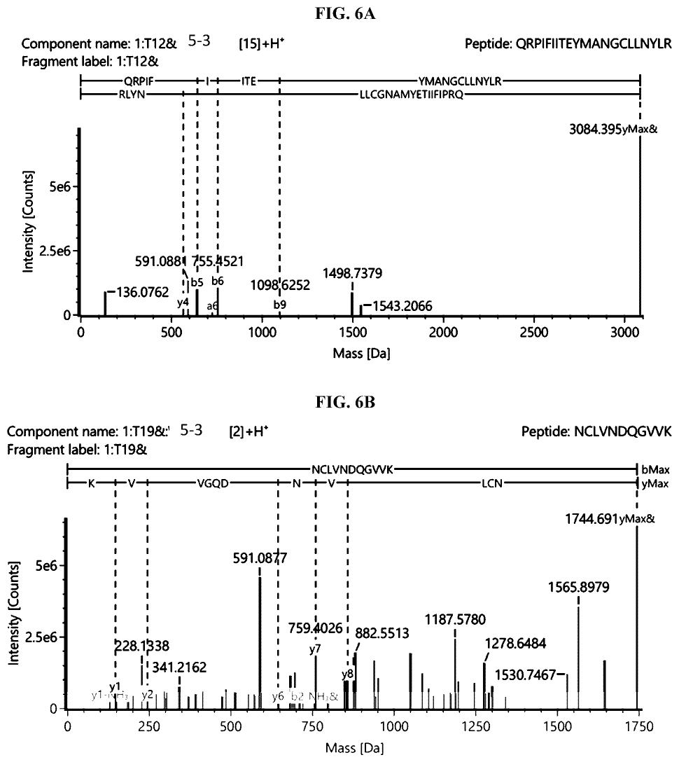

FIG. 6-A shows the MSMS spectrum of peptide 467QRPIFIITEYMANGCLLNYLR487 from example Compound 5-3 treated His-BTK KD digest where C₄₈₁ is modified by one equivalent of Compound 5-3 (observed mass 3084.4 Da).

The alignment of b and y ions confirms that C₄₈₁ is the amino acid that is modified by Compound 5-3.

FIG. 6-B shows the MSMS spectrum of peptide 526NCLVNDQGVVK536 from example Compound 5-3 treated His-BTK KD digest where C₅₂₇ is modified by one equivalent of Compound 5-3 (observed mass 1744.7 Da). The alignment of b and y ions confirms that C₅₂₇ is the amino acid that is modified by equivalent of Compound 5-3.

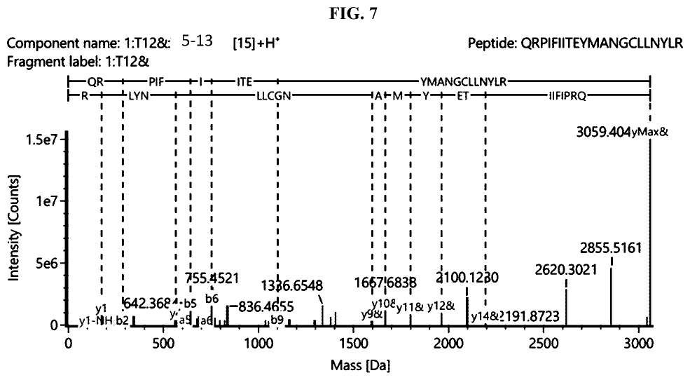

FIG. 7 shows the MSMS spectrum of peptide 467QRPIFIITEYMANGCLLNYLR487 from example Compound 5-13 treated His-BTK KD digest where the Cys is modified by one equivalent of Compound 5-13 (observed mass 3059.4 Da). The alignment of b and y ions confirms that Cys-481 is the amino acid that is modified by Compound 5-13.

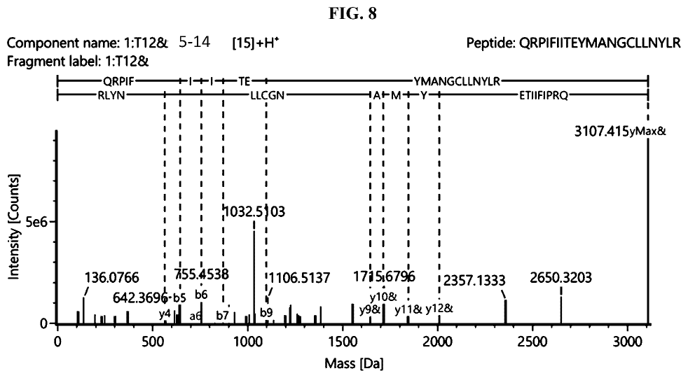

FIG. 8 shows the MSMS spectrum of peptide 467QRPIFIITEYMANGCLLNYLR487 from example Compound 5-14 treated His-BTK KD digest where the Cys is modified by one equivalent of Compound 5-14 (observed mass 3107.4 Da). The alignment of b and y ions confirms that Cys-481 is the amino acid that is modified by Compound 5-14.

Two cysteines present in tryptic digest peptides were labelled by test compounds, cysteine 481 (C₄₈₁) and cysteine 527 (C₅₂₇) confirming irreversible covalent modification of BTK.

| Compound ID | C481 | C527 |
|---|---|---|
| Ibrutinib | ✓ | |
| Compound 5-15 | ✓ | |
| Compound 5-3 | ✓ | ✓ |
| Compound 5-13 | ✓ | |
| Compound 5-14 | ✓ | |

In Vitro Efficacy Studies

IC50 values of Ibrutinib analogs against human cancer cell and normal human cells are shown in Table 14.

TABLE 14

| | | RL | Human |
|---|---|---|---|
| | Ligand | (IC₅₀, | Fibroblast (IC₅₀, |
| Reference | (G) | μM)^{a} | μM)^{b} |
| Ibrutinib | — | 10.26 | 26.8 |
| Compound 5-19 | Ibrutinib | 1.64 | — |
| Compound 5-1 | Ibrutinib | 2.74 | 6.2 |

^{a}RL is a human non-Hodgkin's lymphoma B cell line
^{b}Human fibroblast is a normal human cell line

As a specific comparative example of the biological utility of the technology relative to Ibrutinib in RL cells, compound 1, possessing a PFBS warhead, exhibits greater potency (compound 3 IC₅₀=1.6 μM vs 10.26 μM for ibrutinib). Compound 1 (TI=2.48) also exhibits a similar therapeutic index (TI) than ibrutinib (TI=2.6) as assessed in normal pooled human fibroblasts vs RL cells. Therapeutic index=(IC₅₀ human fibroblasts/IC₅₀ RLcells)

TABLE 9

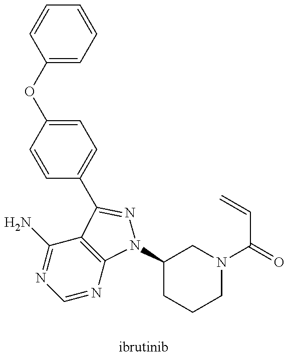 [SMILES: C=CC(=O)N1CCC[C@@H](n2nc(-c3ccc(Oc4ccccc4)cc3)c3c(N)ncnc32)C1]

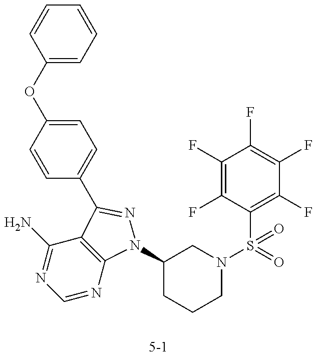 [SMILES: Nc1ncnc2c1c(-c1ccc(Oc3ccccc3)cc1)nn2[C@@H]1CCCN(S(=O)(=O)c2c(F)c(F)c(F)c(F)c2F)C1]

| | | | Human Fibroblast |
|---|---|---|---|
| Reference | Lignad (G) | RL (IC₅₀, μM)^{a} | (IC₅₀, μM)^{b} |
| Ibrutinib | — | 10.26 | 26.8 |
| Compound 5-1 | Ibrutinib | 1.61 | 3.98 |

Warhead Compound 5-1 shows better cell-based efficacy and similar therapeutic window than Ibrutinib (acrylamide) in RL cells vs human fibroblasts.

Total BTK Degradation Assay

A Total BTK-HTRF assay (Cisbio Total BTK cat #63ADK064PEG) was performed to quantitate the ability of test compounds to degrade BTK protein levels in RAMOS cells. This total protein assay monitors the steady state protein level in a sandwich assay format using two different specific antibodies after lysis of the cell-membrane. The antibodies recognise two different epitopes on BTK and are labelled with Eu3⁺-cryptate (donor) and d2 (acceptor). When the dyes are in close proximity, the excitation of the donor with a light source triggers a Fluorescence Resonance Energy Transfer (FRET) towards the acceptor, which in turn fluoresces at a specific wavelength (665 nm). The specific signal modulates positively in proportion to the total concentration of BTK.

Examples: Compounds 5-7 and 5-15 are Able to Reduce Total BTK Levels in a Protein Degradation Assay

In order to evaluate the degradation of BTK by test compounds, a total BTK degradation assay was performed (Cisbio Total BTK cat #63ADK064PEG). The frozen stock solutions of the two different BTK antibodies were diluted 20-fold with the detection buffer and pre-mixed before use in the assay. Supplemented lysis buffer (4×) was prepared by diluting the blocking reagent solution 25-fold with lysis buffer (4×) and mixing gently. RAMOS B lymphocyte cell line (ATCC CRL-1596)

were plated at a density of 50K cells/well (8 uL) in RPMI medium with 10% FBS into 384-well white detection plates. Test compound (4 μL, 5 μM), diluted with assay buffer was dispensed and the plate incubated at 37° C. for 24 hours before addition of supplemented lysis buffer (5 μL). and incubation for 30 mins at RT with shaking. The premixed antibody solution (1:1, 4 μL) was added before covering the plate and incubation for 24 hours at RT. The plate was read on an Biotek Synergy Neo2 plate reader using 330 nm excitation, 620 nm-donor emission and 670 nm-acceptor emission. A ratio is calculated (665/620) and converted to POC relative to control and blank wells. Percentage of residual BTK was calculated as follows: 100−(HTRF ratio without test compound−HTRF ratio with test compound)*100.

The compounds 5-7 and 5-15 were tested in the BTK total degradation assay described above and was found to reduce total BTK levels as shown in the table below:

| | Percent residual BTK after 24 hours |
|---|---|
| Example | with test compound (5 μM) |
| Ibrutinib | 121% |
| Compound 5-7 | 63% |
| Compound 5-15 | 75% |

### III. Preparation of Pharmaceutical Dosage Forms

### Example P1: Solution for Injection

The active ingredient is a compound of Table 1, Table 2, Table 3, Table 4, or Table 5, or a pharmaceutically acceptable salt thereof. A solution for intraperitoneal administration is prepared by mixing 1-1000 mg of active ingredient with 10-50 mL of a solvent mix made up by 25% dimethylacetamide, 50% propylene glycol and 25% Tween 80. Filter through millipore sterilizing filter and then distribute in 1 mL amber glass ampoules, performing all the operations under sterile conditions and under nitrogen atmosphere. 1 mL of such solution is mixed with 100 or 200 mL of sterile 5% glucose solution before using intraperitoneally.

The examples and embodiments described herein are for illustrative purposes only and various modifications or changes suggested to persons skilled in the art are to be included within the spirit and purview of this application and scope of the appended claims.

1. A compound of Formula (I), or a salt or solvate thereof: 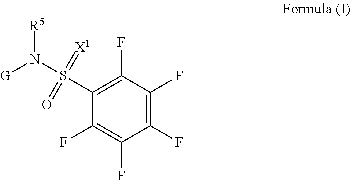
wherein,
G is or comprises substituted or unsubstituted carbocycle, is or comprises substituted or unsubstituted heterocycle, or is -L²-G¹, wherein L² is a >C═X, substituted or unsubstituted unsaturated alkylene, substituted or unsubstituted unsaturated carbocycle, or substituted or unsubstituted unsaturated heterocycle, wherein X is O, S, or NR³, and G¹ is hydrogen or an organic residue, provided that when X¹ is O, then G is not substituted or unsubstituted phenyl;
X¹ is O or NR;
R is hydrogen, substituted or unsubstituted alkyl, substituted or unsubstituted heteroalkyl, substituted or unsubstituted aryl, or substituted or unsubstituted heteroaryl;
R³ is hydrogen, -L¹R⁴, —C(═O)L¹R⁴, —C(═O)OL¹R⁴, or —C(═O)NR⁴L¹R⁴, wherein each L¹ is independently substituted or unsubstituted alkyl, substituted or unsubstituted heteroalkyl, substituted or unsubstituted aryl, or substituted or unsubstituted heteroaryl; and each R⁴ is independently hydrogen, substituted or unsubstituted C₃-C₈ cycloalkyl, substituted or unsubstituted C₂-C₇ heterocycloalkyl, substituted or unsubstituted aryl, or substituted or unsubstituted heteroaryl;
R⁵ is hydrogen, —CN, —C(═O)R⁶, —C(═O)OR⁶, —C(═O)NR³R⁶, substituted or unsubstituted alkyl, substituted or unsubstituted heteroalkyl, substituted or unsubstituted aryl, or substituted or unsubstituted heteroaryl, provided that when X¹ is O, then R⁵ is not substituted or unsubstituted phenyl; and
each R⁶ is independently hydrogen, substituted or unsubstituted alkyl, or substituted or unsubstituted heteroalkyl.

2. The compound of claim 1, or a salt or solvate thereof, wherein G is -L²-G¹, wherein L² is a >C═X, substituted or unsubstituted unsaturated alkylene, substituted or unsubstituted arylene, or substituted or unsubstituted heteroarylene, wherein X is O, S, or NR³, and G¹ is an organic residue (e.g., a natural ligand).

3. The compound of claim 2, or a salt or solvate thereof, wherein L² is a substituted or unsubstituted unsaturated alkylene, substituted or unsubstituted arylene, or substituted or unsubstituted heteroarylene, and G¹ is an organic residue.

4. The compound of claim 1, or a salt or solvate thereof, wherein G is substituted or unsubstituted unsaturated carbocycle or substituted or unsubstituted unsaturated heterocycle, wherein G and R⁵ on a single N are optionally taken together to form a substituted or unsubstituted heterocycloalkyl.

5. (canceled)

6. The compound of claim 1, or a salt or solvate thereof, wherein G comprises two or more cyclic ring systems selected from substituted or unsubstituted unsaturated carbocycles and substituted or unsubstituted unsaturated heterocycles, the two or more cyclic ring systems being connected via one or more linker and/or bond.

7. (canceled)

8. The compound of claim 1, or a salt or solvate thereof, wherein G¹ comprises two or more cyclic ring systems selected from substituted or unsubstituted carbocycles and substituted or unsubstituted heterocycles, the two or more cyclic ring systems being connected via one or more linker and/or bond.

9. (canceled)

10. (canceled)

11. The compound of claim 1, or a salt or solvate thereof, wherein
the linker is —O—, —NR⁷—, —N(R⁷)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁷—, —NR⁷C(═O)—, —OC(═O)NR⁷—, —NR⁷C(═O)O—, —NR⁷C(═O)NR⁷—, —NR⁷S(═O)₂—, —S(═O)₂NR⁷—, —C(═O)NR⁷S(═O)₂—, —S(═O)₂NR⁷C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁷—, —NR⁷—(C₁-C₄ alkylene)-, —(C₁-C₄ alkylene)-N(R⁷)₂⁺—, or —N(R⁷)₂⁺—(C₁-C₄ alkylene)-; and
each R⁷ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

12. The compound of claim 1, or a salt or solvate thereof, wherein the cyclic ring system comprises substituted or unsubstituted monocyclic or bicyclic aryl or substituted or unsubstituted monocyclic or bicyclic heteroaryl.

13. (canceled)

14. (canceled)

15. (canceled)

16. The compound of claim 1, or a salt or solvate thereof, having a structure represented by Formula (Ia): 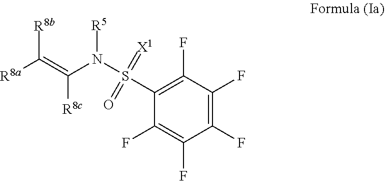
wherein,
each R^{8a}, R^{8b}, and R^{8c} is independently G¹ or R⁹, provided that only one of R^{8a}, R^{8b}, and R^{8c} is G¹; and
each R⁹ is independently hydrogen, substituted or unsubstituted alkyl, substituted or unsubstituted heteroalkyl, substituted or unsubstituted aryl, or substituted or unsubstituted heteroaryl.

17. The compound of claim 16, or a salt or solvate thereof, having a structure represented by Formula (Iaa):

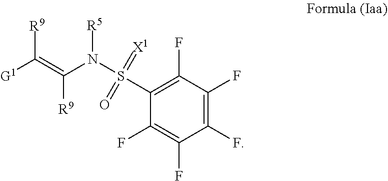

18. The compound of claim 1, or a salt or solvate thereof, having a structure represented by Formula (Ib):

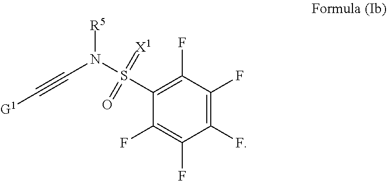

19. The compound of claim 1, or a salt or solvate thereof, wherein R⁵ is hydrogen, —CN, substituted or unsubstituted alkyl, or substituted or unsubstituted heteroalkyl.

20. (canceled)

21. The compound of claim 1, or a salt or solvate thereof, wherein R⁵ is hydrogen, —CN, —CH₃, —CF₃, or cyclopropyl.

22. (canceled)

23. The compound of claim 1, or a salt or solvate thereof, wherein each R⁹ is independently hydrogen, substituted or unsubstituted alkyl, or substituted or unsubstituted heteroalkyl.

24. (canceled)

25. The compound of claim 1, or a salt or solvate thereof, wherein each R⁹ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy.

26. (canceled)

27. The compound of claim 1, or a salt or solvate thereof, wherein X¹ is O, NH, or N (substituted or unsubstituted alkyl).

28. (canceled)

29. (canceled)

30. (canceled)

31. (canceled)

32. A compound or a salt or solvate thereof, wherein the compound is a compound from Table 1, Table 2, Table 3, Table 4, or Table 5.

33. (canceled)

34. (canceled)

35. (canceled)

36. (canceled)

37. (canceled)

38. A pharmaceutically acceptable composition comprising a compound of claim 1, or a salt or solvate thereof and one or more of pharmaceutically acceptable excipients.

39. (canceled)

40. A method of modifying a polypeptide with a compound, comprising contacting the polypeptide with a compound of claim 1, or a salt or solvate thereof, to form a covalent bond with a sulfur atom of a cysteine residue of the polypeptide.

41. A method of binding a compound to a polypeptide, comprising contacting the polypeptide with a compound of claim 1, or a salt or solvate thereof.
